SEQUENCE LISTING (54 sequences)

SEQ ID NO: 1 - DNA, 786 bp, synthetic construct
  misc_feature 1..786: note = Synthetic
  source 1..786: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcctcgtggtgaagaggatagtcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 2 - DNA, 785 bp, synthetic construct
  misc_feature 1..785: note = Synthetic
  source 1..785: mol_type = other DNA
tgatcgaaacctacaaccagacctcaccacgaagtgccgccaccggactgcctattagtatgaaaatctttatgtacctgctgacagtgttcctgatcacccagatgatcggctccgccctgtttgccgtgtacctgcaccggagactggacaagatcgaggatgagcggaacctgcacgaggacttcgtgtttatgaagaccatccagcggtgcaacacaggcgagagaagcctgtccctgctgaattgtgaggagatcaagagccagttcgagggctttgtgaaggacatcatgctgaacaaggaggagacaaagaaggagaacagcttcgagatgcccagaggcgaggaggattcccagatcgccgcccacgtgatctctgaggccagctccaagaccacaagcgtgctgcagtgggccgagaagggctactataccatgtctaacaatctggtgacactggagaacggcaagcagctgaccgtgaagaggcagggcctgtactatatctatgcccaggtgacattctgcagcaatcgcgaggcctctagccaggccccctttatcgccagcctgtgcctgaagagccctggcaggttcgagcgcatcctgctgagagccgccaacacccactcctctgccaagccatgcggacagcagtcaatccacctgggaggcgtgttcgagctgcagccaggagcaagcgtgttcgtgaatgtgactgacccatcacaggtgtctcacggcactggattcacatcatttggactgctgaaactgtga

SEQ ID NO: 3 - protein, 261 aa, synthetic construct
  REGION 1..261: note = Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMPRGEEDSQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - DNA, 723 bp, synthetic construct
  misc_feature 1..723: note = Synthetic
  source 1..723: mol_type = other DNA
atggctcagcatggggctatgggggccttcagggctctgtgcggactggctctgctgtgcgctctgtcactggggcagagaccaacaggaggaccaggatgcggacctggcaggctgctgctgggcaccggcacagacgcaaggtgctgtagagtgcacaccacaaggtgctgtcgcgactaccctggcgaggagtgctgttctgagtgggattgcatgtgcgtgcagccagagtttcactgtggcgatccctgctgtaccacatgccgccaccacccatgtccacctggacagggagtgcagtctcagggcaagttcagctttggcttccagtgcatcgactgtgcaagcggcaccttttccggaggacacgagggacactgcaagccctggaccgattgtacacagtttggcttcctgaccgtgttccctggcaacaagacacacaatgccgtgtgcgtgcctggctccccaccagcagagcccctgggctggctgaccgtggtgctgctggccgtggcagcatgcgtgctgctgctgacaagcgcccagctgggactgcacatctggcagctgcggtcccagtgtatgtggccaagagagacccagctgctgctggaggtgcctccatccacagaggacgcccggtcttgccagttccccgaagaggagaggggggaaagaagtgccgaagaaaagggaaggctgggagacctgtgggtg

SEQ ID NO: 5 - protein, 241 aa, synthetic construct
  REGION 1..241: note = Synthetic
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 6 - DNA, 435 bp, synthetic construct
  misc_feature 1..435: note = Synthetic
  source 1..435: mol_type = other DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 7 - DNA, 435 bp, synthetic construct
  misc_feature 1..435: note = Synthetic
  source 1..435: mol_type = other DNA
atgtggctgcagtctctgctgctgctgggcaccgtcgcctgttctatttccgcacccgctcgctccccttctccctcaactcagccttgggagcacgtgaacgccatccaggaggcccggagactgctgaatctgtcccgggacaccgccgccgagatgaacgagacagtggaagtgatctctgagatgttcgatctgcaggagcccacctgcctgcagacaaggctggagctgtacaagcagggcctgcgcggctctctgaccaagctgaagggcccactgacaatgatggccagccactataagcagcactgcccccctacccccgagacaagctgtgccacccagatcatcacattcgagtcctttaaggagaacctgaaggactttctgctggtcattccatttgattgttgggagcccgtgcaggagtga

SEQ ID NO: 8 - protein, 144 aa, synthetic construct
  REGION 1..144: note = Synthetic
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 9 - DNA, 1710 bp, synthetic construct
  misc_feature 1..1710: note = Synthetic
  source 1..1710: mol_type = other DNA
atgtgccatcagcaactggttatatcttggttcagtctcgtctttctcgcgtcacccttggtcgctatctgggagcttaaaaaagatgtctacgtcgttgaacttgattggtaccctgatgctccgggggaaatggtggttttgacttgcgatacgccagaagaggatggcataacgtggacactggaccagtcttcagaggttctcgggtctggtaagacactcactatacaggtgaaggagtttggtgacgcaggacaatatacttgccataaaggcggcgaggtgctctcccatagccttctgctccttcataaaaaagaggacgggatatggtcaactgacattctgaaggatcagaaagaaccgaagaacaaaactttcctcagatgcgaggcaaagaactattcaggccgctttacttgctggtggctcactaccatcagcactgacctcactttcagcgtcaagagcagtagaggctcaagtgacccacaaggggttacatgcggggccgctacgttgtctgccgagcgagtcaggggagataataaggaatatgagtatagcgttgaatgccaagaagattcagcctgcccagccgcagaagagagtcttcccatagaagttatggtggacgcagttcataaactgaagtatgagaactatacatcttccttctttattcgcgatatcataaagcctgatcctccgaaaaacttgcaactcaagccgttgaagaatagccgacaggtcgaggtctcttgggagtatccagatacgtggtctaccccgcactcctatttcagtctcaccttctgtgtgcaggtgcaggggaaaagtaagcgggaaaaaaaggaccgggtatttactgataagacctccgctacagtgatttgtagaaagaacgcctctatcagcgtgagagcccaggatagatattattctagtagttggtctgagtgggcctccgtcccttgttccggaagcggagccacgaacttctctctgttaaagcaagcaggagatgttgaagaaaaccccgggcctatgtgtccagcgcgcagcctcctccttgtggctaccctggtcctcctggaccacctcagtttggcccgaaacctgccggtcgctacacccgatcctggaatgtttccctgccttcatcacagccagaatctgctgagggcagtcagtaacatgctgcagaaggcgcggcaaactctggagttctatccatgtacctccgaggaaattgatcacgaggacattactaaggataaaacaagtacagtagaagcctgtttgcctcttgagctcactaaaaatgagtcatgcttgaacagtcgagagacgagttttatcactaacggttcatgcttggcgtccaggaagacaagctttatgatggcgctctgcctgtcttctatatatgaagaccttaaaatgtaccaagttgagtttaagaccatgaacgccaaacttttgatggaccccaagaggcagatcttccttgatcagaatatgttggcggtgatcgatgaacttatgcaagctttgaacttcaacagtgagacagtgcctcagaaaagttccttggaggaaccggacttctataagaccaagatcaaactgtgcattttgctgcatgcatttagaattcgagccgttacaatcgaccgggtgatgtcatatttgaatgcatcataa

SEQ ID NO: 10 - protein, 569 aa, synthetic construct
  REGION 1..569: note = Synthetic
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSGATNFSLLKQAGDVEENPGPMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - DNA, 402 bp, synthetic construct
  misc_feature 1..402: note = Synthetic
  source 1..402: mol_type = other DNA
atgtataggatgcagctgctgtcatgtatcgcactgtccctggcactggtgactaactctaactgggtgaatgtgatctccgacctgaagaagatcgaggacctgatccagtctatgcacatcgatgccaccctgtacacagagtccgacgtgcacccctcttgcaaggtgaccgccatgaagtgtttcctgctggagctgcaggtcatcagcctggagagcggcgacgcatccatccacgataccgtggagaacctgatcatcctggccaacaatagcctgagctccaacggcaatgtgacagagtccggctgcaaggagtgtgaggagctggaggagaagaatatcaaagagttcctgcagtcattcgtccatatcgtccagatgtttatcaataccagt

SEQ ID NO: 12 - protein, 134 aa, synthetic construct
  REGION 1..134: note = Synthetic
MYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 1608 bp, synthetic construct
  misc_feature 1..1608: note = Synthetic
  source 1..1608: mol_type = other DNA
atgtgccatcagcagctggtcattagttggtttagcctggtctttctggcctcacccctggtcgcaatctgggaactgaagaaggacgtgtacgtggtggagctggactggtatccagatgcaccaggagagatggtggtgctgacctgcgacacacctgaggaggatggcatcacctggacactggatcagagctccgaggtgctgggcagcggcaagaccctgacaatccaggtgaaggagttcggcgacgccggccagtacacatgtcacaagggcggcgaggtgctgtcccactctctgctgctgctgcacaagaaggaggacggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggctcctctgacccccagggagtgacatgcggagcagccaccctgagcgccgagcgggtgagaggcgataacaaggagtacgagtattctgtggagtgccaggaggacagcgcctgtccagcagcagaggagtccctgcctatcgaagtgatggtggatgccgtgcacaagctgaagtacgagaattatacaagctccttctttatcagggacatcatcaagccagatccccctaagaacctgcagctgaagcccctgaagaatagccgccaggtggaggtgtcctgggagtaccctgacacctggtccacaccacactcttatttcagcctgaccttttgcgtgcaggtgcagggcaagagcaagagggagaagaaggaccgcgtgttcaccgataagacatccgccaccgtgatctgtcggaagaacgccagcatctccgtgagggcccaggatcgctactattctagctcctggagcgagtgggcctccgtgccatgctctggaggaggaggcagcggcggaggaggctccggaggcggcggctctggcggcggcggctccctgggctctcgggccgtgatgctgctgctgctgctgccctggaccgcacagggaagagccgtgccaggaggctctagcccagcatggacacagtgccagcagctgtcccagaagctgtgcaccctggcatggtctgcccaccctctggtgggccacatggacctgagagaggagggcgatgaggagaccacaaacgacgtgcctcacatccagtgcggcgacggctgtgatccacagggcctgagggacaattctcagttctgtctgcagcgcatccaccagggcctgatcttctacgagaagctgctgggcagcgatatctttacaggagagcccagcctgctgcctgactccccagtgggacagctgcacgcctctctgctgggcctgagccagctgctgcagccagagggacaccactgggagacccagcagatcccttctctgagcccatcccagccttggcagcggctgctgctgcggttcaagatcctgagaagcctgcaggcattcgtcgcagtcgcagccagggtgttcgcccacggagccgctactctgagccca

SEQ ID NO: 14 - protein, 536 aa, synthetic construct
  REGION 1..536: note = Synthetic
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 15 - DNA, 342 bp, synthetic construct
  misc_feature 1..342: note = Synthetic
  source 1..342: mol_type = other DNA
atgaggctgctgattctggcactgctgggcatctgctctctgaccgcttacatcgtggaaggagtcggctctgaagtctctgacaagcgcacatgcgtgtctctgaccacacagcgcctgcccgtgagccggatcaagacctacacaatcaccgagggcagcctgagagccgtgatcttcatcacaaagaggggcctgaaggtgtgcgccgaccctcaggcaacctgggtgcgggacgtggtgagaagcatggataggaagtccaacacccggaacaatatgatccagacaaaacccacaggaacccagcagagcactaatacagccgtgacactgaccggg

SEQ ID NO: 16 - protein, 114 aa, synthetic construct
  REGION 1..114: note = Synthetic
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 17 - DNA, 2965 bp, synthetic construct
  misc_feature 1..2965: note = Synthetic
  source 1..2965: mol_type = other DNA
agagtctgagctgtggctgcggttcaaagaactgaccaacgagatgatcgtgaccaagaacggcagacggatgttccccgtgctgaaagtgaacgtgtccggactggaccccaacgccatgtacagctttctgctggacttcgtggtggccgacaaccacagatggaaatacgtgaacggcgagtgggtgccaggcggaaaacctcaactgcaagcccctagctgcgtgtacattcaccctgacagccccaatttcggcgcccactggatgaaggcccctgtgtccttcagcaaagtgaagctgaccaacaagctgaacggcggaggccagatcatgctgaacagcctgcacaaatacgagcccagaatccacatcgtcagagtcggcggaccccagagaatgatcaccagccactgcttccccgagacacagtttatcgccgtgaccgcctaccagaacgaggaaatcaccacactgaagatcaagtacaaccccttcgccaaggccttcctggacgccaaagagcggagcgaccacaaagagatgatcaaagagcccggcgacagccagcagccaggctattctcaatggggatggctgctgccaggcaccagcacattgtgccctccagccaatcctcacagccagtttggaggcgccctgagcctgtctagcacccacagctacgacagataccccacactgcggagccacagaagcagcccctatccttctccttacgctcaccggaacaacagccccacctacagcgataatagccccgcctgtctgagcatgctgcagtcccacgataactggtccagcctgagaatgcctgctcacccttccatgctgcccgtgtctcacaatgcctctccacctaccagcagctctcagtaccctagcctttggagcgtgtccaatggcgccgtgacactgggatctcaggcagccgctgtgtctaatggactgggagcccagttcttcagaggcagccctgctcactacacccctctgacacatcctgtgtctgcccctagcagcagcggcttccctatgtataagggcgctgccgccgctaccgacatcgtggattctcagtatgatgccgccgcacagggacacctgatcgcctcttggacacctgtgtctccaccttccatgagaggcagaaagcggagaagcgacttcctgctgctgcagaaccctgcctctacctgtgtgcctgaaccagcctctcagcacaccctgagatctggccctggatgtctccagcagcctgaacagcagggcgttagagatcctggcggaatctgggccaaactgggagctgccgaagcctctgccgaatgtctgcagggcagaagaagcagaggcgccagcggatctgaacctcaccagatgggaagcgacgtgcacgacctgaatgctctgttgcctgccgtgccatctcttggcggaggcggaggatgtgctttgcctgtttctggtgctgcccagtgggctcccgtgctggattttgctcctcctggcgcttctgcctatggctctcttggaggacctgctcctccaccagctccacctccaccgccgcctccaccacctcacagctttatcaagcaagagccctcctggggcggagccgagcctcacgaaaaacagtgtctgagcgccttcaccgtgcactttttcggccagtttaccggcaccgtgggcgcctgtagatacggcccttttggaccaccaccacctagccaggcttctagcggacaggccagaatgttccccaacgctccttacctgcctagctgcctggaaagccagcctaccatcagaaaccagggcttcagcaccgtgaccttcgacggcatgcctagctatggccacacaccatctcaccacgccgctcagttccccaatcacagcttcaagcacgaggaccctatgggccagcagggatctctgggagagcagcagtatagcgtgccacctcctgtgtacggctgtcacacccctaccgatagctgcacaggcaatcaggctctgctgctgaggatgcctttcagcagcgacaacctgtaccagatgacaagccagctggaatgcatgatttggaaccagatgaacctgggcgccactctgaaaggcgtggccgctggatctagcagctccgtgaaatggacagccggccagagcaatcactccaccggctacgagagcgacaatcacaccatgcctatcctgtgtggggcccagtaccggattcacacacacggcgtgttcaggggcattcaggatgtgcgaagagtgcctggcgtggcccctacacttgtgggatctgccagcgaaaccagcgagaagcaccccttcatgtgcgcctatccaggctgcaacaagcggtacttcaagctgagccacctgaagatgcacagccggaagcacacaggcgagaagctgtaccagtgcgacttcaaggactgcgagcggagattcagctgcagcgaccagctgaagagacaccagagaaggcacaccggcgtgaagccctttcagtgcaagacctgccagcggaccttctcctggtccaaccacctgaaaacccacacaagaacccacaccggcaagaccatcgagaagcccttcagctgtagatggcccagctgccagaagaagttcgcccggtctaacgagctggtgcatcaccacaacatgcaccagaggaacatgaccaaactgcagctggtgctgaggggaagaaagaggcggtccaccgagtacaagctggtggttgttggagccgatggcgtgggaaagagcgccctgacaattcagctgatccagaaccacttcgtgcgcggcagaaagagaagatctacagagtataagctcgtggtcgtgggcgctgtcggagtgggaaaatctgccctgaccatccaactcattcagaatcactttgtgtgatga

SEQ ID NO: 18 - protein, 1033 aa, synthetic construct
  REGION 1..1033: note = Synthetic
MSSPGTESAGKSLQYRVDHLLSAVENELQAGSEKGDPTEHELRVGLEESELWLRFKELTNEMIVTKNGRRMFPVLKVNVSGLDPNAMYSFLLDFVVADNHRWKYVNGEWVPGGKPQLQAPSCVYIHPDSPNFGAHWMKAPVSFSKVKLTNKLNGGGQIMLNSLHKYEPRIHIVRVGGPQRMITSHCFPETQFIAVTAYQNEEITTLKIKYNPFAKAFLDAKERSDHKEMIKEPGDSQQPGYSQWGWLLPGTSTLCPPANPHSQFGGALSLSSTHSYDRYPTLRSHRSSPYPSPYAHRNNSPTYSDNSPACLSMLQSHDNWSSLRMPAHPSMLPVSHNASPPTSSSQYPSLWSVSNGAVTLGSQAAAVSNGLGAQFFRGSPAHYTPLTHPVSAPSSSGFPMYKGAAAATDIVDSQYDAAAQGHLIASWTPVSPPSMRGRKRRSDFLLLQNPASTCVPEPASQHTLRSGPGCLQQPEQQGVRDPGGIWAKLGAAEASAECLQGRRSRGASGSEPHQMGSDVHDLNALLPAVPSLGGGGGCALPVSGAAQWAPVLDFAPPGASAYGSLGGPAPPPAPPPPPPPPPHSFIKQEPSWGGAEPHEKQCLSAFTVHFFGQFTGTVGACRYGPFGPPPPSQASSGQARMFPNAPYLPSCLESQPTIRNQGFSTVTFDGMPSYGHTPSHHAAQFPNHSFKHEDPMGQQGSLGEQQYSVPPPVYGCHTPTDSCTGNQALLLRMPFSSDNLYQMTSQLECMIWNQMNLGATLKGVAAGSSSSVKWTAGQSNHSTGYESDNHTMPILCGAQYRIHTHGVFRGIQDVRRVPGVAPTLVGSASETSEKHPFMCAYPGCNKRYFKLSHLKMHSRKHTGEKLYQCDFKDCERRFSCSDQLKRHQRRHTGVKPFQCKTCQRTFSWSNHLKTHTRTHTGKTIEKPFSCRWPSCQKKFARSNELVHHHNMHQRNMTKLQLVLRGRKRRSTEYKLVVVGADGVGKSALTIQLIQNHFVRGRKRRSTEYKLVVVGAVGVGKSALTIQLIQNHFV

SEQ ID NO: 19 - DNA, 2256 bp, synthetic construct
  misc_feature 1..2256: note = Synthetic
  source 1..2256: mol_type = other DNA
atgtggaatctgctgcacgagacagatagcgccgtggctaccgttagaaggcccagatggctttgtgctggcgctctggttctggctggcggcttttttctgctgggcttcctgttcggctggttcatcaagagcagcaacgaggccaccaacatcacccctaagcacaacatgaaggcctttctggacgagctgaaggccgagaatatcaagaagttcctgtacaacttcacgcacatccctcacctggccggcaccgagcagaattttcagctggccaagcagatccagagccagtggaaagagttcggcctggactctgtggaactggcccactacgatgtgctgctgagctaccccaacaagacacaccccaactacatcagcatcatcaacgaggacggcaacgagatcttcaacaccagcctgttcgagcctccacctcctggctacgagaacgtgtccgatatcgtgcctccattcagcgctttcagcccacagcggatgcctgagggctacctggtgtacgtgaactacgccagaaccgaggacttcttcaagctggaatgggacatgaagatcagctgcagcggcaagatcgtgatcgcccggtacagaaaggtgttccgcgagaacaaagtgaagaacgcccagctggcaggcgccaaaggcgtgatcctgtatagcgaccccgccgactattttgcccctggcgtgaagtcttaccccgacggctggaattttcctggcggcggagtgcagcggcggaacatccttaatcttaacggcgctggcgaccctctgacacctggctatcctgccaatgagtacgcctacagacacggaattgccgaggctgtgggcctgccttctattcctgtgcaccctgtgcggtactacgacgcccagaaactgctggaaaagatgggcggaagcgcccctcctgactcttcttggagaggctctctgaaggtgccctacaatgtcggcccaggcttcaccggcaacttcagcacccagaaagtgaaaatgcacatccacagcaccaacgaagtgacccggatctacaacgtgatcggcacactgagaggcgccgtggaacccgacaaatacgtgatcctcggcggccacagagacagctgggtgttcggaggaatcgaccctcaatctggcgccgctgtggtgtatgagatcgtgcggtctttcggcaccctgaagaaagaaggatggcggcccagacggaccatcctgtttgcctcttgggacgccgaggaatttggcctgctgggatctacagagtgggccgaagagaacagcagactgctgcaagaaagaggcgtggcctacatcaacgccgacagcagcatcgagggcaactacaccctgcggatcgattgcacccctctgatgtacagcctggtgcacaacctgaccaaagagctgaagtcccctgacgagggctttgagggcaagagcctgtacaagagctggaccaagaagtccccatctcctgagttcagcggcatgcccagaatctctaagctggaaagcggcaacaacttcgaggtgttcttccagcggctgggaatcgcctctggaatcgccagatacaccaagaactgggagacaaacaagttctccggctatcccctgtaccacagcgtgtacgagacatacgagctggtggaaaagttctacgaccccatgttcaagtaccacctgacagtggcccaagtgcgcggaggcatggtgttcgaactggccaatagcatcgtgctgcccttcaactgcagagactacgccgtggtgctgcggaagtacgccgacaagatctacagcatcagcatgaagcacccgcaagagatgaagacctacagcgtgtccttcgactccctgttcttcgccgtgaagaacttcaccaagatcgccagcaagttcagcgagcggctgcaggacttcgacaagagcaaccctatcgtgctgaggatgatgaacgaccagctgatgttcctggaacgggccttcatcaaccctctgggactgcccgacagacccttctacaggcacgtgatctgtgcccctagcagccacaacaaatacgccggcgagagcttccccggcatctacgatgccctgttcgacatcgagagcaacgtgaaccctagcaaggcctggggcgaagtgaagagacagatctacgtggccgcattcacagtgcaggccgctgccgaaacactgtctgaggtggcctgatga

SEQ ID NO: 20 - protein, 750 aa, synthetic construct
  REGION 1..750: note = Synthetic
MWNLLHETDSAVATVRRPRWLCAGALVLAGGFFLLGFLFGWFIKSSNEATNITPKHNMKAFLDELKAENIKKFLYNFTHIPHLAGTEQNFQLAKQIQSQWKEFGLDSVELAHYDVLLSYPNKTHPNYISIINEDGNEIFNTSLFEPPPPGYENVSDIVPPFSAFSPQRMPEGYLVYVNYARTEDFFKLEWDMKISCSGKIVIARYRKVFRENKVKNAQLAGAKGVILYSDPADYFAPGVKSYPDGWNFPGGGVQRRNILNLNGAGDPLTPGYPANEYAYRHGIAEAVGLPSIPVHPVRYYDAQKLLEKMGGSAPPDSSWRGSLKVPYNVGPGFTGNFSTQKVKMHIHSTNEVTRIYNVIGTLRGAVEPDKYVILGGHRDSWVFGGIDPQSGAAVVYEIVRSFGTLKKEGWRPRRTILFASWDAEEFGLLGSTEWAEENSRLLQERGVAYINADSSIEGNYTLRIDCTPLMYSLVHNLTKELKSPDEGFEGKSLYKSWTKKSPSPEFSGMPRISKLESGNNFEVFFQRLGIASGIARYTKNWETNKFSGYPLYHSVYETYELVEKFYDPMFKYHLTVAQVRGGMVFELANSIVLPFNCRDYAVVLRKYADKIYSISMKHPQEMKTYSVSFDSLFFAVKNFTKIASKFSERLQDFDKSNPIVLRMMNDQLMFLERAFINPLGLPDRPFYRHVICAPSSHNKYAGESFPGIYDALFDIESNVNPSKAWGEVKRQIYVAAFTVQAAAETLSEVA

SEQ ID NO: 21 - DNA, 84 bp, synthetic construct
  misc_feature 1..84: note = Synthetic
  source 1..84: mol_type = other DNA
accgagtacaagctggtggttgttggagccgatggcgtgggaaagagcgccctgacaattcagctgatccagaaccacttcgtg

SEQ ID NO: 22 - protein, 28 aa, synthetic construct
  REGION 1..28: note = Synthetic
TEYKLVVVGADGVGKSALTIQLIQNHFV

SEQ ID NO: 23 - DNA, 84 bp, synthetic construct
  misc_feature 1..84: note = Synthetic
  source 1..84: mol_type = other DNA
acagagtataagctcgtggtcgtgggcgctgtcggagtgggaaaatctgccctgaccatccaactcattcagaatcactttgtg

SEQ ID NO: 24 - protein, 28 aa, synthetic construct
  REGION 1..28: note = Synthetic
TEYKLVVVGAVGVGKSALTIQLIQNHFV

SEQ ID NO: 25 - DNA, 36 bp, synthetic construct
  misc_feature 1..36: note = Synthetic
  source 1..36: mol_type = other DNA
ggcagccctggcatgggtgtgcatgtgggtgcagcc

SEQ ID NO: 26 - DNA, 21 bp, synthetic construct
  misc_feature 1..21: note = Synthetic
  source 1..21: mol_type = other DNA
tttccaccattagcacgcggg

SEQ ID NO: 27 - DNA, 21 bp, synthetic construct
  misc_feature 1..21: note = Synthetic
  source 1..21: mol_type = other DNA
aatctgatatagctcaatccg

SEQ ID NO: 28 - DNA, 22 bp, synthetic construct
  misc_feature 1..22: note = Synthetic
  source 1..22: mol_type = other DNA
gaagcccttcagctgtagatgg

SEQ ID NO: 29 - DNA, 19 bp, synthetic construct
  misc_feature 1..19: note = Synthetic
  source 1..19: mol_type = other DNA
ctgaattgtcagggcgctc

SEQ ID NO: 30 - DNA, 22 bp, synthetic construct
  misc_feature 1..22: note = Synthetic
  source 1..22: mol_type = other DNA
catgcaccagaggaacatgacc

SEQ ID NO: 31 - DNA, 22 bp, synthetic construct
  misc_feature 1..22: note = Synthetic
  source 1..22: mol_type = other DNA
gagttggatggtcagggcagat

SEQ ID NO: 32 - protein, 7 aa, synthetic construct
  REGION 1..7: note = Synthetic
RGRKRRS

SEQ ID NO: 33 - DNA, 567 bp, synthetic construct
  misc_feature 1..567: note = Synthetic
  source 1..567: mol_type = other DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatg

SEQ ID NO: 34 - protein, 189 aa, synthetic construct [sequence omitted]

SEQ ID NO: 35 - DNA, 1179 bp, synthetic construct
  misc_feature 1..1179: note = Synthetic
  source 1..1179: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 36 - protein, 393 aa, synthetic construct [sequence omitted]

SEQ ID NO: 37 - DNA, 3204 bp, synthetic construct
  misc_feature 1..3204: note = Synthetic
  source 1..3204: mol_type = other DNA
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaac

SEQ ID NO: 38 - protein, 1068 aa, synthetic construct
  REGION 1..1068: note = Synthetic
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 39 - DNA, 2124 bp, synthetic construct
  misc_feature 1..2124: note = Synthetic
  source 1..2124: mol_type = other DNA
atgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtga

SEQ ID NO: 40 - protein, 707 aa, synthetic construct
  REGION 1..707: note = Synthetic
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 41 - DNA, 1659 bp, synthetic construct
  misc_feature 1..1659: note = Synthetic
  source 1..1659: mol_type = other DNA
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactga

SEQ ID NO: 42 - protein, 552 aa, synthetic construct
  REGION 1..552: note = Synthetic
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 43 - DNA, 9171 bp, synthetic construct
  misc_feature 1..9171: note = Synthetic
  source 1..9171: mol_type = other DNA
atgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtga

SEQ ID NO: 44 - protein, 3056 aa, synthetic construct
  REGION 1..3056: note = Synthetic
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 45 - DNA, 2346 bp, synthetic construct
  misc_feature 1..2346: note = Synthetic
  source 1..2346: mol_type = other DNA
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 46 - protein, 781 aa, synthetic construct
  REGION 1..781: note = Synthetic
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 47 - DNA, 4029 bp, synthetic construct
  misc_feature 1..4029: note = Synthetic
  source 1..4029: mol_type = other DNA
atgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaa

SEQ ID NO: 48 - protein, 1342 aa, synthetic construct
  REGION 1..1342: note = Synthetic
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 49 - DNA, 1185 bp, synthetic construct
  misc_feature 1..1185: note = Synthetic
  source 1..1185: mol_type = other DNA
atgggctgcctcgggaacagtaagaccgaggaccagcgcaacgaggagaaggcgcagcgtgaggccaacaaaaagatcgagaagcagctgcagaaggacaagcaggtctaccgggccacgcaccgcctgctgctgctgggtgctggagaatctggtaaaagcaccattgtgaagcagatgaggatcctgcatgttaatgggtttaatggagagggcggcgaagaggacccgcaggctgcaaggagcaacagcgatggtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaa

SEQ ID NO: 50 - protein, 394 aa, synthetic construct
  REGION 1..394: note = Synthetic
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 51 - DNA, 1005 bp, synthetic construct
  misc_feature 1..1005: note = Synthetic
  source 1..1005: mol_type = other DNA
atgaccaccatccactacaactacatgtgcaacagcagctgcatgggcagcatgaactggcggcctatcctgaccatcatcaccctggaagatagccggggcagaaagcggagaagcgtggccatgaacgagttcagcacactgcccctgcctaacctgagaatggttcgaggcacccaggtgtacgacggcaagttcgccatctttgtgcgcggcagaaagaggcggagctacctggattctggcatccactctggcgctacaacaacagccccattcctgagcggcaagggcaaccccgaagaggaagatgtggataccagcagaggccggaagagaagatccgacgtggaaaccggcaactgcatccacacactgacaggccaccagctgctgacctctggcatggaactgaaggacaacatcctggtgtccggcagaggaagaaagcgcagatctaccggcgagtgcattcacaccctgtatggccacaccagcaccgtgcactgcatgcatctgcacgagaagagagtggtgtctggcagcagagacagaggacgcaagcggagatccgagcaagaggccctggaatactttatgaagcagatcaacgacgcctaccacggcggctggactaccaagatggactggatcttccacaccatccgcggacgcaagagaagaagcgtgacacaagaggccgagcgggaagagttcttcgacgagacaagacagctgtgcgacctgcggctgttccagcctttcctgaaagtgatcgagcgcggacggaaaagacggtccaccgagtataagctggtggtcgtgggagcttgtggcgtgggaaaaagcgccctgacaatccagctgatccagaaccacttcgtgcggggaagaaaacggcggagcatggccatctacaagcagagccagcacatgaccgaggtcgtgcggcactgtcctcaccacgagagatgtagcgatagcgacggactggccccttgatga

SEQ ID NO: 52 - protein, 333 aa, synthetic construct
  REGION 1..333: note = Synthetic
MTTIHYNYMCNSSCMGSMNWRPILTIITLEDSRGRKRRSVAMNEFSTLPLPNLRMVRGTQVYDGKFAIFVRGRKRRSYLDSGIHSGATTTAPFLSGKGNPEEEDVDTSRGRKRRSDVETGNCIHTLTGHQLLTSGMELKDNILVSGRGRKRRSTGECIHTLYGHTSTVHCMHLHEKRVVSGSRDRGRKRRSEQEALEYFMKQINDAYHGGWTTKMDWIFHTIRGRKRRSVTQEAEREEFFDETRQLCDLRLFQPFLKVIERGRKRRSTEYKLVVVGACGVGKSALTIQLIQNHFVRGRKRRSMAIYKQSQHMTEVVRHCPHHERCSDSDGLAP

SEQ ID NO: 53 - DNA, 672 bp, synthetic construct
  misc_feature 1..672: note = Synthetic
  source 1..672: mol_type = other DNA
atggaagatagcagcggcaatctgctgggcagaaacagcttcgaagtgtgcgtgtgtgcctgtcctggcagagacagaagaaccgaggaagagaaccggggcagaaagcggagaagcgacaaagagcagctgaaggccatcagcaccagagatcctctgagcaagatcacagagcaagagaaggacttcctgtggtcccaccggcactacagaggccggaagagaagatctaccggccagtgtctgcacgtcctgatgggacatgtggccgccgtgtgttgcgtgcagtacgatggcagaagagtggtttccggcgcctacgacagaggaagaaaaaggcggtcccctatcgtgaccgtggacggctatgttgatccctctggcggcgatcacttctgcctgggccagctgtctaacgtgcacagaaccgaagccatcagaggacggaagcggagatccgagatcagccacatcggcagcagaggcaagtacagcagcggcttctgcaatatcgccgtgaaagagaacctgatcgaactgatggccgacatcagaggtagaaagcggcggagcaagcaggccgattacgtgccatctgaccaggacctgctgagatgccacgtgctgaccagcggcatcttcgagacaaagttccaggtggacaagtgatga

SEQ ID NO: 54 - protein, 222 aa, synthetic construct
  REGION 1..222: note = Synthetic
MEDSSGNLLGRNSFEVCVCACPGRDRRTEEENRGRKRRSDKEQLKAISTRDPLSKITEQEKDFLWSHRHYRGRKRRSTGQCLHVLMGHVAAVCCVQYDGRRVVSGAYDRGRKRRSPIVTVDGYVDPSGGDHFCLGQLSNVHRTEAIRGRKRRSEISHIGSRGKYSSGFCNIAVKENLIELMADIRGRKRRSKQADYVPSDQDLLRCHVLTSGIFETKFQVDK

(19) United States
(12) Patent Application Publication
(10) Pub. No.: US 2025/0302931 A1
(43) Pub. Date: Oct. 2, 2025

(54) COLORECTAL CANCER TUMOR CELL VACCINES

(71) Applicant: NEUVOGEN, INC., San Diego, CA (US)

(72) Inventors: Bernadette Ferraro, San Diego, CA (US); Justin James Arndt, San Diego, CA (US); Todd Merrill Binder, San Diego, CA (US); Matthias Hundt, San Diego, CA (US); Amritha Balakrishnan Lewis, San Diego, CA (US); Kendall M. Mohler, San Diego, CA (US); Daniel Lee Shawler, San Diego, CA (US); Jian Yan, San Diego, CA (US); Mark Bagarazzi, San Diego, CA (US)

(21) Appl. No.: 18/947,958

(22) Filed: Nov. 14, 2024

Related U.S. Application Data
(63) Continuation of application No. 17/516,203, filed on Nov. 1, 2021, now abandoned.
(60) Provisional application No. 63/108,731, filed on Nov. 2, 2020.
(60) Provisional application No. 63/196,075, filed on Jun. 2, 2021.

(51) Int. Cl. A61K 39/00; A61K 35/12; A61K 39/39; A61P 35/00

(52) U.S. Cl. CPC A61K 39/0011; A61K 39/001104; A61K 39/001106; A61K 39/001151; A61K 39/001152; A61K 39/001153; A61K 39/001157; A61K 39/001162; A61K 39/001164; A61K 39/001166; A61K 39/001168; A61K 39/001184; A61K 39/001186; A61K 39/001195; A61K 39/39; A61P 35/00; A61K 35/12; A61K 2039/5152; A61K 2039/5156; A61K 2039/545; A61K 2039/55516; A61K 2039/55527; A61K 2039/55538; A61K 2039/575; A61K 2039/82

(57) ABSTRACT

The present disclosure provides an allogeneic whole cell cancer vaccine platform that includes compositions and methods for treating and preventing colorectal cancer. Provided herein are compositions containing a therapeutically effective amount of cells from one or more cancer cell lines, some or all of which are modified to (i) inhibit or reduce expression of one or more immunosuppressive factors by the cells, and/or (ii) express or increase expression of one or more immunostimulatory factors by the cells, and/or (iii) express or increase expression of one or more tumor-associated antigens (TAAs), including TAAs that have been mutated, and which comprise cancer cell lines that natively express a heterogeneity of tumor associated antigens and/or neoantigens, and/or (iv) express one or more tumor fitness advantage mutations, including but not limited to driver mutations. Also provided herein are methods of making and preparing the colorectal cancer vaccine compositions and methods of use thereof.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “57305_Seqlisting.txt”, which was created on Oct. 28, 2021 and is 169,865 bytes in size. The subject matter of the Sequence Listing is incorporated herein in its entirety by reference.

### BACKGROUND

Cancer is a leading cause of death. Recent breakthroughs in immunotherapy approaches, including checkpoint inhibitors, have significantly advanced the treatment of cancer, but these approaches are neither customizable nor broadly applicable across indications or to all patients within an indication. Furthermore, only a subset of patients are eligible for and respond to these immunotherapy approaches. Therapeutic cancer vaccines have the potential to generate anti-tumor immune responses capable of eliciting clinical responses in cancer patients, but many of these therapies have a single target or are otherwise limited in scope of immunomodulatory targets and/or breadth of antigen specificity. The development of a therapeutic vaccine customized for an indication that targets the heterogeneity of the cells within an individual tumor remains a challenge.

A vast majority of therapeutic cancer vaccine platforms are inherently limited in the number of antigens that can be targeted in a single formulation. The lack of breadth in these vaccines adversely impacts efficacy and can lead to clinical relapse through a phenomenon called antigen escape, with the appearance of antigen-negative tumor cells. While these approaches may somewhat reduce tumor burden, they do not eliminate antigen-negative tumor cells or cancer stem cells. Harnessing a patient's own immune system to target a wide breadth of antigens could reduce tumor burden as well as prevent recurrence through the antigenic heterogeneity of the immune response. Thus, a need exists for improved whole cell cancer vaccines. Provided herein are methods and compositions that address this need.

### SUMMARY

In various embodiments, the present disclosure provides an allogeneic whole cell colorectal cancer vaccine platform that includes compositions and methods for treating and preventing cancer. The present disclosure provides compositions and methods that are customizable for the treatment of colorectal cancer and target the heterogeneity of the cells within an individual tumor. In some embodiments, the present disclosure provides compositions of cancer cell lines that (i) are modified as described herein and (ii) express a sufficient number and amount of tumor associated antigens (TAAs) such that, when administered to a subject afflicted with a colorectal cancer, cancers, or cancerous tumor(s), a TAA-specific immune response is generated.

In one embodiment, the present disclosure provides a composition comprising a therapeutically effective amount of at least 1 modified colorectal cancer cell line, wherein the cell line or a combination of the cell lines comprises cells that express at least 5 tumor associated antigens (TAAs) associated with colorectal cancer, and wherein said composition is capable of eliciting an immune response specific to the at least 5 TAAs, and wherein the cell line or combination of the cell lines have been modified to express at least 1 peptide comprising at least 1 oncogene driver mutation. In another embodiment, the present disclosure provides a composition comprising 1, 2, or 3 modified colorectal cancer cell lines, wherein the cell line or a combination of the cell lines comprises cells that express at least 14 tumor associated antigens (TAAs) associated with colorectal cancer, and wherein said composition is capable of eliciting an immune response specific to the at least 14 TAAs, and wherein the cell line or combination of the cell lines have been modified to express at least 1 peptide comprising at least 1 oncogene driver mutation.

In other embodiments, the present disclosure provides an aforementioned composition wherein the cell line or combination of the cell lines have been modified to express at least 2, 3, 4, 5, 6, 7, 8, 9, 10, 11, 12, 13, 14, 15, 16, 17, 18, 19 or 20 or more peptides, wherein each peptide comprises at least 1 oncogene driver mutation. In still other embodiments, the present disclosure provides an aforementioned composition wherein the cell line or a combination of the cell lines are modified to express or increase expression of at least 1 immunostimulatory factor. In yet other embodiments, the present disclosure provides an aforementioned composition wherein the cell line or a combination of the cell lines are modified to inhibit or decrease expression of at least 1 immunosuppressive factor. In other embodiments, the cell line or a combination of the cell lines are modified to (i) express or increase expression of at least 1 immunostimulatory factor, and (ii) inhibit or decrease expression of at least 1 immunosuppressive factor. In still other embodiments, the cell line or a combination of the cell lines are modified to express or increase expression of at least 1 TAA that is either not expressed or minimally expressed by one or all of the cell lines.

In other embodiments, the present disclosure provides an aforementioned composition wherein the composition is capable of stimulating an immune response in a subject receiving the composition. In one embodiment, the cell line or a combination of the cell lines are modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, (ii) express or increase expression of 1, 2, 3, 4, 5, 6, 7, 8, 9 or 10 immunostimulatory factors, (iii) inhibitor decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines, and wherein at least one of the cell lines is a cancer stem cell line. In another embodiment, the cancer stem line is selected from the group consisting of JHOM-2B, OVCAR-3, OV56, JHOS-4, JHOC-5, OVCAR-4, JHOS-2, EFO-21, CFPAC-1, Capan-1, Panc 02.13, SUIT-2, Panc 03.27, SK-MEL-28, RVH-421, Hs 895.T, Hs 940.T, SK-MEL-1, Hs 936.T, SH-4, COLO 800, UACC-62, NCI-H2066, NCI-H1963, NCI-H209, NCI-H889, COR-L47, NCI-H1092, NCI-H1436, COR-L95, COR-L279, NCI-H1048, NCI-H69, DMS 53, HuH-6, Li7, SNU-182, JHH-7, SK-HEP-1, Hep 3B2.1-7, SNU-1066, SNU-1041, SNU-1076, BICR 18, CAL-33, YD-8, CAL-29, KMBC-2, 253J, 253J-BV, SW780, SW1710, VM-CUB-1, BC-3C, KNS-81, TM-31, NMC-G1, GB-1, SNU-201, DBTRG-05MG, YKG-1, ECC10, RERF-GC-1B, TGBC-11-TKB, SNU-620, GSU, KE-39, HuG1-N, NUGC-4, SNU-16, OCUM-1, C2BBe1, Caco-2, SNU-1033, SW1463, COLO 201, GP2d, LoVo, SW403, CL-14, HCC2157, HCC38, HCC1954, HCC1143, HCC1806, HCC1599, MDA-MB-415, CAL-51, K052, SKNO-1, Kasumi-1, Kasumi-6, MHH-CALL-3, MHH-CALL-2, JVM-2, HNT-34, HOS, OUMS-27, T1-73, Hs 870.T, Hs 706.T, SJSA-1, RD-ES, U20S, SaOS-2, and SK-ES-1. In yet another embodiment, the colorectal cancer cell line or cell lines are selected from the group consisting of LS123, HCT15, SW1463, RKO, HUTU80, HCT116, LOVO, T84, LS411N, SW48, C2BBe1, Caco-2, SNU-1033, COLO 201, GP2d, CL-14, SW403, SW1116, SW837, SK-CO-1, CL-34, NCI-H508, CCK-81, SNU-C2A, GP2d, HT-55, MDST8, RCM-1, CL-40, COLO 678, and LS180. In one embodiment, the cell lines are selected from the group consisting of HCT15, RKO, HUTU80, HCT116, and LS411N.

In other embodiments, the present disclosure provides an aforementioned composition wherein the oncogene driver mutation is in one or more oncogenes selected from the group consisting of APC, TP53, KRAS, PIK3CA, FAT4, LRP1B, FBXW7, BRAF, SMAD4, PCLO, KMT2C, KMT2D, ATM, RNF213, ZFHX3, AMER1, TRRAP, ARID1A, FAT1, EP400, SOX9, RNF43, MKI67, RELN, PTPRS, PDE4DIP, CHD4, PTPRT, ANKRD11, ROBO1, MTOR, CREBBP, LRRK2, TCF7L2, KMT2B, PRKDC, UBR5, ACVR2A, ERBB4, PREX2, CARD11, NOTCH1, PTEN, NCOR2, GRIN2A, KMT2A, ATRX, CACNA1D, ALK, MYH9, NOTCH3, POLE, BCORL1, SPEN, BCL9L, BRCA2, CUX1, ARID1B, CTNNB1, MYH11, SMARCA4, NF1, PIK3CG, PLCG2, AXIN2, MGA, SLX4, FLT4, ERBB3, POLQ, ASXL1, CAD, PTPRK, ARID2, CIC, EP300, EPHA5, NUMA1, CAMTA1, GNAS, LRP5, BCL9, PTPRD, RANBP2, IRS1, MYO5A, ROS1, IRS4, SETD1A, PIK3R1, PTPRC, COL1A1, TP53BP1, DICER1, SETBP1, ZBTB20, KDM2B, B2M, AFDN, ZNF521, and LARP4B. In one embodiment, the one or more oncogenes comprise TP53 (SEQ ID NO: 36), PIK3CA (SEQ ID NO: 38), FBXW7(SEQ ID NO: 40), SMAD4 (SEQ ID NO: 42), GNAS (SEQ ID NO: 50), ATM (SEQ ID NO: 44), KRAS (SEQ ID NO: 34), CTNNB1 (SEQ ID NO: 46), and ERBB3 (SEQ ID NO: 48). In another embodiment, TP53 (SEQ ID NO: 36) comprises driver mutations selected from the group consisting of R175H, R273C, G245S, and R248W; PIK3CA (SEQ ID NO: 38) comprises driver mutations selected from the group consisting of E542K, R88Q, M10431, and H1047Y; FBXW7(SEQ ID NO: 40) comprises driver mutations selected from the group consisting of R505C, S582L and R465H; SMAD4 (SEQ ID NO: 42) comprises driver mutations selected from the group consisting of R361H, GNAS (SEQ ID NO: 50) comprises driver mutations selected from the group consisting of R201H, ATM (SEQ ID NO: 44) comprises driver mutations selected from the group consisting of R337C; KRAS (SEQ ID NO: 34) comprises driver mutations selected from the group consisting of G12D, G12C and G12V; CTNNB1 (SEQ ID NO: 46) comprises driver mutations selected from the group consisting of S45F; and ERBB3 (SEQ ID NO: 48) comprises drive mutation V104M.

In other embodiments, the present disclosure provides an aforementioned composition wherein (a) the at least one immunostimulatory factor is selected from the group consisting of GM-CSF, membrane-bound CD40L, GITR, IL-15, IL-23, and IL-12, and (b) wherein the at least one immunosuppressive factor is selected from the group consisting of CD276, CD47, CTLA4, HLA-E, HLA-G, IDO1, IL-10, TGFβ1, TGFβ2, and TGFβ3.

Compositions comprising cell lines are provided herein. In one embodiment, the present disclosure provides a composition comprising cancer cell line HCT15, wherein the HCT15 cell line is modified in vitro to (i) express at least one immunostimulatory factor, and (ii) decrease expression of at least one immunosuppressive factor. In one embodiment, the HCT15 cell line is modified in vitro to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25).

In one embodiment, the present disclosure provides a composition comprising cancer cell line HUTU80, wherein the HUTU80 cell line is modified in vitro to (i) express at least one immunostimulatory factor, at least one TAA that is either not expressed or minimally expressed by HUTU80, and at least 1 peptide comprising at least 1 oncogene driver mutation; and (ii) decrease expression of at least one immunosuppressive factor. In one embodiment, the HUTU80 cell line is modified in vitro to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361 H of oncogene SMAD4, R201H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25).

In one embodiment, the present disclosure provides a composition comprising cancer cell line LS411 N, wherein the LS411 N cell line is modified in vitro to (i) express at least one immunostimulatory factor, and (ii) decrease expression of at least one immunosuppressive factor. In another embodiment, the LS411 N cell line is modified in vitro to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25).

In one embodiment, the present disclosure provides a composition comprising cancer cell line HCT116, wherein the HCT116 cell line is modified in vitro to (i) express at least one immunostimulatory factor, at least one TAA that is either not expressed or minimally expressed by HCT116, and at least 1 peptide comprising at least 1 oncogene driver mutation; and (ii) decrease expression of at least one immunosuppressive factor. In one embodiment, the HCT116 cell line is modified in vitro to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25).

In one embodiment, the present disclosure provides a composition comprising cancer cell line RKO, wherein the RKO cell line is modified in vitro to (i) express at least one immunostimulatory factor, and at least 1 peptide comprising at least 1 oncogene driver mutation; and (ii) decrease expression of at least one immunosuppressive factor. In one embodiment, the RKO cell line is modified in vitro to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25).

In one embodiment, the present disclosure provides a composition comprising 3 colorectal cancer cell lines, wherein 1, 2 or all 3 of the cell lines is modified in vitro to (i) express at least one immunostimulatory factor; and (ii) decrease expression of at least one immunosuppressive factor; wherein at least 1 of the cell lines is modified to express at least one TAA that is either not expressed or minimally expressed by the cell line; and wherein at least 1 of the cell lines modified in vitro to express at least 1 peptide comprising at least 1 oncogene driver mutation.

The present disclosure also provides, in one embodiment, a composition comprising cancer cell lines HCT15, HUTU80 and LS411N, wherein: (a) HCT15 is modified to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25); (b) HUTU80 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361H of oncogene SMAD4, R201H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25); and (c) LS411N is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25).

In one embodiment, the present disclosure provides a composition comprising 2 colorectal cancer cell lines and one cancer stem cell line, wherein 1, 2 or all 3 of the cell lines is modified in vitro to (i) express at least one immunostimulatory factor; and (ii) decrease expression of at least one immunosuppressive factor; wherein at least 1 of the colorectal cancer cell lines is modified to express at least one TAA that is either not expressed or minimally expressed by the colorectal cancer cell line; and wherein at least 1 of the colorectal cell lines modified in vitro to express at least 1 peptide comprising at least 1 oncogene driver mutation. In one embodiment, a composition is provided comprising cancer cell lines HCT116, RKO and DMS 53 wherein: (a) HCT116 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25); (b) RKO is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25); and (c) DMS 53 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27); and (ii) decrease expression of CD276 using a zinc-finger nuclease targeting CD276 (SEQ ID NO: 25).

In some embodiments, an aforementioned composition is provided wherein the composition comprises approximately 1.0×10⁶-6.0×10⁷ cells of each cell line.

The present disclosure also provides kits as described herein. In one embodiment, a kit is provided comprising one or more of the aforementioned compositions. In another embodiment, a kit comprising at least one vial, said vial containing an aforementioned composition is provided. In one embodiment, the present disclosure provides a kit comprising 6 vials, wherein the vials each contain a composition comprising a cancer cell line, wherein 5 of the 6 vials comprise a modified colorectal cancer cell line, and wherein at least 2 of the 6 vials comprise a cancer cell line that is modified to (i) express or increase expression of at least 2 immunostimulatory factors, (ii) inhibit or decrease expression of at least 2 immunosuppressive factors, and (iii) express at least 1 peptide comprising at least 1 oncogene driver mutation. In another embodiment, the present disclosure provides a kit comprising 6 vials, wherein the vials each contain a composition comprising a cancer cell line, wherein 5 of the 6 vials comprise a modified colorectal cancer cell line, wherein said colorectal cancer cell lines are each modified to (i) express or increase expression of at least 2 immunostimulatory factors, (ii) inhibit or decrease expression of at least 2 immunosuppressive factors; wherein at least 2 of the 5 vials comprise colorectal cancer cell lines are modified to express at least one TAA that is either not expressed or minimally expressed by the colorectal cancer cell lines; and wherein at least 2 of the 5 vials comprise colorectal cancer cell lines are modified to express at least 1 peptide comprising at least 1 oncogene driver mutation.

In still another embodiment, the present disclosure provides a kit comprising 6 vials, wherein the vials each contain a cell line selected from the group consisting of HCT15, HUTU80, LS411N, HCT116, RKO and DMS 53; wherein: (a) HCT15 is modified to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25); (b) HUTU80 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361 H of oncogene SMAD4, R201 H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25); (c) LS411N is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25); (d) HCT116 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25); (e) RKO is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25); and (f) DMS 53 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27); and (ii) decrease expression of CD276 using a zinc-finger nuclease targeting CD276 (SEQ ID NO: 25).

In some embodiments, an aforementioned kit is provided wherein the composition comprises approximately 1.0×10⁶-6.0×10⁷ cells of each cell line.

The present disclosure also provides unit doses. In one embodiment, the present disclosure provides a unit dose of a medicament for treating colorectal cancer comprising at least 4 compositions of different cancer cell lines, wherein the cell lines comprise cells that collectively express at least 15 tumor associated antigens (TAAs) associated with colorectal cancer. In another embodiment, the present disclosure provides a unit dose of a medicament for treating colorectal cancer comprising at least 5 compositions of different cancer cell lines, wherein at least 2 compositions comprise a cell line that is modified to (i) express or increase expression of at least 2 immunostimulatory factors, (ii) inhibit or decrease expression of at least 2 immunosuppressive factors, and (iii) express at least 1 peptide comprising at least 1 oncogene driver mutation. In still another embodiment, the present disclosure provides a unit dose of a medicament for treating colorectal cancer comprising at least 5 compositions of different cancer cell lines, wherein each cell line is modified to (i) express or increase expression of at least 2 immunostimulatory factors, (ii) inhibit or decrease expression of at least 2 immunosuppressive factors, wherein at least 2 compositions comprise a cell line that is modified to increase expression of at least 1 TAA that are either not expressed or minimally expressed by the cancer cell lines, and wherein at least 2 compositions comprise a cell line that is modified to express at least 1 peptide comprising at least 1 oncogene driver mutation.

In some embodiments, an aforementioned unit dose is provided, wherein the unit dose comprises 6 compositions and wherein each composition comprises a different modified cell line. In one embodiment, prior to administration to a subject, 2 compositions are prepared, wherein the 2 compositions each comprises 3 different modified cell lines.

In one embodiment, the present disclosure provides a unit dose of a colorectal cancer vaccine comprising 6 compositions, wherein each composition comprises one cancer cell line selected from the group consisting of HCT15, HUTU80, LS411N, HCT116, RKO and DMS 53; wherein: (a) HCT15 is modified to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25); (b) HUTU80 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361H of oncogene SMAD4, R201H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25); (c) LS411 N is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25); (d) HCT116 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25); (e) RKO is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25); and (f) DMS 53 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27); and (ii) decrease expression of CD276 using a zinc-finger nuclease targeting CD276 (SEQ ID NO: 25). In one embodiment, modified cell lines HCT15, HUTU80 and LS411N are combined into a first vaccine composition, and modified cell lines HCT116, RKO and DMS 53 are combined into a second vaccine composition.

Methods for preparing compositions are also provided in the present disclosure. In one embodiment, the present disclosure provides a method of preparing a composition comprising a modified colorectal cancer cell line, said method comprising the steps of: (a) identifying one or more mutated oncogenes with >5% mutation frequency in colorectal cancer; (b) identifying one or more driver mutations occurring in >0.5% of profiled colorectal patient samples in the mutated oncogenes identified in (a); (c) determining whether a peptide sequence comprising non-mutated oncogene amino acids and the driver mutation identified in (b) comprises a CD4 epitope, a CD8 epitope, or both CD4 and CD8 epitopes; (d) inserting a nucleic acid sequence encoding the peptide sequence comprising the driver mutation of (c) into a lentiviral vector; and (e) introducing the lentiviral vector into a cancer cell line, thereby producing a composition comprising a modified cancer cell line. In one embodiment, the composition is capable of stimulating an immune response in a subject receiving the composition.

The present disclosure provides additional methods as well. In one embodiment, a method of stimulating an immune response in a subject is provided, the method comprising the steps of preparing a composition comprising a modified colorectal cancer cell line comprising the steps of: (a) identifying one or more mutated oncogenes with >5% mutation frequency in colorectal cancer; (b) identifying one or more driver mutations occurring in >0.5% of profiled colorectal patient samples in the mutated oncogenes identified in (a); (c) determining whether a peptide sequence comprising non-mutated oncogene amino acids and the driver mutation identified in (b) comprises a CD4 epitope, a CD8 epitope, or both CD4 and CD8 epitopes; (d) inserting a nucleic acid sequence encoding the peptide sequence comprising the driver mutation of (c) into a lentiviral vector; (e) introducing the lentiviral vector into a cancer cell line, thereby producing a composition comprising a modified colorectal cancer cell line; and (f) administering a therapeutically effective dose of the composition to the subject. In another embodiment, the present disclosure provides a method of treating colorectal cancer in a subject, the method comprising the steps of preparing a composition comprising a modified colorectal cancer cell line comprising the steps of: (a) identifying one or more mutated oncogenes with >5% mutation frequency in colorectal cancer; (b) identifying one or more driver mutations occurring in >0.5% of profiled colorectal patient samples in the mutated oncogenes identified in (a); (c) determining whether a peptide sequence comprising non-mutated oncogene amino acids and the driver mutation identified in (b) comprises a CD4 epitope, a CD8 epitope, or both CD4 and CD8 epitopes; (d) inserting a nucleic acid sequence encoding the peptide sequence comprising the driver mutation of (c) into a lentiviral vector; (e) introducing the lentiviral vector into a cancer cell line, thereby producing a composition comprising a modified cancer cell line; and (f) administering a therapeutically effective dose of the composition to the subject.

In some embodiments, an aforementioned method is provided wherein the cell line is further modified to express or increase expression of at least 1 immunostimulatory factor. In some embodiments, an aforementioned method is provided wherein the cell line is further modified to inhibit or decrease expression of at least 1 immunosuppressive factor. In still other embodiments, an aforementioned method is provided wherein the cell line is further modified to (i) express or increase expression of at least 1 immunostimulatory factor, and (ii) inhibit or decrease expression of at least 1 immunosuppressive factor. In some embodiments, an aforementioned method is provided wherein the cell line is further modified to express increase expression of at least 1 TAA that is either not expressed or minimally expressed by one or all of the cell lines.

In some embodiments, an aforementioned method is provided wherein (a) the at least one immunostimulatory factor is selected from the group consisting of GM-CSF, membrane-bound CD40L, GITR, IL-15, IL-23, and IL-12, and (b) wherein the at least one immunosuppressive factors are selected from the group consisting of CD276, CD47, CTLA4, HLA-E, HLA-G, IDO1, IL-10, TGFβ1, TGFβ2, and TGFβ3. In other embodiments, an aforementioned method is provided wherein the composition comprises 2, 3, 4, 5, 6, 7, 8, 9, or 10 modified colorectal cancer cell lines. In some embodiments, an aforementioned method is provided wherein two compositions, each comprising at least 2 modified cancer cell lines, are administered to the patient. In one embodiment, the two compositions in combination comprise at least 4 different modified colorectal cancer cell lines and wherein one composition further comprises a cancer stem cell or wherein both compositions further comprise a cancer stem cell. In some embodiments, an aforementioned method is provided wherein the one or more mutated oncogenes has a mutation frequency of at least 5% in the cancer. In one embodiment, 1, 2, 3, 4, 5, 6, 7, 8, 9, 10, 11, 12, 13, 14, 15, 16, 17, 18, 19, or 20 or more mutated oncogenes are identified. In some embodiments, an aforementioned method is provided wherein the one or more driver mutations identified in step (b) comprise missense mutations. In one embodiment, missense mutations occurring in the same amino acid position in >0.5% frequency in each mutated oncogene of the cancer are identified in step (b) and selected for steps (c)-(f).

In still other embodiments, an aforementioned method is provided wherein the peptide sequence comprises a driver mutation flanked by approximately 15 non-mutated oncogene amino acids. In one embodiment, the driver mutation sequence is inserted approximately in the middle of the peptide sequence and wherein the peptide sequence is approximately 28-35 amino acids in length. In some embodiments, an aforementioned method is provided wherein the peptide sequence comprises 2 driver mutations are flanked by approximately 8 non-mutated oncogene amino acids. In some embodiments, an aforementioned method is provided wherein the vector is a lentivector. In one embodiment, the lentivector comprises 2, 3, 4, 5, 6, 7, 8, 9, 10, 11, 12, 13, 14, 15, 16, 17, 18, 19 or 20 or more peptide sequences, each comprising one or more driver mutations, wherein each peptide sequence is optionally separated by a cleavage site. In another embodiment, the cleavage site comprises a furin cleavage site. In some embodiments, an aforementioned method is provided wherein the vector is introduced into the at least one cancer cell line by transduction. In some embodiments, the subject is human.

The present disclosure provides, in one embodiment, an aforementioned method wherein the one or more mutated oncogenes is selected from the group consisting of APC, TP53, KRAS, PIK3CA, FAT4, LRP1B, FBXW7, BRAF, SMAD4, PCLO, KMT2C, KMT2D, ATM, RNF213, ZFHX3, AMER1, TRRAP, ARID1A, FAT1, EP400, SOX9, RNF43, MKI67, RELN, PTPRS, PDE4DIP, CHD4, PTPRT, ANKRD11, ROBO1, MTOR, CREBBP, LRRK2, TCF7L2, KMT2B, PRKDC, UBR5, ACVR2A, ERBB4, PREX2, CARD11, NOTCH1, PTEN, NCOR2, GRIN2A, KMT2A, ATRX, CACNA1D, ALK, MYH9, NOTCH3, POLE, BCORL1, SPEN, BCL9L, BRCA2, CUX1, ARID1B, CTNNB1, MYH11, SMARCA4, NF1, PIK3CG, PLCG2, AXIN2, MGA, SLX4, FLT4, ERBB3, POLQ, ASXL1, CAD, PTPRK, ARID2, CIC, EP300, EPHA5, NUMA1, CAMTA1, GNAS, LRP5, BCL9, PTPRD, RANBP2, IRS1, MYO5A, ROS1, IRS4, SETD1A, PIK3R1, PTPRC, COL1A1, TP53BP1, DICER1, SETBP1, ZBTB20, KDM2B, B2M, AFDN, ZNF521, and LARP4B. In one embodiment, the one or more oncogenes comprise TP53 (SEQ ID NO: 36), PIK3CA (SEQ ID NO: 38), FBXW7(SEQ ID NO: 40), SMAD4 (SEQ ID NO: 42), GNAS (SEQ ID NO: 50), ATM (SEQ ID NO: 44), KRAS (SEQ ID NO: 34), CTNNB1 (SEQ ID NO: 46), and ERBB3 (SEQ ID NO: 48). In another embodiment, TP53 (SEQ ID NO: 36) comprises driver mutations selected from the group consisting of R175H, R273C, G245S, and R248W; PIK3CA (SEQ ID NO: 38) comprises driver mutations selected from the group consisting of E542K, R88Q, M10431, and H1047Y; FBXW7(SEQ ID NO: 40) comprises driver mutations selected from the group consisting of R505C, S582L and R465H; SMAD4 (SEQ ID NO: 42) comprises driver mutations selected from the group consisting of R361H, GNAS (SEQ ID NO: 50) comprises driver mutations selected from the group consisting of R201 H, ATM (SEQ ID NO: 44) comprises driver mutations selected from the group consisting of R337C; KRAS (SEQ ID NO: 34) comprises driver mutations selected from the group consisting of G12D, G12C and G12V; CTNNB1 (SEQ ID NO: 46) comprises driver mutations selected from the group consisting of S45F; and ERBB3 (SEQ ID NO: 48) comprises drive mutation V104M. In still another embodiment, peptide sequences comprising the driver mutations R273C of oncogene TP53 (SEQ ID NO: 36), E542K of oncogene PIK3CA (SEQ ID NO: 38), R361H of oncogene SMAD4 (SEQ ID NO: 42), R201H of oncogene GNAS (SEQ ID NO: 50), R505C of oncogene FBXW7 (SEQ ID NO: 40), and R337C of oncogene ATM (SEQ ID NO: 44) are inserted into a first lentiviral vector (SEQ ID NO:54), and peptide sequences comprising the driver mutations R175H, G245S, and R248W of oncogene TP53 (SEQ ID NO: 36), G12C of oncogene KRAS (SEQ ID NO: 34), R88Q, M10431, and H1047Y of oncogene PIK3CA (SEQ ID NO: 38), S582L and R465H of oncogene FBXW7 (SEQ ID NO: 40), S45F of oncogene CTNNB1 (SEQ ID NO: 46), and V104M of oncogene ERBB3 (SEQ ID NO: 48) are inserted into a second lentiviral vector (SEQ ID NO: 52).

In some embodiments, a method of stimulating an immune response in a patient afflicted with colorectal cancer is provided comprising the steps of administering a an aforementioned composition. Inyet another embodiment, a method of treating colorectal cancer in a patient is provided comprising the steps of administering an aforementioned composition. In some embodiments, an aforementioned method is provided wherein each composition comprises approximately 1.0×10⁶-6.0×10⁷ cells. In some embodiments, an aforementioned method is provided further comprising administering to the subject a therapeutically effective dose of one or more additional therapeutics selected from the group consisting of: a chemotherapeutic agent, cyclophosphamide, a checkpoint inhibitor, and all-trans retinoic acid (ATRA). In one embodiment, the method comprises administering to the subject a therapeutically effective dose of a checkpoint inhibitor selected from the group consisting of an antibody that binds PD-1 or PD-L1.

A method of stimulating an immune response in a patient is also provided in the present discosure as one embodiment, comprising administering to said patient a therapeutically effective amount of a unit dose of a colorectal cancer vaccine, wherein said unit dose comprises a composition comprising a cancer stem cell line and at least 3 compositions each comprising a different colorectal cancer cell line; wherein the cell lines are optionally modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, and/or (ii) express or increase expression of 1, 2, 3, 4, 5, 6, 7, 8, 9 or 10 immunostimulatory factors, and/or (iii) inhibitor decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines.

In some embodiments, a method of treating colorectal cancer in a patient is provided comprising administering to said patient a therapeutically effective amount of a unit dose of a colorectal cancer vaccine, wherein said unit dose comprises a composition comprising a cancer stem cell line and at least 3 compositions each comprising a different colorectal cancer cell line; wherein the cell lines are optionally modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, and/or (ii) express or increase expression of 1, 2, 3, 4, 5, 6, 7, 8, 9 or 10 immunostimulatory factors, and/or (iii) inhibitor decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines.

In some embodiments, an aforementioned method is provided wherein the wherein the colorectal cancer cell line or cell lines are selected from the group consisting of LS123, HCT15, SW1463, RKO, HUTU80, HCT116, LOVO, T84, LS411N, SW48, C2BBe1, Caco-2, SNU-1033, COLO 201, GP2d, CL-14, SW403, SW1116, SW837, SK-CO-1, CL-34, NCI-H508, CCK-81, SNU-C2A, GP2d, HT-55, MDST8, RCM-1, CL-40, COLO 678, and LS180. In one embodiment, the unit dose comprises a composition comprising a cancer stem cell line and 5 compositions comprising the cell lines HCT15, RKO, HUTU80, HCT116, and LS411 N.

In one embodiment, the present disclosure provides a method of stimulating an immune response in a patient comprising administering to said patient a therapeutically effective amount of a unit dose of a colorectal cancer vaccine, wherein said unit dose comprises 6 compositions comprising cancer cell lines HCT15, HUTU80, LS411 N, DMS 53, HCT116 and RKO, wherein: (a) HCT15 is modified to (i) express at least one immunostimulatory factor, and (ii) decrease expression of at least one immunosuppressive factor; (b) HUTU80 is modified to (i) express at least one immunostimulatory factor, at least one TAA that is either not expressed or minimally expressed by HUTU80, and at least 1 peptide comprising at least 1 oncogene driver mutation; and (ii) decrease expression of at least one immunosuppressive factor; (c) LS411 N is modified to (i) express at least one immunostimulatory factor, and (ii) decrease expression of at least one immunosuppressive factor; (d) HCT116 is modified to (i) express at least one immunostimulatory factor, at least one TAA that is either not expressed or minimally expressed by HCT116, and at least 1 peptide comprising at least 1 oncogene driver mutation; and (ii) decrease expression of at least one immunosuppressive factor; (e) RKO is modified to (i) express at least one immunostimulatory factor, and at least 1 peptide comprising at least 1 oncogene driver mutation; and (ii) decrease expression of at least one immunosuppressive factor; and (f) DMS 53 is modified to (i) express at least one immunostimulatory factor, and (ii) decrease expression of at least one immunosuppressive factor.

In yet another embodiment, the present disclosure provides a method of treating colorectal cancer in a patient comprising administering to said patient a therapeutically effective amount of a unit dose of a colorectal cancer vaccine, wherein said unit dose comprises 6 compositions comprising cancer cell lines HCT15, HUTU80, LS411 N, DMS 53, HCT116 and RKO, wherein: (a) HCT15 is modified to (i) express at least one immunostimulatory factor, and (ii) decrease expression of at least one immunosuppressive factor; (b) HUTU80 is modified to (i) express at least one immunostimulatory factor, at least one TAA that is either not expressed or minimally expressed by HUTU80, and at least 1 peptide comprising at least 1 oncogene driver mutation; and (ii) decrease expression of at least one immunosuppressive factor; (c) LS411 N is modified to (i) express at least one immunostimulatory factor, and (ii) decrease expression of at least one immunosuppressive factor; (d) HCT116 is modified to (i) express at least one immunostimulatory factor, at least one TAA that is either not expressed or minimally expressed by HCT116, and at least 1 peptide comprising at least 1 oncogene driver mutation; and (ii) decrease expression of at least one immunosuppressive factor; (e) RKO is modified to (i) express at least one immunostimulatory factor, and at least 1 peptide comprising at least 1 oncogene driver mutation; and (ii) decrease expression of at least one immunosuppressive factor; and (f) DMS 53 is modified to (i) express at least one immunostimulatory factor, and (ii) decrease expression of at least one immunosuppressive factor.

In one embodiment, the present disclosure provides a method of stimulating an immune response in a patient comprising administering to said patient a therapeutically effective amount of a unit dose of a colorectal cancer vaccine, wherein said unit dose comprises a first composition comprising cancer cell lines HCT15, HUTU80 and LS411N, and a second composition comprising cancer cell lines DMS 53, HCT116 and RKO wherein: (a) HCT15 is modified to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25); (b) HUTU80 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361 H of oncogene SMAD4, R201H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25); (c) LS411N is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25); (d) HCT116 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25); (e) RKO is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25); and (f) DMS 53 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27); and (ii) decrease expression of CD276 using a zinc-finger nuclease targeting CD276 (SEQ ID NO: 25).

In still another embodiment, the present disclosure provides a method of treating colorectal cancer in a patient comprising administering to said patient a therapeutically effective amount of a unit dose of a colorectal cancer vaccine, wherein said unit dose comprises a first composition comprising cancer cell lines HCT15, HUTU80 and LS411N, and a second composition comprising cancer cell lines DMS 53, HCT116 and RKO wherein: (a) HCT15 is modified to (i) express GM-CSF (SEQ ID NO: 8), IL-12 (SEQ ID NO: 10), membrane-bound CD40L (SEQ ID NO: 3), and TGFβ1 shRNA (SEQ ID NO: 26); and (ii) decrease expression of CD276 using a zinc-finger nuclease targeting CD276 (SEQ ID NO: 25); (b) HUTU80 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27), modPSMA (SEQ ID NO: 20), and peptides comprising one or more driver mutation sequences selected from the group consisting of R273C of oncogene TP53, E542K of oncogene PIK3CA, R361 H of oncogene SMAD4, R201H of oncogene GNAS, R505C of oncogene FBXW7, and R337C of oncogene ATM (SEQ ID NO: 54); and (ii) decrease expression of CD276 using a zinc-finger nuclease targeting CD276 (SEQ ID NO: 25); (c) LS411N is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26); and (ii) decrease expression of CD276 using a zinc-finger nuclease targeting CD276 (SEQ ID NO: 25); (d) HCT116 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), modTBXT (SEQ ID NO: 18), modWT1 (SEQ ID NO: 18), and peptides comprising one or more driver mutation sequences selected from the group consisting of G12D and G12V of oncogene KRAS (SEQ ID NO: 18); and (ii) decrease expression of CD276 using a zinc-finger nuclease targeting CD276 (SEQ ID NO: 25); (e) RKO is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), and peptides comprising one or more driver mutations sequences selected from the group consisting of R175H, G245S, and R248W of oncogene TP53, G12C of oncogene KRAS, R88Q, M10431, and H1047Y of oncogene PIK3CA, S582L and R465H of oncogene FBXW7, S45F of oncogene CTNNB1, and V104M of oncogene ERBB3 (SEQ ID NO: 52); and (ii) decrease expression of CD276 using a zinc-finger nuclease targeting CD276 (SEQ ID NO: 25); and (f) DMS 53 is modified to (i) express GM-CSF (SEQ ID NO: 8), IL-12 (SEQ ID NO: 10), membrane-bound CD40L (SEQ ID NO: 3), TGFβ1 shRNA (SEQ ID NO: 26), TGFβ2 shRNA (SEQ ID NO: 27); and (ii) decrease expression of CD276 using a zinc-finger nuclease targeting CD276 (SEQ ID NO: 25).

### BRIEF DESCRIPTION OF THE DRAWINGS

FIGS. 1A-B show endogenous expression of twenty prioritized CRC antigens by vaccine cell lines (FIG. 1A) and number of these CRC antigens expressed by the vaccine cell lines also expressed by CRC patient tumors (FIG. 1B).

FIGS. 2A-J show the expression of and IFNγ responses to antigens introduced into CRC vaccine cell lines by lentiviral transduction compared to unmodified controls. Expression of modPSMA by HuTu80 (FIG. 2A) and IFNγ responses to PSMA (FIG. 2F) in CRC-vaccine A. Expression of modTBXT, modWT1, KRAS G12D and KRAS G12V by HCT-116 (FIG. 2B-C) and IFNγ responses to TBXT (FIG. 2G), WT1 (FIG. 2H), KRAS G12D (FIG. 2I) and KRAS G12D (FIG. 2J) in CRC-vaccine B.

FIGS. 3A-C show antigen specific IFNγ responses for six HLA-diverse donors induced by the unit dose of the CRC vaccine (FIG. 3A), CRC vaccine-A (FIG. 3B) and CRC vaccine-B (FIG. 3C) compared to unmodified controls.

FIG. 4 shows antigen specific IFNγ responses induced by the unit dose of the CRC vaccine and unmodified controls in the six individual donors summarized in FIG. 3.

FIG. 5 shows IFNγ responses for six HLA-diverse donors to three TP53 driver mutations encoded by two peptides, one KRAS driver mutation encoded by one peptide, three PIK3CA driver mutations encoded by two peptides, two FBXW7 driver mutations encoded by two peptides, one CTNNB1 driver mutation encoded by one peptide and one ERBB3 driver mutation encoded by one peptide expressed by modified RKO and unmodified RKO.

FIG. 6. shows IFNγ responses for six HLA-diverse donors to peptides encoding one TP53 driver mutation by one peptide, one PIK3CA driver mutation by one peptide, one FBXW7 driver mutation by one peptide, one SMAD4 driver mutation by one peptide, one GNAS driver mutation encoded by one peptide and one ATM driver mutation encoded by one peptide expressed by modified Hutu80 compared to unmodified Hutu80.

### DETAILED DESCRIPTION

Embodiments of the present disclosure provide a platform approach to cancer vaccination that provides both breadth, in terms of the types of cancer amenable to treatment by the compositions, methods, and regimens disclosed, and magnitude, in terms of the immune responses elicited by the compositions, methods, and regimens disclosed.

In various embodiments of the present disclosure, intradermal injection of an allogenic whole cancer cell vaccine induces a localized inflammatory response recruiting immune cells to the injection site. Without being bound to any theory or mechanism, following administration of the vaccine, antigen presenting cells (APCs) that are present locally in the skin (vaccine microenvironment, VME), such as Langerhans cells (LCs) and dermal dendritic cells (DCs), uptake vaccine cell components by phagocytosis and then migrate through the dermis to a draining lymph node. At the draining lymph node, DCs or LCs that have phagocytized the vaccine cell line components can prime naïve T cells and B cells. Priming of naïve T and B cells initiates an adaptive immune response to tumor associated antigens (TAAs) expressed by the vaccine cell lines. In some embodiments of the present disclosure, the priming occurs in vivo and not in vitro or ex vivo. In embodiments of the vaccine compositions provided herein, the multitude of TAAs expressed by the vaccine cell lines are also expressed a subject's tumor. Expansion of antigen specific T cells at the draining lymph node and the trafficking of these T cells to the tumor microenvironment (TME) can initiate a vaccine-induced anti-tumor response.

Immunogenicity of an allogenic vaccine can be enhanced through genetic modifications of the cell lines comprising the vaccine composition to introduce TAAs (native/wild-type or designed/mutated) as described herein. Immunogenicity of an allogenic vaccine can be enhanced through genetic modifications of the cell lines comprising the vaccine composition to express one or more tumor fitness advantage mutations, including but not limited to acquired tyrosine kinase inhibitor (TKI) resistance mutations, EGFR activating mutations, and/or modified ALK intracellular domain(s). Immunogenicity of an allogenic vaccine can be enhanced through genetic modifications of the cell lines comprising the vaccine composition to introduce driver mutations as described herein. Immunogenicity of an allogenic vaccine can be further enhanced through genetic modifications of the cell lines comprising the vaccine composition to reduce expression of immunosuppressive factors and/or increase the expression or secretion of immunostimulatory signals. Modulation of these factors can enhance the uptake of vaccine cell components by LCs and DCs in the dermis, facilitate the trafficking of DCs and LCs to the draining lymph node, and enhance effector T cell and B cell priming in the draining lymph node, thereby providing more potent anti-tumor responses.

In various embodiments, the present disclosure provides an allogeneic whole cell cancer vaccine platform that includes compositions and methods for treating cancer, and/or preventing cancer, and/or stimulating an immune response. Criteria and methods according to embodiments of the present disclosure include without limitation: (i) criteria and methods for cell line selection for inclusion in a vaccine composition, (ii) criteria and methods for combining multiple cell lines into a therapeutic vaccine composition, (iii) criteria and methods for making cell line modifications, and (iv) criteria and methods for administering therapeutic compositions with and without additional therapeutic agents. In some embodiments, the present disclosure provides an allogeneic whole cell cancer vaccine platform that includes, without limitation, administration of multiple cocktails comprising combinations of cell lines that together comprise one unit dose, wherein unit doses are strategically administered over time, and additionally optionally includes administration of other therapeutic agents such as cyclophosphamide and additionally optionally a checkpoint inhibitor and additionally optionally a retinoid (e.g., ATRA).

The present disclosure provides, in some embodiments, compositions and methods for tailoring a treatment regimen for a subject based on the subject's tumor type. In some embodiments, the present disclosure provides a cancer vaccine platform whereby allogeneic cell line(s) are identified and optionally modified and administered to a subject. In various embodiments, the tumor origin (primary site) of the cell line(s), the amount and number of TAAs expressed by the cell line(s), the number of cell line modifications, and the number of cell lines included in a unit dose are each customized based on the subject's tumor type, stage of cancer, and other considerations. As described herein, the tumor origin of the cell lines may be the same or different than the tumor intended to be treated. In some embodiments, the cancer cell lines may be cancer stem cell lines.

### Definitions

In this disclosure, “comprises”, “comprising”, “containing”, “having”, and the like have the meaning ascribed to them in U.S. patent law and mean “includes”, “including”, and the like; the terms “consisting essentially of” or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excluding prior art embodiments.

Unless specifically otherwise stated or obvious from context, as used herein, the terms “a”, “an”, and “the” are understood to be singular or plural.

The terms “cell”, “cell line”, “cancer cell line” (e.g., a colorectal cancer cell line), “tumor cell line”, and the like as used interchangeably herein refers to a cell line that originated from a cancerous tumor as described herein, and/or originates from a parental cell line of a tumor originating from a specific source/organ/tissue. In some embodiments the cancer cell line is a cancer stem cell line as described herein. In certain embodiments, the cancer cell line is known to express or does express multiple tumor-associated antigens (TAAs) and/or tumor specific antigens (TSAs). In some embodiments of the disclosure, a cancer cell line is modified to express, or increase expression of, one or more TAAs. In certain embodiments, the cancer cell line includes a cell line following any number of cell passages, any variation in growth media or conditions, introduction of a modification that can change the characteristics of the cell line such as, for example, human telomerase reverse transcriptase (hTERT) immortalization, use of xenografting techniques including serial passage through xenogenic models such as, for example, patient-derived xenograft (PDX) or next generation sequencing (NGS) mice, and/or co-culture with one or more other cell lines to provide a mixed population of cell lines. As used herein, the term “cell line” includes all cell lines identified as having any overlap in profile or segment, as determined, in some embodiments, by Short Tandem Repeat (STR) sequencing, or as otherwise determined by one of skill in the art. As used herein, the term “cell line” also encompasses any genetically homogeneous cell lines, in that the cells that make up the cell line(s) are clonally derived from a single cell such that they are genetically identical. This can be accomplished, for example, by limiting dilution subcloning of a heterogeneous cell line. The term “cell line” also encompasses any genetically heterogeneous cell line, in that the cells that make up the cell line(s) are not expected to be genetically identical and contain multiple subpopulations of cancer cells. Various examples of cell lines are described herein. Unless otherwise specifically stated, the term “cell line” or “cancer cell line” encompasses the plural “cell lines.”

As used herein, the term “tumor” (e.g., a colorectal cancer tumor) refers to an accumulation or mass of abnormal cells. Tumors may be benign (non-cancerous), premalignant (pre-cancerous, including hyperplasia, atypia, metaplasia, dysplasia and carcinoma in situ), or malignant (cancerous). It is well known that tumors may be “hot” or “cold”. By way of example, melanoma and lung cancer, among others, demonstrate relatively high response rates to checkpoint inhibitors and are commonly referred to as “hot” tumors. These are in sharp contrast to tumors with low immune infiltrates called “cold” tumors or non-T-cell-inflamed cancers, such as those from the prostate, pancreas, glioblastoma, and bladder, among others. In some embodiments, the compositions and methods provided herein are useful to treat or prevent cancers with associated hot tumors. In some embodiments, the compositions and methods provided herein are useful to treat or prevent cancers with cold tumors. Embodiments of the vaccine compositions of the present disclosure can be used to convert cold (i.e., treatment-resistant or refractory) cancers or tumors to hot (i.e., amenable to treatment, including a checkpoint inhibition-based treatment) cancers or tumors. Immune responses against cold tumors are dampened because of the lack of neoepitopes associated with low mutational burden. In various embodiments, the compositions described herein comprise a multitude of potential neoepitopes arising from point-mutations that can generate a multitude of exogenous antigenic epitopes. In this way, the patients' immune system can recognize these epitopes as non-self, subsequently break self-tolerance, and mount an anti-tumor response to a cold tumor, including induction of an adaptive immune response to wide breadth of antigens (See Leko, V. et al. J Immunol (2019)).

Cancer stem cells are responsible for initiating tumor development, cell proliferation, and metastasis and are key components of relapse following chemotherapy and radiation therapy. In certain embodiments, a cancer stem cell line or a cell line that displays cancer stem cell characteristics is included in one or more of the vaccine compositions. As used herein, the phrase “cancer stem cell” (CSC) or “cancer stem cell line” refers to a cell or cell line within a tumor that possesses the capacity to self-renew and to cause the heterogeneous lineages of cancer cells that comprise the tumor. CSCs are highly resistant to traditional cancer therapies and are hypothesized to be the leading driver of metastasis and tumor recurrence. To clarify, a cell line that displays cancer stem cell characteristics is included within the definition of a “cancer stem cell”. Exemplary cancer stem cell markers identified by primary tumor site are provided in Table 2 and described herein. Cell lines expressing one or more of these markers are encompassed by the definition of “cancer stem cell line”. Exemplary cancer stem cell lines are described herein, each of which are encompassed by the definition of “cancer stem cell line”.

As used herein, the phrase “each cell line or a combination of cell lines” refers to, where multiple cell lines are provided in a combination, 1, 2, 3, 4, 5, 6, 7, 8, 9, 10 or more or the combination of the cell lines. As used herein, the phrase “each cell line or a combination of cell lines have been modified” refers to, where multiple cell lines are provided in combination, modification of one, some, or all cell lines, and also refers to the possibility that not all of the cell lines included in the combination have been modified. By way of example, the phrase “a composition comprising a therapeutically effective amount of at least 2 cancer cell lines, wherein each cell line or a combination of the cell lines comprises cells that have been modified . . . ” means that each of the two cell lines has been modified or one of the two cell lines has been modified. By way of another example, the phrase “a composition comprising a therapeutically effective amount of at least 3 cancer cell lines, wherein each cell line or a combination of the cell lines comprises cells that have been modified . . . ” means that each (i.e., all three) of the cell lines have been modified or that one or two of the three cell lines have been modified.

The term “oncogene” as used herein refers to a gene involved in tumorigenesis. An oncogene is a mutated (i.e., changed) form of a gene that contributes to the development of a cancer. In their normal, unmutated state, onocgenes are called proto-oncogenes, and they play roles in the regulation of normal cell growth and cell division.

The term “driver mutation” as used herein, for example in the context of an oncogene, refers to a somatic mutation that initiates, alone or in combination with other mutations, tumorogenesis and/or confers a fitness advantage to tumor cells. Driver mutations typically occur early in cancer evolution and are therefore found in all or a subset of tumor cells across cancer pateints (i.e., at a high frequency). The phrase “wherein the oncogene driver mutation is in one or more oncogenes” as used herein means the driver mutation (e.g., the missense mutation) occurs within the polynucleotide sequence (and thus the corresponding amino acid sequence) of the oncogene or oncogenes.

The term “tumor fitness advantage mutation” as used herein refers to one or more mutations that result in or cause a rapid expansion of a tumor (e.g., a collection of tumor cells) or tumor cell (e.g., tumor cell clone) harboring such mutations. In some embodiments, tumor fitness advantage mutations include, but are not limited to, (oncogene) driver mutations as described herein, acquired tyrosine kinase inhibitor (TKI) resistance mutations as described herein, and activating mutations as described herein. The term “acquired tyrosine kinase inhibitor (TKI) resistance mutation” as used herein refers to mutations that account for TKI resistance and cause tumor cells to effectively escape TKI treatment. In some embodiments provided herein, the mutation or mutations occur in the ALK gene (i.e., “ALK acquired tyrosine kinase inhibitor (TKI) resistance mutation”) and/or in the EGFR gene (i.e., “EGFR acquired tyrosine kinase inhibitor (TKI) resistance mutation”). The term “EGFR activating mutation” as used herein refers to a mutation resulting in constitutive activation of EGFR. Exemplary driver/acquired resistance/activating mutations (e.g., point mutations, substitutions, etc.) are provided herein.

The term “modified ALK intracellular domain (modALK-IC)” as used herein refers to neoepitope-constaining ALK C-terminus intracullar tyrosine kinase domain, which mediates the ligand-dependent dimerization and/or oligomerization of ALK, resulting in constitutive kinase activity and promoting downstream signaling pathways involved in the proliferation and survival of tumor cells.

As used herein, the phrase “identifying one or more . . . mutations” for example in the process for preparing compositions useful for stimulating an immune response or treating cancer as described herein, refers to newly identifying, identifying within a database or dataset or otherwise using a series of criteria or one or more components thereof as described herein and, optionally, selecting the oncogene or mutation for use or inclusion in a vaccine composition as described herein.

The phrase “ . . . cells that express at least [n] tumor associated antigens (TAAs) associated with a cancer of a subject intended to receive said composition.” as used herein refers to cells that express, either natively or by way of genetic modification, the designated number of TAAs and wherein said same TAAs are expressed or known to be expressed by cells of a patient's tumor. The expression of specific TAAs by cells of a patient's tumor may be determined by assay, surgical procedures (e.g., biopsy), or other methods known in the art. In other embodiments, a clinician may consult the Cancer Cell Line Encyclopedia (CCLE) and other known resources to identify a list of TAAs known to be expressed by cells of a particular tumor type.

As used herein, the phrase “ . . . wherein the cell lines comprise cells that collectively express at least [15] tumor associated antigens (TAAs) associated with the cancer . . . ” refers to a compotiion or method employing multiple cell lines and wherein the combined total of TAAs expressed by the multiple cell lines is at least the recited number.

As used herein, the phrase “ . . . that is either not expressed or minimally expressed . . . ” means that the referenced gene or protein (e.g., a TAA or an immunosuppressive protein or an immunostimulatory protein) is not expressed by a cell line or is expressed at a low level, where such level is inconsequential to or has a limited impact on immunogenicity. For example, it is readily appreciated in the art that a TAA may be present or expressed in a cell line in an amount insufficient to have a desired impact on the therapeutic effect of a vaccine composition including said cell line. In such a scenario, the present disclosure provides compositions and methods to increase expression of such a TAA. Assays for determining the presence and amount of expression are well known in the art and described herein.

As used herein, the term “equal” generally means the same value+/−10%. In some embodiments, a measurement, such as number of cells, etc., can be +/−1, 2, 3, 4, 5, 6, 7, 8, 9, or 10%. Similarly, as used herein and as related to amino acid position or nucleotide position, the term “approximately” refers to within 1, 2, 3, 4, or 5 such residues. With respect to the number of cells, the term “approximately” refers to +/−1, 2, 3, 4, 5, 6, 7, 8, 9, or 10%.

As used herein, the phrase “ . . . wherein said composition is capable of stimulating a 1.3-fold increase in IFNγ production compared to unmodified cancer cell lines . . . ” means, when compared to a composition of the same cell line or cell lines that has/have not been modified, the composition comprising a modified cell line or modified cell lines is capable of stimulating at least 1.3-fold more IFNγ production. In this example, “at least 1.3” means 1.3, 1.4, 1.5, etc., or higher. This definition is used herein with respect to other values of IFNγ production, including, but not limited to, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, or 5.0-fold or higher increase in IFNγ production compared to unmodified cancer cell lines (e.g., a modified cell line compared to an modified cell line, a composition of 2 or 3 modified cell lines (e.g., a vaccine composition) compared cell lines to the same composition comprising unmodified cell lines, or a unit dose comprising 6 modified cell lines compared to the same unit dose comprising unmodified cell lines). In other embodiments, the IFNγ production is increased by approximately 1, 2, 3, 4, 5, 6, 7, 8, 9, 10, 11, 12, 13, 14, 15, 16, 17, 18, 19, 20, 21, 22, 23, 24, or 25-fold or higher compared to unmodified cancer cell lines. Similarly, in various embodiments, the present disclosure provides compositions of modified cells or cell lines that are compared to unmodified cells or cell lines on the basis of TAA expression, immunostimulatory factor expression, immunosuppressive factor expression, and/or immune response stimulation using the methods provided herein and the methods known in the art including, but not limited to, ELISA, IFNγ ELISpot, and flow cytometry.

As used herein, the phrase “fold increase” refers to the change in units of expression or units of response relative to a control. By way of example, ELISA fold change refers to the level of secreted protein detected for the modified cell line divided by the level of secreted protein detected, or the lower limit of detection, by the unmodified cell line. In another example, fold change in expression of an antigen by flow cytometry refers to the mean fluorescence intensity (MFI) of expression of the protein by a modified cell line divided by the MFI of the protein expression by the unmodified cell line. IFNγ ELISpot fold change refers to the average IFNγ spot-forming units (SFU) induced across HLA diverse donors by the test variable divided by the average IFNγ SFU induced by the control variable. For example, the average total antigen specific IFNγ SFU across donors by a composition of three modified cell lines divided by the IFNγ SFU across the same donors by a composition of the same three unmodified cell lines.

In some embodiments, the fold increase in IFNγ production will increase as the number of modifications (e.g., the number of immunostimulatory factors and the number of immunosuppressive factors) is increased in each cell line. In some embodiments, the fold increase in IFNγ production will increase as the number of cell lines (and thus, the number of TAAs), whether modified or unmodified, is increased. The fold increase in IFNγ production, in some embodiments, is therefore attributed to the number of TAAs and the number of modifications.

As used herein, the term “modified” means genetically modified or changed to express, overexpress, increase, decrease, or inhibit the expression of one or more protein or nucleic acid. As described herein, exemplary proteins include, but are not limited to immunostimulatory factors. Exemplary nucleic acids include sequences that can be used to knockdown (KD) (i.e., decrease expression of) or knockout (KO) (i.e., completely inhibit expression of) immunosuppressive factors. As used herein, the term “decrease” is synonymous with “reduce” or “partial reduction” and may be used in association with gene knockdown. Likewise, the term “inhibit” is synonymous with “complete reduction” and may be used in the context of a gene knockout to describe the complete excision of a gene from a cell.

Unless specifically stated or obvious from context, as used herein, the term “or” is understood to be inclusive.

As used herein, the terms “patient”, “subject”, “recipient”, and the like are used interchangeably herein to refer to any mammal, including humans, non-human primates, domestic and farm animals, and other animals, including, but not limited to dogs, horses, cats, cattle, sheep, pigs, mice, rats, and goats. Exemplary subjects are humans, including adults, children, and the elderly. In some embodiments, the subject can be a donor.

The terms “treat”, “treating”, “treatment”, and the like, as used herein, unless otherwise indicated, refers to reversing, alleviating, inhibiting the process of disease, disorder or condition to which such term applies, or one or more symptoms of such disease, disorder or condition and includes the administration of any of the compositions, pharmaceutical compositions, or dosage forms described herein, to prevent the onset of the symptoms or the complications, alleviate the symptoms or the complications, or eliminate the disease, condition, or disorder. As used herein, treatment can be curative or ameliorating.

As used herein, “preventing” means preventing in whole or in part, controlling, reducing, or halting the production or occurrence of the thing or event to which such term applies, for example, a disease, disorder, or condition to be prevented.

Embodiments of the methods and compositions provided herein are useful for preventing a tumor or cancer, meaning the occurrence of the tumor is prevented or the onset of the tumor is significantly delayed. In some embodiments, the methods and compositions are useful for treating a tumor or cancer, meaning that tumor growth is significantly inhibited as demonstrated by various techniques well-known in the art such as, for example, by a reduction in tumor volume. Tumor volume may be determined by various known procedures, (e.g., obtaining two dimensional measurements with a dial caliper). Preventing and/or treating a tumor can result in the prolonged survival of the subject being treated.

As used herein, the term “stimulating”, with respect to an immune response, is synonymous with “promoting”, “generating”, and “eliciting” and refers to the production of one or more indicators of an immune response. Indicators of an immune response are described herein. Immune responses may be determined and measured according to the assays described herein and by methods well-known in the art.

The phrases “therapeutically effective amount”, “effective amount”, “immunologically effective amount”, “anti-tumor effective amount”, and the like, as used herein, indicate an amount necessary to administer to a subject, or to a cell, tissue, or organ of a subject, to achieve a therapeutic effect, such as an ameliorating or a curative effect. The therapeutically effective amount is sufficient to elicit the biological or medical response of a cell, tissue, system, animal, or human that is being sought by a researcher, veterinarian, medical doctor, clinician, or healthcare provider. For example, a therapeutically effective amount of a composition is an amount of cell lines, whether modified or unmodified, sufficient to stimulate an immune response as described herein. In certain embodiments, a therapeutically effective amount of a composition is an amount of cell lines, whether modified or unmodified, sufficient to inhibit the growth of a tumor as described herein. Determination of the effective amount or therapeutically effective amount is, in certain embodiments, based on publications, data or other information such as, for example, dosing regimens and/or the experience of the clinician.

The terms “administering”, “administer”, “administration”, and the like, as used herein, refer to any mode of transferring, delivering, introducing, or transporting a therapeutic agent to a subject in need of treatment with such an agent. Such modes include, but are not limited to, oral, topical, intravenous, intraarterial, intraperitoneal, intramuscular, intratumoral, intradermal, intranasal, and subcutaneous administration.

As used herein, the term “vaccine composition” refers to any of the vaccine compositions described herein containing one or more (e.g., 2, 3, 4, 5, 6, 7, 8, 9, 10, or more) cell lines. As described herein, one or more of the cell lines in the vaccine composition may be modified. In certain embodiments, one or more of the cell lines in the vaccine composition may not be modified. The terms “vaccine”, “tumor cell vaccine”, “cancer vaccine”, “cancer cell vaccine”, “whole cancer cell vaccine”, “vaccine composition”, “composition”, “cocktail”, “vaccine cocktail”, and the like are used interchangeably herein. In some embodiments, the vaccine compositions described herein are useful to treat or prevent cancer. In some embodiments, the vaccine compositions described herein are useful to stimulate or elicit an immune response. In such embodiments, the term “immunogenic composition” is used. In some embodiments, the vaccine compositions described herein are useful as a component of a therapeutic regimen to increase immunogenicity of said regimen.

The terms “dose” or “unit dose” as used interchangeably herein refer to one or more vaccine compositions that comprise therapeutically effective amounts of one more cell lines. As described herein, a “dose” or “unit dose” of a composition may refer to 1, 2, 3, 4, 5, or more distinct compositions or cocktails. In some embodiments, a unit dose of a composition refers to 2 distinct compositions administered substantially concurrently (i.e., immediate series). In exemplary embodiments, one dose of a vaccine composition comprises 1, 2, 3, 4, 5, 6, 7, 8, 9, or 10 separate vials, where each vial comprises a cell line, and where cell lines, each from a separate vial, are mixed prior to administration. In some embodiments, a dose or unit dose includes 6 vials, each comprising a cell line, where 3 cell lines are mixed and administered at one site, and the other 3 cell lines are mixed and administered at a second site. Subsequent “doses” may be administered similarly. In still other embodiments, administering 2 vaccine cocktails at 2 sites on the body of a subject for a total of 4 concurrent injections is contemplated.

As used herein, the term “cancer” refers to diseases in which abnormal cells divide without control and are able to invade other tissues. Thus, as used herein, the phrase “ . . . associated with a (colorectal) cancer of a subject” refers to the expression of tumor associated antigens, neoantigens, or other genotypic or phenotypic properties of a subject's cancer or cancers. TAAs associated with a cancer are TAAs that expressed at detectable levels in a majority of the cells of the cancer. Expression level can be detected and determined by methods described herein. Cancer types can be grouped into broader categories. In some embodiments, cancers may be grouped as solid (i.e., tumor-forming) cancers and liquid (e.g., cancers of the blood such as leukemia, lymphoma and myeloma) cancers. Other categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system cancers (meaning cancers that begin in the tissues of the brain and spinal cord). The term myelodysplastic syndrome refers to a type of cancer in which the bone marrow does not make enough healthy blood cells (white blood cells, red blood cells, and platelets) and there are abnormal cells in the blood and/or bone marrow. Myelodysplastic syndrome may become acute myeloid leukemia (AML).

All references, patents, and patent applications disclosed herein are incorporated by reference with respect to the subject matter for which each is cited, which in some cases may encompass the entirety of the document.

### Vaccine Compositions

The present disclosure is directed to a platform approach to cancer vaccination that provides breadth, with regard to the scope of cancers and tumor types amenable to treatment with the compositions, methods, and regimens disclosed, as well as magnitude, with regard to the level of immune responses elicited by the compositions and regimens disclosed. Embodiments of the present disclosure provide compositions comprising cancer cell lines. In some embodiments, the cell lines have been modified as described herein.

The compositions of the disclosure are designed to increase immunogenicity and/or stimulate an immune response. For example, in some embodiments, the vaccines provided herein increase IFNγ production and the breadth of immune responses against multiple TAAs (e.g., the vaccines are capable of targeting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more TAAs, indicating the diversity of T cell receptor (TCR) repertoire of these anti-TAA T cell precursors. In some embodiments, the immune response produced by the vaccines provided herein is a response to more than one epitope associated with a given TAA (e.g., the vaccines are capable of targeting 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 epitopes or more on a given TAA), indicating the diversity of TCR repertoire of these anti-TAA T cell precursors.

This can be accomplished in certain embodiments by selecting cell lines that express numerous TAAs associated with the cancer to be treated; knocking down or knocking out expression of one or more immunosuppressive factors that facilitates tumor cell evasion of immune system surveillance; expressing or increasing expression of one or more immunostimulatory factors to increase immune activation within the vaccine microenvironment (VME); increasing expression of one or more tumor-associated antigens (TAAs) to increase the scope of relevant antigenic targets that are presented to the host immune system, optionally where the TAA or TAAs are designed or enhanced (e.g., modified by mutation) and comprise, for example, non-synonymous mutations (NSMs) and/or neoepitopes; administering a vaccine composition comprising at least 1 cancer stem cell; and/or any combination thereof.

As described herein, in some embodiments the cell lines are optionally additionally modified to express tumor fitness advantage mutations, including but not limited to acquired tyrosine kinase inhibitor (TKI) resistance mutations, EGFR activating mutations, and/or modified ALK intracellular domain(s), and/or driver mutations.

The one or more cell lines of the vaccine composition can be modified to reduce production of more than one immunosuppressive factor (e.g., 2, 3, 4, 5, 6, 7, 8, 9, 10, or more immunosuppressive factors). The one or more cell lines of a vaccine can be modified to increase production of more than one immunostimulatory factor (e.g., 2, 3, 4, 5, 6, 7, 8, 9, 10, or more immunostimulatory factors). The one or more cell lines of the vaccine composition can naturally express, or be modified to express more than one TAA, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more TAAs.

The vaccine compositions can comprise cells from 1, 2, 3, 4, 5, 6, 7, 8, 9, 10, or more cell lines. Further, as described herein, cell lines can be combined or mixed, e.g., prior to administration. In some embodiments, production of one or more immunosuppressive factors from one or more or the combination of the cell lines can be reduced or eliminated. In some embodiments, production of one or more immunostimulatory factors from one or more or the combination of the cell lines can be added or increased. In certain embodiments, the one or more or the combination of the cell lines can be selected to express a heterogeneity of TAAs. In some embodiments, the cell lines can be modified to increase the production of one or more immunostimulatory factors, TAAs, and/or neoantigens. In some embodiments, the cell line selection provides that a heterogeneity of HLA supertypes are represented in the vaccine composition. In some embodiments, the cells lines are chosen for inclusion in a vaccine composition such that a desired complement of TAAs are represented.

In various embodiments, the vaccine composition comprises a therapeutically effective amount of cells from at least one cancer cell line, wherein the cell line or the combination of cell lines expresses more than one of the TAAs of Table 9. In some embodiments, a vaccine composition is provided comprising a therapeutically effective amount of cells from at least two cancer cell lines, wherein each cell line or the combination of cell lines expresses at least three, at least four, at least five, at least six, at least seven, at least eight, at least nine, or at least ten of the TAAs of Table 9. In some embodiments, a vaccine composition is provided comprising a therapeutically effective amount of cells from at least one cancer cell line, wherein the at least one cell line is modified to express at least one of the immunostimulatory factors of Table 4, at least two of the immunostimulatory factors of Table 4, or at least three of the immunostimulatory factors of Table 4. In further embodiments, a vaccine composition is provided comprising a therapeutically effective amount of cells from at least one cancer cell line, wherein each cell line or combination of cell lines is modified to reduce at least one of the immunosuppressive factors of Table 8, or at least two of the immunosuppressive factors of Table 8.

In embodiments where the one or more cell lines are modified to increase the production of one or more TAAs, the expressed TAAs may or may not have the native coding sequence of DNA/protein. That is, expression may be codon optimized or modified. Such optimization or modification may enhance certain effects (e.g., may lead to reduced shedding of a TAA protein from the vaccine cell membrane). As described herein, in some embodiments the expressed TAA protein is a designed antigen comprising one or more nonsynonymous mutations (NSMs) identified in cancer patients. In some embodiments, the NSMs introduces CD4, CD8, or CD4 and CD8 neoepitopes.

Any of the vaccine compositions described herein can be administered to a subject in order to treat cancer, prevent cancer, prolong survival in a subject with cancer, and/or stimulate an immune response in a subject.

### Cell Lines

In various embodiments of the disclosure, the cell lines comprising the vaccine compositions and used in the methods described herein originate from parental cancer cell lines.

Cell lines are available from numerous sources as described herein and are readily known in the art. For example, cancer cell lines can be obtained from the American Type Culture Collection (ATCC, Manassas, VA), Japanese Collection of Research Bioresources cell bank (JCRB, Kansas City, MO), Cell Line Service (CLS, Eppelheim, Germany), German Collection of Microorganisms and Cell Cultures (DSMZ, Braunschweig, Germany), RIKEN BioResource Research Center (RCB, Tsukuba, Japan), Korean Cell Line Bank (KCLB, Seoul, South Korea), NIH AIDS Reagent Program (NIH-ARP/Fisher BioServices, Rockland, MD), Bioresearch Collection and Research Center (BCRC, Hsinchu, Taiwan), Interlab Cell Line Collection (ICLC, Genova, Italy), European Collection of Authenticated Cell Cultures (ECACC, Salisbury, United Kingdom), Kunming Cell Bank (KCB, Yunnan, China), National Cancer Institute Development Therapeutics Program (NCI-DTP, Bethesda, MD), Rio de Janeiro Cell Bank (BCRJ, Rio de Janeiro, Brazil), Experimental Zooprophylactic Institute of Lombardy and Emilia Romagna (IZSLER, Milan, Italy), Tohoku University cell line catalog (TKG, Miyagi, Japan), and National Cell Bank of Iran (NCBI, Tehran, Iran). In some embodiments, cell lines are identified through an examination of RNA-seq data with respect to TAAs, immunosuppressive factor expression, and/or other information readily available to those skilled in the art.

In various embodiments, the cell lines in the compositions and methods described herein are from parental cell lines of solid tumors originating from the lung, prostate, testis, breast, urinary tract, colon, rectum, stomach, head and neck, liver, kidney, nervous system, endocrine system, mesothelium, ovaries, pancreas, esophagus, uterus or skin. In certain embodiments, the parental cell lines comprise cells of the same or different histology selected from the group consisting of squamous cells, adenocarcinoma cells, adenosquamous cells, large cell cells, small cell cells, sarcoma cells, carcinosarcoma cells, mixed mesodermal cells, and teratocarcinoma cells. In related embodiments, the sarcoma cells comprise osteosarcoma, chondrosarcoma, leiomyosarcoma, rhabdomyosarcoma, mesothelioma, fibrosarcoma, angiosarcoma, liposarcoma, glioma, gliosarcoma, astrocytoma, myxosarcoma, mesenchymous or mixed mesodermal cells.

In certain embodiments, the cell lines comprise cancer cells originating from lung cancer, non-small cell lung cancer (NSCLC), small cell lung cancer (SCLC), prostate cancer, glioblastoma, colorectal cancer, breast cancer including triple negative breast cancer (TNBC), bladder or urinary tract cancer, squamous cell head and neck cancer (SCCHN), liver hepatocellular (HCC) cancer, kidney or renal cell carcinoma (RCC) cancer, gastric or stomach cancer, ovarian cancer, esophageal cancer, testicular cancer, pancreatic cancer, central nervous system cancers, endometrial cancer, melanoma, and mesothelium cancer.

According to various embodiments, the cell lines are allogeneic cell lines (i.e., cells that are genetically dissimilar and hence immunologically incompatible, although from individuals of the same species.) In certain embodiments, the cell lines are genetically heterogeneous allogeneic. In other embodiments, the cell lines are genetically homogeneous allogeneic.

Allogeneic cell-based vaccines differ from autologous vaccines in that they do not contain patient-specific tumor antigens. Embodiments of the allogeneic vaccine compositions disclosed herein comprise laboratory-grown cancer cell lines known to express TAAs of a specific tumor type. Embodiments of the allogeneic cell lines of the present disclosure are strategically selected, sourced, and modified prior to use in a vaccine composition. Vaccine compositions of embodiments of the present disclosure can be readily mass-produced. This efficiency in development, manufacturing, storage, and other areas can result in cost reductions and economic benefits relative to autologous-based therapies.

Tumors are typically made up of a highly heterogeneous population of cancer cells that evolve and change over time. Therefore, it can be hypothesized that a vaccine composition comprising only autologous cell lines that do not target this cancer evolution and progression may be insufficient in the elicitation of a broad immune response required for effective vaccination. As described in embodiments of the vaccine composition disclosed herein, use of one or more strategically selected allogeneic cell lines with certain genetic modification(s) addresses this disparity.

In some embodiments, the allogeneic cell-based vaccines are from cancer cell lines of the same type (e.g., breast, prostate, lung) of the cancer sought to be treated. In other embodiments, various types of cell lines (i.e., cell lines from different primary tumor origins) are combined (e.g., stem cell, prostate, testes). In some embodiments, the cell lines in the vaccine compositions are a mixture of cell lines of the same type of the cancer sought to be treated and cell lines from different primary tumor origins.

Exemplary cancer cell lines, including, but not limited to those provided in Table 1, below, are contemplated for use in the compositions and methods described herein. The Cell Line Sources identified herein are for exemplary purposes only. The cell lines described in various embodiments herein may be available from multiple sources.

TABLE 1
Exemplary vaccine composition cell lines per indication

| Anatomical Site of | Cell Line | Cell Line | Cell Line Source |
|---|---|---|---|
| Primary Tumor | Common Name | Source | Identification |
| Colorectal | LS123 | ATCC | CCL-255 |
| | HCT15 | ATCC | CCL-225 |
| | SW1463 | ATCC | CCL-234 |
| | RKO | ATCC | CRL-2577 |
| | HUTU80 | ATCC | HTB-40 |
| | HCT116 | ATCC | CCL-247 |
| | LOVO | ATCC | CCL-229 |
| | T84 | ATCC | CCL-248 |
| | LS411N | ATCC | CRL-2159 |
| | SW48 | ATCC | CCL-231 |
| | C2BBe1 | ATCC | CRL-2102 |
| | Caco-2 | ATCC | HTB-37 |
| | SNU-1033 | KCLB | 01033 |
| | COLO 201 | ATCC | CCL-224 |
| | GP2d | ECACC | 95090714 |
| | CL-14 | DSMZ | ACC-504 |
| | SW403 | ATCC | CCL-230 |
| | SW1116 | ATCC | CCL-233 |
| | SW837 | ATCC | CCL-235 |
| | SK-CO-1 | ATCC | HTB-39 |
| | CL-34 | DSMZ | ACC-520 |
| | NCI-H508 | ATCC | CCL-253 |
| | CCK-81 | JCRB | JCRB0208 |
| | SNU-C2A | ATCC | CCL-250.1 |
| | GP2d | ECACC | 95090714 |
| | HT-55 | ECACC | 85061105 |
| | MDST8 | ECACC | 99011801 |
| | RCM-1 | JCRB | JCRB0256 |
| | CL-40 | DSMZ | ACC-535 |
| | COLO 678 | DSMZ | ACC-194 |
| | LS180 | ATCC | CL-187 |

In addition to the cell lines identified in Table 1, the following cell lines are also contemplated in various embodiments.

In some embodiments, one or more colorectal cancer (CRC) cell lines are prepared and used according to the disclosure. By way of example, the following colorectal cancer cell lines are contemplated: HCT-15, RKO, HuTu-80, HCT-116, and LS411 N. Additional colorectal cancer cell lines are also contemplated by the present disclosure. As described herein, inclusion of a cancer stem cell line such as DMS 53 in a vaccine comprising CRC cell lines is also contemplated.

Embodiments of vaccine compositions according to the disclosure are used to treat and/or prevent various types of cancer. In some embodiments, a vaccine composition may comprise cancer cell lines that originated from the same type of cancer. For example, a vaccine composition may comprise 1, 2, 3, 4, 5, 6, 7, 8, 9, 10 or more colorectal cancer cell lines, and such a composition may be useful to treat or prevent colorectal cancer. According to certain embodiments, the vaccine composition comprising colorectal cancer cell lines may be used to treat or prevent cancers other than colorectal cancer, examples of which are described herein.

In some embodiments, a vaccine composition may comprise cancer cell lines that originated from different types of cancer. For example, a vaccine composition may comprise 1, 2, 3, 4, 5, 6, 7, 8, 9, 10 or more colorectal cancer cell lines, plus 1, 2, 3, 4, 5, 6, 7, 8, 9, 10 or more bladder cancer cell lines, optionally plus one or other cancer cell lines, such as cancer stem cell lines, and so on, and such a composition may be useful to treat or prevent colorectal cancer, and/or prostate cancer, and/or breast cancer including triple negative breast cancer (TNBC), and so on. According to some embodiments, the vaccine composition comprising different cancer cell lines as described herein may be used to treat or prevent various cancers. In some embodiments, the targeting of a TAA or multiple TAAs in a particular tumor is optimized by using cell lines derived from different tissues or organs within a biological system to target a cancer of primary origin within the same system.

According to various embodiments of the vaccine compositions, the disclosure provides compositions comprising a combination of cell lines. By way of non-limiting examples, cell line combinations are provided below. In each of the following examples, cell line DMS 53, whether modified or unmodified, is combined with 5 other cancer cell lines in the associated list. One or more of the cell lines within each recited combination may be modified as described herein. In some embodiments, none of the cell lines in the combination of cell lines are modified.

According to various embodiments of the vaccine compositions, the disclosure provides compositions comprising DMS 53, HCT-15, RKO, HuTu-80, HCT-116, and LS411 N for the treatment and/or prevention of colorectal cancer. In some embodiments, the cell lines in the vaccine compositions and methods described herein include one or more cancer stem cell (CSC) cell lines, whether modified or unmodified. One example of a CSC cell line is small cell lung cancer cell line DMS 53, whether modified or unmodified. In some embodiments, DMS 53 is modified to reduce expression of CD276, reduce secretion of TGFβ1 and TGFβ2, and express GM-CSF, membrane bound CD40L and IL-12. In other embodiments, DMS 53 is modified to reduce expression of CD276, reduce secretion of TGFβ2, and express GM-CSF and membrane bound CD40L.

CSCs display unique markers that differ depending on the anatomical origin of the tumor. Exemplary CSC markers include: prominin-1 (CD133), A2B5, aldehyde dehydrogenase (ALDH1), polycomb protein (Bmi-1), integrin-β1 (CD29), hyaluronan receptor (CD44), Thy-1 (CD90), SCF receptor (CD117), TRA-1-60, nestin, Oct-4, stage-specific embryonic antigen-1 (CD15), GD3 (CD60a), stage-specific embryonic antigen-1 (SSEA-1) or (CD15), stage-specific embryonic antigen-4 (SSEA-4), stage-specific embryonic antigen-5 (SSEA-5), and Thomsen-Friedenreich antigen (CD176).

Expression markers that identify cancer cell lines with greater potential to have stem cell-like properties differ depending on various factors including anatomical origin, organ, or tissue of the primary tumor. Exemplary cancer stem cell markers identified by primary tumor site are provided in Table 2 and are disclosed across various references (e.g., Gilbert, CA & Ross, A H. J Cell Biochem. (2009); Karsten, U & Goletz, S. SpringerPlus (2013); Zhao, W et al. Cancer Transl Med. (2017)).

Exemplary cell lines expressing one or more markers of cancer stem cell-like properties specific for the anatomical site of the primary tumor from which the cell line was derived are listed in Table 2. Exemplary cancer stem cell lines are provided in Table 3. Expression of CSC markers was determined using RNA-seq data from the Cancer Cell Line Encyclopedia (CCLE) (retrieved from www.broadinstitute.org/ccle on Nov. 23, 2019; Barretina, J et al. Nature. (2012)). The HUGO Gene Nomenclature Committee gene symbol was entered into the CCLE search and mRNA expression downloaded for each CSC marker. The expression of a CSC marker was considered positive if the RNA-seq value (FPKM) was greater than 0.

TABLE 2
Exemplary CSC markers by primary tumor anatomical origin

| Anatomical Site of | CSC Marker | CSC Marker |
|---|---|---|
| Primary Tumor | Common Name | Gene Symbol |
| Ovaries | Endoglin, CD105 | ENG |
| | CD117, cKIT | KIT |
| | CD44 | CD44 |
| | CD133 | PROM1 |
| | SALL4 | SAL4 |
| | Nanog | NANOG |
| | Oct-4 | POU5F1 |
| Pancreas | ALDH1A1 | ALDH1A1 |
| | c-Myc | MYC |
| | EpCAM, TROP1 | EPCAM |
| | CD44 | CD44 |
| | Cd133 | PROM1 |
| | CXCR4 | CXCR4 |
| | Oct-4 | POU5F1 |
| | Nestin | NES |
| | BMI-1 | BMI1 |
| Skin | CD27 | CD27 |
| | ABCB5 | ABCB5 |
| | ABCG2 | ABCG2 |
| | CD166 | ALCAM |
| | Nestin | NES |
| | CD133 | PROM1 |
| | CD20 | MS4A1 |
| | NGFR | NGFR |
| Lung | ALDH1A1 | ALDH1A1 |
| | EpCAM, TROP1 | EPCAM |
| | CD90 | THY1 |
| | CD117, cKIT | KIT |
| | CD133 | PROM1 |
| | ABCG2 | ABCG2 |
| | SOX2 | SOX2 |
| Liver | Nanog | NANOG |
| | CD90/thy1 | THY1 |
| | CD133 | PROM1 |
| | CD13 | ANPEP |
| | EpCAM, TROP1 | EPCAM |
| | CD117, cKIT | KIT |
| | SALL4 | SAL4 |
| | SOX2 | SOX2 |
| Upper Aerodigestive Tract | ABCG2 | ABCG2 |
| (Head and Neck) | ALDH1A1 | ALDH1A1 |
| | Lgr5, GPR49 | LGR5 |
| | BMI-1 | BMI1 |
| | CD44 | CD44 |
| | cMET | MET |
| Central Nervous System | ALDH1A1 | ALDH1A1 |
| | ABCG2 | ABCG2 |
| | BMI-1 | BMI1 |
| | CD15 | FUT4 |
| | CD44 | CD44 |
| | CD49f, Integrin α6 | ITGA6 |
| | CD90 | THY1 |
| | CD133 | PROM1 |
| | CXCR4 | CXCR4 |
| | CX3CR1 | CX3CR1 |
| | SOX2 | SOX2 |
| | c-Myc | MYC |
| | Musashi-1 | MSI1 |
| | Nestin | NES |
| Stomach | ALDH1A1 | ALDH1A1 |
| | ABCB1 | ABCB1 |
| | ABCG2 | ABCG2 |
| | CD133 | PROM1 |
| | CD164 | CD164 |
| | CD15 | FUT4 |
| | Lgr5, GPR49 | LGR5 |
| | CD44 | CD44 |
| | MUC1 | MUC1 |
| | DLL4 | DLL4 |
| Colon (Large and Small | ALDH1A1 | ALDH1A1 |
| Intestines) | c-myc | MYC |
| | CD44 | CD44 |
| | CD133 | PROM1 |
| | Nanog | NANOG |
| | Musashi-1 | MSI1 |
| | EpCAM, TROP1 | EPCAM |
| | Lgr5, GPR49 | LGR5 |
| | SALL4 | SAL4 |
| Breast | ABCG2 | ABCG2 |
| | ALDH1A1 | ALDH1A1 |
| | BMI-1 | BMI1 |
| | CD133 | PROM1 |
| | CD44 | CD44 |
| | CD49f, Integrin α6 | ITGA6 |
| | CD90 | THY1 |
| | c-myc | MYC |
| | CXCR1 | CXCR1 |
| | CXCR4 | CXCR4 |
| | EpCAM, TROP1 | EPCAM |
| | KLF4 | KLF4 |
| | MUC1 | MUC1 |
| | Nanog | NANOG |
| | SALL4 | SAL4 |
| | SOX2 | SOX2 |
| Urinary Tract | ALDH1A1 | ALDH1A1 |
| | CEACAM6, CD66c | CEACAM6 |
| | Oct4 | OCT4 |
| | CD44 | CD44 |
| | YAP1 | YAP1 |
| Hematopoietic and | BMI-1 | BMI1 |
| Lymphoid Tissue | CD117, c-kit | KIT |
| | CD20 | MS4A1 |
| | CD27, TNFRSF7 | CD27 |
| | CD34 | CD34 |
| | CD38 | CD38 |
| | CD44 | CD44 |
| | CD96 | CD96 |
| | GLI-1 | GLI1 |
| | GLI-2 | GLI2 |
| | IL-3Rα | IL3RA |
| | MICL | CLEC12A |
| | Syndecan-1, CD138 | SDC1 |
| | TIM-3 | HAVCR2 |
| Bone | ABCG2 | ABCG2 |
| | CD44 | CD44 |
| | Endoglin, CD105 | ENG |
| | Nestin | NES |

TABLE 3
Cell lines expressing CSC markers

| Anatomical Site of | Cell Line | Cell Line | Cell Line Source |
|---|---|---|---|
| Primary Tumor | Common Name | Source | Identification |
| Ovaries | JHOM-2B | RCB | RCB1682 |
| | OVCAR-3 | ATCC | HTB-161 |
| | OV56 | ECACC | 96020759 |
| | JHOS-4 | RCB | RCB1678 |
| | JHOC-5 | RCB | RCB1520 |
| | OVCAR-4 | NCI-DTP | OVCAR-4 |
| | JHOS-2 | RCB | RCB1521 |
| | EFO-21 | DSMZ | ACC-235 |
| Pancreas | CFPAC-1 | ATCC | CRL-1918 |
| | Capan-1 | ATCC | HTB-79 |
| | Panc 02.13 | ATCC | CRL-2554 |
| | SUIT-2 | JCRB | JCRB1094 |
| | Panc 03.27 | ATCC | CRL-2549 |
| Skin | SK-MEL-28 | ATCC | HTB-72 |
| | RVH-421 | DSMZ | ACC-127 |
| | Hs 895.T | ATCC | CRL-7637 |
| | Hs 940.T | ATCC | CRL-7691 |
| | SK-MEL-1 | ATCC | HTB-67 |
| | Hs 936.T | ATCC | CRL-7686 |
| | SH-4 | ATCC | CRL-7724 |
| | COLO 800 | DSMZ | ACC-193 |
| | UACC-62 | NCI-DTP | UACC-62 |
| Lung | NCI-H2066 | ATCC | CRL-5917 |
| | NCI-H1963 | ATCC | CRL-5982 |
| | NCI-H209 | ATCC | HTB-172 |
| | NCI-H889 | ATCC | CRL-5817 |
| | COR-L47 | ECACC | 92031915 |
| | NCI-H1092 | ATCC | CRL-5855 |
| | NCI-H1436 | ATCC | CRL-5871 |
| | COR-L95 | ECACC | 96020733 |
| | COR-L279 | ECACC | 96020724 |
| | NCI-H1048 | ATCC | CRL-5853 |
| | NCI-H69 | ATCC | HTB-119 |
| | DMS 53 | ATCC | CRL-2062 |
| Liver | HuH-6 | RCB | RCB1367 |
| | Li7 | RCB | RCB1941 |
| | SNU-182 | ATCC | CRL-2235 |
| | JHH-7 | JCRB | JCRB1031 |
| | SK-HEP-1 | ATCC | HTB-52 |
| | Hep 3B2.1-7 | ATCC | HB-8064 |
| Upper Aerodigestive | SNU-1066 | KCLB | 01066 |
| Tract (Head and Neck) | SNU-1041 | KCLB | 01041 |
| | SNU-1076 | KCLB | 01076 |
| | BICR 18 | ECACC | 06051601 |
| | CAL-33 | DSMZ | ACC-447 |
| | DETROIT 562 | ATCC | CCL-138 |
| | HSC-3 | JCRB | JCRB0623 |
| | HSC-4 | JCRB | JCRB0624 |
| | SCC-9 | ATCC | CRL-1629 |
| | YD-8 | KCLB | 60501 |
| Urinary Tract | CAL-29 | DSMZ | ACC-515 |
| | KMBC-2 | JCRB | JCRB1148 |
| | 253J | KCLB | 80001 |
| | 253J-BV | KCLB | 80002 |
| | SW780 | ATCC | CRL-2169 |
| | SW1710 | DSMZ | ACC-426 |
| | VM-CUB-1 | DSMZ | ACC-400 |
| | BC-3C | DSMZ | ACC-450 |
| Central Nervous System | KNS-81 | JCRB | IFO50359 |
| | TM-31 | RCB | RCB1731 |
| | NMC-G1 | JCRB | IFO50467 |
| | GB-1 | JCRB | IFO50489 |
| | SNU-201 | KCLB | 00201 |
| | DBTRG-05MG | ATCC | CRL-2020 |
| | YKG-1 | JCRB | JCRB0746 |
| Stomach | ECC10 | RCB | RCB0983 |
| | RERF-GC-1B | JCRB | JCRB1009 |
| | TGBC-11-TKB | RCB | RCB1148 |
| | SNU-620 | KCLB | 00620 |
| | GSU | RCB | RCB2278 |
| | KE-39 | RCB | RCB1434 |
| | HuG1-N | RCB | RCB1179 |
| | NUGC-4 | JCRB | JCRB0834 |
| | MKN-45 | JCRB | JCRB0254 |
| | SNU-16 | ATCC | CRL-5974 |
| | OCUM-1 | JCRB | JCRB0192 |
| Colon (Large and Small | C2BBe1 | ATCC | CRL-2102 |
| Intestines) | Caco-2 | ATCC | HTB-37 |
| | SNU-1033 | KCLB | 01033 |
| | SW1463 | ATCC | CCL-234 |
| | COLO 201 | ATCC | CCL-224 |
| | GP2d | ECACC | 95090714 |
| | LoVo | ATCC | CCL-229 |
| | SW403 | ATCC | CCL-230 |
| | CL-14 | DSMZ | ACC-504 |
| Breast | HCC2157 | ATCC | CRL-2340 |
| | HCC38 | ATCC | CRL-2314 |
| | HCC1954 | ATCC | CRL-2338 |
| | HCC1143 | ATCC | CRL-2321 |
| | HCC1806 | ATCC | CRL-2335 |
| | HCC1599 | ATCC | CRL-2331 |
| | MDA-MB-415 | ATCC | HTB-128 |
| | CAL-51 | DSMZ | ACC-302 |
| Hematopoietic and | KO52 | JCRB | JCRB0123 |
| Lymphoid Tissue | SKNO-1 | JCRB | JCRB1170 |
| | Kasumi-1 | ATCC | CRL-2724 |
| | Kasumi-6 | ATCC | CRL-2775 |
| | MHH-CALL-3 | DSMZ | ACC-339 |
| | MHH-CALL-2 | DSMZ | ACC-341 |
| | JVM-2 | ATCC | CRL-3002 |
| | HNT-34 | DSMZ | ACC-600 |
| Bone | HOS | ATCC | CRL-1543 |
| | OUMS-27 | JCRB | IFO50488 |
| | T1-73 | ATCC | CRL-7943 |
| | Hs 870.T | ATCC | CRL-7606 |
| | Hs 706.T | ATCC | CRL-7447 |
| | SJSA-1 | ATCC | CRL-2098 |
| | RD-ES | ATCC | HTB-166 |
| | U2OS | ATCC | HTB-96 |
| | SaOS-2 | ATCC | HTB-85 |
| | SK-ES-1 | ATCC | HTB-86 |

In certain embodiments, the vaccine compositions comprising a combination of cell lines are capable of stimulating an immune response and/or preventing cancer and/or treating cancer. The present disclosure provides compositions and methods of using one or more vaccine compositions comprising therapeutically effective amounts of cell lines.

The amount (e.g., number) of cells from the various individual cell lines in a cocktail or vaccine compositions can be equal (as defined herein) or different. In various embodiments, the number of cells from a cell line or from each cell line (in the case where multiple cell lines are administered) in a vaccine composition, is approximately 1.0×10⁶, 2.0×10⁶, 3.0×10⁶, 4.0×10⁶, 5.0×10⁶, 6.0×10⁶, 7.0×10⁶, 8×10⁶, 9.0×10⁶, 1.0×10⁷, 2.0×10⁷, 3.0×10⁷, 4.0×10⁷, 5.0×10⁷, 6.0×10⁷, 8.0×10⁷, or 9.0×10⁷ cells.

The total number of cells administered to a subject, e.g., per administration site, can range from 1.0×10⁶ to 9.0×10⁷. For example, 2.0×10⁶, 3.0×10⁶, 4.0×10⁶, 5.0×10⁶, 6.0×10⁶, 7.0×10⁶, 8×10⁶, 9.0×10⁶, 1.0×10⁷, 2.0×10⁷, 3.0×10⁷, 4.0×10⁷, 5.0×10⁷, 6.0×10⁷, 8.0×10⁷, 8.6×10⁷, 8.8×10⁷, or 9.0×10⁷ cells are administered.

In certain embodiments, the number of cell lines included in each administration of the vaccine composition can range from 1 to 10 cell lines. In some embodiments, the number of cells from each cell line are not equal and different ratios of cell lines are used. For example, if one cocktail contains 5.0×10⁷ total cells from 3 different cell lines, there could be 3.33×10⁷ cells of one cell line and 8.33×10⁶ of the remaining 2 cell lines.

### HLA Diversity

HLA mismatch occurs when the subject's HLA molecules are different from those expressed by the cells of the administered vaccine compositions. The process of HLA matching involves characterizing 5 major HLA loci, which include the HLA alleles at three Class I loci HLA-A, —B and —C and two class II loci HLA-DRB1 and -DQB1. As every individual expresses two alleles at each loci, the degree of match or mismatch is calculated on a scale of 10, with 10/10 being a perfect match at all 10 alleles.

The response to mismatched HLA loci is mediated by both innate and adaptive cells of the immune system. Within the cells of the innate immune system, recognition of mismatches in HLA alleles is mediated to some extent by monocytes. Without being bound to any theory or mechanism, the sensing of “non-self” by monocytes triggers infiltration of monocyte-derived DCs, followed by their maturation, resulting in efficient antigen presentation to naïve T cells. Alloantigen-activated DCs produce increased amounts of IL-12 as compared to DCs activated by matched syngeneic antigens, and this increased IL-12 production results in the skewing of responses to Th1 T cells and increased IFN gamma production. HLA mismatch recognition by the adaptive immune system is driven to some extent by T cells. Without being bound to any theory or mechanism, 1-10% of all circulating T cells are alloreactive and respond to HLA molecules that are not present in self. This is several orders of magnitude greater than the frequency of endogenous T cells that are reactive to a conventional foreign antigen. The ability of the immune system to recognize these differences in HLA alleles and generate an immune response is a barrier to successful transplantation between donors and patients and has been viewed an obstacle in the development of cancer vaccines.

As many as 945 different HLA-A and -B alleles can be assigned to one of the nine supertypes based on the binding affinity of the HLA molecule to epitope anchor residues. In some embodiments, the vaccine compositions provided herein exhibit a heterogeneity of HLA supertypes, e.g., mixtures of HLA-A supertypes, and HLA-B supertypes. As described herein, various features and criteria may be considered to ensure the desired heterogeneity of the vaccine composition including, but not limited to, an individual's ethnicity (with regard to both cell donor and subject receiving the vaccine). Additional criteria are described in Example 25 of WO/2021/113328 and herein. In certain embodiments, a vaccine composition expresses a heterogeneity of HLA supertypes, wherein at least two different HLA-A and at least two HLA-B supertypes are represented.

In some embodiments, a composition comprising therapeutically effective amounts of multiple cell lines are provided to ensure a broad degree of HLA mismatch on multiple class I and class II HLA molecules between the tumor cell vaccine and the recipient.

In some embodiments, the vaccine composition expresses a heterogeneity of HLA supertypes, wherein the composition expresses a heterogeneity of major histocompatibility complex (MHC) molecules such that two of HLA-A24, HLA-A03, HLA-A01, and two of HLA-B07, HLA-B08, HLA-B27, and HLA-B44 supertypes are represented. In some embodiments, the vaccine composition expresses a heterogeneity HLA supertypes, wherein the composition expresses a heterogeneity of MHC molecules and at least the HLA-A24 is represented. In some exemplary embodiments, the composition expresses a heterogeneity of MHC molecules such that HLA-A24, HLA-A03, HLA-A01, HLA-B07, HLA-B27, and HLA-B44 supertypes are represented. In other exemplary embodiments, the composition expresses a genetic heterogeneity of MHC molecules such that HLA-A01, HLA-A03, HLA-B07, HLA-B08, and HLA-B44 supertypes are represented.

Patients display a wide breadth of HLA types that act as markers of self. A localized inflammatory response that promotes the release of cytokines, such as IFNγ and IL-2, is initiated upon encountering a non-self cell. In some embodiments, increasing the heterogeneity of HLA-supertypes within the vaccine cocktail has the potential to augment the localized inflammatory response when the vaccine is delivered conferring an adjuvant effect. As described herein, in some embodiments, increasing the breadth, magnitude, and immunogenicity of tumor reactive T cells primed by the cancer vaccine composition is accomplished by including multiple cell lines chosen to have mismatches in HLA types, chosen, for example, based on expression of certain TAAs. Embodiments of the vaccine compositions provided herein enable effective priming of a broad and effective anti-cancer response in the subject with the additional adjuvant effect generated by the HLA mismatch. Various embodiments of the cell line combinations in a vaccine composition express the HLA-A supertypes and HLA-B supertypes. Non-limiting examples are provided in Example 25 of WO/2021/113328 and herein.

### Cell Line Modifications

In certain embodiments, the vaccine compositions comprise cells that have been modified. Modified cell lines can be clonally derived from a single modified cell, i.e., genetically homogenous, or derived from a genetically heterogenous population.

Cell lines can be modified to express or increase expression (e.g., relative to an unmodified cell) of one or more immunostimulatory factors, to inhibit or decrease expression of one or more immunosuppressive factors (e.g., relative to an unmodified cell), and/or to express or increase expression of one or more TAAs (e.g., relative to an unmodified cell), including optionally TAAs that have been mutated in order to present neoepitopes (e.g., designed or enhanced antigens with NSMs) as described herein. Additionally, cell lines can be modified to express or increase expression of factors that can modulate pathways indirectly, such expression or inhibition of microRNAs. Further, cell lines can be modified to secrete non-endogenous or altered exosomes. As described herein, in some embodiments the cell lines are optionally additionally modified to express tumor fitness advantage mutations, including but not limited to acquired tyrosine kinase inhibitor (TKI) resistance mutations, EGFR activating mutations, and/or modified ALK intracellular domain(s), and/or driver mutations.

In addition to modifying cell lines to express a TAA or immunostimulatory factor, the present disclosure also contemplates co-administering one or more TAAs (e.g., an isolated TAA or purified and/or recombinant TAA) or immunostimulatory factors (e.g., recombinantly produced therapeutic protein) with the vaccines described herein.

Thus, in various embodiments, the present disclosure provides a unit dose of a vaccine comprising (i) a first composition comprising a therapeutically effective amount of at least 1, 2, 3, 4, 5 or 6 cancer cell lines, wherein the cell line or a combination of the cell lines comprises cells that express at least 5, 10, 15, 20, 25, 30, 35, or 40 tumor associated antigens (TAAs) associated with a cancer of a subject intended to receive said composition, and wherein the composition is capable of eliciting an immune response specific to the at least 5, 10, 15, 20, 25, 30, 35, or 40 TAAs, and (ii) a second composition comprising one or more isolated TAAs. In other embodiments, the first composition comprises a cell line or cell lines that is further modified to (a) express or increase expression of at least 1 immunostimulatory factor, and/or (ii) inhibit or decrease expression of at least 1 immunosuppressive factor.

### Mutations Providing a Fitness Advance to Tumor Cells

Cancers arise as a result of changes that have occurred in genome sequences of cells. Oncogenes as described in detail herein are genes that are involved in tumorigenesis. In tumor cells, oncogenes are often mutated and/or expressed at high levels. The term “driver mutations” as used herein, refers to somatic mutations that confer a growth advantage to the tumor cells carrying them and that have been positively selected during the evolution of the cancer. Driver mutations frequently represent a large fraction of the total mutations in oncogenes, and often dictate cancer phenotype.

As described herein, cancer vaccine platforms can, in some embodiments, be designed to target tumor associated antigens (TAAs) that are overexpressed in tumor cells. Neoepitopes are non-self epitopes generated from somatic mutations arising during tumor growth. The targeting of neoepitopes is a beneficial component of the cancer vaccine platform as described in various embodiments herein at least because neoepitopes are tumor specific and not subject to central tolerance in the thymus.

Based on the information on the number of alleles harboring the mutation and the fraction of tumor cells with the mutation, mutations can be classified as clonal (truncal mutations, present in all tumor cells sequenced) and subclonal (shared and private mutations, present in a subset of regions or cells within a single biopsy) (McGranahan N. et al., Sci. Trans. Med. 7(283): 283ra54, 2015). Unlike the majority of neoepitopes that are private mutations and not found in more than one patient, driver mutations in known driver genes typically occur early in the evolution of the cancer and are found in all or a subset of tumor cells across patients (Jamal-Hanjani, M. et al. Clin Cancer Res. 21(6), 1258-66, 2015). Driver mutations show a tendency to be clonal and give a fitness advantage to the tumor cells that carry them and are crucial for the tumor's transformation, growth and survival (Schumacher T., et al. Science 348:69-74, 2015). As described herein, targeting driver mutations is an effective strategy to overcome intra- and inter-tumor neoantigen heterogeneity and tumor escape. Inclusion of a pool of driver mutations that occur at high frequency in a vaccine can potentially promote potent anti-tumor immune responses.

Mutations that confer a tumor fitness advantage can also occur as the result of targeted therapies. For example, a subset of NSCLC tumors contain tumorigenic amplifications of EGFR or ALK that may be initially treatable with tyrosine kinase inhibitors. NSCLC tumors treated with tyrosine kinase inhibitors often develop mutations resulting in resistance to these therapies enabling tumor growth. (Ricordel, C. et al. Annals of Oncology. 29 (Supplement 1): i28-i37, 2018; Lin, J et al., Cancer Discovery, 7(2):137-155, 2017).

Table 4 describe exemplary tumor fitness advantage mutations that can provide a fitness advantage to solid tumors. Some exemplary mutations are specific the anatomical origin of the tumor, such as prostate cancer mutations in SPOP, while some exemplary mutations, such as some mutations in TP53, can provide a fitness advantage to tumors originating from more than one anatomical site.

TABLE 4
Exemplary mutations providing a fitness advantage to solid tumors
by mutated gene and indication

| Gene (Gene ID) | Mutation | Anantomical origin of the tumor |
|---|---|---|
| AR (367) | H875Y | Prostate |
| | L702H | Prostate |
| | W742C | Prostate |
| ATM (472) | R337C | Colorectal |
| CDH1 (999) | D254Y | Stomach |
| CDKN2A (1029) | H83Y | Pancreas |
| CTNNB1 (1499) | 545F | Colorectal |
| EGFR (1956) | A289D | Central Nervous System |
| | G598V | Central Nervous System |
| | G63R | Central Nervous System |
| | H304Y | Central Nervous System |
| | R108K | Central Nervous System |
| | R252C | Central Nervous System |
| | S645C | Central Nervous System |
| | V774M | Central Nervous System |
| EP300 (2033) | D1399N | Upper Aerodigestive Tract |
| ERBB2 (2064) | R678Q | Stomach |
| | S310F | Stomach, Bladder |
| | V842I | Stomach, Bladder |
| ERBB3 (2065) | D297Y | Stomach |
| | V104L | Bladder |
| | V104M | Stomach, Colorectal |
| ERBB4 (2066) | S1289A | Bladder |
| ERCC2 (2068) | E86Q | Bladder |
| | N2385 | Bladder |
| | S44L | Bladder |
| FBXW7 (55294) | R465H | Stomach, Colorectal |
| | R479Q | Stomach |
| | R505C | Colorectal |
| | R505G | Bladder |
| | S582L | Colorectal |
| FGFR3 (2261) | G370C | Bladder |
| | S249C | Bladder |
| | Y373C | Bladder |
| GNAS (2778) | R201H | Colorectal |
| HRAS (3265) | G13R | Bladder |
| | Q61R | Bladder |
| KRAS (3845) | A59T | Stomach |
| | G12A | Lung |
| | G12C | Pancreas, Colorectal |
| | G12D | Lung, Pancreas |
| | G12V | Lung, Pancreas |
| | G13C | Lung |
| | Q61R | Pancreas |
| PIK3CA (5290) | E542K | Stomach, Bladder, Colorectal, Breast, |
| | | Upper Aerodigestive Tract, Lung |
| | E726K | Bladder, Breast |
| | H1047L | Breast, Upper Aerodigestive Tract |
| | H1047R | Stomach, Bladder, Central Nervous |
| | | System, Lung |
| | H1047Y | Colorectal |
| | M1043I | Colorectal |
| | M1043V | Central Nervous System |
| | N345K | Stomach, Breast |
| | R88Q | Stomach, Bladder, Colorectal |
| PIK3R1 (5295) | G376R | Central Nervous System |
| PTEN (5728) | R130Q | Central Nervous System |
| | G132D | Central Nervous System |
| | R173H | Central Nervous System |
| RHOA1 (387) | L57V | Stomach |
| SMAD4 (4089) | R361H | Colorectal, Pancreas |
| SPOP (8405) | F102V | Prostate |
| | F133L | Prostate |
| | Y87C | Prostate |
| TP53 (7157) | C141Y | Lung |
| | C176F | Stomach, Lung |
| | C176Y | Ovaries |
| | C238Y | Ovaries, Pancreas |
| | C275Y | Central Nervous System, Ovaries |
| | E285K | Bladder |
| | G154V | Lung |
| | G245S | Stomach, Central Nervous System, |
| | | Colorectal, Upper Aerodigestive |
| | | Tract, Pancreas |
| | G245V | Central Nervous System |
| | G266R | Ovaries |
| | H179R | Central Nervous System |
| | H193L | Upper Aerodigestive Tract |
| | H193Y | Ovaries |
| | H214R | Pancreas, Lung |
| | I195T | Ovaries |
| | I251F | Lung |
| | K132N | Bladder |
| | L194R | Ovaries |
| | M237I | Stomach, Lung |
| | P278S | Upper Aerodigestive Tract |
| | R110L | Upper Aerodigestive Tract, Lung |
| | R158H | Central Nervous System |
| | R158L | Lung |
| | R175H | Stomach, Bladder, Central Nervous System, |
| | | Colorectal, Prostate, Pancreas, Lung |
| | R248W | Stomach, Bladder, Central Nervous System, |
| | | Colorectal, Breast, Ovaries, Upper |
| | | Aerodigestive Tract, Pancreas |
| | R249M | Lung |
| | R273C | Pancreas, Prostate, Colorectal, Bladder, |
| | | Stomach |
| | R273H | Central Nervous System, Breast, Upper |
| | | Aerodigestive Tract |
| | R273L | Ovaries, Lung |
| | R280K | Bladder |
| | R337L | Lung |
| | S241F | Bladder |
| | V157F | Ovaries, Upper Aerodigestive Tract, Lung |
| | V216M | Central Nervous System, Ovaries |
| | V272M | Ovaries |
| | Y163C | Ovaries, Upper Aerodigestive Tract |
| | Y220C | Stomach, Prostate, Breast, Ovaries, |
| | | Pancreas, Lung |
| | Y234C | Lung, Ovaries |

As described herein, one or more cell lines of the cancer vaccines are modified to express one or more peptides comprising one or more driver mutation sequences. The driver mutation modification design process is described in detail herein. In general, the design process includes identifying frequently mutated oncogenes for a given indication, identifying driver mutations in selected oncogenes, and selecting driver mutations to be engineered into a component of the vaccine platform based on, for example, the presence of CD4, CD8 or CD4 and CD8 epitopes. Additional steps may also be performed as provided herein.

“Frequently mutated oncogenes” as used herein can refer to, for example, oncogenes that contain more mutations relative to other known oncogenes in a set of patient tumor samples for a specific tumor type. Mutations in the oncogene may occur at the same amino acid position in multiple tumor samples. Some or all of the oncogene mutations may be private mutations and occur at different amino acid locations. The frequency of oncogene mutations varies based on the tumor mutational burden of the specific tumor type. Immunologically “cold” tumors in general tend to have fewer oncogenes with lower frequency of mutations, while immunologically “hot” tumors generally tend to have more oncogenes with greater frequency of mutations. Frequently mutated oncogenes may be similar for different tumor indications, such as TP53, or be indication specific, such as SPOP in prostate cancer. Among the 10 indications specifically described herein, the highest frequency of mutated oncogene is 69.7% (TP53, Ovarian). Oncogenes with lower than 5% mutation frequency are unlikely to possess an individual mutation occurring in greater than 0.5% of profiled patient tumor samples, and thus in one embodiment of the present disclosure, a mutation frequency of greater than or equal to 5% mutation is observed and selected. In various embodiments, a frequency of greater than or equal to 5, 10, 20, 30, 40, 50, 60, 70, 80, 90 or 100% mutation is provided.

A list of frequently mutated colorectal cancer oncogenes (>5%) is provided in Table 5.

TABLE 5
Frequently mutated oncogenes in colorectal cancer tumors

| Anatomical Site of Primary | |
|---|---|
| Tumor | NCBI Gene Symbol (Gene ID) |
| Colorectal | ACVR2A (92) |
| | AFDN (4301) |
| | ALK (238) |
| | AMER1 (139285) |
| | ANKRD11 (29123) |
| | APC (324) |
| | ARID1A (8289) |
| | ARID1B (57492) |
| | ARID2 (196528) |
| | ASXL1 (171023) |
| | ATM (472) |
| | ATRX (546) |
| | AXIN2 (8313) |
| | B2M (567) |
| | BCL9 (607) |
| | BCL9L (283149) |
| | BCORL1 (63035) |
| | BRAF (673) |
| | BRCA2 (675) |
| | CACNA1D (776) |
| | CAD (790) |
| | CAMTA1 (23261) |
| | CARD11 (84433) |
| | CHD4 (1108) |
| | CIC (23152) |
| | COL1A1 (1277) |
| | CREBBP (1387) |
| | CTNNB1 (1499) |
| | CUX1 (1523) |
| | DICER1 (23405) |
| | EP300 (2033) |
| | EP400 (57634) |
| | EPHA5 (2044) |
| | ERBB3 (2065) |
| | ERBB4 (2066) |
| | FAT1 (2195) |
| | FAT4 (79633) |
| | FBXW7 (55294) |
| | FLT4 (2324) |
| | GNAS (2778) |
| | GRIN2A (2903) |
| | IRS1 (3667) |
| | IRS4 (8471) |
| | KDM2B (84678) |
| | KMT2A (4297) |
| | KMT2B (9757) |
| | KMT2C (58508) |
| | KMT2D (8085) |
| | KRAS (3845) |
| | LARP4B (23185) |
| | LRP1B (53353) |
| | LRP5 (4041) |
| | LRRK2 (120892) |
| | MGA (23269) |
| | MKI67 (4288) |
| | MTOR (2475) |
| | MYH11 (4629) |
| | MYH9 (4627) |
| | MYO5A (4644) |
| | NCOR2 (9612) |
| | NF1 (4763) |
| | NOTCH1 (4851) |
| | NOTCH3 (4854) |
| | NUMA1 (4926) |
| | PCLO (27445) |
| | PDE4DIP (9659) |
| | PIK3CA (5290) |
| | PIK3CG (5294) |
| | PIK3R1 (5295) |
| | PLCG2 (5336) |
| | POLE (5426) |
| | POLQ (10721) |
| | PREX2 (80243) |
| | PRKDC (5591) |
| | PTEN (5728) |
| | PTPRC (5788) |
| | PTPRD (5789) |
| | PTPRK (5796) |
| | PTPRS (5802) |
| | PTPRT (11122) |
| | RANBP2 (5903) |
| | RELN (5649) |
| | RNF213 (57674) |
| | RNF43 (54894) |
| | ROBO1 (6091) |
| | ROS1 (6098) |
| | SETBP1 (26040) |
| | SETD1A (9739) |
| | SLX4 (84464) |
| | SMAD4 (4089) |
| | SMARCA4 (6597) |
| | SOX9 (6662) |
| | SPEN (23013) |
| | TCF7L2 (6934) |
| | TP53 (7157) |
| | TP53BP1 (7158) |
| | TRRAP (8295) |
| | UBR5 (51366) |
| | ZBTB20 (26137) |
| | ZFHX3 (463) |
| | ZNF521 (25925) |

Following identification of one or more frequently mutated oncogenes, driver mutations within the oncogenes are identified and selected. In various embodiments, driver mutations occurring in the same amino acid position in 0.5% of profiled patient tumor samples in each mutated oncogene are selected. In various embodiments, driver mutations occurring in the same amino acid position in >0.75, 1.0 or 1.5% of profiled patient tumor samples in each mutated oncogene are selected.

In various embodiments, the driver mutation is a missense (substitution), insertion, in-frame insertion, deletion, in-frame deletion, or gene amplification mutation. In various embodiments, one or more driver mutation sequences, once identified and prioritized as described herein, are inserted into a vector. In some embodiments, the vector is a lentiviral vector (lentivector).

In various embodiments of the present disclosure, a peptide sequence containing MHC class I and II epitopes and a given driver mutation that is 28-35 amino acid in length is generated to induce a potent driver mutation-specific immune response (e.g., cytotoxic and T helper cell responses). In some embodiments, a respective driver mutation is placed in the middle of a 28-35-mer peptide, flanked by roughly 15 aa on either side taken from the respective non-mutated, adjacent, natural human protein backbone. In some embodiments, when two (or more) driver mutations occur within 9 amino acids of a protein sequence, a long peptide sequence containing two (or more) driver mutations is also generated so long as there are at least 8 amino acids before and after each driver mutation. In various embodiments, up to 20 driver mutation-containing long peptides are assembled into one insert, separated by the furin and/or P2A cleavage site.

In some embodiments, the cell lines of the vaccine composition can be modified (e.g., genetically modified) to express, overexpress, or increase the expression of one or more peptides comprising one or more of the driver mutations in one or more of the oncogenes selected from Table 5. For example, at least one (i.e., 1, 2, 3, 4, 5, 6, 7, 8, 9, 10, or more) of the cancer cell lines in any of the vaccine compositions described herein may be genetically modified to express, overexpress, or increase the expression of one or more peptides comprising one or more of the driver mutations in one or more of the oncogenes selected from Table 5. The driver mutations expressed by the cells within the composition may all be the same, may all be different, or any combination thereof.

In some embodiments, a vaccine composition comprises a therapeutically effective amount of cells from at least one cancer cell line, wherein the at least one cell line is modified to express, overexpress, or increase the expression of one or more peptides comprising one or more of the driver mutations in one or more of the oncogenes selected from Table 5. In some embodiments, the composition comprises a therapeutically effective amount of cells from 2, 3, 4, 5, 6, 7, 8, 9, or 10 cancer cell lines.

In various embodiments, the cell line or cell lines modified to express, overexpress, or increase the expression of one or more peptides comprising one or more of the driver mutations in one or more of the oncogenes selected from Table 5 are colorectal cancer cell lines selected from the group consisting of HCT-15, RKO, HuTu-80, HCT-116, and LS411 N.

In some embodiments, a vaccine composition comprises a therapeutically effective amount of cells from at least one cancer cell line, wherein the at least one cell line is modified to express 1, 2, 3, 4, 5, 6, 7, 8, 9, 10, or more peptides comprising one or more driver mutation sequences. In some embodiments, the composition comprises a therapeutically effective amount of cells from 2, 3, 4, 5, 6, 7, 8, 9, or 10 cancer cell lines. In some embodiments, the at least one cell line is modified to increase the production of at least 2, 3, 4, 5, 6, 7, 8, 9 or 10 peptides comprising one or more driver mutation sequences.

In some embodiments, a driver mutation may satisfy the selection criteria described in the methods herein but is already present in a given cell or has been added to a cell line (e.g., via an added TAA) and are optionally included or optionally not included among the cell line modifications for a given vaccine.

### Immunostimulatory Factors

An immunostimulatory protein is one that is membrane bound, secreted, or both that enhances and/or increases the effectiveness of effector T cell responses and/or humoral immune responses. Without being bound to any theory, immunostimulatory factors can potentiate antitumor immunity and increase cancer vaccine immunogenicity. There are many factors that potentiate the immune response. For example, these factors may impact the antigen-presentation mechanism or the T cell mechanism. Insertion of the genes for these factors may enhance the responses to the vaccine composition by making the vaccine more immunostimulatory of anti-tumor response.

Without being bound to any theory or mechanism, expression of immunostimulatory factors by the combination of cell lines included in the vaccine in the vaccine microenvironment (VME) can modulate multiple facets of the adaptive immune response. Expression of secreted cytokines such as GM-CSF and IL-15 by the cell lines can induce the differentiation of monocytes, recruited to the inflammatory environment of the vaccine delivery site, into dendritic cells (DCs), thereby enriching the pool of antigen presenting cells in the VME. Expression of certain cytokines can also mature and activate DCs and Langerhans cells (LCs) already present. Expression of certain cytokines can promote DCs and LCs to prime T cells towards an effector phenotype. DCs that encounter vaccine cells expressing IL-12 in the VME should prime effector T cells in the draining lymph node and mount a more efficient anti-tumor response. In addition to enhancing DC maturation, engagement of certain immunostimulatory factors with their receptors on DCs can promote the priming of T cells with an effector phenotype while suppressing the priming of T regulatory cells (Tregs). Engagement of certain immunostimulatory factors with their receptors on DCs can promote migration of DCs and T cell mediated acquired immunity.

In some embodiments of the vaccine compositions provided herein, modifications to express the immunostimulatory factors are not made to certain cell lines or, in other embodiments, all of the cell lines present in the vaccine composition.

Provided herein are embodiments of vaccine compositions comprising a therapeutically effective amount of cells from at least one cancer cell line (e.g., colorectal cell line), wherein the cell line is modified to increase production of at least one (e.g., 2, 3, 4, 5, 6, 7, 8, 9, 10 or more) immunostimulatory factors. In some embodiments, the immunostimulatory factors are selected from those presented in Table 6. Also provided are exemplary NCBI Gene IDs that can be utilized by a skilled artisan to determine the sequences to be introduced in the vaccine compositions of the disclosure. These NCBI Gene IDs are exemplary only.

TABLE 6
Exemplary immunostimulatory factors

| Factor | NCBI Gene Symbol (Gene ID) |
|---|---|
| CCL5 | CCL5 (6352) |
| XCL1 | XCL1 (6375) |
| Soluble CD40L (CD154) | CD40LG (959) |
| Membrane-bound CD40L | CD40LG (959) |
| CD36 | CD36 (948) |
| GITR | TNFRSF18 (8784) |
| GM-CSF | CSF2 (1437) |
| OX-40 | TNFRSF4 (7293) |
| OX-40L | TNFSF4 (7292) |
| CD137 (41BB) | TNFRSF9 (13604) |
| CD80 (B7-1) | CD80 (941) |
| IFNγ | IFNG (3458) |
| IL-1β | IL1B (3553) |
| IL-2 | IL2 (3558) |
| IL-6 | IL6 (3569) |
| IL-7 | IL7 (3574) |
| IL-9 | IL9 (3578) |
| IL-12 | IL12A (3592) IL12B (3593) |
| IL-15 | IL15 (3600) |
| IL-18 | IL-18 (3606) |
| IL-21 | IL21 (59067) |
| IL-23 | IL23A (51561) IL12B (3593) |
| TNFα | TNF (7124) |

In some embodiments, the cell lines of the vaccine composition can be modified (e.g., genetically modified) to express, overexpress, or increase the expression of one or more immunostimulatory factors selected from Table 6. In certain embodiments, the immunostimulatory sequence can be a native human sequence. In some embodiments, the immunostimulatory sequence can be a genetically engineered sequence. The genetically engineered sequence may be modified to increase expression of the protein through codon optimization, or to modify the cellular location of the protein (e.g., through mutation of protease cleavage sites).

For example, at least one (i.e., 1, 2, 3, 4, 5, 6, 7, 8, 9, 10, or more) of the cancer cell lines in any of the vaccine compositions described herein may be genetically modified to express or increase expression of one or more immunostimulatory factors. The immunostimulatory factors expressed by the cells within the composition may all be the same, may all be different, or any combination thereof.

In some embodiments, a vaccine composition comprises a therapeutically effective amount of cells from at least one cancer cell line, wherein the at least one cell line is modified to express 1, 2, 3, 4, 5, 6, 7, 8, 9, 10, or more of the immunostimulatory factors of Table 6. In some embodiments, the composition comprises a therapeutically effective amount of cells from 2, 3, 4, 5, 6, 7, 8, 9, or 10 cancer cell lines. In some embodiments, the at least one cell line is modified to increase the production of at least 2, 3, 4, 5, 6, 7, 8, 9 or 10 immunostimulatory factors of Table 7. In some embodiments, the composition comprises a therapeutically effective amount of cells from 2, 3, 4, 5, 6, 7, 8, 9, or 10 cancer cell lines, and each cell line is modified to increase the production of at least 2, 3, 4, 5, 6, 7, 8, 9 or 10 immunostimulatory factors of Table 6.

In some embodiments, the composition comprises a therapeutically effective amount of cells from 3 cancer cells lines wherein 1, 2, or all 3 of the cell lines have been modified to express or increase expression of GM-CSF, membrane bound CD40L, and IL-12.

Exemplary combinations of modifications, e.g., where a cell line or cell lines have been modified to express or increase expression of more than one immunostimulatory factor include but are not limited to: GM-CSF+IL-12; CD40L+IL-12; GM-CSF+CD40L; GM-CSF+IL-12+CD40L; GM-CSF+IL-15; CD40L+IL-15; GM-CSF+CD40L; and GM-CSF+IL-15+CD40L, among other possible combinations.

In certain instances, tumor cells express immunostimulatory factors including the IL-12A (p35 component of IL-12), GM-CSF (kidney cell lines), and CD40L (leukemia cell lines). Thus, in some embodiments, cell lines may also be modified to increase expression of one or more immunostimulatory factors.

In some embodiments, the cell line combination of or cell lines that have been modified as described herein to express or increase expression of one or more immunostimulatory factors will express the immunostimulatory factor or factors at least 2, 3, 4, 5, 6, 7, 8, 9, 10-fold or more relative to the same cell line or combination of cell lines that have not been modified to express or increase expression of the one or more immunostimulatory factors.

Methods to increase immunostimulatory factors in the vaccine compositions described herein include, but are not limited to, introduction of the nucleotide sequence to be expressed by way of a viral vector or DNA plasmid. The expression or increase in expression of the immunostimulatory factors can be stable expression or transient expression.

In some embodiments, the cancer cells in any of the vaccine compositions described herein are genetically modified to express CD40 ligand (CD40L). In some embodiments, the CD40L is membrane bound. In some embodiments, the CD40L is not membrane bound. Unless stated otherwise, as used herein CD4L refers to membrane bound CD40L. In some embodiments, the cancer cells in any of the vaccine compositions described herein are genetically modified to express GM-CSF, membrane bound CD40L, GITR, IL-12, and/or IL-15. Exemplary amino acid and nucleotide sequences useful for expression of the one or more of the immunostimulatory factors provided herein are presented in Table 7.

TABLE 7
Sequences of exemplary immunostimulatory factors

| Factor | Sequence |
|---|---|
| CD154 (CD40L) | atgatcgaaacatacaaccaaacttctccccgatctgcggccactggactgcccatcagcatgaaaatttttatgtatttacttactgtttttcttatca |
| (membrane bound) | cccagatgattgggtcagcactttttgctgtgtatcttcatagaaggttggacaagatagaagatgaaaggaatcttcatgaagattttgtattcatg |
| (SEQ ID NO: 1) | aaaacgatacagagatgcaacacaggagaaagatccttatccttactgaactgtgaggagattaaaagccagtttgaaggctttgtgaaggat |
| | ataatgttaaacaaagaggagacgaagaaagaaaacagctttgaaatgcctcgtggtgaagaggatagtcaaattgcggcacatgtcataa |
| | gtgaggccagcagtaaaacaacatctgtgttacagtgggctgaaaaaggatactacaccatgagcaacaacttggtaaccctggaaaatgg |
| | gaaacagctgaccgttaaaagacaaggactctattatatctatgcccaagtcaccttctgttccaatcgggaagcttcgagtcaagctccatttat |
| | agccagcctctgcctaaagtcccccggtagattcgagagaatcttactcagagctgcaaatacccacagttccgccaaaccttgcgggcaac |
| | aatccattcacttgggaggagtatttgaattgcaaccaggtgcttcggtgtttgtcaatgtgactgatccaagccaagtgagccatggcactggctt |
| | cacgtctttggcttactcaaactctga |
| CD154 (CD40L) | Atgatcgaaacctacaaccagacctcaccacgaagtgccgccaccggactgcctattagtatgaaaatctttatgtacctgctgacagtgttcct |
| (membrane bound) | gatcacccagatgatcggctccgccctgtttgccgtgtacctgcaccggagactggacaagatcgaggatgagcggaacctgcacgaggact |
| (codon-optimized) | tcgtgtttatgaagaccatccagcggtgcaacacaggcgagagaagcctgtccctgctgaattgtgaggagatcaagagccagttcgagggc |
| (SEQ ID NO: 2) | tttgtgaaggacatcatgctgaacaaggaggagacaaagaaggagaacagcttcgagatgcccagaggcgaggaggattcccagatcgc |
| | cgcccacgtgatctctgaggccagctccaagaccacaagcgtgctgcagtgggccgagaagggctactataccatgtctaacaatctggtga |
| | cactggagaacggcaagcagctgaccgtgaagaggcagggcctgtactatatctatgcccaggtgacattctgcagcaatcgcgaggcctct |
| | agccaggccccctttatcgccagcctgtgcctgaagagccctggcaggttcgagcgcatcctgctgagagccgccaacacccactcctctgcc |
| | aagccatgcggacagcagtcaatccacctgggaggcgtgttcgagctgcagccaggagcaagcgtgttcgtgaatgtgactgacccatcac |
| | aggtgtctcacggcactggattcacatcatttggactgctgaaactgtga |
| CD154 (CD40L) | MIETYNQTSPRSAATGLPISMKIFMYLLTVFLITQMIGSALFAVYLHRRLDKIEDERNLHEDFVFMKTIQR |
| (membrane bound) | CNTGERSLSLLNCEEIKSQFEGFVKDIMLNKEETKKENSFEMPRGEEDSQIAAHVISEASSKTTSVLQ |
| (SEQ ID NO: 3) | WAEKGYYTMSNNLVTLENGKQLTVKRQGLYYIYAQVTFCSNREASSQAPFIASLCLKSPGRFERILLR |
| | AANTHSSAKPCGQQSIHLGGVFELQPGASVFVNVTDPSQVSHGTGFTSFGLLKL |
| GITR (SEQ ID NO: 4) | Atggctcagcatggggctatgggggccttcagggctctgtgcggactggctctgctgtgcgctctgtcactggggcagagaccaacaggagg |
| | accaggatgcggacctggcaggctgctgctgggcaccggcacagacgcaaggtgctgtagagtgcacaccacaaggtgctgtcgcgacta |
| | ccctggcgaggagtgctgttctgagtgggattgcatgtgcgtgcagccagagtttcactgtggcgatccctgctgtaccacatgccgccaccacc |
| | catgtccacctggacagggagtgcagtctcagggcaagttcagctttggcttccagtgcatcgactgtgcaagcggcaccttttccggaggaca |
| | cgagggacactgcaagccctggaccgattgtacacagtttggcttcctgaccgtgttccctggcaacaagacacacaatgccgtgtgcgtgcct |
| | ggctccccaccagcagagcccctgggctggctgaccgtggtgctgctggccgtggcagcatgcgtgctgctgctgacaagcgcccagctggg |
| | actgcacatctggcagctgcggtcccagtgtatgtggccaagagagacccagctgctgctggaggtgcctccatccacagaggacgcccggt |
| | cttgccagttccccgaagaggagaggggggaaagaagtgccgaagaaaagggaaggctgggagacctgtgggtg |
| GITR (SEQ ID NO: 5) | MAQHGAMGAFRALCGLALLCALSLGQRPTGGPGCGPGRLLLGTGTDARCCRVHTTRCCRDYPGEE |
| | CCSEWDCMCVQPEFHCGDPCCTTCRHHPCPPGQGVQSQGKFSFGFQCIDCASGTFSGGHEGHCK |
| | PINTDCTQFGFLTVFPGNKTHNAVCVPGSPPAEPLGWLTVVLLAVAACVLLLTSAQLGLHIWQLRSQC |
| | MWPRETQLLLEVPPSTEDARSCQFPEEERGERSAEEKGRLGDLINV |
| GM-CSF (SEQ ID NO: 6) | atgtggctgcagagcctgctgctcttgggcactgtggcctgcagcatctctgcacccgcccgctcgcccagccccagcacgcagccctgggag |
| | catgtgaatgccatccaggaggcccggcgtctcctgaacctgagtagagacactgctgctgagatgaatgaaacagtagaagtcatctcaga |
| | aatgtttgacctccaggagccgacctgcctacagacccgcctggagctgtacaagcagggcctgcggggcagcctcaccaagctcaaggg |
| | ccccttgaccatgatggccagccactacaagcagcactgccctccaaccccggaaacttcctgtgcaacccagattatcacctttgaaagtttc |
| | aaagagaacctgaaggactttctgcttgtcatcccctttgactgctgggagccagtccaggagtga |
| GM-CSF | atgtggctgcagtctctgctgctgctgggcaccgtcgcctgttctatttccgcacccgctcgctccccttctccctcaactcagccttgggagcacgt |
| (codon-optimized) | gaacgccatccaggaggcccggagactgctgaatctgtcccgggacaccgccgccgagatgaacgagacagtggaagtgatctctgagat |
| (SEQ ID NO: 7) | gttcgatctgcaggagcccacctgcctgcagacaaggctggagctgtacaagcagggcctgcgcggctctctgaccaagctgaagggccca |
| | ctgacaatgatggccagccactataagcagcactgcccccctacccccgagacaagctgtgccacccagatcatcacattcgagtcctttaag |
| | gagaacctgaaggactttctgctggtcattccatttgattgttgggagcccgtgcaggagtga |
| GM-CSF (SEQ ID NO: 8) | MWLQSLLLLGTVACSISAPARSPSPSTQPWEHVNAIQEARRLLNLSRDTAAEMNETVEVISEMFDLQE |
| | PTCLQTRLELYKQGLRGSLTKLKGPLTMMASHYKQHCPPTPETSCATQIITFESFKENLKDFLLVIPFD |
| | CWEPVQE |
| IL-12 (SEQ ID NO: 9) | atgtgccatcagcaactggttatatcttggttcagtctcgtctttctcgcgtcacccttggtcgctatctgggagcttaaaaaagatgtctacgtcgttg |
| | aacttgattggtaccctgatgctccgggggaaatggtggttttgacttgcgatacgccagaagaggatggcataacgtggacactggaccagtc |
| | ttcagaggttctcgggtctggtaagacactcactatacaggtgaaggagtttggtgacgcaggacaatatacttgccataaaggcggcgaggtg |
| | ctctcccatagccttctgctccttcataaaaaagaggacgggatatggtcaactgacattctgaaggatcagaaagaaccgaagaacaaaact |
| | ttcctcagatgcgaggcaaagaactattcaggccgctttacttgctggtggctcactaccatcagcactgacctcactttcagcgtcaagagcagt |
| | agaggctcaagtgacccacaaggggttacatgcggggccgctacgttgtctgccgagcgagtcaggggagataataaggaatatgagtata |
| | gcgttgaatgccaagaagattcagcctgcccagccgcagaagagagtcttcccatagaagttatggtggacgcagttcataaactgaagtatg |
| | agaactatacatcttccttctttattcgcgatatcataaagcctgatcctccgaaaaacttgcaactcaagccgttgaagaatagccgacaggtcg |
| | aggtctcttgggagtatccagatacgtggtctaccccgcactcctatttcagtctcaccttctgtgtgcaggtgcaggggaaaagtaagcgggaa |
| | aaaaaggaccgggtatttactgataagacctccgctacagtgatttgtagaaagaacgcctctatcagcgtgagagcccaggatagatattatt |
| | ctagtagttggtctgagtgggcctccgtcccttgttccggaagcggagccacgaacttctctctgttaaagcaagcaggagatgttgaagaaaac |
| | cccgggcctatgtgtccagcgcgcagcctcctccttgtggctaccctggtcctcctggaccacctcagtttggcccgaaacctgccggtcgctac |
| | acccgatcctggaatgtttccctgccttcatcacagccagaatctgctgagggcagtcagtaacatgctgcagaaggcgcggcaaactctgg a |
| | gttctatccatgtacctccgaggaaattgatcacgaggacattactaaggataaaacaagtacagtagaagcctgtttgcctcttgagctcacta |
| | aaaatgagtcatgcttgaacagtcgagagacgagttttatcactaacggttcatgcttggcgtccaggaagacaagctttatgatggcgctctgc |
| | ctgtcttctatatatgaagaccttaaaatgtaccaagttgagtttaagaccatgaacgccaaacttttgatggaccccaagaggcagatcttccttg |
| | atcagaatatgttggcggtgatcgatgaacttatgcaagctttgaacttcaacagtgagacagtgcctcagaaaagttccttggaggaaccgg a |
| | cttctataagaccaagatcaaactgtgcattttgctgcatgcatttagaattcgagccgttacaatcgaccgggtgatgtcatatttgaatgcatcat |
| | aa |
| IL-12 SEQ ID NO: 10) | MCHQQLVISWFSLVFLASPLVAIWELKKDVYVVELDWYPDAPGEMWLTCDTPEEDGITWTLDQSSE |
| | VLGSGKTLTIQVKEFGDAGQYTCHKGGEVLSHSLLLLHKKEDGIWSTDILKDQKEPKNKTFLRCEAKN |
| | YSGRFTCIMNLTTISTDLTFSVKSSRGSSDPQGVTCGAATLSAERVRGDKEYEYSVECQEDSACPA |
| | AEESLPIEVMVDAVHKLKYENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSWSEWASVPCSGSGATNFSL |
| | LKQAGDVEENPGPMCPARSLLLVATLVLLDHLSLARNLPVATPDPGMFPCLHHSQNLLRAVSNMLQK |
| | ARQTLEFYPCTSEEIDHEDITKDKTSTVEACLPLELTKNESCLNSRETSFITNGSCLASRKTSFMMALC |
| | LSSIYEDLKMYQVEFKTMNAKLLMDPKRQIFLDQNMLAVIDELMQALNFNSETVPQKSSLEEPDFYKT |
| | KIKLCILLHAFRIRAVTIDRVMSYLNAS |
| IL-15 (SEQ ID NO: 11) | atgtataggatgcagctgctgtcatgtatcgcactgtccctggcactggtgactaactctaactgggtgaatgtgatctccgacctgaagaagatc |
| | gaggacctgatccagtctatgcacatcgatgccaccctgtacacagagtccgacgtgcacccctcttgcaaggtgaccgccatgaagtgtttcc |
| | tgctggagctgcaggtcatcagcctggagagcggcgacgcatccatccacgataccgtggagaacctgatcatcctggccaacaatagcctg |
| | agctccaacggcaatgtgacagagtccggctgcaaggagtgtgaggagctggaggagaagaatatcaaagagttcctgcagtcattcgtcc |
| | atatcgtccagatgtttatcaataccagt |
| IL-15 (SEQ ID NO: 12) | MYRMQLLSCIALSLALVTNSNINVNVISDLKKIEDLIQSMHIDATLYTESDVHPSCKVTAMKCFLLELQVI |
| | SLESGDASIHDTVENLIILANNSLSSNGNVTESGCKECEELEEKNIKEFLQSFVHIVQMFINTS |
| IL-23 (SEQ ID NO: 13) | atgtgccatcagcagctggtcattagttggtttagcctggtctttctggcctcacccctggtcgcaatctgggaactgaagaaggacgtgtacgtgg |
| | tggagctggactggtatccagatgcaccaggagagatggtggtgctgacctgcgacacacctgaggaggatggcatcacctggacactgga |
| | tcagagctccgaggtgctgggcagcggcaagaccctgacaatccaggtgaaggagttcggcgacgccggccagtacacatgtcacaagg |
| | gcggcgaggtgctgtcccactctctgctgctgctgcacaagaaggaggacggcatctggtccacagacatcctgaaggatcagaaggagcc |
| | aaagaacaagaccttcctgcggtgcgaggccaagaattatagcggccggttcacctgttggtggctgaccacaatctccaccgatctgacattt |
| | tctgtgaagtctagcaggggctcctctgacccccagggagtgacatgcggagcagccaccctgagcgccgagcgggtgagaggcgataac |
| | aaggagtacgagtattctgtggagtgccaggaggacagcgcctgtccagcagcagaggagtccctgcctatcgaagtgatggtggatgccgt |
| | gcacaagctgaagtacgagaattatacaagctccttctttatcagggacatcatcaagccagatccccctaagaacctgcagctgaagcccct |
| | gaagaatagccgccaggtggaggtgtcctgggagtaccctgacacctggtccacaccacactcttatttcagcctgaccttttgcgtgcaggtgc |
| | agggcaagagcaagagggagaagaaggaccgcgtgttcaccgataagacatccgccaccgtgatctgtcggaagaacgccagcatctcc |
| | gtgagggcccaggatcgctactattctagctcctggagcgagtgggcctccgtgccatgctctggaggaggaggcagcggcggaggagg ct |
| | ccggaggcggcggctctggcggcggcggctccctgggctctcgggccgtgatgctgctgctgctgctgccctggaccgcacagggaagagc |
| | cgtgccaggaggctctagcccagcatggacacagtgccagcagctgtcccagaagctgtgcaccctggcatggtctgcccaccctctggtgg |
| | gccacatggacctgagagaggagggcgatgaggagaccacaaacgacgtgcctcacatccagtgcggcgacggctgtgatccacaggg |
| | cctgagggacaattctcagttctgtctgcagcgcatccaccagggcctgatcttctacgagaagctgctgggcagcgatatctttacaggagag |
| | cccagcctgctgcctgactccccagtgggacagctgcacgcctctctgctgggcctgagccagctgctgcagccagagggacaccactggg |
| | agacccagcagatcccttctctgagcccatcccagccttggcagcggctgctgctgcggttcaagatcctgagaagcctgcaggcattcgtcgc |
| | agtcgcagccagggtgttcgcccacggagccgctactctgagccca |
| IL-23 (SEQ ID NO: 14) | MCHQQLVISWFSLVFLASPLVAIWELKKDVYVVELDWYPDAPGEMWLTCDTPEEDGITWTLDQSSE |
| | VLGSGKTLTIQVKEFGDAGQYTCHKGGEVLSHSLLLLHKKEDGIWSTDILKDQKEPKNKTFLRCEAKN |
| | YSGRFTCIMNLTTISTDLTFSVKSSRGSSDPQGVTCGAATLSAERVRGDNKEYEYSVECQEDSACPA |
| | AEESLPIEVMVDAVHKLKYENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSWSEWASVPCSGGGGSGGG |
| | GSGGGGSGGGGSLGSRAVMLLLLLPWTAQGRAVPGGSSPAWTQCQQLSQKLCTLAWSAHPLVGH |
| | MDLREEGDEETTNDVPHIQCGDGCDPQGLRDNSQFCLQRIHQGLIFYEKLLGSDIFTGEPSLLPDSPV |
| | GQLHASLLGLSQLLQPEGHHWETQQIPSLSPSQPWQRLLLRFKILRSLQAFVAVAARVFAHGAATLSP |
| XCL1 (SEQ ID NO: 15) | atgaggctgctgattctggcactgctgggcatctgctctctgaccgcttacatcgtggaaggagtcggctctgaagtctctgacaagcgcacatg |
| | cgtgtctctgaccacacagcgcctgcccgtgagccggatcaagacctacacaatcaccgagggcagcctgagagccgtgatcttcatcacaa |
| | agaggggcctgaaggtgtgcgccgaccctcaggcaacctgggtgcgggacgtggtgagaagcatggataggaagtccaacacccggaac |
| | aatatgatccagacaaaacccacaggaacccagcagagcactaatacagccgtgacactgaccggg |
| XCL1 (SEQ ID NO: 16) | MRLLILALLGICSLTAYIVEGVGSEVSDKRTCVSLTTQRLPVSRIKTYTITEGSLRAVIFITKRGLKVCADP |
| | QATINVRDWRSMDRKSNTRNNMIQTKPTGTQQSTNTAVTLTG |

Provided herein is a GITR protein comprising the amino acid sequence of SEQ ID NO: 4, or a nucleic acid sequence encoding the same, e.g., SEQ ID NO: 5. Provided herein is a vaccine composition comprising one or more cell lines expressing the same. Provided herein is a GM-CSF protein comprising the amino acid sequence of SEQ ID NO: 8, or a nucleic acid sequence encoding the same, e.g., SEQ ID NO: 6 or SEQ ID NO: 7. Provided herein is a vaccine composition comprising one or more cell lines expressing the same. Provided herein is an IL-12 protein comprising the amino acid sequence of SEQ ID NO: 10, or a nucleic acid sequence encoding the same, e.g., SEQ ID NO: 9. Provided herein is a vaccine composition comprising one or more cell lines expressing the same. Provided herein is an IL-15 protein comprising the amino acid sequence of SEQ ID NO: 12, or a nucleic acid sequence encoding the same, e.g., SEQ ID NO: 11. Provided herein is a vaccine composition comprising one or more cell lines expressing the same. Provided herein is an IL-23 protein comprising the amino acid sequence of SEQ ID NO: 14, or a nucleic acid sequence encoding the same, e.g., SEQ ID NO: 13. Provided herein is a vaccine composition comprising one or more cell lines expressing the same. Provided herein is a XCL1 protein comprising the amino acid sequence of SEQ ID NO: 16, or a nucleic acid sequence encoding the same, e.g., SEQ ID NO: 15. Provided herein is a vaccine composition comprising one or more cell lines expressing the same.

In some embodiments, the cancer cells in any of the vaccine compositions described herein are genetically modified to express one or more of CD28, B7-H2 (ICOS LG), CD70, CX3CL1, CXCL10(IP10), CXCL9, LFA-1(ITGB2), SELP, ICAM-1, ICOS, CD40, CD27(TNFRSF7), TNFRSF14(HVEM), BTN3A1, BTN3A2, ENTPD1, GZMA, and PERF1.

In some embodiments, vectors contain polynucleotide sequences that encode immunostimulatory molecules. Exemplary immunostimulatory molecules may include any of a variety of cytokines. The term “cytokine” as used herein refers to a protein released by one cell population that acts on one or more other cells as an intercellular mediator. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1 through IL-36, including, IL-1, IL-1alpha, IL-2, IL-3, IL-7, IL-8, IL-9, IL-11, IL-12; IL-15, IL-18, IL-21, IL-23, IL-27, TNF; and other polypeptide factors including LIF and kit ligand (KL). Other immunomodulatory molecules contemplated for use herein include IRF3, B7.1, B7.2, 4-1 BB, CD40 ligand (CD40L), drug-inducible CD40 (iCD40), and the like.

In certain embodiments, polynucleotides encoding the immunostimulatory factors are under the control of one or more regulatory elements that direct the expression of the coding sequences. In various embodiments, more than one (i.e., 2, 3, or 4) immunostimulatory factors are encoded on one expression vector. In some embodiments, more than one (i.e., 2, 3, 4, 5, or 6) immunostimulatory factors are encoded on separate expression vectors. Lentivirus containing a gene or genes of interest (e.g., GM-CSF, CD40L, or IL-12 and other immunostimulatory molecules as described herein) are produced in various embodiments by transient co-transfection of 293T cells with lentiviral transfer vectors and packaging plasmids (OriGene) using LipoD293TM In

### Vitro DNA Transfection Reagent (SignaGen Laboratories).

For lentivirus infection, in some embodiments, cell lines are seeded in a well plate (e.g., 6-well, 12-well) at a density of 1-10×10⁵ cells per well to achieve 50-80% cell confluency on the day of infection. Eighteen—24 hours after seeding, cells are infected with lentiviruses in the presence of 10 μg/mL of polybrene. Eighteen—24 hours after lentivirus infection, cells are detached and transferred to larger vessel. After 24-120 hours, medium is removed and replaced with fresh medium supplemented with antibiotics.

### Immunosuppressive Factors

An immunosuppressive factor is a protein that is membrane bound, secreted, or both and capable of contributing to defective and reduced cellular responses. Various immunosuppressive factors have been characterized in the context of the tumor microenvironment (TME). In addition, certain immunosuppressive factors can negatively regulate migration of LCs and DCs from the dermis to the draining lymph node.

TGFβ1 is a suppressive cytokine that exerts its effects on multiple immune cell subsets in the periphery as well as in the TME. In the VME, TGFβ1 negatively regulates migration of LCs and DCs from the dermis to the draining lymph node. Similarly, TGFβ2 is secreted by most tumor cells and exerts immunosuppressive effects similar to TGFβ1. Modification of the vaccine cell lines to reduce TGFβ1 and/or TGFβ2 secretion in the VME ensures the vaccine does not further TGFβ-mediated suppression of LC or DC migration.

Within the TME, CD47 expression is increased on tumor cells as a mode of tumor escape by preventing macrophage phagocytosis and tumor clearance. DCs also express SIRPα, and ligation of SIRPα on DCs can suppress DC survival and activation. Additional immunosuppressive factors in the vaccine that could play a role in the TME and VME include CD276 (B7-H3) and CTLA4. DC contact with a tumor cell expressing CD276 or CTLA4 in the TME dampens DC stimulatory capabilities resulting in decreased T cell priming, proliferation, and/or promotes proliferation of T cells. Expression of CTLA4 and/or CD276 on the vaccine cell lines could confer the similar suppressive effects on DCs or LCs in the VME.

In certain embodiments of the vaccine compositions, production of one or more immunosuppressive factors can be inhibited or decreased in the cells of the cell lines contained therein. In some embodiments, production (i.e., expression) of one or more immunosuppressive factors is inhibited (i.e., knocked out or completely eliminated) in the cells of the cell lines contained in the vaccine compositions. In some embodiments, the cell lines can be genetically modified to decrease (i.e., reduce) or inhibit expression of the immunosuppressive factors. In some embodiments, the immunosuppressive factor is excised from the cells completely. In some embodiments, one or more of the cell lines are modified such that one or more immunosuppressive factor is produced (i.e., expressed) at levels decreased or reduced (e.g., relative to an unmodified cell) by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, the one or more immunosuppressive factors is selected from the group presented in Table 8.

Simultaneously, production of one or more immunostimulatory factors, TAAs, and/or neoantigens can be increased in the vaccine compositions as described herein. In some embodiments of the vaccine compositions, in addition to the partial reduction or complete (e.g., excision and/or expression at undetectable levels) inhibition of expression of one or more immunosuppressive factors by the cell, one or more (i.e., 1, 2, 3, 4, 5, 6, 7, 8, 9, 10, or more) of the cell types within the compositions also can be genetically modified to increase the immunogenicity of the vaccine, e.g., by ensuring the expression of certain immunostimulatory factors, and/or TAAs.

Any combinations of these actions, modifications, and/or factors can be used to generate the vaccine compositions described herein. By way of non-limiting example, the combination of decreasing or reducing expression of immunosuppressive factors by at least 5, 10, 15, 20, 25, or 30% and increasing expression of immunostimulatory factors at least 2-fold higher than an unmodified cell line may be effective to improve the anti-tumor response of tumor cell vaccines. By way of another non-limiting example, the combination of reducing immunosuppressive factors by at least 5, 10, 15, 20, 25, or 30% and modifying cells to express certain TAAs in the vaccine composition, may be effective to improve the anti-tumor response of tumor cell vaccines.

In some embodiments, a cancer vaccine comprises a therapeutically effective amount of cells from at least one cancer cell line, wherein the cell line is modified to reduce production of at least one immunosuppressive factor by the cell line, and wherein the at least one immunosuppressive factor is CD47 or CD276. In some embodiments, expression of CTLA4, HLA-E, HLA-G, TGFβ1, and/or TGFβ2 are also reduced. In some embodiments, one or more, or all, cell lines in a vaccine composition are modified to inhibit or reduce expression of CD276, TGFβ1, and TGFβ2. In another embodiment, a vaccine composition is provided comprising three cell lines that have each been modified to inhibit (e.g., knockout) expression of CD276, and reduce expression of (e.g., knockdown) TGFβ1 and TGFβ2.

In some embodiments, a cancer vaccine composition comprises a therapeutically effective amount of cells from a cancer cell line wherein the cell line is modified to reduce expression of at least CD47. In some embodiments, the CD47 is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, CD47 is excised from the cells or is produced at levels reduced by at least 90%. Production of additional immunosuppressive factors can be reduced in one or more cell lines. In some embodiments, expression of CD276, CTLA4, HLA-E, HLA-G, TGFβ1, and/or TGFβ2 are also reduced or inhibited. Production of one or more immunostimulatory factors, TAAs, or neoantigens can be increased in one or more cell lines in these vaccine compositions.

In some embodiments, provided herein is a cancer vaccine composition comprising a therapeutically effective amount of cells from a cancer cell line wherein the cell line is modified to reduce production of at least CD276. In some embodiments, the CD276 is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, CD276 is excised from the cells or is produced at levels reduced by at least 90%. Production of additional immunosuppressive factors can be reduced in one or more cell lines. In some embodiments, expression of CD47, CTLA4, HLA-E, HLA-G, TGFβ1, and/or TGFβ2 are also reduced or inhibited. Production of one or more immunostimulatory factors, TAAs, or neoantigens can be increased in one or more cell lines in these vaccine compositions.

In some embodiments, provided herein is a cancer vaccine composition comprising a therapeutically effective amount of cells from a cancer cell line wherein the cell line is modified to reduce production of at least HLA-G. In some embodiments, the HLA-G is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, HLA-G is excised from the cells or is produced at levels reduced by at least 90%. Production of additional immunosuppressive factors can be reduced in one or more cell lines. In some embodiments, expression of CD47, CD276, CTLA4, HLA-E, TGFβ1, and/or TGFβ2 are also reduced or inhibited. Production of one or more immunostimulatory factors, TAAs, or neoantigens can be increased in one or more cell lines in these vaccine compositions.

In some embodiments, provided herein is a cancer vaccine composition comprising a therapeutically effective amount of cells from a cancer cell line wherein the cell line is modified to reduce production of at least CTLA4. In some embodiments, the CTLA4 is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, CTLA4 is excised from the cells or is produced at levels reduced by at least 90%. Production of additional immunosuppressive factors can be reduced in one or more cell lines. In some embodiments, expression of CD47, CD276, HLA-E, TGFβ1, and/or TGFβ2 are also reduced or inhibited. Production of one or more immunostimulatory factors, TAAs, or neoantigens can be increased in one or more cell lines in these vaccine compositions.

In some embodiments, provided herein is a cancer vaccine composition comprising a therapeutically effective amount of cells from a cancer cell line wherein the cell line is modified to reduce production of at least HLA-E. In some embodiments, the HLA-E is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments, HLA-E is excised from the cells or is produced at levels reduced by at least 90%. Production of additional immunosuppressive factors can be reduced in one or more cell lines. In some embodiments, expression of CD47, CD276, CTLA4, TGFβ1, and/or TGFβ2 are also reduced or inhibited. Production of one or more immunostimulatory factors, TAAs, or neoantigens can be increased in one or more cell lines in these vaccine compositions.

In some embodiments, provided herein is a cancer vaccine composition comprising a therapeutically effective amount of cells from a cancer cell line wherein the cell line is modified to reduce production of TGFβ1, TGFβ2, or both TGFβ1 and TGFβ2. In some embodiments, TGFβ1, TGFβ2, or both TGFβ1 and TGFβ2 is excised from the cells or is produced at levels reduced by at least 5, 10, 15, 20, 25, or 30% (i.e., at least 5, 10, 15, 20, 25, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%). In some embodiments of the vaccine composition, TGFβ1, TGFβ2, or both TGFβ1 and TGFβ2 is excised from the cells or is produced at levels reduced by at least 90%.

In some embodiments, TGFβ1, TGFβ2, or both TGFβ1 and TGFβ2 expression is reduced via a short hairpin RNA (shRNA) delivered to the cells using a lentiviral vector. Production of additional immunosuppressive factors can be reduced. In some embodiments, expression of CD47, CD276, CTLA4, HLA-E, and/or HLA-G are also reduced in one or more cell lines where TGFβ1, TGFβ2, or both TGFβ1 and TGFβ2 expression is reduced. Production of one or more immunostimulatory factors, TAAs, or neoantigens can also be increased in one or more cell lines in embodiments of these vaccine compositions.

In some embodiments, the immunosuppressive factor selected for knockdown or knockout may be encoded by multiple native sequence variants. Accordingly, the reduction or inhibition of immunosuppressive factors can be accomplished using multiple gene editing/knockdown approaches known to those skilled in the art. As described herein, in some embodiments, complete knockout of one or more immunosuppressive factors may be less desirable than knockdown. For example, TGFβ1 contributes to the regulation of the epithelial-mesenchymal transition, so complete lack of TGFβ1 (e.g., via knockout) may induce a less immunogenic phenotype in tumor cells.

Table 8 provides exemplary immunosuppressive factors that can be incorporated or modified as described herein, and combinations of the same. Also provided are exemplary NCBI Gene IDs that can be utilized for a skilled artisan to determine the sequence to be targeted for knockdown strategies. These NCBI Gene IDs are exemplary only.

TABLE 8
Exemplary immunosuppressive factors

| | NCBI Gene Symbol |
|---|---|
| Factor | (Gene ID) |
| B7-H3 (CD276) | CD276 (80381) |
| BST2 (CD317) | BST2 (684) |
| CD200 | CD200 (4345) |
| CD39 (ENTPD1) | ENTPD1 (953) |
| CD47 | CD47 (961) |
| CD73 (NT5E) | NT5E (4907) |
| COX-2 | PTGS2 (5743) |
| CTLA4 | CTLA4 (1493) |
| HLA-E | HLA-E (3133) |
| HLA-G | HLA-G (3135) |
| IDO (indoleamine 2,3-dioxygenase) | IDO1 (3620) |
| IL-10 | IL10 (3586) |
| PD-L1 (CD274) | CD274 (29126) |
| TGFβ1 | TGFB1 (7040) |
| TGFβ2 | TGFB2 (7042) |
| TGFβ3 | TGFB3 (7043) |
| VISTA (VSIR) | VSIR (64115) |
| M-CSF | CSF1 (1435) |
| B751 (B7H4) | VTCN1 (79679) |
| PTPN2 | PTPN2 (5771) |

In exemplary embodiments, the production of the following combination of immunosuppressive factors is reduced or inhibited in the vaccine composition: CD47+TGFβ1, CD47+TGFβ2, or CD47+TGFβ1+TGFβ2. In exemplary embodiments, the production of the following combination of immunosuppressive factors is reduced or inhibited in the vaccine composition: CD276+TGFβ1, CD276+TGFβ2, or CD276+TGFβ1+TGFβ2. In exemplary embodiments, the production of the following combination of immunosuppressive factors is reduced or inhibited in the vaccine composition: CD47+TGFβ1+CD276, CD47+TGFβ2+CD276, or CD47+TGFβ1+TGFβ2+CD276. In exemplary embodiments, the production of the following combination of immunosuppressive factors is reduced or inhibited in the vaccine composition: CD47+TGFβ1+B7-H3, CD47+TGFβ2+CD276, or CD47+TGFβ1+TGFβ2+CD276. In exemplary embodiments, the production of the following combination of immunosuppressive factors is reduced or inhibited in the vaccine composition: CD47+TGFβ1+CD276+BST2, CD47+TGFβ2+CD276+BST2, or CD47+TGFβ1+TGFβ2+CD276+BST2. In exemplary embodiments, the production of the following combination of immunosuppressive factors is reduced or inhibited in the vaccine composition: CD47+TGFβ1+CD276+CTLA4, CD47+TGFβ2+CD276+CTLA4, or CD47+TGFβ1+TGFβ2+CD276+CTLA4. In exemplary embodiments, the production of the following combination of immunosuppressive factors is reduced or inhibited in the vaccine composition: CD47+TGFβ1+CD276+CTLA4, CD47+TGFβ2+CD276+CTLA4, or CD47+TGFβ1+TGFβ2+CD276+CTLA4.

In exemplary embodiments, the production of the following combination of immunosuppressive factors is reduced or inhibited in the vaccine composition: CD47+TGFβ1+CD276+CTLA4, CD47+TGFβ2+CD276+CTLA4, or CD47+TGFβ1+TGFβ2+CD276+CTLA4, CD47+TGFβ2 or TGFβ1+CTLA4, or CD47+TGFβ1+TGFβ2+CD276+HLA-E or CD47+TGFβ1+TGFβ2+CD276+HLA-G, or CD47+TGFβ1+TGFβ2+CD276+HLA-G+CTLA-4, or CD47+TGFβ1+TGFβ2+CD276+HLA-E+CTLA-4.

In still other embodiments, the production of the following combination of immunosuppressive factors is reduced or inhibited in the vaccine composition: TGFβ1+TGFβ2+CD276, TGFβ1+CD276, or TGFβ2+CD276.

Those skilled in the art will recognize that in embodiments of the vaccine compositions described herein, at least one (i.e., 1, 2, 3, 4, 5, 6, 7, 8, 9, 10, or more) of the cell lines within the composition has a knockdown or knockout of at least one immunosuppressive factor (e.g., one or more of the factors listed in Table 8). The cell lines within the composition may have a knockdown or knockout of the same immunosuppressive factor, or a different immunosuppressive factor for each cell line, or of some combination thereof.

Optionally, 1, 2, 3, 4, 5, 6, 7, 8, 9, 10, or more of the cell lines within the composition may be further genetically modified to have a knockdown or knockout of one or more additional immunosuppressive factors (e.g., one or more of the factors listed in Table 8). For example, 1, 2, 3, 4, 5, 6, 7, 8, 9, 10, or more of the cell lines within the composition may be further genetically modified to have a knockdown or knockout of the same additional immunosuppressive factor, of a different additional immunosuppressive factor for each cell line, or of some combination thereof.

In some embodiments, provided herein is a cancer vaccine composition comprising a therapeutically effective amount of cells from a cancer cell line wherein the cell line is modified to reduce production of SLAMF7, BTLA, EDNRB, TIGIT, KIR2DL1, KIR2DL2, KIR2DL3, TIM3(HAVCR2), LAG3, ADORA2A and ARG1.

At least one of the cells within any of the vaccine compositions described herein may undergo one or more (i.e., 1, 2, 3, 4, 5, 6, 7, 8, 9, 10 or more) genetic modifications in order to achieve the partial or complete knockdown of immunosuppressive factor(s) described herein and/or the expression (or increased expression) of immunostimulatory factors described herein, TAAs, and/or neoantigens. In some embodiments, at least one cell line in the vaccine composition undergoes less than 5 (i.e., less than 4, less than 3, less than 2, 1, or 0) genetic modifications. In some embodiments, at least one cell in the vaccine composition undergoes no less than 5 genetic modifications.

Numerous methods of reducing or inhibiting expression of one or more immunosuppressive factors are known and available to those of ordinary skill in the art, embodiments of which are described herein.

Cancer cell lines are modified according to some embodiments to inhibit or reduce production of immunosuppressive factors. Provided herein are methods and techniques for selection of the appropriate technique(s) to be employed in order to inhibit production of an immunosuppressive factor and/or to reduce production of an immunosuppressive factor. Partial inhibition or reduction of the expression levels of an immunosuppressive factor may be accomplished using techniques known in the art.

In some embodiments, the cells of the cancer lines are genetically engineered in vitro using recombinant DNA techniques to introduce the genetic constructs into the cells. These DNA techniques include, but are not limited to, transduction (e.g., using viral vectors) or transfection procedures (e.g., using plasmids, cosmids, yeast artificial chromosomes (YACs), electroporation, liposomes). Any suitable method(s) known in the art to partially (e.g., reduce expression levels by at least 5, 10, 15, 20, 25, or 30%) or completely inhibit any immunosuppressive factor production by the cells can be employed.

In some embodiments, genome editing is used to inhibit or reduce production of an immunosuppressive factor by the cells in the vaccine. Non-limiting examples of genome editing techniques include meganucleases, zinc finger nucleases (ZFNs), transcription activator-like effector-based nucleases (TALEN), and the CRISPR-Cas system. In certain embodiments, the reduction of gene expression and subsequently of biological active protein expression can be achieved by insertion/deletion of nucleotides via non-homologous end joining (NHEJ) or the insertion of appropriate donor cassettes via homology directed repair (HDR) that lead to premature stop codons and the expression of non-functional proteins or by insertion of nucleotides.

In some embodiments, spontaneous site-specific homologous recombination techniques that may or may not include the Cre-Lox and FLP-FRT recombination systems are used. In some embodiments, methods applying transposons that integrate appropriate donor cassettes into genomic DNA with higher frequency, but with little site/gene-specificity are used in combination with required selection and identification techniques. Non-limiting examples are the piggyBac and Sleeping Beauty transposon systems that use TTAA and TA nucleotide sequences for integration, respectively.

Furthermore, combinatorial approaches of gene editing methods consisting of meganucleases and transposons can be used.

In certain embodiments, techniques for inhibition or reduction of immunosuppressive factor expression may include using antisense or ribozyme approaches to reduce or inhibit translation of mRNA transcripts of an immunosuppressive factor; triple helix approaches to inhibit transcription of the gene of an immunosuppressive factor; or targeted homologous recombination.

Antisense approaches involve the design of oligonucleotides (either DNA or RNA) that are complementary to mRNA of an immunosuppressive factor. The antisense oligonucleotides bind to the complementary mRNA transcripts of an immunosuppressive factor and prevent translation. Absolute complementarity may be preferred but is not required. A sequence “complementary” to a portion of an RNA, as referred to herein, means a sequence having sufficient complementarity to be able to hybridize with the RNA, forming a stable duplex. In the case of double-stranded antisense nucleic acids, a single strand of the duplex DNA may be tested, or triplex formation may be assayed. The ability to hybridize depends on both the degree of complementarity and the length of the antisense nucleic acid. In some embodiments, oligonucleotides complementary to either the 5′ or 3′-non-translated, non-coding regions of an immunosuppressive factor could be used in an antisense approach to inhibit translation of endogenous mRNA of an immunosuppressive factor. In some embodiments, inhibition or reduction of an immunosuppressive factor is carried out using an antisense oligonucleotide sequence within a short-hairpin RNA.

In some embodiments, lentivirus-mediated shRNA interference is used to silence the gene expressing the immunosuppressive factor. (See Wei et al., J. Immunother. 2012 35(3)267-275 (2012), incorporated by reference herein.)

MicroRNAs (miRNA) are stably expressed RNAi hairpins that may also be used for knocking down gene expression. In some embodiments, ribozyme molecules-designed to catalytically cleave mRNA transcripts are used to prevent translation of an immunosuppressive factor mRNA and expression. In certain embodiments, ribozymes that cleave mRNA at site specific recognition sequences can be used to destroy mRNAs. In some embodiments, the use of hammerhead ribozymes that cleave mRNAs at locations dictated by flanking regions that form complementary base pairs with the target mRNA are used. RNA endoribonucleases can also be used.

In some embodiments, endogenous gene expression of an immunosuppressive factor is reduced by inactivating or “knocking out” the gene or its promoter, for example, by using targeted homologous recombination. The percent reduction could, in some embodiments, be 100% (e.g., complete reduction). In other embodiments, the percent reduction is 90% or more. In some embodiments, endogenous gene expression is reduced by targeting deoxyribonucleotide sequences complementary to the regulatory region of the promoter and/or enhancer genes of an immunosuppressive factor to form triple helical structures that prevent transcription of the immunosuppressive factor gene in target cells. In some embodiments, promoter activity is inhibited by a nuclease dead version of Cas9 (dCas9) and its fusions with KRAB, VP64 and p65 that cannot cleave target DNA. The dCas9 molecule retains the ability to bind to target DNA based on the targeting sequence. This targeting of dCas9 to transcriptional start sites is sufficient to reduce or knockdown transcription by blocking transcription initiation.

In some embodiments, the activity of an immunosuppressive factor is reduced using a “dominant negative” approach in which genetic constructs that encode defective immunosuppressive factors are used to diminish the immunosuppressive activity on neighboring cells.

In some embodiments, the administration of genetic constructs encoding soluble peptides, proteins, fusion proteins, or antibodies that bind to and “neutralize” intracellularly any other immunosuppressive factors are used. To this end, genetic constructs encoding peptides corresponding to domains of immunosuppressive factor receptors, deletion mutants of immunosuppressive factor receptors, or either of these immunosuppressive factor receptor domains or mutants fused to another polypeptide (e.g., an IgFc polypeptide) can be utilized. In some embodiments, genetic constructs encoding anti-idiotypic antibodies or Fab fragments of anti-idiotypic antibodies that mimic the immunosuppressive factor receptors and neutralize the immunosuppressive factor are used. Genetic constructs encoding these immunosuppressive factor receptor peptides, proteins, fusion proteins, anti-idiotypic antibodies or Fabs can be administered to neutralize the immunosuppressive factor.

Likewise, genetic constructs encoding antibodies that specifically recognize one or more epitopes of an immunosuppressive factor, or epitopes of conserved variants of an immunosuppressive factor, or peptide fragments of an immunosuppressive factor can also be used. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, and epitope binding fragments of any of the above. Any technique(s) known in the art can be used to produce genetic constructs encoding suitable antibodies.

In some embodiments, the enzymes that cleave an immunosuppressive factor precursor to the active isoforms are inhibited to block activation of the immunosuppressive factor. Transcription or translation of these enzymes may be blocked by a means known in the art.

In further embodiments, pharmacological inhibitors can be used to reduce enzyme activities including, but not limited to COX-2 and IDO to reduce the amounts of certain immunosuppressive factors.

### Tumor Associated Antigens (TAAs)

Vector-based and protein-based vaccine approaches are limited in the number of TAAs that can be targeted in a single formulation. In contrast, embodiments of the allogenic whole cell vaccine platform as described herein allow for the targeting of numerous, diverse TAAs. The breadth of responses can be expanded and/or optimized by selecting allogenic cell line(s) that express a range of TAAs and optionally genetically modifying the cell lines to express additional antigens, including neoantigens or nonsynonymous mutations (NSMs), of interest for a desired therapeutic target (e.g., cancer type).

As used herein, the term “TAA” refers to tumor-associated antigen(s) and can refer to “wildtype” antigens as naturally expressed from a tumor cell or can optionally refer to a mutant antigen, e.g., a design antigen or designed antigen or enhanced antigen or engineered antigen, comprising one or more mutations such as a neoepitope or one or more NSMs as described herein.

TAAs are proteins that can be expressed in normal tissue and tumor tissue, but the expression of the TAA protein is significantly higher in tumor tissue relative to healthy tissue. TAAs may include cancer testis antigens (CTs), which are important for embryonic development but restricted to expression in male germ cells in healthy adults. CTs are often expressed in tumor cells.

Neoantigens or neoepitopes are aberrantly mutated genes expressed in cancer cells. In many cases, a neoantigen can be considered a TAA because it is expressed by tumor tissue and not by normal tissue. Targeting neoepitopes has many advantages since these neoepitopes are truly tumor specific and not subject to central tolerance in thymus. A cancer vaccine encoding full length TAAs with neoepitopes arising from nonsynonymous mutations (NSMs) has potential to elicit a more potent immune response with improved breadth and magnitude.

As used herein, a nonsynonymous mutation (NSM) is a nucleotide mutation that alters the amino acid sequence of a protein. In some embodiments, a missense mutation is a change in one amino acid in a protein, arising from a point mutation in a single nucleotide. A missense mutation is a type of nonsynonymous substitution in a DNA sequence. Additional mutations are also contemplated, including but limited to truncations, frameshifts, or any other mutation that change the amino acid sequence to be different than the native antigen protein.

As described herein, in some embodiments, an antigen is designed by (i) referencing one or more publicly-available databases to identify NSMs in a selected TAA; (ii) identiifying NSMs that occur in greater than 2 patients; (iii) introducing each NSM identified in step (ii) into the related TAA sequence; (iv) identifying HLA-A and HLA-B supertype-restricted MHC class I epitopes in the TAA that now includes the NSM; and and (v) including the NSMs that create new epitopes (SB and/or WB) or increases peptide-MHC affinity into a final TAA sequence. Exemplary NSMs predicted to create HLA-A and HLA-B supertype-restricted neoepitopes have been described in Example 40 of PCT/US2020/062840 (Pub. No. WO/2021/113328) and is incorporated by reference herein.

In some embodiments, an NSM identified in one patient tumor sample is included in the designed antigen (i.e., the mutant antigen arising from the introduction of the one or more NSMs). In various embodiments, 1, 2, 3, 4, 5, 6, 7, 8, 9, 10, 11, 12, 13, 14, 15, 16, 17, 18, 19, 20 or more NSMs are introduced into a TAA to generate the designed antigen. In some embodiments, target antigens could have a lower number NSMs and may need to use NSMs occurring only 1 time to reach the targeted homology to native antigen protein range (94-97%). In other embodiments, target antigens could have a high number of NSMs occurring at the >2 occurrence cut-off and may need to use NSMs occurring 3 times to reach the targeted homology to native antigen protein range (94-97%). Including a high number NSMs in the designed antigen would decrease the homology of the designed antigen to the native antigen below the target homology range (94-98%).

In some embodiments, 1, 2, 3, 4, 5 or 6 cell lines of a tumor cell vaccine according to the present disclosure comprise 1, 2, 3, 4, 5, 6, 7, 8, 9, 10, 11, 12, 13, 14, 15, 16, 17, 18, 19, 20 or more NSMs (and thus 1, 2, 3, 4, 5, 6, 7, 8, 9, 10, 11, 12, 13, 14, 15, 16, 17, 18, 19, 20 or more designed antigens) in at least one TAA.

In various embodiments, the sequence homology of the mutant (e.g., designed antigen) to the native full-length protein is 80%, 81%, 82%, 83%, 84%, 85%, 86%, 87%, 88%, 89%, 90%, 91%, 92%, 93%, 94%, 95%, 96%, 97%, 98%, 99% over the full length of the antigen.

In some embodiments, the designed antigen is incorporated into a therapeutic allogenic whole cell cancer vaccine to induce antigen-specific immune responses to the designed TAAs and existing TAAs.

In some embodiments, the vaccine can be comprised of a therapeutically effective amount of at least one cancer cell line, wherein the cell line or the combination of the cell lines express at least one designed TAA. In other embodiments, the vaccine comprises a therapeutically effective amount of at least one cancer cell line, wherein the cell line or the combination of the cell lines expresses at least 2, 3, 4, 5, 6, 7, 8, 9 10 or more designed TAAs.

Provided herein are embodiments of vaccine compositions comprising a therapeutically effective amount of cells from at least one cancer cell line, wherein the at least one cancer cell line expresses (either natively, or is designed to express) one or more TAAs, neoantigens (including TAAs comprising one or more NSMs), CTs, and/or TAAs. In some embodiments, the cells are transduced with a recombinant lentivector encoding one or more TAAs, including TAAs comprising one or more NSMs, to be expressed by the cells in the vaccine composition.

In some embodiments, the TAAs, including TAAs comprising one or more NSMs or neoepitopes, and/or other antigens may endogenously be expressed on the cells selected for inclusion in the vaccine composition. In some embodiments, the cell lines may be modified (e.g., genetically modified) to express selected TAAs, including TAAs comprising one or more NSMs, and/or other antigens (e.g., CTs, TSAs, neoantigens).

Any of the tumor cell vaccine compositions described herein may present one or more TAAs, including TAAs comprising one or more NSMs or neoepitopes, and induce a broad antitumor response in the subject. Ensuring such a heterogeneous immune response may obviate some issues, such as antigen escape, that are commonly associated with certain cancer monotherapies.

According to various embodiments of the vaccine composition provided herein, at least one cell line of the vaccine composition may be modified to express one or more neoantigens, e.g., neoantigens implicated in colorectal cancer. In some embodiments, one or more of the cell lines expresses an unmutated portion of a neoantigen protein. In some embodiments, one or more of the cell lines expresses a mutated portion of a neoantigen protein.

In some embodiments, at least one of the cancer cells in any of the vaccine compositions described herein may naturally express, or be modified to express one or more TAAs, including TAAs comprising one or more NSMs, CTs, or TSAs/neoantigens. In certain embodiments, more than one (e.g., 2, 3, 4, 5, 6, 7, 8, 9, 10, or more) of the cancer cell lines in the vaccine composition may express, or may be genetically modified to express, one or more of the TAAs, including TAAs comprising one or more NSMs, CTs, or TSAs/neoantigens. The TAAs, including TAAs comprising one or more NSMs, CTs, or TSAs/neoantigens expressed by the cell lines within the composition may all be the same, may all be different, or any combination thereof.

Because the vaccine compositions may contain multiple (i.e., 2, 3, 4, 5, 6, 7, 8, 9, 10, or more) cancer cell lines of different types and histology, a wide range and variety of TAAs, including TAAs comprising one or more NSMs, and/or neoantigens may be present in the composition (Table 9-25). The number of TAAs that can be targeted using a combination of cell lines (e.g., 5-cell line combination, 6-cell line combination, 7-cell line combination, 8-cell line combination, 9-cell line combination, or 10-cell line combination) and expression levels of the TAAs is higher for the cell line combination compared to individual cell lines in the combination.

In embodiments of the vaccine compositions provided herein, at least one (i.e., 1, 2, 3, 4, 5, 6, 7, 8, 9, 10, or more) of the cancer cells in any of the vaccine compositions described herein may express, or be modified to express one or more TAAs, including TAAs comprising one or more NSMs or neoepitopes. The TAAs, including TAAs comprising one or more NSMs, expressed by the cells within the composition may all be the same, may all be different, or any combination thereof. Table 9 below lists exemplary non-small cell lung cancer TAAs, and exemplary subsets of lung cancer TAAs. In some embodiments, the TAAs are specific to colorectal cancer.

In some embodiments, presented herein is a vaccine composition comprising a therapeutically effective amount of engineered cells from least one cancer cell line, wherein the cell lines or combination of cell lines express at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more of the TAAs in Table 9. In other embodiments, the TAAs in Table 9 are modified to include one or more NSM as described herein.

In some embodiments, a vaccine composition is provided comprising a therapeutically effective amount of engineered cells from at least one cancer cell line, wherein the cell lines express at least 2, 3, 4, 5, 6, 7, 8, 9, 10 of the TAAs in Table 9 (or the TAAs in Table 9 that have been modified to include one or more NSM). As provided herein, in various embodiments the cell lines express at least 2, 3, 4, 5, 6, 7, 8, 9, 10 of the TAAs in Table 9 (or the TAAs in Table 9 that have been modified to include one or more NSM) and are optionally modified to express or increase expression of one or more immunostimulatory factors of Table 6, and/or inhibit or decrease expression of one or more immunosuppressive factors in Table 8.

TABLE 9
Exemplary TAAs expressed in colorectal cancer

| TAA Name | NCBI Gene Symbol (Gene ID) |
|---|---|
| Survivin | BIRC5 (332) |
| B-RAF | BRAF (673) |
| CEA | CEACAM5 (1048) |
| βHCG | CGB3 (1082) |
| NYESO1 | CTAG1B (1485) |
| EPCAM | EPCAM (4072) |
| EPH receptor A2 | EPHA2 (1969) |
| Her2 | ERBB2 (2064) |
| GUCY2C | GUCY2C (2984) |
| PSMA | FOLH1 (2346) |
| KRAS | KRAS (3845) |
| MAGE-A1 | MAGEA1 (4100) |
| MAGE-A3 | MAGEA3 (4102) |
| MAGE-A4 | MAGEA4 (4103) |
| MAGE-A6 | MAGEA6 (4105) |
| Mesothelin | MSLN (10232) |
| MUC1 | MUC1 (4582) |
| FRAME | PRAME (23532) |
| CD133 | PROM1 (8842) |
| RNF43 | RNF43 (54894) |
| SART3 | SART3 (9733) |
| STEAP1 | STEAP1 (26872) |
| Brachyury/TBXT | T (6862) |
| TROP2 | TACSTD2 (4070) |
| hTERT | TERT (7015) |
| TOMM34 | TOMM34 (10953) |
| 5T4 | TPBG (7162) |
| WT1 | WT1 (7490) |

In some embodiments of the vaccine compositions provided herein, 1, 2, 3, 4, 5, 6, 7, 8, 9, 10, or more of the cell lines within the composition may be genetically modified to express or increase expression of the same immunostimulatory factor, TAA, including TAAs comprising one or more NSMs, and/or neoantigen; of a different immunostimulatory factor, TAA, and/or neoantigen; or some combination thereof. In some embodiments, the TAA sequence can be the native, endogenous, human TAA sequence. In some embodiments, the TAA sequence can be a genetically engineered sequence of the native endogenous, human TAA sequence. The genetically engineered sequence may be modified to increase expression of the TAA through codon optimization or the genetically engineered sequence may be modified to change the cellular location of the TAA (e.g., through mutation of protease cleavage sites).

Exemplary NCBI Gene IDs are presented in Table 9. As provided herein, these Gene IDs can be used to express (or overexpress) certain TAAs in one or more cell lines of the vaccine compositions of the disclosure.

In various embodiments, one or more of the cell lines in a composition described herein is modified to express mesothelin (MSLN), CT83 (kita-kyushu lung cancer antigen 1) TERT, PSMA, MAGEA1, EGFRvIII, hCMV pp65, TBXT, BORIS, FSHR, MAGEA10, MAGEC2, WT1, FBP, TDGF1, Claudin 18, LY6K, PRAME, HPV16/18 E6/E7, FAP, or mutated versions thereof (Table 10). The phrase “or mutated versions thereof” refers to sequences of the TAAs provided herein, that comprise one or more mutations (e.g., 1, 2, 3, 4, 5, 6, 7, 8, 9, or 10 or more substitution mutations), including neopepitopes or NSMs, as described herein. Thus, in various embodiments, one or more of the cell lines in a composition described herein is modified to express modMesothelin (modMSLN), modTERT, modPSMA, modMAGEA1, EGFRvIII, hCMV pp65, modTBXT, modBORIS, modFSHR, modMAGEA10, modMAGEC2, modWT1, modFBP, modTDGF1, modClaudin 18, modLY6K, modFAP, modPRAME, KRAS G12D mutation, KRAS G12V mutation, and/or HPV16/18 E6/E7. In other embodiments, the TAA or “mutated version thereof” may comprise fusions of 1, 2, or 3 or more of the TAAs or mutated versions provided herein. In some embodiments, the fusions comprise a native or wild-type sequence fused with a mutated TAA. In some embodiments, the individual TAAs in the fusion construct are separated by a cleavage site, such as a furin cleavage site. The present disclosure provides TAA fusion proteins such as, for example, modMAGEA1-EGFRvIll-pp65, modTBXT-modBORIS, modFSHR-modMAGEA10, modTBXT-modMAGEC2, modTBXT-modWT1, modTBXT-modWT1 (KRAS), modWT1-modFBP, modPSMA-modTDGF1, modWT1-modClaudin 18, modPSMA-modLY6K, modFAP-modClaudin 18, and modPRAME-modTBXT. Sequences for native TAAs can be readily obtained from the NCBI database (www.ncbi.nlm.nih.gov/protein). Sequences for some of the TAAs provided herein, mutated versions, and fusions thereof are provided in Table 10.

TABLE 10
Sequences of Exemplary Designed Antigens

| TAA | Sequence |
|---|---|
| modTBXT_modWT1_ | agagtctgagctgtggctgcggttcaaagaactgaccaacgagatgatcgtgaccaagaacggcagacggatgttccccgtgctgaaa |
| (KRAS Mutations) | gtgaacgtgtccggactggaccccaacgccatgtacagctttctgctggacttcgtggtggccgacaaccacagatggaaatacgtgaac |
| (SEQ ID NO: 17) | ggcgagtgggtgccaggcggaaaacctcaactgcaagcccctagctgcgtgtacattcaccctgacagccccaatttcggcgcccactg |
| | gatgaaggcccctgtgtccttcagcaaagtgaagctgaccaacaagctgaacggcggaggccagatcatgctgaacagcctgcacaa |
| | atacgagcccagaatccacatcgtcagagtcggcggaccccagagaatgatcaccagccactgcttccccgagacacagtttatcgcc |
| | gtgaccgcctaccagaacgaggaaatcaccacactgaagatcaagtacaaccccttcgccaaggccttcctggacgccaaagagcg |
| | gagcgaccacaaagagatgatcaaagagcccggcgacagccagcagccaggctattctcaatggggatggctgctgccaggcacca |
| | gcacattgtgccctccagccaatcctcacagccagtttggaggcgccctgagcctgtctagcacccacagctacgacagataccccaca |
| | ctgcggagccacagaagcagcccctatccttctccttacgctcaccggaacaacagccccacctacagcgataatagccccgcctgtct |
| | gagcatgctgcagtcccacgataactggtccagcctgagaatgcctgctcacccttccatgctgcccgtgtctcacaatgcctctccaccta |
| | ccagcagctctcagtaccctagcctttggagcgtgtccaatggcgccgtgacactgggatctcaggcagccgctgtgtctaatggactggg |
| | agcccagttcttcagaggcagccctgctcactacacccctctgacacatcctgtgtctgcccctagcagcagcggcttccctatgtataagg |
| | gcgctgccgccgctaccgacatcgtggattctcagtatgatgccgccgcacagggacacctgatcgcctcttggacacctgtgtctccacc |
| | ttccatgagaggcagaaagcggagaagcgacttcctgctgctgcagaaccctgcctctacctgtgtgcctgaaccagcctctcagcacac |
| | cctgagatctggccctggatgtctccagcagcctgaacagcagggcgttagagatcctggcggaatctgggccaaactgggagctgccg |
| | aagcctctgccgaatgtctgcagggcagaagaagcagaggcgccagcggatctgaacctcaccagatgggaagcgacgtgcacga |
| | cctgaatgctctgttgcctgccgtgccatctcttggcggaggcggaggatgtgctttgcctgtttctggtgctgcccagtgggctcccgtgctgg |
| | attttgctcctcctggcgcttctgcctatggctctcttggaggacctgctcctccaccagctccacctccaccgccgcctccaccacctcacag |
| | ctttatcaagcaagagccctcctggggcggagccgagcctcacgaaaaacagtgtctgagcgccttcaccgtgcactttttcggccagttt |
| | accggcaccgtgggcgcctgtagatacggcccttttggaccaccaccacctagccaggcttctagcggacaggccagaatgttccccaa |
| | cgctccttacctgcctagctgcctggaaagccagcctaccatcagaaaccagggcttcagcaccgtgaccttcgacggcatgcctagcta |
| | tggccacacaccatctcaccacgccgctcagttccccaatcacagcttcaagcacgaggaccctatgggccagcagggatctctggga |
| | gagcagcagtatagcgtgccacctcctgtgtacggctgtcacacccctaccgatagctgcacaggcaatcaggctctgctgctgaggatg |
| | cctttcagcagcgacaacctgtaccagatgacaagccagctggaatgcatgatttggaaccagatgaacctgggcgccactctgaaag |
| | gcgtggccgctggatctagcagctccgtgaaatggacagccggccagagcaatcactccaccggctacgagagcgacaatcacacc |
| | atgcctatcctgtgtggggcccagtaccggattcacacacacggcgtgttcaggggcattcaggatgtgcgaagagtgcctggcgtggcc |
| | cctacacttgtgggatctgccagcgaaaccagcgagaagcaccccttcatgtgcgcctatccaggctgcaacaagcggtacttcaagct |
| | gagccacctgaagatgcacagccggaagcacacaggcgagaagctgtaccagtgcgacttcaaggactgcgagcggagattcagct |
| | gcagcgaccagctgaagagacaccagagaaggcacaccggcgtgaagccctttcagtgcaagacctgccagcggaccttctcctggt |
| | ccaaccacctgaaaacccacacaagaacccacaccggcaagaccatcgagaagcccttcagctgtagatggcccagctgccagaa |
| | gaagttcgcccggtctaacgagctggtgcatcaccacaacatgcaccagaggaacatgaccaaactgcagctggtgctgaggggaag |
| | aaagaggcggtccaccgagtacaagctggtggttgttggagccgatggcgtgggaaagagcgccctgacaattcagctgatccagaac |
| | cacttcgtgcgcggcagaaagagaagatctacagagtataagctcgtggtcgtgggcgctgtcggagtgggaaaatctgccctgaccat |
| | ccaactcattcagaatcactttgtgtgatga |
| modTBXT_modWT1_ | MSSPGTESAGKSLQYRVDHLLSAVENELQAGSEKGDPTEHELRVGLEESELWLRFKELTNEMIVT |
| (KRAS Mutations) | KNGRRMFPVLKVNVSGLDPNAMYSFLLDFVVADNHRWKYVNGEINVPGGKPQLQAPSCVYIHPD |
| (SEQ ID NO: 18) | SPNFGAHWMKAPVSFSKVKLTNKLNGGGQIMLNSLHKYEPRIHIVRVGGPQRMITSHCFPETQFIA |
| | VTAYQNEEITTLKIKYNPFAKAFLDAKERSDHKEMIKEPGDSQQPGYSQWGWLLPGTSTLCPPAN |
| | PHSQFGGALSLSSTHSYDRYPTLRSHRSSPYPSPYAHRNNSPTYSDNSPACLSMLQSHDNWSSL |
| | RMPAHPSMLPVSHNASPPTSSSQYPSLWSVSNGAVTLGSQAAAVSNGLGAQFFRGSPAHYTPL |
| | THPVSAPSSSGFPMYKGAAAATDIVDSQYDAAAQGHLIASWTPVSPPSMRGRKRRSDFLLLQNP |
| | ASTCVPEPASQHTLRSGPGCLQQPEQQGVRDPGGIWAKLGAAEASAECLQGRRSRGASGSEPH |
| | QMGSDVHDLNALLPAVPSLGGGGGCALPVSGAAQWAPVLDFAPPGASAYGSLGGPAPPPAPPP |
| | PPPPPPHSFIKQEPSWGGAEPHEKQCLSAFTVHFFGQFTGTVGACRYGPFGPPPPSQASSGQA |
| | RMFPNAPYLPSCLESQPTIRNQGFSTVTFDGMPSYGHTPSHHAAQFPNHSFKHEDPMGQQGSL |
| | GEQQYSVPPPVYGCHTPTDSCTGNQALLLRMPFSSDNLYQMTSQLECMIWNQMNLGATLKGVA |
| | AGSSSSVKWTAGQSNHSTGYESDNHTMPILCGAQYRIHTHGVFRGIQDVRRVPGVAPTLVGSAS |
| | ETSEKHPFMCAYPGCNKRYFKLSHLKMHSRKHTGEKLYQCDFKDCERRFSCSDQLKRHQRRHT |
| | GVKPFQCKTCQRTFSWSNHLKTHTRTHTGKTIEKPFSCRWPSCQKKFARSNELVHHHNMHQRN |
| | MTKLQLVLRGRKRRSTEYKLVVVGADGVGKSALTIQLIQNHFVRGRKRRSTEYKLWVGAVGVGK |
| | SALTIQLIQNHFV |
| modPSMA | atgtggaatctgctgcacgagacagatagcgccgtggctaccgttagaaggcccagatggctttgtgctggcgctctggttctggctggcg |
| (SEQ ID NO: 19) | gcttttttctgctgggcttcctgttcggctggttcatcaagagcagcaacgaggccaccaacatcacccctaagcacaacatgaaggccttt |
| | ctggacgagctgaaggccgagaatatcaagaagttcctgtacaacttcacgcacatccctcacctggccggcaccgagcagaattttca |
| | gctggccaagcagatccagagccagtggaaagagttcggcctggactctgtggaactggcccactacgatgtgctgctgagctacccca |
| | acaagacacaccccaactacatcagcatcatcaacgaggacggcaacgagatcttcaacaccagcctgttcgagcctccacctcctgg |
| | ctacgagaacgtgtccgatatcgtgcctccattcagcgctttcagcccacagcggatgcctgagggctacctggtgtacgtgaactacgcc |
| | agaaccgaggacttcttcaagctggaatgggacatgaagatcagctgcagcggcaagatcgtgatcgcccggtacagaaaggtgttcc |
| | gcgagaacaaagtgaagaacgcccagctggcaggcgccaaaggcgtgatcctgtatagcgaccccgccgactattttgcccctggcgt |
| | gaagtcttaccccgacggctggaattttcctggcggcggagtgcagcggcggaacatccttaatcttaacggcgctggcgaccctctgac |
| | acctggctatcctgccaatgagtacgcctacagacacggaattgccgaggctgtgggcctgccttctattcctgtgcaccctgtgcggtact |
| | acgacgcccagaaactgctggaaaagatgggcggaagcgcccctcctgactcttcttggagaggctctctgaaggtgccctacaatgtc |
| | ggcccaggcttcaccggcaacttcagcacccagaaagtgaaaatgcacatccacagcaccaacgaagtgacccggatctacaacgt |
| | gatcggcacactgagaggcgccgtggaacccgacaaatacgtgatcctcggcggccacagagacagctgggtgttcggaggaatcg |
| | accctcaatctggcgccgctgtggtgtatgagatcgtgcggtctttcggcaccctgaagaaagaaggatggcggcccagacggaccatc |
| | ctgtttgcctcttgggacgccgaggaatttggcctgctgggatctacagagtgggccgaagagaacagcagactgctgcaagaaagag |
| | gcgtggcctacatcaacgccgacagcagcatcgagggcaactacaccctgcggatcgattgcacccctctgatgtacagcctggtgcac |
| | aacctgaccaaagagctgaagtcccctgacgagggctttgagggcaagagcctgtacaagagctggaccaagaagtccccatctcctg |
| | agttcagcggcatgcccagaatctctaagctggaaagcggcaacaacttcgaggtgttcttccagcggctgggaatcgcctctggaatcg |
| | ccagatacaccaagaactgggagacaaacaagttctccggctatcccctgtaccacagcgtgtacgagacatacgagctggtggaaa |
| | agttctacgaccccatgttcaagtaccacctgacagtggcccaagtgcgcggaggcatggtgttcgaactggccaatagcatcgtgctgc |
| | ccttcaactgcagagactacgccgtggtgctgcggaagtacgccgacaagatctacagcatcagcatgaagcacccgcaagagatga |
| | agacctacagcgtgtccttcgactccctgttcttcgccgtgaagaacttcaccaagatcgccagcaagttcagcgagcggctgcaggactt |
| | cgacaagagcaaccctatcgtgctgaggatgatgaacgaccagctgatgttcctggaacgggccttcatcaaccctctgggactgcccg |
| | acagacccttctacaggcacgtgatctgtgcccctagcagccacaacaaatacgccggcgagagcttccccggcatctacgatgccctg |
| | ttcgacatcgagagcaacgtgaaccctagcaaggcctggggcgaagtgaagagacagatctacgtggccgcattcacagtgcaggcc |
| | gctgccgaaacactgtctgaggtggcctgatga |
| modPSMA | MWNLLHETDSAVATVRRPRWLCAGALVLAGGFFLLGFLFGWFIKSSNEATNITPKHNMKAFLDEL |
| (SEQ ID NO: 20) | KAENIKKFLYNFTHIPHLAGTEQNFQLAKQIQSQWKEFGLDSVELAHYDVLLSYPNKTHPNYISIINE |
| | DGNEIFNTSLFEPPPPGYENVSDIVPPFSAFSPQRMPEGYLVYVNYARTEDFFKLEWDMKISCSG |
| | KIVIARYRKVFRENKVKNAQLAGAKGVILYSDPADYFAPGVKSYPDGWNFPGGGVQRRNILNLNG |
| | AGDPLTPGYPANEYAYRHGIAEAVGLPSIPVHPVRYYDAQKLLEKMGGSAPPDSSWRGSLKVPY |
| | NVGPGFTGNFSTQKVKMHIHSTNEVTRIYNVIGTLRGAVEPDKYVILGGHRDSWVFGGIDPQSGA |
| | AWYEIVRSFGTLKKEGWRPRRTILFASWDAEEFGLLGSTEWAEENSRLLQERGVAYINADSSIEG |
| | NYTLRIDCTPLMYSLVHNLTKELKSPDEGFEGKSLYKSWTKKSPSPEFSGMPRISKLESGNNFEVF |
| | FQRLGIASGIARYTKNWETNKFSGYPLYHSVYETYELVEKFYDPMFKYHLTVAQVRGGMVFELAN |
| | SIVLPFNCRDYAWLRKYADKIYSISMKHPQEMKTYSVSFDSLFFAVKNFTKIASKFSERLQDFDKS |
| | NPIVLRMMNDQLMFLERAFINPLGLPDRPFYRHVICAPSSHNKYAGESFPGIYDALFDIESNVNPSK |
| | AWGEVKRQIYVAAFTVQAAAETLSEVA |
| KRAS G12D mutation | accgagtacaagctggtggttgttggagccgatggcgtgggaaagagcgccctgacaattcagctgatccagaaccacttcgtg |
| (SEQ ID NO: 21) | |
| KRAS G12D mutation | TEYKLWVGADGVGKSALTIQLIQNHFV |
| (SEQ ID NO: 22) | |
| KRAS G12V mutation | acagagtataagctcgtggtcgtgggcgctgtcggagtgggaaaatctgccctgaccatccaactcattcagaatcactttgtg |
| (SEQ ID NO: 23) | |
| KRAS G12V mutation | TEYKLWVGAVGVGKSALTIQLIQNHFV |
| (SEQ ID NO: 24) | |

In some embodiments, provided herein is a vaccine composition comprising a therapeutically effective amount of cells from at least two cancer cell lines, wherein each cell line or a combination of the cell lines expresses at least 1, 2, 3, 4, 5, 6, 7, 8, 9, or 10 of the TAAs of Table 9. In other embodiments, the TAAs in Table 9 are modified to include one or more NSMs as described herein. In some embodiments, at least one cell line is modified to increase production of at least 1, 2, or 3 immunostimulatory factors, e.g., immunostimulatory factors from Table 6. In some embodiments, a vaccine composition is provided comprising a therapeutically effective amount of the cells from at least one cancer cell line, wherein each cell line or combination of cell lines is modified to reduce at least 1, 2, or 3 immunosuppressive factors, e.g., immunosuppressive factors from Table 8. In some embodiments, a vaccine composition is provided comprising two cocktails, wherein each cocktail comprises three cell lines modified to express 1, 2, or 3 immunostimulatory factors and to inhibit or reduce expression of 1, 2, or 3 immunosuppressive factors, and wherein each cell line expresses at least 10 TAAs or TAAs comprising one or more NSMs.

Methods and assays for determining the presence or expression level of a TAA in a cell line according to the disclosure or in a tumor from a subject are known in the art. By way of example, Warburg-Christian method, Lowry Assay, Bradford Assay, spectrometry methods such as high performance liquid chromatography (HPLC), liquid chromatography-mass spectrometry (LC/MS), immunoblotting and antibody-based techniques such as western blot, ELISA, immunoelectrophoresis, protein immunoprecipitation, flow cytometry, and protein immunostaining are all contemplated by the present disclosure.

The antigen repertoire displayed by a patient's tumor can be evaluated in some embodiments in a biopsy specimen using next generation sequencing and antibody-based approaches. Similarly, in some embodiments, the antigen repertoire of potential metastatic lesions can be evaluated using the same techniques to determine antigens expressed by circulating tumor cells (CTCs). Assessment of antigen expression in tumor biopsies and CTCs can be representative of a subset of antigens expressed. In some embodiments, a subset of the antigens expressed by a patient's primary tumor and/or CTCs are identified and, as described herein, informs the selection of cell lines to be included in the vaccine composition in order to provide the best possible match to the antigens expressed in a patient's tumor and/or metastatic lesions.

Embodiments of the present disclosure provides compositions of cell lines that (i) are modified as described herein and (ii) express a sufficient number and amount of TAAs such that, when administered to a patient afflicted with a cancer, cancers, or cancerous tumor(s), a TAA-specific immune response is generated.

### Methods of Stimulating an Immune Response and Methods of Treatment

The vaccine compositions described herein may be administered to a subject in need thereof. Provided herein are methods for inducing an immune response in a subject, which involve administering to a subject an immunologically effective amount of the genetically modified cells. Also provided are methods for preventing or treating a tumor in a subject by administering an anti-tumor effective amount of the vaccine compositions described herein. Such compositions and methods may be effective to prolong the survival of the subject.

According to various embodiments, administration of any one of the vaccine compositions provided herein can increase pro-inflammatory cytokine production (e.g., IFNγ secretion) by leukocytes. In some embodiments, administration of any one of the vaccine compositions provided herein can increase pro-inflammatory cytokine production (e.g., IFNγ secretion) by leukocytes by at least 1.5-fold, 1.6-fold, 1.75-fold, 2-fold, 2.5-fold, 3.0-fold, 3.5-fold, 4.0-fold, 4.5-fold, 5.0-fold or more. In other embodiments, the IFNγ production is increased by approximately 1, 2, 3, 4, 5, 6, 7, 8, 9, 10, 11, 12, 13, 14, 15, 16, 17, 18, 19, 20, 21, 22, 23, 24, or 25-fold or higher compared to unmodified cancer cell lines. Assays for determining the amount of cytokine production are well-known in the art and described herein. Without being bound to any theory or mechanism, the increase in pro-inflammatory cytokine production (e.g., IFNγ secretion) by leukocytes is a result of either indirect or direct interaction with the vaccine composition.

In some embodiments, administration of any one of the vaccine compositions provided herein comprising one or more modified cell lines as described herein can increase the uptake of cells of the vaccine composition by phagocytic cells, e.g., by at least 1.1-fold, 1.2-fold, 1.3-fold, 1.4-fold, 1.5-fold, 2-fold, 2.5-fold or more, as compared to a composition that does not comprise modified cells.

In some embodiments, the vaccine composition is provided to a subject by an intradermal injection. Without being bound to any theory or mechanism, the intradermal injection, in at least some embodiments, generates a localized inflammatory response recruiting immune cells to the injection site. Following administration of the vaccine, antigen presenting cells (APCs) in the skin, such as Langerhans cells (LCs) and dermal dendritic cells (DCs), uptake the vaccine cell line components by phagocytosis and then migrate through the dermis to the draining lymph node. At the draining lymph node, DCs or LCs that have phagocytized the vaccine cell line components are expected to prime naïve T cells and B cells. Priming of naïve T and B cells is expected to initiate an adaptive immune response to tumor associated antigens (TAAs) expressed by the vaccine cell line components. Certain TAAs expressed by the vaccine cell line components are also expressed by the patient's tumor. Expansion of antigen specific T cells at the draining lymph node and trafficking of these T cells to the tumor microenvironment (TME) is expected to generate a vaccine-induced anti-tumor response.

According to various embodiments, immunogenicity of the allogenic vaccine composition can be further enhanced through genetic modifications that reduce expression of immunosuppressive factors while increasing the expression or secretion of immunostimulatory signals. Modulation of these factors aims to enhance the uptake vaccine cell line components by LCs and DCs in the dermis, trafficking of DCs and LCs to the draining lymph node, T cell and B cell priming in the draining lymph node, and, thereby resulting in more potent anti-tumor responses.

In some embodiments, the breadth of TAAs targeted in the vaccine composition can be increased through the inclusion of multiple cell lines. For example, different histological subsets within a certain tumor type tend to express different TAA subsets. The magnitude and breadth of the adaptive immune response induced by the vaccine composition can, according to some embodiments of the disclosure, be enhanced through the inclusion of additional cell lines expressing the same or different immunostimulatory factors. For example, expression of an immunostimulatory factor, such as IL-12, by one cell line within a cocktail of three cell lines can act locally to enhance the immune responses to all cell lines delivered into the same site. The expression of an immunostimulatory factor by more than one cell line within a cocktail, such as GM-CSF, can increase the amount of the immunostimulatory factor in the injection site, thereby enhancing the immune responses induced to all components of the cocktail. The degree of HLA mismatch present within a vaccine cocktail may further enhance the immune responses induced by that cocktail.

As described herein, in various embodiments, a method of stimulating an immune response specific to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more TAAs in a subject is provided comprising administering a therapeutically effective amount of a vaccine composition comprising modified cancer cell lines.

An “immune response” is a response of a cell of the immune system, such as a B cell, T cell, or monocyte, to a stimulus, such as a cell or antigen (e.g., formulated as an antigenic composition or a vaccine). An immune response can be a B cell response, which results in the production of specific antibodies, such as antigen specific neutralizing antibodies. An immune response can also be a T cell response, such as a CD4+response or a CD8+response. B cell and T cell responses are aspects of a “cellular” immune response. An immune response can also be a “humoral” immune response, which is mediated by antibodies. In some cases, the response is specific for a particular antigen (that is, an “antigen specific response”), such as one or more TAAs, and this specificity can include the production of antigen specific antibodies and/or production of a cytokine such as interferon gamma which is a key cytokine involved in the generation of a Thi T cell response and measurable by ELISpot and flow cytometry.

Vaccine efficacy can be tested by measuring the T cell response CD4+ and CD8+after immunization, using flow cytometry (FACS) analysis, ELISpot assay, or other method known in the art. Exposure of a subject to an immunogenic stimulus, such as a cell or antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen (or cell) specific response, (e.g., by increasing antibody titer and/or affinity, by increasing the frequency of antigen specific B or T cells, by inducing maturation effector function, or a combination thereof).

The immune responses that are monitored/assayed or stimulated by the methods described herein include, but not limited to: (a) antigen specific or vaccine specific IgG antibodies; (b) changes in serum cytokine levels that may include and is not limited to: IL-1β, IL-4, IL-5, IL-6, IL-8, IL-10, IL-12, IL-17A, IL-20, IL-22, TNFα, IFNγ, TGFβ, CCL5, CXCL10; (c) IFNγ responses determined by ELISpot for CD4 and CD8 T cell vaccine and antigen specific responses; (d) changes in IFNγ responses to TAA or vaccine cell components; (e) increased T cell production of intracellular cytokines in response to antigen stimulation: IFNγ, TNFα, and IL-2 and indicators of cytolytic potential: Granzyme A, Granzyme B, Perforin, and CD107a; (f) decreased levels of regulatory T cells (Tregs), mononuclear monocyte derived suppressor cells (M-MDSCs), and polymorphonuclear derived suppressor cells (PMN-MDSCs); (g) decreased levels of circulating tumor cells (CTCs); (h) neutrophil to lymphocyte ratio (NLR) and platelet to lymphocyte ratio (PLR); (i) changes in immune infiltrate in the TME; and (j) dendritic cell maturation.

Assays for determining the immune responses are described herein and well known in the art. DC maturation can be assessed, for example, by assaying for the presence of DC maturation markers such as CD80, CD83, CD86, and MHC II. (See Dudek, A., et al., Front. Immunol., 4:438 (2013)). Antigen specific or vaccine specific IgG antibodies can be assessed by ELISA or flow cytometry. Serum cytokine levels can be measured using a multiplex approach such as Luminex or Meso Scale Discovery Electrochemiluminescence (MSD). T cell activation and changes in lymphocyte populations can be measured by flow cytometry. CTCs can be measured in PBMCs using a RT-PCR based approach. The NLR and PLR ratios can be determined using standard complete blood count (CBC) chemistry panels. Changes in immune infiltrate in the TME can be assessed by flow cytometry, tumor biopsy and next-generation sequencing (NGS), or positron emission tomography (PET) scan of a subject.

Given the overlap in TAA expression between cancers and tumors of different types, the present disclosure provides, in certain embodiments, compositions that can treat multiple different cancers. For example, one vaccine composition comprising two cocktails of three cell lines each may be administered to a subject suffering from two or more types of cancers and said vaccine composition is effective at treating both, additional or all types of cancers. In exemplary embodiments, and in consideration of the TAA expression profile, the same vaccine composition comprising modified cancer cell lines is used to treat prostate cancer and testicular cancer, gastric and esophageal cancer, or endometrial, ovarian, and breast cancer in the same patient (or different patients). TAA overlap can also occur within subsets of hot tumors or cold tumors. In some embodiments, cell lines included in the vaccine composition can be selected from two tumor types of similar immune landscape to treat one or both of the tumor types in the same individual.

As used herein, changes in or “increased production” of, for example a cytokine such as IFNγ, refers to a change or increase above a control or baseline level of production/secretion/expression and that is indicative of an immunostimulatory response to an antigen or vaccine component.

### Combination Treatments and Regimens

### Formulations, Adjuvants, and Additional Therapeutic Agents

The compositions described herein may be formulated as pharmaceutical compositions. The term “pharmaceutically acceptable” as used herein refers to a pharmaceutically acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with tissue, organs or other human component without excessive toxicity, irritation, allergic response, immunogenicity, or other problems or complications, commensurate with a reasonable benefit/risk ratio. (See Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004)).

Embodiments of the pharmaceutical composition of the disclosure is formulated to be compatible with its intended route of administration (i.e., parenteral, intravenous, intra-arterial, intradermal, subcutaneous, oral, inhalation, transdermal, topical, intratumoral, transmucosal, intraperitoneal or intra-pleural, and/or rectal administration). Solutions or suspensions used for parenteral, intradermal, or subcutaneous application can include the following components: a sterile diluent such as water, saline solution, fixed oils, polyethylene glycols, glycerine, propylene glycol or other synthetic solvents; dimethyl sulfoxide (DMSO); antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, or one or more vials comprising glass or polymer (e.g., polypropylene). The term “vial” as used herein means any kind of vessel, container, tube, bottle, or the like that is adapted to store embodiments of the vaccine composition as described herein.

In some embodiments, the composition further comprises a pharmaceutically acceptable carrier. The term “carrier” as used herein encompasses diluents, excipients, adjuvants, and combinations thereof. Pharmaceutically acceptable carriers are well known in the art (See Remington: The Science and Practice of Pharmacy, 21st Edition). Exemplary “diluents” include sterile liquids such as sterile water, saline solutions, and buffers (e.g., phosphate, tris, borate, succinate, or histidine). Exemplary “excipients” are inert substances that may enhance vaccine stability and include but are not limited to polymers (e.g., polyethylene glycol), carbohydrates (e.g., starch, glucose, lactose, sucrose, or cellulose), and alcohols (e.g., glycerol, sorbitol, or xylitol).

In various embodiments, the vaccine compositions and cell line components thereof are sterile and fluid to the extent that the compositions and/or cell line components can be loaded into one or more syringes. In various embodiments, the compositions are stable under the conditions of manufacture and storage and preserved against the contaminating action of microorganisms such as bacteria and fungi. In some embodiments, the carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, by the use of surfactants, and by other means known to one of skill in the art. Prevention of the action of microorganisms can be achieved by various antibacterial and antifungal agents, for example, parabens, chlorobutanol, phenol, ascorbic acid, thimerosal, and the like. In some embodiments, it may be desirable to include isotonic agents, for example, sugars, polyalcohols such as manitol, sorbitol, and/or sodium chloride in the composition. In some embodiments, prolonged absorption of the injectable compositions can be brought about by including in the composition an agent that delays absorption, for example, aluminum monostearate and gelatin.

In some embodiments, sterile injectable solutions can be prepared by incorporating the active compound(s) in the required amount(s) in an appropriate solvent with one or a combination of ingredients enumerated above, as required, followed by filtered sterilization. In certain embodiments, dispersions are prepared by incorporating the active compound into a sterile vehicle that contains a basic dispersion medium and the required other ingredients from those enumerated herein. In the case of sterile powders for the preparation of sterile injectable solutions, embodiments of methods of preparation include vacuum drying and freeze-drying that yield a powder of the active ingredient plus any additional desired ingredient from a previously sterile-filtered solution thereof.

The innate immune system comprises cells that provide defense in a non-specific manner to infection by other organisms. Innate immunity in a subject is an immediate defense, but it is not long-lasting or protective against future challenges. Immune system cells that generally have a role in innate immunity are phagocytic, such as macrophages and dendritic cells. The innate immune system interacts with the adaptive (also called acquired) immune system in a variety of ways.

In some embodiments, the vaccine compositions alone activate an immune response (i.e., an innate immune response, an adaptive immune response, and/or other immune response). In some embodiments, one or more adjuvants are optionally included in the vaccine composition or are administered concurrently or strategically in relation to the vaccine composition, to provide an agent(s) that supports activation of innate immunity in order to enhance the effectiveness of the vaccine composition. An “adjuvant” as used herein is an “agent” or substance incorporated into the vaccine composition or administered simultaneously or at a selected time point or manner relative to the administration of the vaccine composition. In some embodiments, the adjuvant is a small molecule, chemical composition, or therapeutic protein such as a cytokine or checkpoint inhibitor. A variety of mechanisms have been proposed to explain how different agents function (e.g., antigen depots, activators of dendritic cells, macrophages). An agent may act to enhance an acquired immune response in various ways and many types of agents can activate innate immunity. Organisms, like bacteria and viruses, can activate innate immunity, as can components of organisms, chemicals such as 2′-5′ oligo A, bacterial endotoxins, RNA duplexes, single stranded RNA and other compositions. Many of the agents act through a family of molecules referred to herein as “toll-like receptors” (TLRs). Engaging a TLR can also lead to production of cytokines and chemokines and activation and maturation of dendritic cells, components involved in development of acquired immunity. The TLR family can respond to a variety of agents, including lipoprotein, peptidoglycan, flagellin, imidazoquinolines, CpG DNA, lipopolysaccharide and double stranded RNA. These types of agents are sometimes called pathogen (or microbe)-associated molecular patterns. In some embodiments, the adjuvant is a TLR4 agonist.

One adjuvant that in some embodiments may be used in the vaccine compositions is a monoacid lipid A (MALA) type molecule. An exemplary MALA is MPL® adjuvant as described in, e.g., Ulrich J. T. and Myers, K. R., Chapter 21 in Vaccine Design, the Subunit and Adjuvant Approach, Powell, M. F. and Newman, M. J., eds. Plenum Press, NY (1995).

In other embodiments, the adjuvant may be “alum”, where this term refers to aluminum salts, such as aluminum phosphate and aluminum hydroxide.

In some embodiments, the adjuvant may be an emulsion having vaccine adjuvant properties. Such emulsions include oil-in-water emulsions. Incomplete Freund's adjuvant (IFA) is one such adjuvant. Another suitable oil-in-water emulsion is MF-59™ adjuvant which contains squalene, polyoxyethylene sorbitan monooleate (also known as Tween® 80 surfactant) and sorbitan trioleate. Other suitable emulsion adjuvants are Montanide™ adjuvants (Seppic Inc., Fairfield NJ) including Montanide™ ISA 50V which is a mineral oil-based adjuvant, Montanide™ ISA 206, and Montanide™ IMS 1312. While mineral oil may be present in the adjuvant, in one embodiment, the oil component(s) of the compositions of the present disclosure are all metabolizable oils.

In some embodiments, the adjuvant may be AS02™ adjuvant or AS04TM adjuvant. AS02TM adjuvant is an oil-in-water emulsion that contains both MPL™ adjuvant and QS-21™ adjuvant (a saponin adjuvant discussed elsewhere herein). AS04™ adjuvant contains MPL™ adjuvant and alum. The adjuvant may be Matrix-M™ adjuvant. The adjuvant may be a saponin such as those derived from the bark of the Quillaja saponaria tree species, or a modified saponin, see, e.g., U.S. Pat. Nos. 5,057,540; 5,273,965; 5,352,449; 5,443,829; and 5,560,398. The product QS-21™ adjuvant sold by Antigenics, Inc. (Lexington, MA) is an exemplary saponin-containing co-adjuvant that may be used with embodiments of the composition described herein. In other embodiments, the adjuvant may be one or a combination of agents from the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

In some embodiments, the adjuvant or agent may be a cytokine that functions as an adjuvant, see, e.g., Lin R. et al. Clin. Infec. Dis. 21(6):1439-1449 (1995); Taylor, C. E., Infect. Immun. 63(9):3241-3244 (1995); and Egilmez, N. K., Chap. 14 in Vaccine Adjuvants and Delivery Systems, John Wiley & Sons, Inc. (2007). In various embodiments, the cytokine may be, e.g., granulocyte-macrophage colony-stimulating factor (GM-CSF); see, e.g., Change D. Z. et al. Hematology 9(3):207-215 (2004), Dranoff, G. Immunol. Rev. 188:147-154 (2002), and U.S. Pat. No. 5,679,356; or an interferon, such as a type I interferon, e.g., interferon-α (IFN-α) or interferon-β (IFN-β), or a type II interferon, e.g., interferon-γ (IFNγ), see, e.g., Boehm, U. et al. Ann. Rev. Immunol. 15:749-795 (1997); and Theofilopoulos, A. N. et al. Ann. Rev. Immunol. 23:307-336 (2005); an interleukin, specifically including interleukin-1a (IL-1a), interleukin-1p (IL-1β), interleukin-2 (IL-2); see, e.g., Nelson, B. H., J. Immunol. 172(7): 3983-3988 (2004); interleukin-4 (IL-4), interleukin-7 (IL-7), interleukin-12 (IL-12); see, e.g., Portielje, J. E., et al., Cancer Immunol. Immunother. 52(3): 133-144 (2003) and Trinchieri. G. Nat. Rev. Immunol. 3(2):133-146 (2003); interleukin-15 (Il-15), interleukin-18 (IL-18); fetal liver tyrosine kinase 3 ligand (FIt3L), or tumor necrosis factor α (TNFα).

In some embodiments, the adjuvant may be unmethylated CpG dinucleotides, optionally conjugated to the antigens described herein.

Examples of immunopotentiators that may be used in the practice of the compositions and methods described herein as adjuvants include: MPL™; MDP and derivatives; oligonucleotides; double-stranded RNA; alternative pathogen-associated molecular patterns (PAMPS); saponins; small-molecule immune potentiators (SMIPs); cytokines; and chemokines.

When two or more adjuvants or agents are utilized in combination, the relative amounts of the multiple adjuvants may be selected to achieve the desired performance properties for the composition which contains the adjuvants, relative to the antigen alone. For example, an adjuvant combination may be selected to enhance the antibody response of the antigen, and/or to enhance the subject's innate immune system response. Activating the innate immune system results in the production of chemokines and cytokines, which in turn may activate an adaptive (acquired) immune response. An important consequence of activating the adaptive immune response is the formation of memory immune cells so that when the host re-encounters the antigen, the immune response occurs quicker and generally with better quality. In some embodiments, the adjuvant(s) may be pre-formulated prior to their combination with the compositions described herein.

Embodiments of the vaccine compositions described herein may be administered simultaneously with, prior to, or after administration of one or more other adjuvants or agents, including therapeutic agents. In certain embodiments, such agents may be accepted in the art as a standard treatment or prevention for a particular cancer. Exemplary agents contemplated include cytokines, growth factors, steroids, NSAIDs, DMARDs, anti-inflammatories, immune checkpoint inhibitors, chemotherapeutics, radiotherapeutics, or other active and ancillary agents. In other embodiments, the agent is one or more isolated TAA as described herein.

In some embodiments, a vaccine composition provided herein is administered to a subject that has not previously received certain treatment or treatments for cancer or other disease or disorder. As used herein, the phrase “wherein the subject refrains from treatment with other vaccines or therapeutic agents” refers to a subject that has not received a cancer treatment or other treatment or procedure prior to receiving a vaccine of the present disclosure. In some embodiments, the subject refrains from receiving one or more therapeutic vaccines (e.g., flu vaccine, covid-19 vaccine such as AZD1222, BNT162b2, mRNA-1273, and the like) prior to the administration of the therapeutic vaccine as described in various embodiments herein. In some embodiments, the subject refrains from receiving one or more antibiotics prior to the administration of the therapeutic vaccine as described in various embodiments herein. “Immune tolerance” is a state of unresponsiveness of the immune system to substances, antigens, or tissues that have the potential to induce an immune response. The vaccine compositions of the present disclosure, in certain embodiments, are administered to avoid the induction of immune tolerance or to reverse immune tolerance.

In various embodiments, the vaccine composition is administered in combination with one or more active agents used in the treatment of cancer, including one or more chemotherapeutic agents. Examples of such active agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and paclitaxel protein-bound particles (ABRAXANE®) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoid or retinoic acid or retinoic acid derivative such as all-trans retinoic acid (ATRA), VESANOID® (tretinoin), ACCUTANE® (isotretinoin, 9-cis-retinoid, 13-cis-retinoic acid), vitamin A acid) TARGRETIN™ (bexarotene), PANRETIN™ (alitretinoin); and ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer active agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors.

In further embodiments, the vaccine composition is administered in combination with a TLR4 agonist, TLR8 agonist, or TLR9 agonist. Such an agonist may be selected from peptidoglycan, polyl:C, CpG, 3M003, flagellin, and Leishmania homolog of eukaryotic ribosomal elongation and initiation factor 4a (LeIF).

In some embodiments, the vaccine composition is administered in combination with a cytokine as described herein. In some embodiments, the compositions disclosed herein may be administered in conjunction with molecules targeting one or more of the following: Adhesion: MAdCAM1, ICAM1, VCAM1, CD103; Inhibitory Mediators: IDO, TDO; MDSCs/Tregs: NOS1, arginase, CSFR1, FOXP3, cyclophosphamide, PI3Kgamma, PI3Kdelta, tasquinimod; Immunosuppression: TGFβ, IL-10; Priming and Presenting: BATF3, XCR1/XCL1, STING, INFalpha; Apoptotic Recycling: IL-6, surviving, IAP, mTOR, MCL1, PI3K; T-Cell Trafficking: CXCL9/10/11, CXCL1/13, CCL2/5, anti-LIGHT, anti-CCR5; Oncogenic Activation: WNT-beta-cat, MEK, PPARgamma, FGFR3, TKls, MET; Epigenetic Reprogramming: HDAC, HMA, BET; Angiogenesis immune modulation: VEGF(alpha, beta, gamma); Hypoxia: HIF1alpha, adenosine, anit-ADORA2A, anti-CD73, and anti-CD39.

In certain embodiments, the compositions disclosed herein may be administered in conjunction with a histone deacetylase (HDAC) inhibitor. HDAC inhibitors include hydroxamates, cyclic peptides, aliphatic acids and benzamides. Illustrative HDAC inhibitors contemplated for use herein include, but are not limited to, Suberoylanilide hydroxamic acid (SAHA/Vorinostat/Zolinza), Trichostatin A (TSA), PXD-101, Depsipeptide (FK228/romidepsin/ISTODAX®), panobinostat (LBH589), MS-275, Mocetinostat (MGCD0103), ACY-738, TMP195, Tucidinostat, valproic acid, sodium phenylbutyrate, 5-aza-2′-deoxycytidine (decitabine). See e.g., Kim and Bae, Am J Transl Res 2011; 3(2):166-179; Odunsi et al., Cancer Immunol Res. 2014 Jan. 1; 2(1): 37-49. Other HDAC inhibitors include Vorinostat (SAHA, MK0683), Entinostat (MS-275), Panobinostat (LBH589), Trichostatin A (TSA), Mocetinostat (MGCD0103), ACY-738, Tucidinostat (Chidamide), TMP195, Citarinostat (ACY-241), Belinostat (PXD101), Romidepsin (FK228, Depsipeptide), MC1568, Tubastatin A HCl, Givinostat (ITF2357), Dacinostat (LAQ824), CUDC-101, Quisinostat (JNJ-26481585) 2HCI, Pracinostat (SB939), PCI-34051, Droxinostat, Abexinostat (PCI-24781), RGFP966, AR-42, Ricolinostat (ACY-1215), Valproic acid sodium salt (Sodium valproate), Tacedinaline (C1994), CUDC-907, Sodium butyrate, Curcumin, M344, Tubacin, RG2833 (RGFP109), Resminostat, Divalproex Sodium, Scriptaid, and Tubastatin A.

In certain embodiments, the vaccine composition is administered in combination with chloroquine, a lysosomotropic agent that prevents endosomal acidification and which inhibits autophagy induced by tumor cells to survive accelerated cell growth and nutrient deprivation. More generally, the compositions comprising heterozygous viral vectors as described herein may be administered in combination with active agents that act as autophagy inhibitors, radiosensitizers or chemosensitizers, such as chloroquine, misonidazole, metronidazole, and hypoxic cytotoxins, such as tirapazamine. In this regard, such combinations of a heterozygous viral vector with chloroquine or other radio or chemo sensitizer, or autophagy inhibitor, can be used in further combination with other cancer active agents or with radiation therapy or surgery.

In other embodiments, the vaccine composition is administered in combination with one or more small molecule drugs that are known to result in killing of tumor cells with concomitant activation of immune responses, termed “immunogenic cell death”, such as cyclophosphamide, doxorubicin, oxaliplatin and mitoxantrone. Furthermore, combinations with drugs known to enhance the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, furthermore proteasome inhibitors (e.g., bortezomib), shikonin (the major constituent of the root of Lithospermum erythrorhizon,) and oncolytic viruses, such as TVec (talimogene laherparepvec). In some embodiments, the compositions comprising heterozygous viral vectors as described herein may be administered in combination with epigenetic therapies, such as DNA methyltransferase inhibitors (e.g., decitabine, 5-aza-2′-deoxycytidine) which may be administered locally or systemically.

In other embodiments, the vaccine composition is administered in combination with one or more antibodies that increase ADCC uptake of tumor by DCs. Thus, embodiments of the present disclosure contemplate combining cancer vaccine compositions with any molecule that induces or enhances the ingestion of a tumor cell or its fragments by an antigen presenting cell and subsequent presentation of tumor antigens to the immune system. These molecules include agents that induce receptor binding (e.g., Fc or mannose receptors) and transport into the antigen presenting cell such as antibodies, antibody-like molecules, multi-specific multivalent molecules and polymers. Such molecules may either be administered intratumorally with the composition comprising heterozygous viral vector or administered by a different route. For example, a composition comprising heterozygous viral vector as described herein may be administered intratumorally in conjunction with intratumoral injection of rituximab, cetuximab, trastuzumab, Campath, panitumumab, ofatumumab, brentuximab, pertuzumab, Ado-trastuzumab emtansine, Obinutuzumab, anti-HER1, -HER2, or -HER3 antibodies (e.g., MEHD7945A; MM-111; MM-151; MM-121; AMG888), anti-EGFR antibodies (e.g., nimotuzumab, ABT-806), or other like antibodies. Any multivalent scaffold that is capable of engaging Fc receptors and other receptors that can induce internalization may be used in the combination therapies described herein (e.g., peptides and/or proteins capable of binding targets that are linked to Fc fragments or polymers capable of engaging receptors).

In certain embodiments, the vaccine composition may be further combined with an inhibitor of ALK, PARP, VEGFRs, EGFR, FGFR1-3, HIF1α, PDGFR1-2, c-Met, c-KIT, Her2, Her3, AR, PR, RET, EPHB4, STAT3, Ras, HDAC1-11, mTOR, and/or CXCR4.

In certain embodiments, a cancer vaccine composition may be further combined with an antibody that promotes a co-stimulatory signal (e.g., by blocking inhibitory pathways), such as anti-CTLA-4, or that activates co-stimulatory pathways such as an anti-CD40, anti-CD28, anti-ICOS, anti-OX40, anti-CD27, anti-ICOS, anti-CD127, anti-GITR, IL-2, IL-7, IL-15, IL-21, GM-CSF, IL-12, and INFα.

### Retinoic Acid

In certain embodiments, a retinoid, retinoic acid or retinoic acid derivative such as all-trans retinoic acid (ATRA), VESANOID® (tretinoin), ACCUTANE® (isotretinoin, 9-cis-retinoid, 13-cis-retinoic acid, vitamin A acid), TARGRETIN™ (bexarotene), PANRETIN™ (alitretinoin), and ONTAK™ (denileukin diftitox) is administered in combination with the vaccine compositions described herein.

Various studies, including clinical trials, have looked at the use of retinoic acid in the treatment of cancers, including glioblastoma. (See, e.g., Penas-Prado M, et al., Neuro Oncol., 2014, 17(2):266-273; Butowski N, et al., Int J Radiat Oncol Biol Phys., 2005, 61(5):1454-1459; Jaeckle K A, et al., J Clin Oncol., 2003, 21(12): 2305-2311; Yung W K, et al., Clin Cancer Res., 1996, 2(12):1931-1935; and SJ, Levin V A, et al., Neuro Oncol., 2004, 6(3):253-258.) Embodiments of the present disclosure provide concomitant use of ATRA and/or related retinoids in combination with allogeneic tumor cell vaccines to improve immune response and efficacy by altering the tumor microenvironment. In some embodiments, ATRA is administered at a dose of 25-100 mg per square meter of body surface area per day. In various embodiments, 25, 30, 35, 40, 45, 50, 55, 60, 65, 70, 75, 80, 85, 90, 95, 100, 110, 115, 120, 125, 130, 135, 140, 145 or 150 mg per square meter of body surface area per day is administered. In one embodiment, ATRA is administered orally and is optionally administered in accordance with the dosing frequency of other concomitant anti-tumor agents as described herein. In one embodiment, ATRA is administered twice in one day. PK studies of ATRA have demonstrated that the drug auto-catalyzes and serum levels decrease with continuous dosing. Thus, in certain embodiments, the ATRA dosing schedule includes one or two weeks on and one or two weeks off.

In one exemplary embodiment, in combination with allogeneic tumor cell vaccines described herein, ATRA is administered at doses of 25-100 mg per square meter per day in two divided doses for 7 continuous days, followed by 7 days without administration of ATRA, followed by the same cycle of 7 days on and 7 days off for as long as the vaccine therapy is being administered. In another embodiment, ATRA is administered at the same time as cyclophosphamide as described herein.

In some embodiments, ATRA is administered in combination with a vaccine composition as described herein for the treatment of colorectal cancer.

### Checkpoint Inhibitors

In certain embodiments, a checkpoint inhibitor molecule is administered in combination with the vaccine compositions described herein. Immune checkpoints refer to a variety of inhibitory pathways of the immune system that are crucial for maintaining self-tolerance and for modulating the duration and amplitude of an immune responses. Tumors use certain immune-checkpoint pathways as a major mechanism of immune resistance, particularly against T cells that are specific for tumor antigens. (See Pardoll, 2012 Nature 12:252; Chen and Mellman Immunity 39:1 (2013)). Immune checkpoint inhibitors include any agent that blocks or inhibits in a statistically significant manner, the inhibitory pathways of the immune system. Such inhibitors may include antibodies, or antigen binding fragments thereof, that bind to and block or inhibit immune checkpoint receptors or antibodies that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, BTLA, SIGLEC9, 2B4 (belongs to the CD2 family of molecules and is expressed on all NK, γδ, and memory CD8+ (αβ) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, BTLA, SIGLEC9, 2B4, CD160, and CGEN-15049.

Illustrative immune checkpoint inhibitors include anti-PD1, anti-PDL1, and anti-PDL2 agents such as A167, AB122, ABBV-181, ADG-104, AK-103, AK-105, AK-106, AGEN2034, AM0001, AMG-404, ANB-030, APL-502, APL-501, zimberelimab, atezolizumab, AVA-040, AVA-040-100, avelumab, balstilimab, BAT-1306, BCD-135, BGB-A333, BI-754091, budigalimab, camrelizumab, CB-201, CBT-502, CCX-4503, cemiplimab, cosibelimab, cetrelimab, CS-1001, CS-1003, CX-072, CX-188, dostarlimab, durvalumab, envafolimab, sugemalimab, HBM9167, F-520, FAZ-053, genolimzumab, GLS-010, GS-4224, hAB21, HLX-10, HLX-20, HS-636, HX-008, IMC-001, IMM-25, INCB-86550, JS-003, JTX-4014, JYO-34, KL-A167, LBL-006, lodapolimab, LP-002, LVGN-3616, LYN-00102, LMZ-009, MAX-10181, MEDI-0680, MGA-012 (Retifanlimab), MSB-2311, nivolumab, pembrolizumab, prolgolimab, prololimab, sansalimab, SCT-110A, SG-001, SHR-1316, sintilimab, spartalizumab, RG6084, RG6139, RG6279, CA-170, CA-327, STI-3031, toleracyte, toca 521, Sym-021, TG-1501, tislelizumab, toripalimab, TT-01, ZKAB-001, and the anti-PD-1 antibodies capable of blocking interaction with its ligands PD-L1 and PD-L2 described in WO/2017/124050.

Illustrative multi-specific immune checkpoint inhibitors, where at least one target is anti-PD1, anti-PDL1, or anti-PDL2, include ABP-160 (CD47×PD-L1), AK-104 (PD-1×CTLA-4), AK-112 (PD-1×VEGF), ALPN-202 (PD-L1×CTLA-4×CD28), AP-201 (PD-L1×OX-40), AP-505 (PD-L1×VEGF), AVA-0017 (PD-L1×LAG-3), AVA-0021 (PD-L1×LAG-3), AUPM-170 (PD-L1×VISTA), BCD-217 (PD-1×CTLA-4), BH-2950 (PD-1×HER2), BH-2996h (PD-1×PD-L1), BH-29xx (PD-L1×CD47), bintrafusp alfa (PD-L1×TGFβ), CB-213 (PD-1×LAG-3), CDX-527 (CD27×PD-L1), CS-4100 (PD-1×PD-L1), DB-001 (PD-L1×HER2), DB-002 (PD-L1×CTLA-4), DSP-105 (PD-1×4-1BBL), DSP-106, (PD-1×CD70), FS-118 (LAG-3×PD-L1), FS-222 (CD137/4-1BB×PD-L1), GEN-1046 (PD-L1×CD137/4-1BB), IBI-318 (PD-1×PD-L1), IBI-322 (PD-L1×CD-47), KD-033 (PD-L1×IL-15), KN-046 (PD-L1×CTLA-4), KY-1043 (PD-L1×IL-2), LY-3434172 (PD-1×PD-L1), MCLA-145 (PD-L1×CD137), MEDI-5752 (PD-1×CTLA-4), MGD-013 (PD-1×LAG-3), MGD-019 (PD-1×CTLA-4), ND-021 (PD-L1×4-1BB×HSA), OSE-279 (PD-1×PD-L1), PRS-332 (PD-1×HER2), PRS-344 (PD-L1×CD137), PSB-205 (PD-1×CTLA-4), R-7015 (PD-L1×TGFβ), RO-7121661 (PD-1×TIM-3), RO-7247669 (PD-1×LAG-3), SHR-1701 (PD-L1×TGFβ2), SL-279252 (PD-1×OX40L), TSR-075 (PD-1×LAG-3), XmAb-20717 (CTLA-4×PD-1), XmAb-23104 (PD-1×ICOS), and Y-111 (PD-L1×CD-3).

Additional illustrative immune checkpoint inhibitors include anti-CTLA4 agents such as: ADG-116, AGEN-2041, BA-3071, BCD-145, BJ-003, BMS-986218, BMS-986249, BPI-002, CBT-509, CG-0161, Olipass-1, HBM-4003, HLX-09, IBI-310, ipilimumab, JS-007, KN-044, MK-1308, ONC-392, REGN-4659, RP-2, tremelimumab, and zalifrelimab. Additional illustrative multi-specific immune checkpoint inhibitors, where at least one target is anti-CTLA4, include: AK-104 (PD-1×CTLA-4), ALPN-202 (PD-L1×CTLA-4×CD28), ATOR-1015 (CTLA-4×OX40), ATOR-1144 (CTLA-4×GITR), BCD-217 (PD-1×CTLA-4), DB-002 (PD-L1×CTLA-4), FPT-155 (CD28×CTLA-4), KN-046 (PD-L1×CTLA-4), ), MEDI-5752 (PD-1×CTLA-4), MGD-019 (PD-1×CTLA-4), PSB-205 (PD-1×CTLA-4), XmAb-20717 (CTLA-4×PD-1), and XmAb-22841 (CTLA-4×LAG-3). Additional illustrative immune checkpoint inhibitors include anti-LAG3 agents such as BI-754111, BJ-007, eftilagimod alfa, GSK-2831781, HLX-26, IBI-110, IMP-701, IMP-761, INCAGN-2385, LBL-007, MK-4280, REGN-3767, relatlimab, Sym-022, TJ-A3, and TSR-033. Additional illustrative multi-specific immune checkpoint inhibitors, where at least one target is anti-LAG3, include: CB-213 (PD-1×LAG-3), FS-118 (LAG-3×PD-L1), MGD-013 (PD-1×LAG-3), AVA-0017 (PD-L1×LAG-3), AVA-0021 (PD-L1×LAG-3), RO-7247669 (PD-1×LAG-3), TSR-075 (PD-1×LAG-3), and XmAb-22841 (CTLA-4×LAG-3). Additional illustrative immune checkpoint inhibitors include anti-TIGIT agents such as AB-154, ASP8374, BGB-A1217, BMS-986207, CASC-674, COM-902, EOS-884448, HLX-53, IBI-939, JS-006, MK-7684, NB-6253, RXI-804, tiragolumab, and YH-29143. Additional illustrative multi-specific immune checkpoint inhibitors, where at least one target is anti-TIGIT are contemplated. Additional illustrative immune checkpoint inhibitors include anti-TIM3 agents such as: BGB-A425, BMS-986258, ES-001, HLX-52, INCAGN-2390, LBL-003, LY-3321367, MBG-453, SHR-1702, Sym-023, and TSR-022. Additional illustrative multi-specific immune checkpoint inhibitors, where at least one target is anti-TIM3, include: AUPM-327 (PD-L1×TIM-3), and RO-7121661 (PD-1×TIM-3). Additional illustrative immune checkpoint inhibitors include anti-VISTA agents such as: HMBD-002, and PMC-309. Additional illustrative multi-specific immune checkpoint inhibitors, where at least one target is anti-VISTA, include CA-170 (PD-L1×VISTA). Additional illustrative immune checkpoint inhibitors include anti-BTLA agents such as: JS-004. Additional illustrative multi-specific immune checkpoint inhibitors, where at least one target is anti-BTLA are contemplated. Illustrative stimulatory immune checkpoints include anti-OX40 agents such as ABBV-368, GSK-3174998, HLX-51, IBI-101, INBRX-106, INCAGN-1949, INV-531, JNJ-6892, and KHK-4083. Additional illustrative multi-specific stimulatory immune checkpoints, where at least one target is anti-OX40, include AP-201 (PD-L1×OX-40), APVO-603 (CD138/4-1 BB×OX-40), ATOR-1015 (CTLA-4×OX-40), and FS-120 (OX40×CD137/4-1BB). Additional illustrative stimulatory immune checkpoints include anti-GITR agents such as BMS-986256, CK-302, GWN-323, INCAGN-1876, MK-4166, PTZ-522, and TRX-518. Additional illustrative multi-specific stimulatory immune checkpoints, where at least one target is anti-GITR, include ATOR-1144 (CTLA-4×GITR). Additional illustrative stimulatory immune checkpoints include anti-CD137/4-1BB agents such a: ADG-106, AGEN-2373, AP-116, ATOR-1017, BCY-3814, CTX-471, EU-101, LB-001, LVGN-6051, RTX-4-1BBL, SCB-333, urelumab, utomilumab, and WTiNT. Additional illustrative multi-specific stimulatory immune checkpoints, where at least one target is anti-CD137/4-1BB, include ALG.APV-527 (CD137/4-1BB×5T4), APVO-603 (CD137/4-1BB×OX40), BT-7480 (Nectin-4×CD137/4-1BB), CB-307 (CD137/4-1BB×PSMA), CUE-201 (CD80×CD137/4-1BB), DSP-105 (PD-1×CD137/4-1BB), FS-120 (OX40×CD137/4-1BB), FS-222 (PD-L1×CD137/4-1BB), GEN-1042 (CD40×CD137/4-1BB), GEN-1046 (PD-L1×CD137/4-1BB), INBRX-105 (PD-L1×CD137/4-1BB), MCLA-145 (PD-L1×CD137/4-1BB), MP-0310 (CD137/4-1BB×FAP), ND-021 (PD-L1×CD137/4-1BB×HSA), PRS-343 (CD137/4-1BBx HER2), PRS-342 (CD137/4-1BB×GPC3), PRS-344 (CD137/4-1BB×PD-L1), RG-7827 (FAP×4-1BBL), and RO-7227166 (CD-19×4-1BBL).

Additional illustrative stimulatory immune checkpoints include anti-ICOS agents such as BMS-986226, GSK-3359609, KY-1044, and vopratelimab. Additional illustrative multi-specific stimulatory immune checkpoints, where at least one target is anti-ICOS, include XmAb-23104 (PD-1×ICOS). Additional illustrative stimulatory immune checkpoints include anti-CD127 agents such as MD-707 and OSE-703. Additional illustrative multi-specific stimulatory immune checkpoints, where at least one target is anti-CD127 are contemplated. Additional illustrative stimulatory immune checkpoints include anti-CD40 agents such as ABBV-428, ABBV-927, APG-1233, APX-005M, BI-655064, bleselumab, CD-40GEX, CDX-1140, LVGN-7408, MEDI-5083, mitazalimab, and selicrelumab. Additional Illustrative multi-specific stimulatory immune checkpoints, where at least one target is anti-CD40, include GEN-1042 (CD40×CD137/4-1BB). Additional illustrative stimulatory immune checkpoints include anti-CD28 agents such as FR-104 and theralizumab. Additional illustrative multi-specific stimulatory immune checkpoints, where at least one target is anti-CD28, include ALPN-101 (CD28×ICOS), ALPN-202 (PD-L1×CD28), CUE-201 (CD80×CD137/4-1BB), FPT-155 (CD28×CTLA-4), and REGN-5678 (PSMA×CD28). Additional illustrative stimulatory immune checkpoints include anti-CD27 agents such as: HLX-59 and varlilumab. Additional illustrative multi-specific stimulatory immune checkpoints, where at least one target is anti-CD27, include DSP-160 (PD-L1×CD27/CD70) and CDX-256 (PD-L1×CD27). Additional illustrative stimulatory immune checkpoints include anti-IL-2 agents such as ALKS-4230, BNT-151, CUE-103, NL-201, and THOR-707. Additional illustrative multi-specific stimulatory immune checkpoints, where at least one target is anti-IL-2, include CUE-102 (IL-2×WT1). Additional illustrative stimulatory immune checkpoints include anti-IL-7 agents such as BNT-152. Additional illustrative multi-specific stimulatory immune checkpoints, where at least one target is anti-IL-7 are contemplated. Additional illustrative stimulatory immune checkpoints include anti-IL-12 agents such as AK-101, M-9241, and ustekinumab. Additional illustrative multi-specific stimulatory immune checkpoints, where at least one target is antilL-12 are contemplated.

As described herein, the present disclosure provides methods of administering vaccine compositions, cyclophosphamide, checkpoint inhibitors, retinoids (e.g., ATRA), and/or other therapeutic agents such as Treg inhibitors. Treg inhibitors are known in the art and include, for example, bempegaldesleukin, fludarabine, gemcitabine, mitoxantrone, Cyclosporine A, tacrolimus, paclitaxel, imatinib, dasatinib, bevacizumab, idelalisib, anti-CD25, anti-folate receptor 4, anti-CTLA4, anti-GITR, anti-OX40, anti-CCR4, anti-CCR5, anti-CCR8, or TLR8 ligands.

### Dosing

A “dose” or “unit dose” as used herein refers to one or more vaccine compositions that comprise therapeutically effective amounts of one more cell lines. A dose can be a single vaccine composition, two separate vaccine compositions, or two separate vaccine compositions plus one or more compositions comprising one or more therapeutic agents described herein. When in separate compositions, the two or more compositions of the “dose” are meant to be administered “concurrently”. In some embodiments, the two or more compositions are administered at different sites on the subject (e.g., arm, thigh, or back). As used herein, “concurrent” administration of two compositions or therapeutic agents indicates that within about 30 minutes of administration of a first composition or therapeutic agent, the second composition or therapeutic agent is administered. In cases where more than two compositions and/or therapeutic agents are administered concurrently, each composition or agent is administered within 30 minutes, wherein timing of such administration begins with the administration of the first composition or agent and ends with the beginning of administration of the last composition or agent. In some cases, concurrent administration can be completed (i.e., administration of the last composition or agent begins) within about 30 minutes, or within 15 minutes, or within 10 minutes, or within 5 minutes of start of administration of first composition or agent. Administration of a second (or multiple) therapeutic agents or compositions “prior to” or “subsequent to” administration of a first composition means that the administration of the first composition and another therapeutic agent is separated by at least 30 minutes, e.g., at least 1 hour, at least 2 hours, at least 4 hours, at least 6 hours, at least 8 hours, at least 10 hours, at least 12 hours, at least 18 hours, at least 24 hours, or at least 48 hours.

The amount (e.g., number) of cells from the various individual cell lines in the vaccine compositions can be equal (as defined herein), approximately (as defined herein) equal, or different. In various embodiments, each cell line of a vaccine composition is present in an approximately equal amount. In other embodiments, 2 or 3 cell lines of one vaccine composition are present in approximately equal amounts and 2 or 3 different cell lines of a second composition are present in approximately equal amounts.

In some embodiments, the number of cells from each cell line (in the case where multiple cell lines are administered), is approximately 5.0×10⁵, 1.0×10⁶, 2.0×10⁶, 3.0×10⁶, 4.0×10⁶, 5.0×10⁶, 6.0×10⁶, 7.0×10⁶, 8×10⁶, 9.0×10⁶, 1.0×10⁷, 2.0×10⁷, 3.0×10⁷, 4.0×10⁷, 5.0×10⁷, 6.0×10⁷, 8.0×10⁷, 9.0×10⁷, 1.0×10⁸, 2.0×10⁸, 3.0×10⁸, 4.0×10⁸ or 5.0×10⁸ cells. In one embodiment, approximately 10 million (e.g., 1.0×10⁷) cells from one cell line are contemplated. In another embodiment, where 6 separate cell lines are administered, approximately 10 million cells from each cell line, or 60 million (e.g., 6.0×10⁷) total cells are contemplated.

The total number of cells administered in a vaccine composition, e.g., per administration site, can range from 1.0×10⁶ to 3.0×10⁸. For example, in some embodiments, 2.0×10⁶, 3.0×10⁶, 4.0×10⁶, 5.0×10⁶, 6.0×10⁶, 7.0×10⁶, 8×10⁶, 9.0×10⁶, 1.0×10⁷, 2.0×10⁷, 3.0×10⁷, 4.0×10⁷, 5.0×10⁷, 6.0×10⁷, 8.0×10⁷, 9.0×10⁷, 1.0×10⁸, 2.0×10⁸, or 3.0×10⁸ cells are administered.

As described herein, the number of cell lines contained with each administration of a cocktail or vaccine composition can range from 1 to 10 cell lines. In some embodiments, the number of cells from each cell line are not equal, and different ratios of cell lines are included in the cocktail or vaccine composition. For example, if one cocktail contains 5.0×10⁷ total cells from 3 different cell lines, there could be 3.33×10⁷ cells of one cell line and 8.33×10⁶ of the remaining 2 cell lines.

The vaccine compositions and compositions comprising additional therapeutic agents (e.g., chemotherapeutic agents, checkpoint inhibitors, and the like) may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial and sublingual injection or infusion techniques. Also envisioned are embodiments where the vaccine compositions and compositions comprising additional therapeutic agents (e.g., chemotherapeutic agents, checkpoint inhibitors, and the like) are administered intranodally or intratumorally.

In some embodiments, the vaccine compositions are administered intradermally. In related embodiments, the intradermal injection involves injecting the cocktail or vaccine composition at an angle of administration of 5 to 15 degrees.

The injections (e.g., intradermal or subcutaneous injections), can be provided at a single site (e.g. arm, thigh or back), or at multiple sites (e.g. arms and thighs). In some embodiments, the vaccine composition is administered concurrently at two sites, where each site receives a vaccine composition comprising a different composition (e.g., cocktail). For example, in some embodiments, the subject receives a composition comprising three cell lines in the arm, and three different, or partially overlapping cell lines in the thigh. In some embodiments, the subject receives a composition comprising one or more cell lines concurrently in each arm and in each thigh.

In some embodiments, the subject receives multiple doses of the cocktail or vaccine composition and the doses are administered at different sites on the subject to avoid potential antigen competition at certain (e.g., draining) lymph nodes. In some embodiments, the multiple doses are administered by alternating administration sites (e.g., left arm and right arm, or left thigh and right thigh) on the subject between doses. In some embodiments, the multiple doses are administered as follows: a first dose is administered in one arm, and second dose is administered in the other arm; subsequent doses, if administered, continue to alternate in this manner. In some embodiments, the multiple doses are administered as follows: a first dose is administered in one thigh, and second dose is administered in the other thigh; subsequent doses, if administered, continue to alternate in this manner. In some embodiments, the multiple doses are administered as follows: a first dose is administered in one thigh, and second dose is administered in one arm; subsequent doses if administered can alternate in any combination that is safe and efficacious for the subject. In some embodiments, the multiple doses are administered as follows: a first dose is administered in one thigh and one arm, and second dose is administered in the other arm and the other thigh; subsequent doses if administered can alternate in any combination that is safe and efficacious for the subject.

In some embodiments, the subject receives, via intradermal injection, a vaccine composition comprising a total of six cell lines (e.g., HCT15, HUTU80, LS411 N, HCT116, RKO and DMS 53 or other 6-cell line combinations described herein) in one, two or more separate cocktails, each cocktail comprising one or a mixture two or more of the 6-cell lines. In some embodiments, the subject receives, via intradermal injection, a vaccine composition comprising a mixture of three cell lines (e.g., three of HCT15, HUTU80, LS411 N, HCT116, RKO and DMS 53 or three cell lines from other 6-cell line combinations described herein). In some embodiments, the subject receives, via intradermal injection to the arm (e.g., upper arm), a vaccine composition comprising a mixture of three cell lines, comprising HCT15, HUTU80 and LS411N; and the subject concurrently receives, via intradermal injection to the leg (e.g., thigh), a vaccine composition comprising a mixture of three cell lines, comprising HCT116, RKO and DMS 53.

Where an additional therapeutic agent is administered, the doses or multiple doses may be administered via the same or different route as the vaccine composition(s). By way of example, a composition comprising a checkpoint inhibitor is administered in some embodiments via intravenous injection, and the vaccine composition is administered via intradermal injection. In some embodiments, cyclophosphamide is administered orally, and the vaccine composition is administered intradermally. In other embodiments, ATRA is administered orally, and the vaccine composition is administered intradermally.

### Regimens

The vaccine compositions according to the disclosure may be administered at various administration sites on a subject, at various times, and in various amounts. The efficacy of a tumor cell vaccine may be impacted if the subject's immune system is in a state that is amenable to the activation of antitumor immune responses. For example, the vaccine efficacy may be impacted if the subject is undergoing or has received radiation therapy, chemotherapy or other prior treatments. In some embodiments, therapeutic efficacy will require inhibition of immunosuppressive elements of the immune system and fully functional activation and effector elements. In addition to the immunosuppressive factors described herein, other elements that suppress antitumor immunity include, but are not limited to, T regulatory cells (Tregs) and checkpoint molecules such as CTLA-4, PD-1 and PD-L1.

In some embodiments, timing of the administration of the vaccine relative to previous chemotherapy and radiation therapy cycles is set in order to maximize the immune permissive state of the subject's immune system prior to vaccine administration. The present disclosure provides methods for conditioning the immune system with one or low dose administrations of a chemotherapeutic agent such as cyclophosphamide prior to vaccination to increase efficacy of whole cell tumor vaccines. In some embodiments, metronomic chemotherapy (e.g., frequent, low dose administration of chemotherapy drugs with no prolonged drug-free break) is used to condition the immune system. In some embodiments, metronomic chemotherapy allows for a low level of the drug to persist in the blood, without the complications of toxicity and side effects often seen at higher doses. By way of example, administering cyclophosphamide to condition the immune system includes, in some embodiments, administration of the drug at a time before the receipt of a vaccine dose (e.g., 15 days to 1 hour prior to administration of a vaccine composition) in order to maintain the ratio of effector T cells to regulatory T cells at a level less than 1.

In some embodiments, a chemotherapy regimen (e.g., myeloablative chemotherapy, cyclophosphamide, and/or fludarabine regimen) may be administered before some, or all of the administrations of the vaccine composition(s) provided herein. Cyclophosphamide (CYTOXAN™, NEOSAR™) is a well-known cancer medication that interferes with the growth and spread of cancer cells in the body. Cyclophosphamide may be administered as a pill (oral), liquid, or via intravenous injection. Numerous studies have shown that cyclophosphamide can enhance the efficacy of vaccines. (See, e.g., Machiels et al., Cancer Res., 61:3689, 2001; Greten, T. F., et al., J. Immunother., 2010, 33:211; Ghiringhelli et al., Cancer Immunol. Immunother., 56:641, 2007; Ge et al., Cancer Immunol. Immunother., 61:353, 2011; Laheru et al., Clin. Cancer Res., 14:1455, 2008; and Borch et al., Oncolmmunol, e1207842, 2016). “Low dose” cyclophosphamide as described herein, in some embodiments, is effective in depleting Tregs, attenuating Treg activity, and enhancing effector T cell functions. In some embodiments, intravenous low dose administration of cyclophosphamide includes 40-50 mg/kg in divided doses over 2-5 days. Other low dose regimens include 1-15 mg/kg every 7-10 days or 3-5 mg/kg twice weekly. Low dose oral administration, in accordance with some embodiments of the present disclosure, includes 1-5 mg/kg per day for both initial and maintenance dosing. Dosage forms for the oral tablet are 25 mg and 50 mg. In some embodiments, cyclophosphamide is administered as an oral 50 mg tablet for the 7 days leading up to the first and optionally each subsequent doses of the vaccine compositions described herein.

In some embodiments, cyclophosphamide is administered as an oral 50 mg tablet on each of the 7 days leading up to the first, and optionally on each of the 7 days preceding each subsequent dose(s) of the vaccine compositions. In another embodiment, the patient takes or receives an oral dose of 25 mg of cyclophosphamide twice daily, with one dose being the morning upon rising and the second dose being at night before bed, 7 days prior to each administration of a cancer vaccine cocktail or unit dose. In certain embodiments, the vaccine compositions are administered intradermally multiple times over a period of years. In some embodiments, a checkpoint inhibitor is administered every two weeks or every three weeks following administration of the vaccine composition(s).

In another embodiment, the patient receives a single intravenous dose of cyclophosphamide of 200, 250, 300, 500 or 600 mg/m² at least one day prior to the administration of a cancer vaccine cocktail or unit dose of the vaccine composition. In another embodiment, the patient receives an intravenous dose of cyclophosphamide of 200, 250, 300, 500 or 600 mg/m² at least one day prior to the administration vaccine dose number 4, 8, 12 of a cancer vaccine cocktail or unit dose. In another embodiment, the patient receives a single dose of cyclophosphamide at 1000 mg/kg as an intravenous injection at least one hour prior to the administration of a cancer vaccine cocktail or unit dose. In some embodiments, an oral high dose of 200 mg/kg or an IV high dose of 500-1000 mg/m² of cyclophosphamide is administered.

The administration of cyclophosphamide can be via any of the following: oral (e.g., as a capsule, powder for solution, or a tablet); intravenous (e.g., administered through a vein (IV) by injection or infusion); intramuscular (e.g., via an injection into a muscle (IM)); intraperitoneal (e.g., via an injection into the abdominal lining (IP)); and intrapleural (e.g., via an injection into the lining of the lung).

In some embodiments, immunotherapy checkpoint inhibitors (e.g., anti-CTLA4, anti-PD-1 antibodies such as pembrolizumab, and nivolumab, anti-PDL1 such as durvalumab) may be administered before, concurrently, or after the vaccine composition. In certain embodiments, pembrolizumab is administered 2 mg/kg every 3 weeks as an intravenous infusion over 60 minutes. In some embodiments, pembrolizumab is administered 200 mg every 3 weeks as an intravenous infusion over 30 minutes. In some embodiments pembrolizumab is administered 400 mg every 6 weeks as an intravenous infusion over 30 minutes. In some embodiments, durvalumab is administered 10 mg/kg every two weeks. In some embodiments, nivolumab is administered 240 mg every 2 weeks (or 480 mg every 4 weeks). In some embodiments, nivolumab is administered 1 mg/kg followed by ipilimumab on the same day, every 3 weeks for 4 doses, then 240 mg every 2 weeks (or 480 mg every 4 weeks). In some embodiments, nivolumab is administered 3 mg/kg followed by ipilimumab 1 mg/kg on the same day every 3 weeks for 4 doses, then 240 mg every 2 weeks (or 480 mg every 4 weeks). In some embodiments, nivolumab is administered or 3 mg/kg every 2 weeks.

In some embodiments, durvalumab or pembrolizumab is administered every 2, 3, 4, 5, 6, 7 or 8 weeks for up to 8 administrations and then reduced to every 6, 7, 8, 9, 10, 11 or 12 weeks as appropriate.

In other embodiments, the present disclosure provides that PD-1 and PD-L1 inhibitors are administered with a fixed dosing regimen (i.e., not weight-based). In non-limiting examples, a PD-1 inhibitor is administered weekly or at weeks 2, 3, 4, 6 and 8 in an amount between 100-1200 mg. In non-limiting examples, a PD-L1 inhibitor is administered weekly or at weeks 2, 3, 4, 6 and 8 in an mount between 250-2000 mg.

In some embodiments, a vaccine composition or compositions as described herein is administered concurrently or in combination with a PD-1 inhibitor dosed either Q1W, Q2W, Q3W, Q4W, Q6W, or Q8W, between 100 mg and 1500 mg fixed or 0.5 mg/kg and 15 mg/kg based on weight. In another embodiment, a vaccine composition or compositions as described herein is administered concurrently in combination with PD-L1 inhibitor dosed either Q2W, Q3W, or Q4W between 250 mg and 2000 mg fixed or 2 mg/kg and 30 mg/kg based on weight. In other embodiments, the aforementioned regimen is administered but the compositions are administered in short succession or series such that the patient receives the vaccine composition or compositions and the checkpoint inhibitor during the same visit.

The plant Cannabis sativa L. has been used as an herbal remedy for centuries and is an important source of phytocannabinoids. The endocannabinoid system (ECS) consists of receptors, endogenous ligands (endocannabinoids) and metabolizing enzymes, and plays a role in different physiological and pathological processes. Phytocannabinoids and synthetic cannabinoids can interact with the components of ECS or other cellular pathways and thus may affect the development or progression of diseases, including cancer. In cancer patients, cannabinoids can be used as a part of palliative care to alleviate pain, relieve nausea and stimulate appetite. In addition, numerous cell culture and animal studies have demonstrated antitumor effects of cannabinoids in various cancer types. (For a review, see Daris, B., et al., Bosn. J. Basic. Med. Sci., 19(1):14-23 (2019).) Phytocannabinoids are a group of C21 terpenophenolic compounds predominately produced by the plants from the genus Cannabis. There are several different cannabinoids and related breakdown products. Among these are tetrahydrocannabinol (THC), cannabidiol (CBD), cannabinol (CBN), cannabichromene (CBC), A8-THC, cannabidiolic acid (CBDA), cannabidivarin (CBDV), and cannabigerol (CBG).

In certain embodiments of the present disclosure, use of all phytocannabinoids is stopped prior to or concurrent with the administration of a Treg cell inhibitor such as cyclophosphamide, and/or is otherwise stopped prior to or concurrent with the administration of a vaccine composition according to the present disclosure. In some embodiments, where multiple administrations of cyclophosphamide or vaccine compositions occur, the cessation optionally occurs prior to or concurrent with each administration. In certain embodiments, use of phytocannabinoids is not resumed until a period of time after the administration of the vaccine composition(s). For example, abstaining from cannabinoid administration for at least 1, 2, 3, 4, 5, 6, 7, 8, 9 or 10 days prior to administration and at least 1, 2, 3, 4, 5, 6, 7, 8, 9, or 10 days after administration of cyclophosphamide or a vaccine dose is contemplated.

In some embodiments, patients will receive the first dose of the vaccine within 6-12 weeks after completion of chemotherapy. High dose chemotherapy used in cancer treatment ablates proliferating cells and depletes immune cell subsets. Upon completion of chemotherapy, the immune system will begin to reconstitute. The time span for T cells to recur is roughly 2-3 weeks. Because T cells are an immunological cell subset targeted for activation, in some embodiments, the cancer vaccine is administered within a window where there are sufficient T cells to prime, yet the subject remains lymphopenic. This environment, in which there are less cells occupying the niche will allow the primed T cells to rapidly divide, undergoing “homeostatic proliferation” in response to increased availability of cytokines (e.g., IL7 and IL15). Thus, by dosing the vaccine at this window, the potential efficacy of embodiments of the cancer vaccine platform as described herein is maximized to allow for the priming of antigen specific T cells and expansion of the vaccine associated T cell response.

### Methods of Selecting Cell Lines and Preparing Vaccines

### Cell Line Selection

For a given cancer or in instances where a patient is suffering from more than one cancer, a cell line or combination of cell lines is identified for inclusion in a vaccine composition based on several criteria. In some embodiments, selection of cell lines is performed stepwise as provided below. Not all cancer indications will require all of the selection steps and/or criteria.
Step 1. Cell lines for each indication are selected based on the availability of RNA-seq data such as for example in the Cancer Cell Line Encyclopedia (CCLE) database. RNA-seq data allows for the identification of candidate cell lines that have the potential to display the greatest breadth of antigens specific to a cancer indication of interest and informs on the potential expression of immunosuppressive factors by the cell lines. If the availability of RNA-seq data in the CCLE is limited, RNA-seq data may be sourced from the European Molecular Biology Laboratory-European Bioinformatics Institute (EMBL-EBI) database or other sources known in the art. In some embodiments, potential expression of a protein of interest (e.g., a TAA) based on RNA-seq data is considered “positive” when the RNA-seq value is >0.
Step 2. For all indications, cell lines derived from metastatic sites are prioritized to diversify antigenic breadth and to more effectively target later-stage disease in patients with metastases. Cell lines derived from primary tumors are included in some embodiments to further diversify breadth of the vaccine composition. The location of the metastases from which the cell line are derived is also considered in some embodiments. For example, in some embodiments, cell lines can be selected that are derived from lymph node, ascites, and liver metastatic sites instead of all three cell lines derived from liver metastatic sites.
Step 3. Cell lines are selected to cover a broad range of classifications of cancer types. For example, tubular adenocarcinoma is a commonly diagnosed classification of gastric cancer. Thus, numerous cell lines may be chosen matching this classification. For indications where primary tumor sites vary, cell lines can be selected to meet this diversity. For example, cell lines originating from small and large intestinal track can be chosen for CRC. These selection criteria enable targeting a heterogeneous population of patient tumor types. In some embodiments, cell lines are selected to encompass an ethnically diverse population to generate a cell line candidate pool derived from diverse histological and ethnical backgrounds.
Step 4. In some embodiments, cell lines are selected based on additional factors. For example, in metastatic colorectal cancer (mCRC), cell lines reported as both microsatellite instable high (MSI-H) and microsatellite stable (MSS) may be included. As another example, for indications that are viral driven, cell lines encoding viral genomes may be excluded for safety and/or manufacturing complexity concerns.
Step 5. In some embodiments, cell lines are selected to cover a varying degree of genetic complexity in driver mutations or indication-associated mutations. Heterogeneity of cell line mutations can expand the antigen repertoire to target a larger population within patients with one or more tumor types. Each cancer type has a complex genomic landscape and, as a result, cell lines are selected for similar gene mutations for specific indications. For example, melanoma tumors most frequently harbor alterations in BRAF, CDKN2A, NRAS and TP53, therefore selected melanoma cell lines, in some embodiments, contain genetic alterations in one or more of these genes.
Step 6. In some embodiments, cell lines are further narrowed based on the TAA, TSA, and/or cancer/testis antigen expression based on RNA-seq data. An antigen or collection of antigens associated with a particular tumor or tumors is identified using search approaches evident to persons skilled in the art (See, e.g., such as www.ncbi.nlm.nih.gov/pubmed/, and clinicaltrials.gov). In some embodiments, antigens can be included if associated with a positive clinical outcome or identified as highly expressed by the specific tumor or tumor types while expressed at lower levels in normal tissues.
Step 7. After Steps 1 through 6 are completed, in some embodiments, the list of remaining cell line candidates are consolidated based on cell culture properties and considerations such as doubling time, adherence, size, and serum requirements. For example, cell lines with a doubling time of less than 80 hours or cell lines requiring media serum (FBS, FCS)<10% can be selected. In some embodiments, adherent or suspension cell lines that do not form aggregates can be selected to ensure proper cell count and viability.
Step 8. In some embodiments, cell lines are selected based on the expression of immunosuppressive factors (e.g., based on RNA-seq data sourced from CCLE or EMBL as described in Step 1).

In some embodiments, a biopsy of a patient's tumor and subsequent TAA expression profile of the biopsied sample will assist in the selection of cell lines. Embodiments of the present disclosure therefore provide a method of preparing a vaccine composition comprising the steps of determining the TAA expression profile of the subject's tumor; selecting cancer cell lines; modifying cancer cell lines; and irradiating cell lines prior to administration to prevent proliferation after administration to patients.

### Preparing Vaccine Compositions

In certain embodiments, after expansion in manufacturing, all of the cells in a modified cell line are irradiated, suspended, and cryopreserved. In some embodiments, cells are irradiated 10,000 cGy. According to some embodiments, cells are irradiated at 7,000 to 15,000 cGy. According to some embodiments, cells are irradiated at 7,000 to 15,000 cGy.

In certain embodiments, each vial contains a volume of 120±10 μL (1.2×10⁷ cells). In some embodiments, the total volume injected per site is 300 NL or less. In some embodiments, the total volume injected per site is 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μL. Where, for example, the total volume injected is 300 μL, the present disclosure provides, in some embodiments that 3×100 μL volumes, or 2×150 μL, are injected, for a total of 300 μL.

In some embodiments, the vials of the component cell lines are stored in the liquid nitrogen vapor phase until ready for injection. In some embodiments, each of the component cell lines are packaged in separate vials.

As described herein, prior to administration, in some embodiments the contents of two vials are removed by needle and syringe and are injected into a third vial for mixing. In some embodiments, this mixing is repeated for each cocktail. In other embodiments, the contents of six vials are divided into two groups—A and B, where the contents of three vials are combined or mixed, optionally into a new vial (A), and the contents of the remaining three vials are combined or mixed, optionally into a new vial (B).

In certain embodiments, the cells will be irradiated prior to cryopreservation to prevent proliferation after administration to patients. In some embodiments, cells are irradiated at 7,000 to 15,000 cGy in order to render the cells proliferation incompetent.

In some embodiments, cell lines are grown separately and in the same growth culture media. In some embodiments, cell lines are grown separately and in different cell growth culture media.

### Xeno-Free Conversion of Whole Tumor Cell Vaccine Component Cell Lines

Analysis of antibody responses in subjects treated with a whole tumor cell vaccine has suggested a negative correlation between survival and the development of IgG antibody responses to the bovine α-Gal antigen. (See Xia et al., Cell Chem Biol 23(12):1515-1525 (2016)). This is significant because most whole tumor cell vaccines are comprised of tumor cell lines that have been expanded and cryopreserved in media containing fetal bovine serum (FBS), which contains the bovine α-Gal antigen.

In some embodiments, to prevent the immune response to foreign antigens that are present in FBS, the cell lines disclosed herein are adapted to xeno-free media composed of growth factors and supplements essential for cell growth that are from human source, prior to large scale cGMP manufacturing. As used herein, the terms “adapting” and “converting” or “conversion” are used interchangeably to refer to transferring/changing cells to a different media as will be appreciated by those of skill in the art. The xeno-free media formulation chosen can be, in some embodiments, the same across all cell lines or, in other embodiments, can be different for different cell lines. In some embodiments, the media composition will not contain any non-human materials and can include human source proteins as a replacement for FBS alone, or a combination of human source proteins and human source recombinant cytokines and growth factors (e.g., EGF). Additionally, the xeno-free media compositions can, in some embodiments, also contain additional supplements (e.g., amino acids, energy sources) that enhance the growth of the tumor cell lines. The xeno-free media formulation will be selected for its ability to maintain cell line morphology and doubling time no greater than twice the doubling time in FBS and the ability to maintain expression of transgenes comparable to that in FBS.

A number of procedures may be instituted to minimize the possibility of inducing IgG, IgA, IgE, IgM and IgD antibodies to bovine antigens. These include but are not limited to: cell lines adapted to growth in xeno-free media; cell lines grown in FBS and placed in xeno-free media for a period of time (e.g., at least three days) prior to harvest; cell lines grown in FBS and washed in xeno-free media prior to harvest and cryopreservation; cell lines cryopreserved in media containing Buminate (a USP-grade pharmaceutical human serum albumin) as a substitute for FBS; and/or cell lines cryopreserved in a medial formulation that is xeno-free, and animal-component free (e.g., CryoStor). In some embodiments, implementation of one or more of these procedures may reduce the risk of inducing anti-bovine antibodies by removing the bovine antigens from the vaccine compositions.

According to one embodiment, the vaccine compositions described herein do not comprise non-human materials. In some embodiments, the cell lines described herein are formulated in xeno-free media. Use of xeno-free media avoids the use of immunodominant xenogeneic antigens and potential zoonotic organisms, such as the BSE prion. By way of example, following gene modification, the cell lines are transitioned to xeno-free media and are expanded to generate seed banks. The seed banks are cryopreserved and stored in vapor-phase in a liquid nitrogen cryogenic freezer.

### In Vitro Assays

The ability of allogeneic whole cell cancer vaccines such as those described herein, to elicit anti-tumor immune responses, and to demonstrate that modifications to the vaccine cell lines enhance vaccine-associated immune responses, can be modelled with in vitro assays. Without being bound by any theory, the genetic modifications made to the vaccine cell line components augment adaptive immune responses through enhancing dendritic cell (DC) function in the vaccine microenvironment. The potential effects of expression of TAAs, immunosuppressive factors, and/or immunostimulatory factors can be modelled in vitro, for example, using flow cytometry-based assays and the IFNγ ELISpot assay.

In some embodiments, to model the effects of modifications to the vaccine cell line components in vitro, DCs are derived from monocytes isolated from healthy donor peripheral blood mononuclear cells (PBMCs) and used in downstream assays to characterize immune responses in the presence or absence of one or more immunostimulatory or immunosuppressive factors. The vaccine cell line components are phagocytized by donor-derived immature DCs during co-culture with the unmodified parental vaccine cell line (control) or the modified vaccine cell line components. The effect of modified vaccine cell line components on DC maturation, and thereby subsequent T cell priming, can be evaluated using flow cytometry to detect changes in markers of DC maturation such as CD40, CD83, CD86, and HLA-DR. Alternatively, the immature DCs are matured after co-culture with the vaccine cell line components, the mature DCs are magnetically separated from the vaccine cell line components, and then co-cultured with autologous CD14− PBMCs for 6 days to mimic in vivo presentation and stimulation of T cells. IFNγ production, a measurement of T cell stimulatory activity, is measured in the IFNγ ELISpot assay or the proliferation and characterization of immune cell subsets is evaluated by flow cytometry. In the IFNγ ELISpot assay, PBMCs are stimulated with autologous DCs loaded with the unmodified parental vaccine cell line components to assess potential responses against unmodified tumor cells in vivo.

The IFNγ ELISpot assay can be used to evaluate the potential of the allogenic vaccine to drive immune responses to clinically relevant TAAs expressed by the vaccine cell lines. To assess TAA-specific responses in the IFNγ ELISpot assay, following co-culture with DCs, the PBMCs are stimulated with peptide pools comprising known diverse MHC-I epitopes for TAAs of interest. In various embodiments, the vaccine composition may comprise 3 cell lines that induce IFNγ responses to at least 3, 4, 5, 6, 7, 8, 9, 10, or 11 non-viral antigens, or at least 30%, 40%, 50%, 60%, 70%, 80%, 90%, or 100% of the antigens evaluated for an IFNγ response. In some embodiments, the vaccine composition may be a unit dose of 6 cell lines that induce IFNγ responses to at least 5, 6, 7, 8, 9, 10 or 11 non-viral antigens, or at least 60%, 70%, 80%, 90%, or 100% of the antigens evaluated for an IFNγ response.

### In Vivo Mouse Models

Induction of antigen specific T cells by the allogenic whole cell vaccine can be modeled in vivo using mouse tumor challenge models. The vaccines provided in embodiments herein may not be administered directly to mouse tumor model due to the diverse xenogeneic homology of TAAs between mouse and human. However, a murine homolog of the vaccines can be generated using mouse tumor cell lines. Some examples of additional immune readouts in a mouse model are: characterization of humoral immune responses specific to the vaccine or TAAs, boosting of cellular immune responses with subsequent immunizations, characterization of DC trafficking and DC subsets at draining lymph nodes, evaluation of cellular and humoral memory responses, reduction of tumor burden, and determining vaccine-associated immunological changes in the TME, such as the ratio of tumor infiltrating lymphocytes (TILs) to Tregs. Standard immunological methods such as ELISA, IFNγ ELISpot, and flow cytometry will be used.

### Kits

The vaccine compositions described herein may be used in the manufacture of a medicament, for example, a medicament for treating or prolonging the survival of a subject with cancer, e.g., colorectal cancer.

Also provided are kits for treating or prolonging the survival of a subject with cancer containing any of the vaccine compositions described herein, optionally along with a syringe, needle, and/or instructions for use. Articles of manufacture are also provided, which include at least one vessel or vial containing any of the vaccine compositions described herein and instructions for use to treat or prolong the survival of a subject with cancer. Any of the vaccine compositions described herein can be included in a kit comprising a container, pack, or dispenser together with instructions for administration.

In some embodiments, provided herein is a kit comprising at least two vials, each vial comprising a vaccine composition (e.g., cocktail A and cocktail B), wherein each vial comprises at least 1, 2, 3, 4, 5, 6, 7, 8, 9, or 10 or more cell lines, wherein the cell lines are modified to inhibit or reduce production of one or more immunosuppressive factors, and/or express or increase expression of one or more immunostimulatory factors, and/or express a heterogeneity of tumor associated antigens, or neoantigens.

By way of example, a kit comprising 6 separate vials is provided, wherein each vial comprises one of the following cell lines: DMS 53, HCT-15, HuTu80, LS411N, HCT-116 and RKO.

In some embodiments, provided herein is a kit comprising at least two vials, each vial comprising a vaccine composition (e.g., cocktail A and cocktail B), wherein each vial comprises at least three cell lines, wherein the cell lines are modified to reduce production or expression of one or more immunosuppressive factors, and/or modified to increase expression of one or more immunostimulatory factors, and/or express a heterogeneity of tumor associated antigens, or neoantigens. The two vials in these embodiments together are a unit dose. Each unit dose can have from about 5×10⁶ to about 5×10⁷ cells per vial, e.g., from about 5×10⁶ to about 3×10⁷ cells per vial.

In some embodiments, provided herein is a kit comprising at least six vials, each vial comprising a vaccine composition, wherein each vaccine composition comprises one cell line, wherein the cell line is modified to inhibit or reduce production of one or more immunosuppressive factors, and/or modified to express or increase expression of one or more immunostimulatory factors, and/or expresses a heterogeneity of tumor associated antigens, or neoantigens. Each of the at least six vials in the embodiments provided herein can be a unit dose of the vaccine composition. Each unit dose can have from about 2×10⁶ to about 50×10⁶ cells per vial, e.g., from about 2×10⁶ to about 10×10⁶ cells per vial.

In some embodiments, provided herein is a kit comprising separate vials, each vial comprising a vaccine composition, wherein each vaccine composition comprises one cell line, wherein the cell line is modified to inhibit or reduce production of one or more immunosuppressive factors, and/or modified to express or increase expression of one or more immunostimulatory factors, and/or expresses, a heterogeneity of tumor associated antigens, or neoantigens. Each of the vials in the embodiments provided herein can be a unit dose of the vaccine composition. Each unit dose can have from about 2×10⁶ to about 50×10⁶ cells per vial, e.g., from about 2×10⁶ to about 10×10⁶ cells per vial.

In one exemplary embodiment, a kit is provide comprising two cocktails of 3 cell lines each (i.e., total of 6 cell lines in 2 different vaccine compositions) as follows: 8×10⁶ cells per cell line; 2.4×10⁷ cells per injection; and 4.8×10⁷ cells total dose. In another exemplary embodiment, 1×10⁷ cells per cell line; 3.0×10⁷ cells per injection; and 6.0×10⁷ cells total dose is provided. In some embodiments, a vial of any of the kits disclosed herein contains about 0.1, 0.2, 0.3, 0.4, 0.5, 0.6, 0.7, 0.8, 0.9, or 1.0 mL of a vaccine composition of the disclosure. In some embodiments, the concentration of cells in a vial is about 5×10⁷ cells/mL to about 5×10⁸/cells mL.

The kits as described herein can further comprise needles, syringes, and other accessories for administration.

Described herein and in the co-filed sequence listing are various polynucleotide and polypeptide sequences. If there are discrepencies, the sequences provided in the text control.

### EXAMPLES

International patent application number PCT/US2020/062840 (Pub. No. WO/2021/113328) describes numerous methods and materials related to modified, whole cell cancer vaccines, which are incorporated by reference herein in their entirety. In some embodiments, the present disclosure including the following Examples provide additional and/or alternative cancer cell and cell line modifications.

Example 28 of PCT/US2020/062840 (Pub. No. WO/2021/113328) demonstrates that the reduction of TGFβ1, TGFβ2, and CD276 expression with concurrent overexpression of GM-CSF, CD40L, and IL-12 in a NSCLC vaccine comprising two cocktails, each cocktail composed of three cell line components, a total of 6 component cell lines, significantly increases the antigenic breadth and magnitude of cellular immune responses compared to belagenpumatucel-L.

Cancer immunotherapy through induction of anti-tumor cellular immunity has become a promising approach targeting cancer. Many therapeutic cancer vaccine platforms are targeting tumor associated antigens (TAAs) that are overexpressed in tumor cells, however, a cancer vaccine using these antigens must be potent enough to break tolerance. The cancer vaccines described in various embodiments herein are designed with the capacity to elicit broad and robust cellular responses against tumors. Neoepitopes are non-self epitopes generated from somatic mutations arising during tumor growth. Tumor types with higher mutational burden are correlated with durable clinical benefit in response to checkpoint inhibitor therapies. Targeting neoepitopes has many advantages because these neoepitopes are truly tumor specific and not subject to central tolerance in the thymus. A cancer vaccine encoding full length TAAs with neoepitopes arising from nonsynonymous mutations (NSMs) has potential to elicit a more potent immune response with improved breadth and magnitude. Example 40 of PCT/US2020/062840 (Pub. No. WO/2021/113328) describes improving breadth and magnitude of vaccine-induced cellular immune responses by introducing non-synonymous mutations (NSM) into prioritized full-length tumor associated antigens (TAAs).

### Example 1: Driver Mutation Identification and Design Process

Based on the number of alleles harboring a mutation and the fraction of tumor cells with the mutation, mutations can be classified as clonal (truncal mutations, present in all tumor cells sequenced) and subclonal (shared and private mutations, present in a subset of regions or cells within a single biopsy). Unlike the majority of neoepitopes that are private mutations and not found in more than one patient, driver mutations in known driver genes typically occur early in cancer evolution and are found in all or a subset of tumor cells across patients. Driver mutations show a tendency to be clonal and give a fitness advantage to the tumor cells that carry them and are crucial for the tumor's transformation, growth and survival. In various embodiments, the present disclosure provides methods for selecting and targeting driver mutations as an effective strategy to overcome intra- and inter-tumor neoantigen heterogeneity and tumor escape. Inclusion of a pool of driver mutations that occur at high frequency in a vaccine can promote potent anti-tumor immune responses.

The following Example provides the process for identifying and selecting driver mutations for inclusion in a cancer vaccine according to the present disclosure. This process was followed for the Examples described herein.

### Identification of Frequently Mutated Oncogenes for Each Indication

Oncogenes have the potential to initiate and maintain cancer phenotype and are often mutated in tumor cells. Missense driver mutations represent a greater fraction of the total mutations in oncogenes, and these driver mutations are implicated in oncogenesis by deregulating the control of normal cell proliferation, differentiation, and death, leading to growth advantage for the malignant clone.

To identify frequently mutated oncogenes for each indication, the dataset of “curated set of non-redundant studies” specific for each indication was first selected and explored from the publicly available database cBioPortal. Then a complete list of mutated genes was downloaded from the indication-specific dataset, and the cancer genes (oncogenes) were sorted out from the list and ranked by the percentage of samples with one or more mutations (mutation frequency). Any oncogenes with greater than 5% mutation frequency were selected for driver mutation identification and selection.

### Identification of Driver Mutations in Selected Oncogenes

Once the oncogenes were selected, the non-redundant data set was queried with the HUGO Gene Nomenclature Committee gene symbol for the oncogene of interest. Missense mutations occurring in the target oncogene were downloaded and sorted by frequency of occurrence. Missense mutations occurring in the same amino acid position in >0.5% of profiled patient samples in each selected oncogene were included as driver mutations for further prioritization.

### Prioritization and Selection of Identified Driver Mutations

Previous studies have shown that long peptide-based vaccine could potentially include MHC class I and II epitopes, thus eliciting robust cytotoxic and T helper cell responses. Therefore, a long peptide sequence containing a given driver mutation that is 28-35 amino acid in length was generated for CD4 and CD8 epitope analysis. A respective driver mutation was placed in the middle of a 28-35-mer peptide, and flanked by roughly 15 aa on either side taken from the respective non-mutated, adjacent, natural human protein backbone. When two (or more) driver mutations occur within 9 amino acids of a protein sequence, a long peptide sequence containing two (or more) driver mutations was also generated for CD4 and CD8 epitope analysis so long as there were at least 8 amino acids before and after each driver mutation.

These driver mutation-containing long peptide sequences were first evaluated based on the number of CD8 epitopes introduced by inclusion of a driver mutation using the publicly available NetMHCpan 4.0 (http://www.cbs.dtu.dk/services/NetMHCpan-4.0/) database. Then the selected driver mutations from the CD8 epitope analysis were further prioritized based on the number of CD4 epitopes introduced by inclusion of a driver mutation using the publicly available NetMHCllpan 4.0 (http://www.cbs.dtu.dk/services/NetMHCllpan/) database. The final list of driver mutations was generated based on the collective info on CD4 and CD8 epitope analysis and frequencies of these driver mutations.

For the CD8 epitope prediction, the HLA class I supertypes included are HLA-A*01:01, HLA-A*02:01, HLA-A*03:01, HLA-A*24:02, HLA-A*26:01, HLA-B*07:02, HLA-B*08:01, HLA-B*27:05, HLA-B*39:01, HLA-B*40:01, HLA-B*58:01, and HLA-B*15:01 (Table 1-1). The threshold for strong binder was set at the recommended threshold of 0.5, which means any peptides with predicted % rank lower than 0.5 will be annotated as strong binders. The threshold for weak binder was set at the recommended 2.0, which means any peptides with predicted % rank lower than 2.0 but higher than 0.5 would be annotated as weak binders. Only epitopes that contain the driver mutation are included in the analysis.

TABLE 1-1
HLA Class I supertypes used to predict CD8 epitopes

| Supertype | Representative |
|---|---|
| A01 | HLA-A*01:01 |
| A02 | HLA-A*02:01 |
| A03 | HLA-A*03:01 |
| A24 | HLA-A*24:02 |
| A26 | HLA-A*26:01 |
| B07 | HLA-B*07:02 |
| B08 | HLA-B*08:01 |
| B27 | HLA-B*27:05 |
| B39 | HLA-B*39:01 |
| B44 | HLA-B*40:01 |
| B58 | HLA-B*58:01 |
| B62 | HLA-B*15:01 |

For the CD4 epitope prediction, forty-six HLA Class II alleles are included and shown in Table 1-2. The threshold for strong binder was set at the recommended threshold of 2, which means any peptides with predicted % rank lower than 2 will be annotated as strong binders. The threshold for weak binder was set at the recommended 10, which means any peptides with predicted % rank lower than 10 but higher than 2 will be annotated as weak binders. For each driver mutation-containing sequence, all strong or weak binder CD4 epitopes that are 13, 14, 15, 16 and 17 amino acids in length were analyzed and recorded, respectively. Only epitopes that contain the driver mutation are included in the analysis. The highest number of CD4 epitopes for an allele predicted for 13, 14, 15, 16 or 17 amino acid epitopes was used for further analysis. The maximum number of strong or weak binders for each Class II allele was determined and the sum of the total predicted epitopes for each locus DRB1, DRB 3/4/5, DQA1/DQB and DPB1 were recorded. The total number of CD4 epitopes is the sum of the number of epitopes in each locus (DRB1+DRB 3/4/5+DQAD/DQBQ+DPBD).

TABLE 1-2
HLA Class II alleles used to predict CD4 epitopes

| DRB1 | DRB3/4/5 | DQA1/DQB1 | DPB1 |
|---|---|---|---|
| DRB1*0101 | DRB3*0101 | DQA1*0501/DQB1*0201 | DPA1*0201/DPB1*0101 |
| DRB1*0301 | DRB3*0202 | DQA1*0201/DQB1*0201 | DPA1*0103/DPB1*0201 |
| DRB1*0302 | DRB3*0301 | DQA1*0501/DQB1*0301 | DPA1*0103/DPB1*0401 |
| DRB1*0401 | DRB4*0101 | DQA1*0301/DQB1*0302 | DPA1*0103/DPB1*0402 |
| DRB1*0402 | DRB5*0101 | DQA1*0401/DQB1*0402 | DPA1*0202/DPB1*0501 |
| DRB1*0403 | DRB5*0102 | DQA1*0101/DQB1*0501 | DPA1*0201/DPB1*1401 |
| DRB1*0404 | | DQA1*0102/DQB1*0502 | |
| DRB1*0405 | | DQA1*0102/DQB1*0602 | |
| DRB1*0407 | | | |
| DRB1*0411 | | | |
| DRB1*0701 | | | |
| DRB1*0802 | | | |
| DRB1*0901 | | | |
| DRB1*1101 | | | |
| DRB1*1102 | | | |
| DRB1*1103 | | | |
| DRB1*1104 | | | |
| DRB1*1201 | | | |
| DRB1*1301 | | | |
| DRB1*1302 | | | |
| DRB1*1303 | | | |
| DRB1*1304 | | | |
| DRB1*1401 | | | |
| DRB1*1402 | | | |
| DRB1*1501 | | | |
| DRB1*1601 | | | |

The general criteria of driver mutation down selection are:
1. If there is only one driver mutation at certain position, this driver mutation will be selected if inclusion of this mutation results in >1 CD8 epitope. Driver mutations that introduce zero CD8 epitope will be excluded.
2. If there are more than one driver mutation at the same position, the driver mutation that introduces greater number of CD8 epitopes will be selected.
3. If two driver mutations at the same position introduce the same number of CD8 epitopes, the mutation with higher frequency will be selected.
4. If two driver mutations at the same position have the similar number of CD8 epitopes and similar frequencies, the mutation with greater number of CD4 epitopes will be selected.
5. When two driver mutations occur within 9 amino acids of a protein sequence, each driver mutation was evaluated alone and combined.

### Patient Sample Coverage by Selected Driver Mutations

After driver mutations were prioritized and selected for each indication, the sequences encoding these driver mutations were assembled, separated by furin cleavage site to generate construct inserts. Each insert could potentially include up to 20 driver mutation-containing sequences. Once construct inserts were assembled, the analysis of patient sample coverage by each insert was performed. Briefly, the dataset of “curated set of non-redundant studies” specific for each indication was queried with the HUGO Gene Nomenclature Committee gene symbol for the oncogenes with identified driver mutations. Expression data was downloaded and Patient Samples that were “not profiled” for the oncogene containing the driver mutation were omitted. If a Patient ID was associated with more than one sample from different anatomical sites, for example from the primary tumor and a metastatic site, expression for both samples was retained in the final data set. The remaining samples was used to calculate the frequency of a driver mutation. The patient sample coverage by each insert was calculated based on the collective information of the total number of samples with one selected driver mutation, the total number of samples with >2 driver mutations from same antigen and the total number of samples with >2 driver mutations from different antigens.

### Example 2: Preparation of Colorectal Cancer (CRC) Vaccines

Example 2 demonstrates reduction of TGFβ1, TGFβ2, and CD276 expression with concurrent introduction of GM-CSF, membrane bound CD40L, and IL-12 expression in a vaccine composition of two cocktails, each cocktail composed of three cell lines for a total of six cell lines, significantly increased the magnitude of cellular immune responses against at least nine CRC-associated antigens in an HLA-diverse population. Example 2 also describes the process for identification, selection, and design of driver mutations expressed by CRC patient tumors. As described here in, expression of peptides encoding these mutations in certain cell lines of the of the CRC vaccine, described above and herein, also generate potent immune responses in an HLA diverse population.

As described herein, the first cocktail, CRC vaccine-A, is composed of cell line HCT-15, cell line HuTu-80 also modified to express modPSMA and peptides encoding one TP53 driver mutation, one PIK3CA driver mutation, one FBXW7 driver mutation, one SMAD4 driver mutation, one GNAS driver mutation and one ATM driver mutation, and cell line LS411 N.

The second cocktail, CRC vaccine-B, is composed of cell line HCT-116 also modified to express modTBXT, modWT1 and peptides encoding two KRAS driver mutations, cell line RKO also modified to express peptides encoding three TP53 driver mutations, one KRAS driver mutation, three PIK3CA driver mutations, two FBXW7 driver mutations, one CTNNB1 driver mutation and one ERBB3 driver mutation, and cell line DMS 53.

The six component cell lines collectively express at least twenty full-length antigens and twenty driver mutations that can provide an anti-CRC tumor response. Table 2-23, below, provides a summary of each cell line and the modifications associated with each cell line.

### CRC Vaccine Components

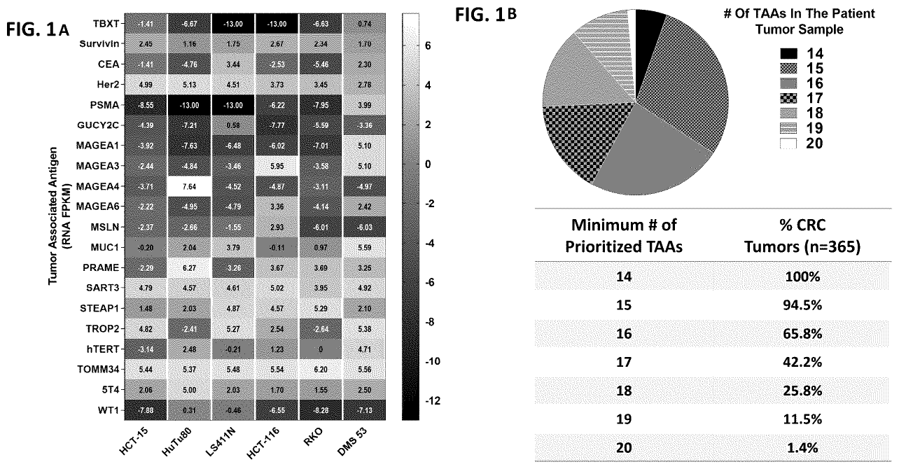

Example 30 of WO/2021/113328 first described selection of the cell lines comprising the CRC vaccine described herein. CRC vaccine cell lines were selected to express a wide array of TAAs, including those known to be important specifically for CRC antitumor responses, such as CEA, and TAAs known to be important for targets for CRC and other solid tumors, such as TERT. Expression of TAAs by vaccine cell lines was determined using RNA expression data sourced from the Broad Institute Cancer Cell Line Encyclopedia (CCLE). The HGNC gene symbol was included in the CCLE search and mRNA expression was downloaded for each TAA. Expression of a TAA by a cell line was considered positive if the RNA-seq value was >0.5. The six component cell lines expressed twelve to eighteen TAAs (FIG. 1A).

As shown herein, to further enhance antigenic breadth, HuTu80 was transduced with a gene encoding modPSMA and HCT-116 was transduced with genes encoding modTBXT, modWT1, and two 28 amino acid peptides spanning KRAS mutations G12D and G12V. Identification and design of antigen sequences inserted by lentiviral transduction into the CRC vaccine is described in Example 40 of WO/2021/113328 and herein. Identification, selection, and design of driver mutations was completed as described in Example 1 and herein.

RNA abundance of twenty prioritized CRC TAAs, identified as described in Example 40 of WO/2021/113328, was evaluated in 365 CRC patient samples Fourteen of the prioritized CRC TAAs were expressed by 100% of samples, 15 TAAs were expressed by 94.5% of samples, 16 TAAs were expressed by 65.8% of samples, 17 TAAs were expressed by 42.2% of samples, 18 TAAs were expressed by 25.8% of samples, 19 TAAs were expressed by 11.5% of samples and 20 TAAs were expressed by 1.4% samples (FIG. 1B).

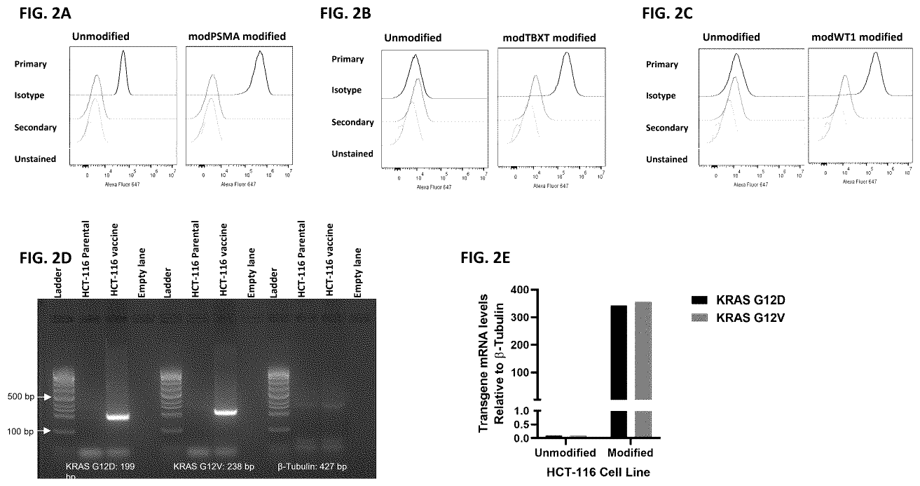

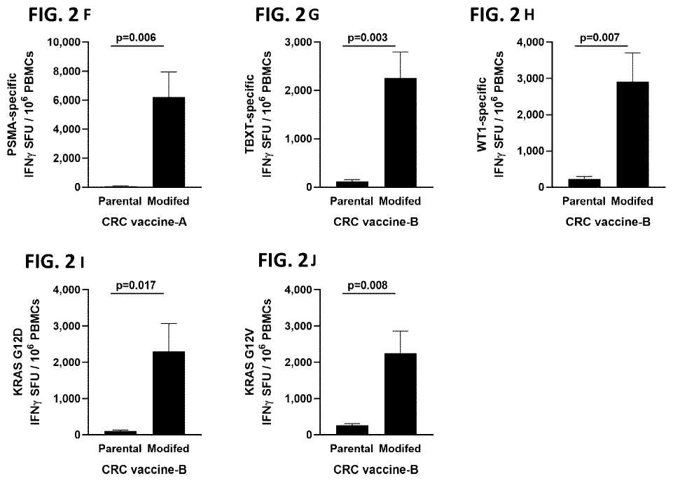

Expression of lentiviral transduced antigens modPSMA (FIG. 2A) (SEQ ID NO: 19; SEQ ID NO: 20) by HuTu80, modTBXT (FIG. 2B) (SEQ ID NO: 17; SEQ ID NO: 18) and modWT1 (FIG. 2C) (SEQ ID NO: 17; SEQ ID NO: 18) by HCT-116 was detected by flow cytometry described herein. Expression of the genes encoding KRAS G12D (FIG. 2D, 2E) (SEQ ID NO: 21; SEQ ID NO: 22) and G12V (FIG. 2D, 2E) (SEQ ID NO: 23; SEQ ID NO: 24) peptides were detected by RT-PCR as described herein. Genes encoding modTBXT, modWT1, KRAS G12D and KRAS G12V were subcloned into the same lentiviral transfer vector separated by furin cleavage sequences (SEQ ID: 32). PSMA was endogenously expressed in one of the six component cell lines at >0.5 FPKM as described below. TBXT and WT1 were not expressed endogenously >0.5 FPKM by any of the six component CRC vaccine components (FIG. 1A). Endogenous expression of KRAS driver mutations is described herein.

Provided herein are two compositions comprising, three cancer cell lines, wherein the combination of the cell lines express at least 14 TAAs associated with a subset of CRC cancer subjects intended to receive said composition. The cell lines identified in Table 2-1 comprise the present CRC vaccine.

TABLE 2-1
CRC vaccine cell lines and histology

| Cocktail | Cell Line Name | Histology |
|---|---|---|
| A | HCT-15 | Colorectal Adenocarcinoma |
| A | HuTu-80 | Duodenum Adenocarcinoma |
| A | LS411N | Colorectal Adenocarcinoma |
| B | HCT-116 | Colorectal Carcinoma |
| B | RKO | Colorectal Carcinoma |
| B | DMS 53 | Lung Small Cell Carcinoma |

### Reduction of CD276 Expression

Unmodified, parental HCT-15, HuTu-80, LS411N, HCT-116, RKO and DMS 53 component cell lines expressed CD276. Expression of CD276 was knocked out by electroporation with a zinc finger nuclease (ZFN) pair specific for CD276 targeting the genomic DNA sequence: GGCAGCCCTGGCATGggtgtgCATGTGGGTGCAGCC. (SEQ ID NO: 25). Following ZFN-mediated knockout of CD276, the cell lines were surface stained with PE α-human CD276 antibody (BioLegend, clone DCN.70) and full allelic knockout cells were enriched by cell sorting (BioRad S3e Cell Sorter). Sorted cells were plated in an appropriately sized vessel, based on the number of recovered cells, and expanded in culture. After cell enrichment for full allelic knockouts, cells were passaged 2-5 times and CD276 knockout percentage determined by flow cytometry. Expression of CD276 was determined by extracellular staining of CD276 modified and unmodified parental cell lines with PE α-human CD276 (BioLegend, clone DCN.70). Unstained cells and isotype control PE α-mouse IgG1 (BioLegend, clone MOPC-21) stained parental and CD276 KO cells served as controls. To determine the percent reduction of CD276 expression in the modified cell line, the MFI of the isotype control was subtracted from recorded MFI values of both the parental and modified cell lines. Percent reduction of CD276 expression is expressed as: (1-(MFI of the CD276KO cell line/MFI of the parental))×100). Reduction of CD276 expression by component cell lines is described in Table 2-2. These data demonstrate that gene editing of CD276 with ZFN resulted in greater than 96.9% knockout of CD276 in the six NSCLC vaccine component cell lines.

TABLE 2-2
Reduction of CD276 expression

| | Unmodified | Modified | % |
|---|---|---|---|
| | Cell Line | Cell Line | Reduction |
| Cell line | MFI | MFI | CD276 |
| HCT-15 | 6,737 | 26 | 99.6 |
| HuTu-80 | 10,389 | 0 | 100.0 |
| LS411N | 34,278 | 4 | ≥99.9 |
| HCT-116 | 12,782 | 0 | 100.0 |
| RKO | 3,632 | 0 | 100.0 |
| DMS 53 | 4,479 | 0 | 100.0 |

MFI is reported with isotype controls subtracted

### Cytokine Secretion Assays for TGFβ1, TGFβ2, GM-CSF, and IL-12

Cell lines were ×-ray irradiated at 100 Gy prior to plating in 6-well plates at 2 cell densities (5.0e5 and 7.5e5) in duplicate. The following day, cells were washed with PBS and the media was changed to Secretion Assay Media (Base Media+5% CTS). After 48 hours, media was collected for ELISAs. The number of cells per well was counted using the Luna cell counter (Logos Biosystems). Total cell count and viable cell count were recorded. The secretion of cytokines in the media, as determined by ELISA, was normalized to the average number of cells plated in the assay for all replicates.

TGFβ1 secretion was determined by ELISA according to manufacturer's instructions (Human TGFβ1 Quantikine ELISA, R&D Systems #SB100B). Four dilutions were plated in duplicate for each supernatant sample. If the results of the ELISA assay were below the LLD, the percentage decrease relative to parental cell lines was estimated by the number of cells recovered from the assay and the lower limit of detection, 15.4 μg/mL. If TGFβ1 was detected in >2 samples or dilutions the average of the positive values was reported with the n of samples run.

TGFβ2 secretion was determined by ELISA according to manufacturer's instructions (Human TGFβ2 Quantikine ELISA, R&D Systems #SB250). Four dilutions were plated in duplicate for each supernatant sample. If the results of the ELISA assay were below the LLD, the percentage decrease relative to parental cell lines was estimated by the number of cells recovered from the assay and the lower limit of detection, 7.0 μg/mL. If TGFβ2 was detected in >2 samples or dilutions the average of the positive values was reported with the n of samples run.

GM-CSF secretion was determined by ELISA according to manufacturer's instructions (GM-CSF Quantikine ELISA, R&D Systems #SGM00). Four dilutions were plated in duplicate for each supernatant sample. If the results of the ELISA assay were below the LLD, the percentage increase relative to parental cell lines was estimated by the number of cells recovered from the assay and the lower limit of detection, 3.0 μg/mL. If GM-CSF was detected in >2 samples or dilutions the average of the positive values was reported with the n of samples run.

IL-12 secretion was determined by ELISA according to manufacturer's instructions (LEGEND MAX Human IL-12 (p70) ELISA, Biolegend #431707). Four dilutions were plated in duplicate for each supernatant sample. If the results of the ELISA assay were below the LLD, the percentage increase was estimated by the number of cells recovered from the assay and the lower limit of detection, 1.2 μg/mL. If IL-12 was detected in >2 samples or dilutions the average of the positive values was reported with the n of samples run.

shRNA Downregulates TGF-β Secretion

Following knockout of CD276, TGFβ1 and/or TGFβ2 secretion levels were reduced using shRNA and resulting secretion levels determined as described above. All unmodified CRC vaccine-A components secreted measurable levels of TGFβ1. HuTu80 also secreted detectable levels of TGFβ2. CRC vaccine-B cell lines HCT-116 and RKO secreted measurable levels of TGFβ1 but not TGFβ2 and DMS 53 secreted measurable levels of TGFβ1 and TGFβ2.

The five CRC-derived vaccine cell lines were transduced with the lentiviral particles encoding both TGFβ1 shRNA (shTGFβ1, mature antisense sequence: TTTCCACCATTAGCACGCGGG (SEQ ID NO: 26)) and the gene for expression of membrane bound CD40L (SEQ ID NO: 3) under the control of a different promoter. This allowed for simultaneous reduction of TGFβ1 and introduction of expression of membrane bound CD40L. Cell lines genetically modified to reduce TGFβ1, and not TGFβ2, are described by the clonal designation DK2.

HuTu80 was subsequently transduced with lentiviral particles encoding both TGFβ2 shRNA (mature antisense sequence: AATCTGATATAGCTCAATCCG (SEQ ID NO: 27) and GM-CSF (SEQ ID NO: 8) under the control of a different promoter. This allowed for simultaneous reduction of TGFβ2 and introduction of expression of GM-CSF. DMS 53 was concurrently transduced with both lentiviral particles encoding TGFβ1 shRNA and membrane bound CD40L with lentiviral particles encoding TGFβ2 shRNA and GM-CSF. This allowed for simultaneous reduction of TGFβ1 and TGFβ2 secretion and expression of GM-CSF. Cell lines genetically modified to decrease secretion of TGFβ1 and TGFβ2 are described by the clonal designation DK6.

Table 2-3 shows the percent reduction of TGFβ1 and/or TGFβ2 secretion by gene modified component cell lines compared to matched, unmodified cell lines. Gene modification resulted in at least 49% reduction of TGFβ1 secretion. Gene modification of TGFβ2 resulted in at least 97% reduction in secretion of TGFβ2.

TABLE 2-3
TGF-β Secretion (pg/10⁶ cells/24 hr) in Component Cell Lines

| Cell Line | Cocktail | Clone | TGFβ1 | TGFβ2 |
|---|---|---|---|---|
| HCT-15 | A | Wild type | 369 | 21 |
| HCT-15 | A | DK2 | 189 | NA |
| HCT-15 | A | Percent reduction | 49% | NA |
| HuTu-80 | A | Wild type | 2,529 | 4,299 |
| HuTu-80 | A | DK6 | 327 | 115 |
| HuTu-80 | A | Percent reduction | 87% | 97% |
| LS411N | A | Wild type | 413 | * ≤9 |
| LS411N | A | DK2 | 89 | NA |
| LS411N | A | Percent reduction | 78% | NA |
| HCT-116 | B | Wild type | 2,400 | * ≤8 |
| HCT-116 | B | DK2 | 990 | NA |
| HCT-116 | B | Percent reduction | 59% | NA |
| RKO | B | Wild type | 971 | * ≤6 |
| RKO | B | DK2 | 206 | NA |
| RKO | B | Percent reduction | 79% | NA |
| DMS 53 | B | Wild type | 205 | 806 |
| DMS 53 | B | DK6 | * ≤14 | * ≤16 |
| DMS 53 | B | Percent reduction | ≥93% | ≥99% |

DK6: TGFβ1/TGFβ2 double knockdown;
DK2: TGFβ1 single knockdown;
* estimated using LLD, not detected;
NA = not applicable

Based on a dose of 5×10⁵ of each component cell line, the total TGFβ1 and TGFβ2 secretion by CRC vaccine-A and CRC vaccine-B and respective unmodified parental controls are shown in Table 2-4. Secretion of TGFβ1 by CRC vaccine-A was reduced by 82% and TGFβ2 by 97% pg/dose/24 hr. Secretion of TGFβ1 by CRC vaccine-B was reduced by 66% and TGFβ2 by 98% pg/dose/24 hr.

TABLE 2-4
TGF-β Secretion (pg/dose/24 hr) by CRC
vaccine-A and CRC vaccine-B

| Cocktail | Clones | TGFβ1 | TGFβ2 |
|---|---|---|---|
| A | Unmodified | 1,656 | 2,165 |
| | DK2/DK6 | 303 | 58 |
| | Percent Reduction | 82% | 97% |
| B | Unmodified | 1,788 | 410 |
| | DK2/DK6 | 605 | 8 |
| | Percent Reduction | 66% | 98% |

### Membrane Bound CD40L (CD154) Expression

All CRC vaccine cell lines were transduced with lentiviral particles to reduced TGFβ1 secretion and to express membrane bound CD40L as described above and herein. Cells were analyzed for cell surface expression CD40L expression by flow cytometry. Unmodified and modified cells were stained with PE-conjugated human α-CD40L (BD Biosciences, clone TRAP1) or Isotype Control PE α-mouse IgG1 (BioLegend, clone MOPC-21). The MFI of the isotype control was subtracted from the CD40L MFI of both the unmodified and modified cell lines. If subtraction of the MFI of the isotype control resulted in a negative value, an MFI of 1.0 was used to calculate the fold increase in expression of CD40L by the modified component cell line relative to the unmodified cell line. Expression of membrane bound CD40L by all six vaccine component cell lines is described in Table 2-5. The results described below demonstrate CD40L membrane expression was substantially increased by all six cell CRC vaccine cell lines.

TABLE 2-5
Increase in membrane-bound CD40L (mCD40L) expression

| | Unmodified | Modified | Fold |
|---|---|---|---|
| | Cell Line | Cell Line | Increase |
| Cell line | MFI | MFI | mCD40L |
| HCT-15 | 0 | 669 | 669 |
| HuTu80 | 5 | 5,890 | 1,178 |
| LS411N | 0 | 4,703 | 4,703 |
| HCT-116 | 0 | 21,549 | 21,549 |
| RKO | 0 | 7,107 | 7,107 |
| DMS 53 | 0 | 4,317 | 4,317 |

MFI is reported with isotype controls subtracted

### Gm-CSF Expression

HuTu80 and DMS 53 were transduced with lentiviral particles encoding both TGFβ2 shRNA and the gene to express GM-CSF (SEQ ID NO: 8) under the control of a different promoter. The HCT-15, LS411 N, HCT-116 and RKO cell lines were transduced with lentiviral particles to only express GM-CSF (SEQ ID NO: 8). GM-CSF expression level by the CRC vaccine cell lines are described in Error! Reference source not found. 2-6 and herein.

TABLE 2-6
GM-CSF Secretion in Component Cell Lines

| | GM-CSF | GM-CSF |
|---|---|---|
| Cell Line | (ng/10⁶ cells/24 hr) | (ng/dose/24 hr) |
| HCT-15 | 59 | 30 |
| HuTu80 | 101 | 51 |
| LS411N | 145 | 73 |
| Cocktail A Total | 305 | 154 |
| HCT-116 | 342 | 171 |
| RKO | 131 | 66 |
| DMS 53 | 30 | 15 |
| Cocktail B Total | 503 | 252 |

Based on a dose of 5×10⁵ of each component cell line, the total GM-CSF secretion for CRC vaccine-A was 154 ng per dose per 24 hours. The total GM-CSF secretion for CRC vaccine-B was 252 ng per dose per 24 hours. The total GM-CSF secretion per dose was therefore 406 ng per 24 hours.

### Il-12 Expression

All vaccine cell lines were transduced with the lentivirus particles resulting in stable expression of IL-12 p70. Expression of IL-12 by components cell lines was determined as described above and the results are shown in Table 2-7.

TABLE 2-7
IL-12 expression by CRC vaccine-A and CRC vaccine-B

| | IL-12 | IL-12 |
|---|---|---|
| Cell Line | (ng/10⁶ cells/24 hr) | (ng/dose/24 hr) |
| HCT-15 | 27 | 14 |
| HuTu80 | 51 | 26 |
| LS411N | 26 | 13 |
| Cocktail A Total | 104 | 52 |
| HCT-116 | 186 | 93 |
| RKO | 43 | 22 |
| DMS 53 | 28 | 14 |
| Cocktail B Total | 257 | 129 |

Based on a dose of 5×10⁵ of each component cell line per cocktail IL-12 secretion by CRC vaccine-A was 52 ng per dose per 24 hours and 129 ng per dose per 24 hours by CRC vaccine-B. Total IL-12 secretion per unit dose 181 ng per 24 hours.

Stable Expression of modPSMA (SEQ ID NO: 20) by the HuTu80 Cell Line

CRC vaccine-A cell line HuTu80 modified to reduce expression of CD276, secretion of TGFβ1 and TGFβ2, and express GM-CSF, membrane bound CD40L, and IL-12 was transduced with lentiviral particles expressing the gene encoding modPSMA. Expression of PSMA was characterized by flow cytometry. Unmodified and antigen modified cells were stained intracellularly with 0.06 μg/test anti-mouse IgG1 anti-PSMA antibody (AbCam ab268061, Clone FOLH1/3734) followed by 0.125 ug/test AF647-conjugated goat anti-mouse IgG1 antibody (Biolegend #405322). The MFI of isotype control stained modPSMA transduced and antigen unmodified cells was subtracted from the MFI of cells stained for PSMA. Fold increase in antigen expression was calculated as: (background subtracted modified MFI/background subtracted parental MFI). Expression of PSMA increased by the antigen modified cell line (756,908 MFI) 9.1-fold over that of the cell line not modified to express modPSMA (82,993 MFI) (FIG. 2A).

Stable Expression of modTBXT, modWT1, KRAS G12D and KRAS G12V (SEQ ID NO: 18) by the HCT-116 Cell Line

CRC vaccine-B cell line HCT-116 modified to reduce the expression of CD276, reduce secretion of TGFβ1, and express GM-CSF, membrane bound CD40L, and IL-12 was transduced with lentiviral particles to express the genes encoding modTBXT, modWT1, and peptides encoding KRAS driver mutations G12D and G12V. Expression of TBXT and WT1 were confirmed by flow cytometry. Unmodified and antigen modified cells were stained intracellularly to detect the expression of each antigen as follows. For detection of modTBXT, cells were stained with rabbit anti-human TBXT antibody (Abcam ab209665, Clone EPR18113) (0.06 μg/test) or Rabbit Polyclonal Isotype Control (Biolegend 910801) followed by AF647-conjugated donkey anti-rabbit IgG antibody (Biolegend 406414) (0.125 μg/test). For detection of modWT1, cells were stained with rabbit anti-human WT1 antibody (AbCam ab89901, Clone CAN-R9) (0.06 μg/test) or Rabbit Polyclonal Isotype Control (Biolegend 910801) followed by AF647-conjugated donkey anti-rabbit IgG antibody (Biolegend 406414) (0.125 μg/test). The MFI of cells stained with the isotype control was subtracted from the MFI of the cells stained for TBXT or WT1. Fold increase in antigen expression was calculated as: (background subtracted modified MFI/background subtracted parental MFI). Expression of modTBXT increased by the antigen modified cell line (356,691 MFI) 356,691-fold over that of the antigen unmodified cell line (0 MFI) (FIG. 2B). Subtraction of the MFI of the isotype control from the MFI of the TBXT stained unmodified cell line resulted in negative value and fold increase of modTBXT expression by the antigen modified HCT-116 cell line was calculated using 1 MFl. Expression of modTBXT increased by TBXT expression (356,691 MFI) 356,691-fold over that of the antigen unmodified cell line (0 MFI) (FIG. 16B). Expression of modWT1 by increased WT-1 expression (362,698 MFI) 69.3-fold over the that of the antigen unmodified cell line (5,235 MFI) (FIG. 2C).

Expression of peptides encoding KRAS driver mutations G12D and G12V by HCT-116 was confirmed by RT-PCR. For KRAS G12D, the forward primer designed to anneal at the 2786-2807 base pair (bp) position of the transgene (GAAGCCCTTCAGCTGTAGATGG (SEQ ID NO: 28) and reverse primer designed to anneal at 2966-2984 bp position in the transgene (CTGAATTGTCAGGGCGCTC (SEQ ID NO: 29) and yield 199 bp product. For KRAS G12V, the forward primer was designed to anneal at the 2861-2882 bp location in the transgene (CATGCACCAGAGGAACATGACC (SEQ ID NO: 30) and reverse primer designed to anneal at the 3071-3094 bp location in the transgene (GAGTTGGATGGTCAGGGCAGAT (SEQ ID NO: 31) and yield 238 bp product. Gene products for both KRAS G12D and KRAS G12V were detected at the expected size, 199 bp and 238 bp, respectively (FIG. 2D). KRAS G12D mRNA increased 3,127-fold and KRAS G12V mRNA increased 4,095-fold relative to parental controls (FIG. 2E).

### Immune Responses to PSMA (SEQ ID NO: 20) by CRC-Vaccine A

IFNγ responses to PSMA were evaluated in the context of the CRC-vaccine A for six HLA diverse donors (Table 2-8). Specifically, 5×10⁵ of unmodified or CRC vaccine-A HCT-15, HuTu80 and LS411N cell lines, a total of 1.5×10⁶ total modified cells, were co-cultured with 1.5×10⁶ iDCs from six HLA diverse donors (n=4/donor). CD14-PBMCs were isolated from co-culture with DCs on day 6 and stimulated with peptide pools, 15-mers overlapping by 9 amino acids, spanning the native PSMA protein (Thermo Scientific Custom Peptide Service) the IFNγ ELISpot assay for 24 hours prior to detection of IFNγ producing cells. CRC vaccine-A (6,204±1,744 SFU) induced significantly stronger PSMA specific IFNγ responses compared to unmodified CRC vaccine -A (69±36 SFU) (p=0.006, Mann-Whitney U test) (FIG. 2F).

TABLE 2-8
Healthy Donor MHC-I characteristics

| Donor # | HLA-A | HLA-B | HLA-C |
|---|---|---|---|
| 1 | *02:01 *11:01 | *07:02 *37:02 | *06:02 *07:02 |
| 2 | *03:01 *25:01 | *15:01 *44:02 | *03:03 *05:01 |
| 3 | *02:01 *24:01 | *08:01 *44:02 | *05:01 *07:01 |
| 4 | *29:01 *29:02 | *44:03 *50:01 | *06:02 *16:01 |
| 5 | *11:01 *29:02 | *18:01 *44:03 | *07:01 *11:01 |
| 6 | *02:01 *03:01 | *07:02 *41:02 | *07:02 *17:01 |

### Immune Responses to TBXT, WTI, and KRAS Mutations (SEQ ID NO: 18) by CRC-Vaccine B

IFNγ responses to TBXT, WT1, KRAS G12D and KRAS G12V antigens were evaluated in the context of the CRC-vaccine B for six HLA diverse donors (n=4/donor) (Table 2-8). Specifically, 5×10⁵ of unmodified or CRC vaccine-B HCT-116, RKO and DMS 53 cell lines, a total of 1.5×10⁶ total modified cells, were co-cultured with 1.5×10⁶ iDCs from six HLA diverse donors. CD14-PBMCs were isolated from co-culture with DCs on day 6 and stimulated with peptide pools of 15-mer peptides, overlapping by 11 amino acids covering for the full-length protein sequences of TBXT (JPT, PM-BRAC) or WT1 (JPT, PM-WT1). KRAS G12D and G12V 15-mers overlapping by 9 amino acids, were purchased from Thermo Scientific Custom Peptide Service. IFNγ responses to TBXT significantly increased by modified CRC vaccine-B (2,257±538 SFU) compared to unmodified CRC vaccine-B (121±35 SFU) (p=0.003) (FIG. 2G). WT1 specific IFNγ responses were significantly increased by modified CRC vaccine-B (2,910±794 SFU) compared unmodified CRC vaccine-B (277±78 SFU) (p=0.007) (FIG. 2H). KRAS G12D specific IFNγ responses significantly increased with modified CRC vaccine-B (2,302±771 SFU) compared unmodified CRC vaccine-B (123±30 SFU) (p=0.017) (FIG. 2I). KRAS G12V specific IFNγ responses significantly increased with modified CRC vaccine-B (2,246±612 SFU) compared unmodified CRC vaccine-B (273±37 SFU) (p=0.008) (FIG. 2J). Statistical significance was determined by Student's T test.

### Cocktails Induce Immune Responses Against Prioritized TAAs

IFNγ responses generated by CRC vaccine-A and CRC vaccine-B against nine prioritized CRC antigens was measured by ELISpot as described above and herein. CD14-PBMCs from six HLA-diverse healthy donors (Table 2-8) were co-cultured with autologous DCs loaded with unmodified control cocktails, CRC vaccine-A or CRC vaccine-B for 6 days prior to stimulation with TAA-specific specific peptide pools designed to cover the full-length native antigen protein. Antigen specific IFNγ responses against PSMA, WT1, TBXT, KRAS G12D and KRAS G12V were evaluated in ELISpot by stimulating primed CD14-PBMCs with peptide pools described above. Additional peptide pools were sourced as follows: Survivin (thinkpeptides, 7769_001-011), PRAME (Miltenyi Biotech, 130-097-286), STEAP (PM-STEAP1), TERT (JPT, PM-TERT), MUC1 (JPT, PM-MUC1), and CEACAM (CEA) (JPT, PM-CEA).

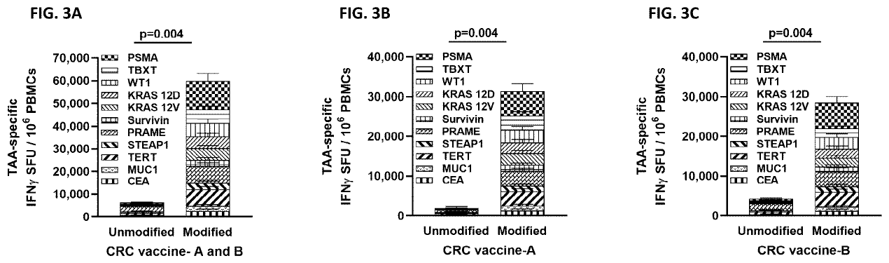

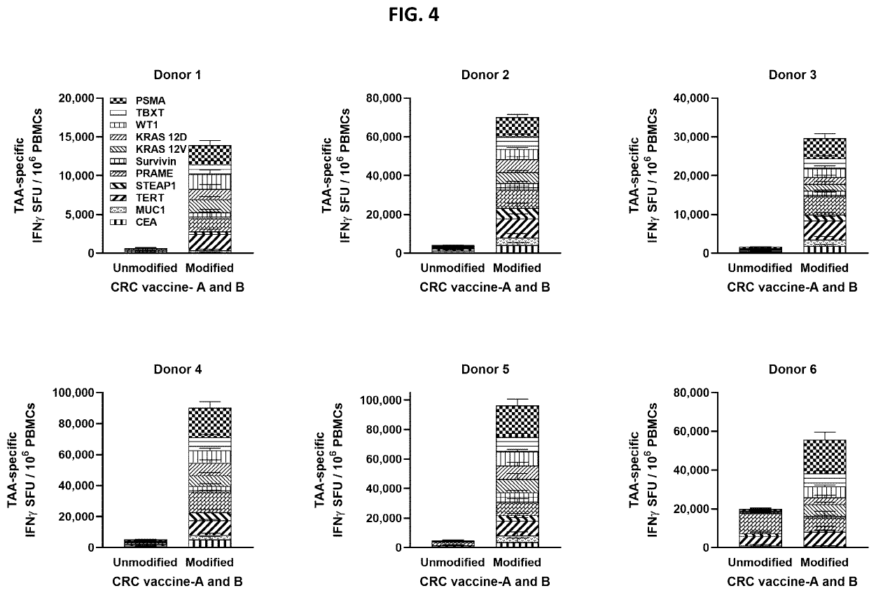

FIG. 3 demonstrates the CRC vaccine can induce antigen specific IFNγ responses in six HLA-diverse donors significantly more robust (59,976±13,542 SFU) compared to unmodified parental controls (6,247±2,891 SFU) (p=0.004) (FIG. 3A). CRC vaccine-A and CRC vaccine-B independently demonstrated antigen specific responses significantly greater compared to parental controls. Specifically, CRC vaccine-A elicited 31,489±7,103 SFU compared to the unmodified controls (1,931±1,333 SFU) (p=0.004) (FIG. 3B). CRC vaccine-B significantly increased antigen specific IFNγ ELISpot (28,487±7,156 SFU) compared to parental controls (4,316±1,645 SFU) (p=0.004) (FIG. 30). Immune responses by individual donors is described in FIG. 4 and Table 2-9). Statistical significance was determined by the Mann-Whitney U test.

TABLE 2-9
IFNγ Responses to TAAs induced by the unmodified and modified CRC vaccine

| Donor | Unmodified(SFU ± SEM) | | | Modified (SFU ± SEM) | | |
|---|---|---|---|---|---|---|
| (n = 4) | CRC vaccine-A | CRC vaccine-B | CRC vaccine | CRC vaccine-A | CRC vaccine-B | CRC vaccine |
| 1 | 135 ± 8 | 370 ± 18 | 505 ± 23 | 6,753 ± 129 | 6,993 ± 134 | 13,745 ± 242 |
| 2 | 1,150 ± 44 | 3,258 ± 78 | 4,408 ± 114 | 32,930 ± 333 | 37,335 ± 460 | 70,265 ± 734 |
| 3 | 630 ± 22 | 1,050 ± 25 | 1,680 ± 36 | 14,193 ± 244 | 14,715 ± 253 | 28,908 ± 469 |
| 4 | 1,150 ± 27 | 4,328 ± 92 | 5,478 ± 107 | 42,350 ± 646 | 47,860 ± 755 | 90,210 ± 1,376 |
| 5 | 0 ± 0 | 5,308 ± 221 | 5,308 ± 221 | 50,855 ± 677 | 46,260 ± 830 | 97,115 ± 1,461 |
| 6 | 8,520 ± 396 | 11,583 ± 581 | 20,103 ± 963 | 41,855 ± 982 | 17,758 ± 617 | 59,613 ± 1,562 |
| Ave. | 1,931 ± 1,333 | 4,316 ± 1,645 | 6,247 ± 2,891 | 31,489 ± 7,103 | 28,487 ± 7,156 | 59,976 ± 13,542 |

Identification of frequently mutated oncogenes in Colorectal Cancer (CRC)

Driver mutations for CRC were identified, selected and constructs designed as described as described in Example 1 and herein. As described herein, expression of selected driver mutations by CRC vaccine-A cell line Hutu80 and CRC vaccine-B cell lines HCT-116 and RKO can generate a CRC anti-tumor response in an HLA diverse population. Table 2-10 describes oncogenes that exhibit greater than 5% mutation frequency (percentage of samples with one or more mutations) in 1363 profiled CRC patient samples.

TABLE 2-10
Oncogenes in CRC with greater than 5% mutation frequency

| | | Number | | Percentage | |
|---|---|---|---|---|---|
| | Total | of samples | | of samples | Is Cancer |
| | number | with one | | with one | Gene |
| | of | or more | Profiled | or more | (source: |
| Gene | mutations | mutations | Samples | mutations | OncoKB) |
| APC | 1385 | 902 | 1363 | 66.20% | Yes |
| TP53 | 835 | 785 | 1363 | 57.60% | Yes |
| KRAS | 514 | 504 | 1363 | 37.00% | Yes |
| PIK3CA | 382 | 328 | 1363 | 24.10% | Yes |
| FAT4 | 409 | 250 | 1363 | 18.30% | Yes |
| LRP1B | 357 | 207 | 1363 | 15.20% | Yes |
| FBXW7 | 242 | 203 | 1363 | 14.90% | Yes |
| BRAF | 214 | 201 | 1363 | 14.70% | Yes |
| SMAD4 | 198 | 176 | 1363 | 12.90% | Yes |
| PCLO | 261 | 171 | 1363 | 12.50% | Yes |
| KMT2C | 209 | 159 | 1363 | 11.70% | Yes |
| KMT2D | 203 | 155 | 1363 | 11.40% | Yes |
| ATM | 212 | 150 | 1363 | 11.00% | Yes |
| RNF213 | 174 | 143 | 1363 | 10.50% | Yes |
| ZFHX3 | 164 | 138 | 1363 | 10.10% | Yes |
| AMER1 | 143 | 135 | 1363 | 9.90% | Yes |
| TRRAP | 173 | 132 | 1363 | 9.70% | Yes |
| ARID1A | 150 | 130 | 1363 | 9.50% | Yes |
| FAT1 | 191 | 129 | 1363 | 9.50% | Yes |
| EP400 | 157 | 129 | 1363 | 9.50% | Yes |
| SOX9 | 145 | 128 | 1363 | 9.40% | Yes |
| RNF43 | 162 | 126 | 1363 | 9.20% | Yes |
| MKI67 | 146 | 119 | 1363 | 8.70% | Yes |
| RELN | 172 | 119 | 1363 | 8.70% | Yes |
| PTPRS | 133 | 116 | 1363 | 8.50% | Yes |
| PDE4DIP | 157 | 114 | 1363 | 8.40% | Yes |
| CHD4 | 138 | 111 | 1363 | 8.10% | Yes |
| PTPRT | 126 | 109 | 1363 | 8.00% | Yes |
| ANKRD11 | 131 | 108 | 1363 | 7.90% | Yes |
| ROBO1 | 128 | 107 | 1363 | 7.90% | Yes |
| MTOR | 118 | 103 | 1363 | 7.60% | Yes |
| CREBBP | 122 | 102 | 1363 | 7.50% | Yes |
| LRRK2 | 144 | 102 | 1363 | 7.50% | Yes |
| TCF7L2 | 105 | 100 | 1363 | 7.30% | Yes |
| KMT2B | 126 | 100 | 1363 | 7.30% | Yes |
| PRKDC | 146 | 99 | 1363 | 7.30% | Yes |
| UBR5 | 121 | 99 | 1363 | 7.30% | Yes |
| ACVR2A | 110 | 98 | 1363 | 7.20% | Yes |
| ERBB4 | 114 | 98 | 1363 | 7.20% | Yes |
| PREX2 | 127 | 98 | 1363 | 7.20% | Yes |
| CARD11 | 107 | 97 | 1363 | 7.10% | Yes |
| NOTCH1 | 106 | 94 | 1363 | 6.90% | Yes |
| PTEN | 119 | 92 | 1363 | 6.70% | Yes |
| NCOR2 | 108 | 92 | 1363 | 6.70% | Yes |
| GRIN2A | 110 | 91 | 1363 | 6.70% | Yes |
| KMT2A | 124 | 91 | 1363 | 6.70% | Yes |
| ATRX | 126 | 90 | 1363 | 6.60% | Yes |
| CACNA1D | 121 | 90 | 1363 | 6.60% | Yes |
| ALK | 101 | 89 | 1363 | 6.50% | Yes |
| MYH9 | 112 | 89 | 1363 | 6.50% | Yes |
| NOTCH3 | 105 | 89 | 1363 | 6.50% | Yes |
| POLE | 113 | 89 | 1363 | 6.50% | Yes |
| BCORL1 | 105 | 89 | 1363 | 6.50% | Yes |
| SPEN | 119 | 88 | 1363 | 6.50% | Yes |
| BCL9L | 101 | 88 | 1363 | 6.50% | Yes |
| BRCA2 | 137 | 86 | 1363 | 6.30% | Yes |
| CUX1 | 97 | 86 | 1363 | 6.30% | Yes |
| ARID1B | 100 | 85 | 1363 | 6.20% | Yes |
| CTNNB1 | 101 | 84 | 1363 | 6.20% | Yes |
| MYH11 | 107 | 84 | 1363 | 6.20% | Yes |
| SMARCA4 | 94 | 84 | 1363 | 6.20% | Yes |
| NF1 | 100 | 82 | 1363 | 6.00% | Yes |
| PIK3CG | 95 | 82 | 1363 | 6.00% | Yes |
| PLCG2 | 92 | 82 | 1363 | 6.00% | Yes |
| AXIN2 | 96 | 82 | 1363 | 6.00% | Yes |
| MGA | 104 | 81 | 1363 | 5.90% | Yes |
| SLX4 | 92 | 81 | 1363 | 5.90% | Yes |
| FLT4 | 88 | 80 | 1363 | 5.90% | Yes |
| ERBB3 | 85 | 79 | 1363 | 5.80% | Yes |
| POLQ | 107 | 79 | 1363 | 5.80% | Yes |
| ASXL1 | 83 | 79 | 1363 | 5.80% | Yes |
| CAD | 87 | 78 | 1363 | 5.70% | Yes |
| PTPRK | 92 | 78 | 1363 | 5.70% | Yes |
| ARID2 | 106 | 78 | 1363 | 5.70% | Yes |
| CIC | 84 | 77 | 1363 | 5.60% | Yes |
| EP300 | 89 | 76 | 1363 | 5.60% | Yes |
| EPHA5 | 86 | 76 | 1363 | 5.60% | Yes |
| NUMA1 | 87 | 76 | 1363 | 5.60% | Yes |
| CAMTA1 | 84 | 76 | 1363 | 5.60% | Yes |
| GNAS | 79 | 75 | 1363 | 5.50% | Yes |
| LRP5 | 84 | 75 | 1363 | 5.50% | Yes |
| BCL9 | 87 | 74 | 1363 | 5.40% | Yes |
| PTPRD | 94 | 74 | 1363 | 5.40% | Yes |
| RANBP2 | 96 | 74 | 1363 | 5.40% | Yes |
| IRS1 | 83 | 73 | 1363 | 5.40% | Yes |
| MYO5A | 84 | 73 | 1363 | 5.40% | Yes |
| ROS1 | 113 | 73 | 1363 | 5.40% | Yes |
| IRS4 | 86 | 73 | 1363 | 5.40% | Yes |
| SETD1A | 87 | 73 | 1363 | 5.40% | Yes |
| PIK3R1 | 87 | 72 | 1363 | 5.30% | Yes |
| PTPRC | 90 | 72 | 1363 | 5.30% | Yes |
| COL1A1 | 75 | 71 | 1363 | 5.20% | Yes |
| TP53BP1 | 96 | 71 | 1363 | 5.20% | Yes |
| DICER1 | 88 | 71 | 1363 | 5.20% | Yes |
| SETBP1 | 90 | 71 | 1363 | 5.20% | Yes |
| ZBTB20 | 77 | 71 | 1363 | 5.20% | Yes |
| KDM2B | 78 | 71 | 1363 | 5.20% | Yes |
| B2M | 104 | 70 | 1363 | 5.10% | Yes |
| AFDN | 88 | 70 | 1363 | 5.10% | Yes |
| ZNF521 | 85 | 69 | 1363 | 5.10% | Yes |
| LARP4B | 77 | 68 | 1363 | 5.00% | Yes |

The CRC driver mutations in TP53, KRAS, PIKKCA, FBXW7, BRAF, SMAD4, ATM, CTNNB, ERBB3 and GNAS occurring in ≥0.5% of profiled patient samples are shown in Table 2-11. There were no missense mutations occurring in 0.5% of profiled patient samples for the rest of CRC oncogenes listed in Table 2-10.

TABLE 2-11
Identification of driver mutations in selected CRC oncogenes

| | | Number | | |
|---|---|---|---|---|
| | | of samples | Total | |
| | Driver | with | number | |
| Gene | mutation | mutation | of samples | Frequency |
| TP53 | G245S | 15 | 1363 | 1.1% |
| | R273H | 31 | 1363 | 2.3% |
| | R248W | 34 | 1363 | 2.5% |
| | R273C | 37 | 1363 | 2.7% |
| | R248Q | 41 | 1363 | 3.0% |
| | R282W | 41 | 1363 | 3.0% |
| | R175H | 93 | 1363 | 6.8% |
| KRAS | G12S | 16 | 1363 | 1.2% |
| | G12A | 21 | 1363 | 1.5% |
| | A146T | 27 | 1363 | 2.0% |
| | G12C | 44 | 1363 | 3.2% |
| | G12V | 97 | 1363 | 7.1% |
| | G13D | 99 | 1363 | 7.3% |
| | G12D | 142 | 1363 | 10.4% |
| PIK3CA | M1043I | 7 | 1363 | 0.5% |
| | H1047Y | 7 | 1363 | 0.5% |
| | C420R | 9 | 1363 | 0.7% |
| | E546K | 11 | 1363 | 0.8% |
| | R88Q | 26 | 1363 | 1.9% |
| | E542K | 37 | 1363 | 2.7% |
| | H1047R | 43 | 1363 | 3.2% |
| | E545K | 64 | 1363 | 4.7% |
| FBXW7 | S582L | 8 | 1363 | 0.6% |
| | R505C | 11 | 1363 | 0.8% |
| | R465H | 23 | 1363 | 1.7% |
| | R465C | 31 | 1363 | 2.3% |
| BRAF | V600E | 165 | 1363 | 12.1% |
| SMAD4 | R361C | 11 | 1363 | 0.8% |
| | R361H | 20 | 1363 | 1.5% |
| ATM | R337C | 7 | 1363 | 0.5% |
| CTNNB1 | S45F | 8 | 1363 | 0.6% |
| ERBB3 | V104M | 8 | 1363 | 0.6% |
| GNAS | R201H | 14 | 1363 | 1.0% |

Prioritization and Selection of/Ident/Fled CRC Driver Mutations

HLA-A and HLA-B supertype-restricted 9-mer CD8 epitopes analysis was performed as described in Example 1. Based on the CD8 epitope analysis result and the frequency (%) of each mutation, a list of mutations was selected to be either included in the final constructs or obtain further CD4 epitope analysis. The results are shown in Table 2-12.

TABLE 2-12
Prioritization and selection of identified CRC driver mutations
based on CD8 epitope analysis and frequency of each mutation

| | | Number of | | |
|---|---|---|---|---|
| | | total CD8 | | Included |
| | Driver | epitopes | Frequency | Yes (Y) or |
| Gene | mutation | (SB + WB) | (%) | No (N) |
| TP53 | R175H | 2 | 6.8 | Y |
| | G245S | 3 | 1.1 | N |
| | R248W | 3 | 2.5 | N |
| | R248Q | 0 | 3 | N |
| | G245S | 3 | 3.6 | Y |
| | R248W | | | |
| | R273C | 1 | 2.7 | Y |
| | R273H | 1 | 2.3 | N |
| | R282W | 0 | 3 | N |
| KRAS | G12S | 1 | 1.2 | N |
| | G12A | 2 | 1.5 | CD4 analysis |
| | G12C | 1 | 3.2 | CD4 analysis |
| | G12V | 3 | 7.1 | Y |
| | G12D | 1 | 10.4 | Y |
| | G13D | 0 | 7.3 | N |
| | A146T | 0 | 2 | N |
| PIK3CA | R88Q | 6 | 1.9 | Y |
| | C420R | 0 | 0.7 | N |
| | E542K | 1 | 2.7 | Y |
| | E545K | 0 | 4.7 | N |
| | Q546K | 0 | 0.9 | N |
| | M1043I | 1 | 0.5 | N |
| | H1047Y | 4 | 0.5 | CD4 analysis |
| | M1043I | 4 | 1 | CD4 analysis |
| | H1047Y | | | |
| | H1047R | 2 | 3.2 | RKO and HCT116 |
| FBXW7 | R465H | 3 | 1.7 | CD4 analysis |
| | R465C | 2 | 2.3 | CD4 analysis |
| | R505C | 3 | 0.8 | Y |
| | S582L | 5 | 0.6 | Y |
| BRAF | V600E | 0 | 12.1 | N |
| SMAD4 | R361C | 0 | 0.8 | N |
| | R361H | 1 | 1.5 | Y |
| ATM | R337C | 2 | 0.5 | Y |
| CTNNB1 | S45F | 3 | 0.6 | Y |
| ERBB3 | V104M | 7 | 0.6 | Y |
| GNAS | R201H | 2 | 1 | Y |

CD4 epitopes analysis was performed as described in Example 1 to complete the final selection of CRC driver mutations described in Table 2-13.

Among the identified mutations, PIK3CA H1047R was endogenously expressed by CRC vaccine component cell lines RKO and HCT-116, and therefore was excluded from the final driver mutation insert design. KRAS G12D and KRAS G12V, the two most frequently occurring KRAS mutations, were excluded from the final driver mutation insert design because these driver mutations were previously inserted into the CRC vaccine component cell line HCT-116 as described above and herein. If KRAS G12D and KRAS G12V were not inserted into HCT-116 they would be included in the current insert.

Taken together, as shown in Table 2-13, 17 CRC driver mutations encoded by 15 peptide sequences were selected and included as driver mutation vaccine targets.

TABLE 2-13
Final selection of identified CRC driver mutations based on
CD4 epitope analysis and frequency of each mutation

| | | Number of total | | Included |
|---|---|---|---|---|
| | Driver | CD4 epitopes | Frequency | Yes (Y) or |
| Gene | mutation | (SB + WB) | (%) | No (N) |
| TP53 | R175H | 0 | 6.8 | Y |
| | G245S | 28 | 3.6 | Y |
| | R248W | | | |
| | R273C | 0 | 2.7 | Y |
| KRAS | G12A | 0 | 1.5 | N |
| | G12C | 0 | 3.2 | Y |
| | G12V | 7 | 7.1 | Y |
| | G12D | 11 | 10.4 | Y |
| PIK3CA | R88Q | 21 | 1.9 | Y |
| | E542K | 0 | 2.7 | Y |
| | H1047Y | 47 | 0.5 | N |
| | M1043I | 80 | 1 | Y |
| | H1047Y | | | |
| | H1047R | 8 | 3.2 | RKO and |
| | | | | HCT116 |
| FBXW7 | R465H | 0 | 1.7 | Y |
| | R465C | 0 | 2.3 | N |
| | R505C | 0 | 0.8 | Y |
| | S582L | 6 | 0.6 | Y |
| SMAD4 | R361H | 0 | 1.5 | Y |
| ATM | R337C | 0 | 0.5 | Y |
| CTNNB1 | S45F | 45 | 0.6 | Y |
| ERBB3 | V104M | 2 | 0.6 | Y |
| GNAS | R201H | 0 | 1 | Y |

The total number of CD8 epitopes for each HLA-A and HLA-B supertype introduced by 17 selected CRC driver mutations encoded by 15 peptide sequences was determined as described in Example 1. Results of the epitope prediction analysis are shown in Table 2-14.

TABLE 2-14
CD8 epitopes introduced by 17 selected CRC driver
mutations encoded by 15 peptide sequences

| | | | | Total |
|---|---|---|---|---|
| | | HLA-A | HLA-B | number |
| | Driver | Supertypes | Supertypes | of CD8 |
| Gene | mutation | (n = 5) | (n = 7) | epitopes |
| TP53 | R175H | 1 | 1 | 2 |
| | G245S | 1 | 2 | 3 |
| | R248W | | | |
| | R273C | 0 | 1 | 1 |
| KRAS | G12C | 1 | 0 | 1 |
| PIK3CA | R88Q | 1 | 5 | 6 |
| | E542K | 1 | 0 | 1 |
| | M1043I | 2 | 2 | 4 |
| | H1047Y | | | |
| FBXW7 | R465H | 2 | 1 | 3 |
| | R505C | 1 | 2 | 3 |
| | S582L | 2 | 3 | 5 |
| SMAD4 | R361H | 1 | 0 | 1 |
| ATM | R337C | 2 | 0 | 2 |
| CTNNB1 | S45F | 2 | 1 | 3 |
| ERBB3 | V104M | 1 | 6 | 7 |
| GNAS | R201H | 0 | 2 | 2 |

The total number of CD4 epitopes for Class II locus DRB1, DRB 3/4/5, DQA1/DQB1 and DPB1 introduced by 17 selected CRC driver mutations encoded by 15 peptide sequences was determined as described in Example 1 and the results are shown in Table 2-15.

TABLE 2-15
CD4 epitopes introduced by 17 selected CRC driver mutations encoded by 15 peptide sequences

| | | DRB1 | DRB3/4/5 | DQA1/DQB1 | DPB1 | Total number of |
|---|---|---|---|---|---|---|
| Gene | Driver mutation | (n = 26) | (n = 6) | (n = 8) | (n = 6) | CD4 epitopes |
| TP53 | R175H | 0 | 0 | 0 | 0 | 0 |
| | G245S R248W | 10 | 8 | 1 | 9 | 28 |
| | R273C | 0 | 0 | 0 | 0 | 0 |
| KRAS | G12C | 0 | 0 | 0 | 0 | 0 |
| PIK3CA | R88Q | 16 | 1 | 0 | 4 | 21 |
| | E542K | 0 | 0 | 0 | 0 | 0 |
| | M1043I H1047Y | 34 | 12 | 1 | 33 | 80 |
| FBXW7 | R465H | 0 | 0 | 0 | 0 | 0 |
| | R505C | 0 | 0 | 0 | 0 | 0 |
| | S582L | 0 | 0 | 0 | 6 | 6 |
| SMAD4 | R361H | 0 | 0 | 0 | 0 | 0 |
| ATM | R337C | 0 | 0 | 0 | 0 | 0 |
| CTNNB1 | S45F | 10 | 8 | 0 | 27 | 45 |
| ERBB3 | V104M | 0 | 0 | 0 | 2 | 2 |
| GNAS | R201H | 0 | 0 | 0 | 0 | 0 |

### CRC Patient Sample Coverage by Selected Driver Mutations

As shown in Table 2-16, the 17 selected CRC driver mutations were assembled into two construct inserts. Once two construct inserts were assembled, the analysis of CRC patient sample coverage by each insert was performed. The results indicated that the CRC patient sample coverage by construct encoded driver mutations was 36.2% (Table 2-17). When the driver mutations endogenously expressed by the CRC vaccine component cell lines were also included, the total CRC patient sample coverage was 37.5% (Table 2-18).

TABLE 2-16
Generation of two constructs encoding 17 selected CRC driver mutations

| | Gene | Driver mutation | Frequency (%) | Total CD8 | Total CD4 | CD4 & CD8 |
|---|---|---|---|---|---|---|
| CRC | TP53 | R175H | 6.8 | 2 | 0 | 2 |
| Construct 1 | TP53 | G245S R248W | 3.6 | 3 | 28 | 31 |
| Insert | KRAS | G12C | 3.2 | 1 | 0 | 1 |
| | PIK3CA | R88Q | 1.9 | 6 | 21 | 27 |
| | FBXW7 | R465H | 1.7 | 3 | 0 | 3 |
| | PIK3CA | M1043I H1047Y | 1 | 4 | 80 | 84 |
| | FBXW7 | S582L | 0.6 | 5 | 6 | 11 |
| | CTNNB1 | S45F | 0.6 | 3 | 45 | 48 |
| | ERBB3 | V104M | 0.6 | 7 | 2 | 9 |
| CRC | TP53 | R273C | 2.7 | 1 | 0 | 1 |
| Construct 2 | PIK3CA | E542K | 2.7 | 1 | 0 | 1 |
| Insert | SMAD4 | R361H | 1.5 | 1 | 0 | 1 |
| | GNAS | R201H | 1 | 2 | 0 | 2 |
| | FBXW7 | R505C | 0.8 | 3 | 0 | 3 |
| | ATM | R337C | 0.5 | 2 | 0 | 2 |

TABLE 2-17
CRC patient sample coverage by the construct encoded driver mutations

| Coverage | | | | | | | | | | | |
|---|---|---|---|---|---|---|---|---|---|---|---|
| (Construct Insert | | | | | | | | | | | % of |
| Only) | Driver Mutation Target Gene | | | | | | | | | | patients |
| Sample Description | TP53 | KRAS | PIK3CA | FBXW7 | SMAD4 | ATM | CTNNB1 | ERBB3 | GNAS | Total | (n = 3056) |
| Samples with one | 271 | 470 | 60 | 47 | 20 | 8 | 4 | 15 | 11 | 906 | 29.6 |
| driver mutation | | | | | | | | | | | |
| Samples with ≥2 DMs | 2 | 2 | 1 | 0 | 0 | 0 | 0 | 0 | 0 | 5 | 0.2 |
| from same antigen | | | | | | | | | | | |
| Samples with ≥2 DMs | | | | | | | | | | 194 | 6.3 |
| from different antigens | | | | | | | | | | | |

| | | |
|---|---|---|
| Total | 1105 | 36.2 |

TABLE 2-18
CRC patient sample coverage by construct and cell encoded driver mutations

| Coverage | | | | | | | | | | | |
|---|---|---|---|---|---|---|---|---|---|---|---|
| (Construct Insert, | | | | | | | | | | | % of |
| RKO, HCT-116) | Driver Mutation Target Gene | | | | | | | | | | patients |
| Sample Description | TP53 | KRAS | PIK3CA | FBXW7 | SMAD4 | ATM | CTNNB1 | ERBB3 | GNAS | Total | (n = 3056) |
| Samples with one | 267 | 450 | 100 | 47 | 20 | 8 | 4 | 13 | 11 | 906 | 30.1 |
| driver mutation | | | | | | | | | | | |
| Samples with ≥2 DMs | 2 | 2 | 3 | 0 | 0 | 0 | 0 | 0 | 0 | 6 | 0.2 |
| from same antigen | | | | | | | | | | | |
| Samples with ≥2 DMs | | | | | | | | | | 220 | 7.2 |
| from different antigens | | | | | | | | | | | |

| | | |
|---|---|---|
| Total | 1105 | 37.5 |

### Oncogene Sequences and Insert Sequences of the CRC Driver Mutation Constructs

Native DNA and protein sequences of FBXW7, CTNNB1, ERBB3, SMAD4, GNAS, ATM, TP53, PIK3CA and KRAS oncogenes and inserts encoding driver mutations are included in Table 2-19.

The CRC driver mutation Construct 1 (SEQ ID NO: 51 and SEQ ID NO: 52; encoding driver mutation sequences from oncogenes TP53, KRAS, PIK3CA, FBXW7, CTNNB1 and ERBB3) insert gene encodes 333 amino acids containing the gene encoding driver mutation peptides separated by the furin cleavage sequence RGRKRRS (SEQ ID NO: 32). The CRC driver mutation Construct 2 (SEQ ID NO: 53 and SEQ ID NO: 54; encoding driver mutation sequences from oncogenes TP53, PIK3CA, SMAD4, GNAS, FBXW7 and ATM) insert gene encodes 222 amino acids containing the gene encoding driver mutation peptides separated by the furin cleavage sequence RGRKRRS (SEQ ID NO: 32).

TABLE 2-19
Oncogene sequences and insert sequences for CRC driver
mutation Constructs 1 and Construct 2

| KRAS (SEQ ID | DNA Sequence |
|---|---|
| NO: 33) | 1 ATGACTGAAT ATAAACTTGT GGTAGTTGGA GCTGGTGGCG TAGGCAAGAG TGCCTTGACG |
| | 61 ATACAGCTAA TTCAGAATCA TTTTGTGGAC GAATATGATC CAACAATAGA GGATTCCTAC |
| | 121 AGGAAGCAAG TAGTAATTGA TGGAGAAACC TGTCTCTTGG ATATTCTCGA CACAGCAGGT |
| | 181 CAAGAGGAGT ACAGTGCAAT GAGGGACCAG TACATGAGGA CTGGGGAGGG CTTTCTTTGT |
| | 241 GTATTTGCCA TAAATAATAC TAAATCATTT GAAGATATTC ACCATTATAG AGAACAAATT |
| | 301 AAAAGAGTTA AGGACTCTGA AGATGTACCT ATGGTCCTAG TAGGAAATAA ATGTGATTTG |
| | 361 CCTTCTAGAA CAGTAGACAC AAAACAGGCT CAGGACTTAG CAAGAAGTTA TGGAATTCCT |
| | 421 TTTATTGAAA CATCAGCAAA GACAAGACAG AGAGTGGAGG ATGCTTTTTA TACATTGGTG |
| | 481 AGAGAGATCC GACAATACAG ATTGAAAAAA ATCAGCAAAG AAGAAAAGAC TCCTGGCTGT |
| | 541 GTGAAAATTA AAAAATGCAT TATAATG |
| KRAS SEQ ID | Protein Sequence |
| NO: 34) | 1 MTEYKLVVVG AGGVGKSALT IQLIQNHFVD EYDPTIEDSY RKQVVIDGET CLLDILDTAG |
| | 61 QEEYSAMRDQ YMRTGEGFLC VFAINNTKSF EDIHHYREQI KRVKDSEDVP MVLVGNKCDL |
| | 121 PSRTVDTKQA QDLARSYGIP FIETSAKTRQ RVEDAFYTLV REIRQYRLKK ISKEEKTPGC |
| | 181 VKIKKCIIM |
| TP53 | DNA Sequence |
| (SEQ ID NO: | 1 ATGGAGGAGC CGCAGTCAGA TCCTAGCGTC GAGCCCCCTC TGAGTCAGGA AACATTTTCA |
| 35) | 61 GACCTATGGA AACTACTTCC TGAAAACAAC GTTCTGTCCC CCTTGCCGTC CCAAGCAATG |
| | 121 GATGATTTGA TGCTGTCCCC GGACGATATT GAACAATGGT TCACTGAAGA CCCAGGTCCA |
| | 181 GATGAAGCTC CCAGAATGCC AGAGGCTGCT CCCCCCGTGG CCCCTGCACC AGCAGCTCCT |
| | 241 ACACCGGCGG CCCCTGCACC AGCCCCCTCC TGGCCCCTGT CATCTTCTGT CCCTTCCCAG |
| | 301 AAAACCTACC AGGGCAGCTA CGGTTTCCGT CTGGGCTTCT TGCATTCTGG GACAGCCAAG |
| | 361 TCTGTGACTT GCACGTACTC CCCTGCCCTC AACAAGATGT TTTGCCAACT GGCCAAGACC |
| | 421 TGCCCTGTGC AGCTGTGGGT TGATTCCACA CCCCCGCCCG GCACCCGCGT CCGCGCCATG |
| | 481 GCCATCTACA AGCAGTCACA GCACATGACG GAGGTTGTGA GGCGCTGCCC CCACCATGAG |
| | 541 CGCTGCTCAG ATAGCGATGG TCTGGCCCCT CCTCAGCATC TTATCCGAGT GGAAGGAAAT |
| | 601 TTGCGTGTGG AGTATTTGGA TGACAGAAAC ACTTTTCGAC ATAGTGTGGT GGTGCCCTAT |
| | 661 GAGCCGCCTG AGGTTGGCTC TGACTGTACC ACCATCCACT ACAACTACAT GTGTAACAGT |
| | 721 TCCTGCATGG GCGGCATGAA CCGGAGGCCC ATCCTCACCA TCATCACACT GGAAGACTCC |
| | 781 AGTGGTAATC TACTGGGACG GAACAGCTTT GAGGTGCGTG TTTGTGCCTG TCCTGGGAGA |
| | 841 GACCGGCGCA CAGAGGAAGA GAATCTCCGC AAGAAAGGGG AGCCTCACCA CGAGCTGCCC |
| | 901 CCAGGGAGCA CTAAGCGAGC ACTGCCCAAC AACACCAGCT CCTCTCCCCA GCCAAAGAAG |
| | 961 AAACCACTGG ATGGAGAATA TTTCACCCTT CAGATCCGTG GGCGTGAGCG CTTCGAGATG |
| | 1021 TTCCGAGAGC TGAATGAGGC CTTGGAACTC AAGGATGCCC AGGCTGGGAA GGAGCCAGGG |
| | 1081 GGGAGCAGGG CTCACTCCAG CCACCTGAAG TCCAAAAAGG GTCAGTCTAC CTCCCGCCAT |
| | 1141 AAAAAACTCA TGTTCAAGAC AGAAGGGCCT GACTCAGAC |
| TP53 | Protein Sequence |
| (SEQ ID NO: | 1 MEEPQSDPSV EPPLSQETFS DLWKLLPENN VLSPLPSQAM DDLMLSPDDI EQWFTEDPGP |
| 36) | 61 DEAPRMPEAA PPVAPAPAAP TPAAPAPAPS WPLSSSVPSQ KTYQGSYGFR LGFLHSGTAK |
| | 121 SVTCTYSPAL NKMFCQLAKT CPVQLWVDST PPPGTRVRAM AIYKQSQHMT EVVRRCPHHE |
| | 181 RCSDSDGLAP PQHLIRVEGN LRVEYLDDRN TFRHSVVVPY EPPEVGSDCT TIHYNYMCNS |
| | 241 SCMGGMNRRP ILTIITLEDS SGNLLGRNSF EVRVCACPGR DRRTEEENLR KKGEPHHELP |
| | 301 PGSTKRALPN NTSSSPQPKK KPLDGEYFTL QIRGRERFEM FRELNEALEL KDAQAGKEPG |
| | 361 GSRAHSSHLK SKKGQSTSRH KKLMFKTEGP DSD |
| PIK3CA | DNA Sequence |
| (SEQ ID NO: | 1 ATGCCTCCAC GACCATCATC AGGTGAACTG TGGGGCATCC ACTTGATGCC CCCAAGAATC |
| 37) | 61 CTAGTAGAAT GTTTACTACC AAATGGAATG ATAGTGACTT TAGAATGCCT CCGTGAGGCT |
| | 121 ACATTAATAA CCATAAAGCA TGAACTATTT AAAGAAGCAA GAAAATACCC CCTCCATCAA |
| | 181 CTTCTTCAAG ATGAATCTTC TTACATTTTC GTAAGTGTTA CTCAAGAAGC AGAAAGGGAA |
| | 241 GAATTTTTTG ATGAAACAAG ACGACTTTGT GACCTTCGGC TTTTTCAACC CTTTTTAAAA |
| | 301 GTAATTGAAC CAGTAGGCAA CCGTGAAGAA AAGATCCTCA ATCGAGAAAT TGGTTTTGCT |
| | 361 ATCGGCATGC CAGTGTGTGA ATTTGATATG GTTAAAGATC CAGAAGTACA GGACTTCCGA |
| | 421 AGAAATATTC TGAACGTTTG TAAAGAAGCT GTGGATCTTA GGGACCTCAA TTCACCTCAT |
| | 481 AGTAGAGCAA TGTATGTCTA TCCTCCAAAT GTAGAATCTT CACCAGAATT GCCAAAGCAC |
| | 541 ATATATAATA AATTAGATAA AGGGCAAATA ATAGTGGTGA TCTGGGTAAT AGTTTCTCCA |
| | 601 AATAATGACA AGCAGAAGTA TACTCTGAAA ATCAACCATG ACTGTGTACC AGAACAAGTA |
| | 661 ATTGCTGAAG CAATCAGGAA AAAAACTCGA AGTATGTTGC TATCCTCTGA ACAACTAAAA |
| | 721 CTCTGTGTTT TAGAATATCA GGGCAAGTAT ATTTTAAAAG TGTGTGGATG TGATGAATAC |
| | 781 TTCCTAGAAA AATATCCTCT GAGTCAGTAT AAGTATATAA GAAGCTGTAT AATGCTTGGG |
| | 841 AGGATGCCCA ATTTGATGTT GATGGCTAAA GAAAGCCTTT ATTCTCAACT GCCAATGGAC |
| | 901 TGTTTTACAA TGCCATCTTA TTCCAGACGC ATTTCCACAG CTACACCATA TATGAATGGA |
| | 961 GAAACATCTA CAAAATCCCT TTGGGTTATA AATAGTGCAC TCAGAATAAA AATTCTTTGT |
| | 1021 GCAACCTACG TGAATGTAAA TATTCGAGAC ATTGATAAGA TCTATGTTCG AACAGGTATC |
| | 1081 TACCATGGAG GAGAACCCTT ATGTGACAAT GTGAACACTC AAAGAGTACC TTGTTCCAAT |
| | 1141 CCCAGGTGGA ATGAATGGCT GAATTATGAT ATATACATTC CTGATCTTCC TCGTGCTGCT |
| | 1201 CGACTTTGCC TTTCCATTTG CTCTGTTAAA GGCCGAAAGG GTGCTAAAGA GGAACACTGT |
| | 1261 CCATTGGCAT GGGGAAATAT AAACTTGTTT GATTACACAG ACACTCTAGT ATCTGGAAAA |
| | 1321 ATGGCTTTGA ATCTTTGGCC AGTACCTCAT GGATTAGAAG ATTTGCTGAA CCCTATTGGT |
| | 1381 GTTACTGGAT CAAATCCAAA TAAAGAAACT CCATGCTTAG AGTTGGAGTT TGACTGGTTC |
| | 1441 AGCAGTGTGG TAAAGTTCCC AGATATGTCA GTGATTGAAG AGCATGCCAA TTGGTCTGTA |
| | 1501 TCCCGAGAAG CAGGATTTAG CTATTCCCAC GCAGGACTGA GTAACAGACT AGCTAGAGAC |
| | 1561 AATGAATTAA GGGAAAATGA CAAAGAACAG CTCAAAGCAA TTTCTACACG AGATCCTCTC |
| | 1621 TCTGAAATCA CTGAGCAGGA GAAAGATTTT CTATGGAGTC ACAGACACTA TTGTGTAACT |
| | 1681 ATCCCCGAAA TTCTACCCAA ATTGCTTCTG TCTGTTAAAT GGAATTCTAG AGATGAAGTA |
| | 1741 GCCCAGATGT ATTGCTTGGT AAAAGATTGG CCTCCAATCA AACCTGAACA GGCTATGGAA |
| | 1801 CTTCTGGACT GTAATTACCC AGATCCTATG GTTCGAGGTT TTGCTGTTCG GTGCTTGGAA |
| | 1861 AAATATTTAA CAGATGACAA ACTTTCTCAG TATTTAATTC AGCTAGTACA GGTCCTAAAA |
| | 1921 TATGAACAAT ATTTGGATAA CTTGCTTGTG AGATTTTTAC TGAAGAAAGC ATTGACTAAT |
| | 1981 CAAAGGATTG GGCACTTTTT CTTTTGGCAT TTAAAATCTG AGATGCACAA TAAAACAGTT |
| | 2041 AGCCAGAGGT TTGGCCTGCT TTTGGAGTCC TATTGTCGTG CATGTGGGAT GTATTTGAAG |
| | 2101 CACCTGAATA GGCAAGTCGA GGCAATGGAA AAGCTCATTA ACTTAACTGA CATTCTCAAA |
| | 2161 CAGGAGAAGA AGGATGAAAC ACAAAAGGTA CAGATGAAGT TTTTAGTTGA GCAAATGAGG |
| | 2221 CGACCAGATT TCATGGATGC TCTACAGGGC TTTCTGTCTC CTCTAAACCC TGCTCATCAA |
| | 2281 CTAGGAAACC TCAGGCTTGA AGAGTGTCGA ATTATGTCCT CTGCAAAAAG GCCACTGTGG |
| | 2341 TTGAATTGGG AGAACCCAGA CATCATGTCA GAGTTACTGT TTCAGAACAA TGAGATCATC |
| | 2401 TTTAAAAATG GGGATGATTT ACGGCAAGAT ATGCTAACAC TTCAAATTAT TCGTATTATG |
| | 2461 GAAAATATCT GGCAAAATCA AGGTCTTGAT CTTCGAATGT TACCTTATGG TTGTCTGTCA |
| | 2521 ATCGGTGACT GTGTGGGACT TATTGAGGTG GTGCGAAATT CTCACACTAT TATGCAAATT |
| | 2581 CAGTGCAAAG GCGGCTTGAA AGGTGCACTG CAGTTCAACA GCCACACACT ACATCAGTGG |
| | 2641 CTCAAAGACA AGAACAAAGG AGAAATATAT GATGCAGCCA TTGACCTGTT TACACGTTCA |
| | 2701 TGTGCTGGAT ACTGTGTAGC TACCTTCATT TTGGGAATTG GAGATCGTCA CAATAGTAAC |
| | 2761 ATCATGGTGA AAGACGATGG ACAACTGTTT CATATAGATT TTGGACACTT TTTGGATCAC |
| | 2821 AAGAAGAAAA AATTTGGTTA TAAACGAGAA CGTGTGCCAT TTGTTTTGAC ACAGGATTTC |
| | 2881 TTAATAGTGA TTAGTAAAGG AGCCCAAGAA TGCACAAAGA CAAGAGAATT TGAGAGGTTT |
| | 2941 CAGGAGATGT GTTACAAGGC TTATCTAGCT ATTCGACAGC ATGCCAATCT CTTCATAAAT |
| | 3001 CTTTTCTCAA TGATGCTTGG CTCTGGAATG CCAGAACTAC AATCTTTTGA TGACATTGCA |
| | 3061 TACATTCGAA AGACCCTAGC CTTAGATAAA ACTGAGCAAG AGGCTTTGGA GTATTTCATG |
| | 3121 AAACAAATGA ATGATGCACA TCATGGTGGC TGGACAACAA AAATGGATTG GATCTTCCAC |
| | 3181 ACAATTAAAC AGCATGCATT GAAC |
| FIK3CA | Protein Sequence |
| (SEQ ID NO: | 1 MPPRPSSGEL WGIHLMPPRI LVECLLPNGM IVTLECLREA TLITIKHELF KEARKYPLHQ |
| 38) | 61 LLQDESSYIF VSVTQEAERE EFFDETRRLC DLRLFQPFLK VIEPVGNREE KILNREIGFA |
| | 121 IGMPVCEFDM VKDPEVQDFR RNILNVCKEA VDLRDLNSPH SRAMYVYPPN VESSPELPKH |
| | 181 IYNKLDKGQI IVVIWVIVSP NNDKQKYTLK INHDCVPEQV IAEAIRKKTR SMLLSSEQLK |
| | 241 LCVLEYQGKY ILKVCGCDEY FLEKYPLSQY KYIRSCIMLG RMPNLMLMAK ESLYSQLPMD |
| | 301 CFTMPSYSRR ISTATPYMNG ETSTKSLWVI NSALRIKILC ATYVNVNIRD IDKIYVRTGI |
| | 361 YHGGEPLCDN VNTQRVPCSN PRWNEWLNYD IYIPDLPRAA RLCLSICSVK GRKGAKEEHC |
| | 421 PLAWGNINLF DYTDTLVSGK MALNLWPVPH GLEDLLNPIG VTGSNPNKET PCLELEFDWF |
| | 481 SSVVKFPDMS VIEEHANWSV SREAGFSYSH AGLSNRLARD NELRENDKEQ LKAISTRDPL |
| | 541 SEITEQEKDF LWSHRHYCVT IPEILPKLLL SVKWNSRDEV AQMYCLVKDW PPIKPEQAME |
| | 601 LLDCNYPDPM VRGFAVRCLE KYLTDDKLSQ YLIQLVQVLK YEQYLDNLLV RFLLKKALTN |
| | 661 QRIGHFFFWH LKSEMHNKTV SQRFGLLLES YCRACGMYLK HLNRQVEAME KLINLTDILK |
| | 721 QEKKDETQKV QMKFLVEQMR RPDFMDALQG FLSPLNPAHQ LGNLRLEECR IMSSAKRPLW |
| | 781 LNWENPDIMS ELLFQNNEII FKNGDDLRQD MLTLQIIRIM ENIWQNQGLD LRMLPYGCLS |
| | 841 IGDCVGLIEV VRNSHTIMQI QCKGGLKGAL QFNSHTLHQW LKDKNKGEIY DAAIDLFTRS |
| | 901 CAGYCVATFI LGIGDRHNSN IMVKDDGQLF HIDFGHFLDH KKKKFGYKRE RVPFVLTQDF |
| | 961 LIVISKGAQE CTKTREFERF QEMCYKAYLA IRQHANLFIN LFSMMLGSGM PELQSFDDIA |
| | 1021 YIRKTLALDK TEQEALEYFM KQMNDAHHGG WTTKMDWIFH TIKQHALN |
| FID3V7 | DNA Sequence |
| (SEQ ID | 1 ATGAATCAGG AACTGCTCTC TGTGGGCAGC AAAAGACGAC GAACTGGAGG CTCTCTGAGA |
| NO: 39) | 61 GGTAACCCTT CCTCAAGCCA GGTAGATGAA GAACAGATGA ATCGTGTGGT AGAGGAGGAA |
| | 121 CAGCAACAGC AACTCAGACA ACAAGAGGAG GAGCACACTG CAAGGAATGG TGAAGTTGTT |
| | 181 GGAGTAGAAC CTAGACCTGG AGGCCAAAAT GATTCCCAGC AAGGACAGTT GGAAGAAAAC |
| | 241 AATAATAGAT TTATTTCGGT AGATGAGGAC TCCTCAGGAA ACCAAGAAGA ACAAGAGGAA |
| | 301 GATGAAGAAC ATGCTGGTGA ACAAGATGAG GAGGATGAGG AGGAGGAGGA GATGGACCAG |
| | 361 GAGAGTGACG ATTTTGATCA GTCTGATGAT AGTAGCAGAG AAGATGAACA TACACATACT |
| | 421 AACAGTGTCA CGAACTCCAG TAGTATTGTG GACCTGCCCG TTCACCAACT CTCCTCCCCA |
| | 481 TTCTATACAA AAACAACAAA AATGAAAAGA AAGTTGGACC ATGGTTCTGA GGTCCGCTCT |
| | 541 TTTTCTTTGG GAAAGAAACC ATGCAAAGTC TCAGAATATA CAAGTACCAC TGGGCTTGTA |
| | 601 CCATGTTCAG CAACACCAAC AACTTTTGGG GACCTCAGAG CAGCCAATGG CCAAGGGCAA |
| | 661 CAACGACGCC GAATTACATC TGTCCAGCCA CCTACAGGCC TCCAGGAATG GCTAAAAATG |
| | 721 TTTCAGAGCT GGAGTGGACC AGAGAAATTG CTTGCTTTAG ATGAACTCAT TGATAGTTGT |
| | 781 GAACCAACAC AAGTAAAACA TATGATGCAA GTGATAGAAC CCCAGTTTCA ACGAGACTTC |
| | 841 ATTTCATTGC TCCCTAAAGA GTTGGCACTC TATGTGCTTT CATTCCTGGA ACCCAAAGAC |
| | 901 CTGCTACAAG CAGCTCAGAC ATGTCGCTAC TGGAGAATTT TGGCTGAAGA CAACCTTCTC |
| | 961 TGGAGAGAGA AATGCAAAGA AGAGGGGATT GATGAACCAT TGCACATCAA GAGAAGAAAA |
| | 1021 GTAATAAAAC CAGGTTTCAT ACACAGTCCA TGGAAAAGTG CATACATCAG ACAGCACAGA |
| | 1081 ATTGATACTA ACTGGAGGCG AGGAGAACTC AAATCTCCTA AGGTGCTGAA AGGACATGAT |
| | 1141 GATCATGTGA TCACATGCTT ACAGTTTTGT GGTAACCGAA TAGTTAGTGG TTCTGATGAC |
| | 1201 AACACTTTAA AAGTTTGGTC AGCAGTCACA GGCAAATGTC TGAGAACATT AGTGGGACAT |
| | 1261 ACAGGTGGAG TATGGTCATC ACAAATGAGA GACAACATCA TCATTAGTGG ATCTACAGAT |
| | 1321 CGGACACTCA AAGTGTGGAA TGCAGAGACT GGAGAATGTA TACACACCTT ATATGGGCAT |
| | 1381 ACTTCCACTG TGCGTTGTAT GCATCTTCAT GAAAAAAGAG TTGTTAGCGG TTCTCGAGAT |
| | 1441 GCCACTCTTA GGGTTTGGGA TATTGAGACA GGCCAGTGTT TACATGTTTT GATGGGTCAT |
| | 1501 GTTGCAGCAG TCCGCTGTGT TCAATATGAT GGCAGGAGGG TTGTTAGTGG AGCATATGAT |
| | 1561 TTTATGGTAA AGGTGTGGGA TCCAGAGACT GAAACCTGTC TACACACGTT GCAGGGGCAT |
| | 1621 ACTAATAGAG TCTATTCATT ACAGTTTGAT GGTATCCATG TGGTGAGTGG ATCTCTTGAT |
| | 1681 ACATCAATCC GTGTTTGGGA TGTGGAGACA GGGAATTGCA TTCACACGTT AACAGGGCAC |
| | 1741 CAGTCGTTAA CAAGTGGAAT GGAACTCAAA GACAATATTC TTGTCTCTGG GAATGCAGAT |
| | 1801 TCTACAGTTA AAATCTGGGA TATCAAAACA GGACAGTGTT TACAAACATT GCAAGGTCCC |
| | 1861 AACAAGCATC AGAGTGCTGT GACCTGTTTA CAGTTCAACA AGAACTTTGT AATTACCAGC |
| | 1921 TCAGATGATG GAACTGTAAA ACTATGGGAC TTGAAAACGG GTGAATTTAT TCGAAACCTA |
| | 1981 GTCACATTGG AGAGTGGGGG GAGTGGGGGA GTTGTGTGGC GGATCAGAGC CTCAAACACA |
| | 2041 AAGCTGGTGT GTGCAGTTGG GAGTCGGAAT GGGACTGAAG AAACCAAGCT GCTGGTGCTG |
| | 2101 GACTTTGATG TGGACATGAA GTGA |
| FID3V7 | Protein Sequence |
| (SEQ ID NO: | 1 MNQELLSVGS KRRRTGGSLR GNPSSSQVDE EQMNRVVEEE QQQQLRQQEE EHTARNGEVV |
| 40) | 61 GVEPRPGGQN DSQQGQLEEN NNRFISVDED SSGNQEEQEE DEEHAGEQDE EDEEEEEMDQ |
| | 121 ESDDFDQSDD SSREDEHTHT NSVTNSSSIV DLPVHQLSSP FYTKTTKMKR KLDHGSEVRS |
| | 181 FSLGKKPCKV SEYTSTTGLV PCSATPTTFG DLRAANGQGQ QRRRITSVQP PTGLQEWLKM |
| | 241 FQSWSGPEKL LALDELIDSC EPTQVKHMMQ VIEPQFQRDF ISLLPKELAL YVLSFLEPKD |
| | 301 LLQAAQTCRY WRILAEDNLL WREKCKEEGI DEPLHIKRRK VIKPGFIHSP WKSAYIRQHR |
| | 361 IDTNWRRGEL KSPKVLKGHD DHVITCLQFC GNRIVSGSDD NTLKVWSAVT GKCLRTLVGH |
| | 421 TGGVWSSQMR DNIIISGSTD RTLKVWNAET GECIHTLYGH TSTVRCMHLH EKRVVSGSRD |
| | 481 ATLRVWDIET GQCLHVLMGH VAAVRCVQYD GRRVVSGAYD FMVKVWDPET ETCLHTLQGH |
| | 541 TNRVYSLQFD GIHVVSGSLD TSIRVWDVET GNCIHTLTGH QSLTSGMELK DNILVSGNAD |
| | 601 STVKIWDIKT GQCLQTLQGP NKHQSAVTCL QFNKNFVITS SDDGTVKLWD LKTGEFIRNL |
| | 661 VTLESGGSGG VVWRIRASNT KLVCAVGSRN GTEETKLLVL DFDVDMK |
| SMAD4 | DNA Sequence |
| (SEQ ID | 1 ATGGACAATA TGTCTATTAC GAATACACCA ACAAGTAATG ATGCCTGTCT GAGCATTGTG |
| NO: 41) | 61 CATAGTTTGA TGTGCCATAG ACAAGGTGGA GAGAGTGAAA CATTTGCAAA AAGAGCAATT |
| | 121 GAAAGTTTGG TAAAGAAGCT GAAGGAGAAA AAAGATGAAT TGGATTCTTT AATAACAGCT |
| | 181 ATAACTACAA ATGGAGCTCA TCCTAGTAAA TGTGTTACCA TACAGAGAAC ATTGGATGGG |
| | 241 AGGCTTCAGG TGGCTGGTCG GAAAGGATTT CCTCATGTGA TCTATGCCCG TCTCTGGAGG |
| | 301 TGGCCTGATC TTCACAAAAA TGAACTAAAA CATGTTAAAT ATTGTCAGTA TGCGTTTGAC |
| | 361 TTAAAATGTG ATAGTGTCTG TGTGAATCCA TATCACTACG AACGAGTTGT ATCACCTGGA |
| | 421 ATTGATCTCT CAGGATTAAC ACTGCAGAGT AATGCTCCAT CAAGTATGAT GGTGAAGGAT |
| | 481 GAATATGTGC ATGACTTTGA GGGACAGCCA TCGTTGTCCA CTGAAGGACA TTCAATTCAA |
| | 541 ACCATCCAGC ATCCACCAAG TAATCGTGCA TCGACAGAGA CATACAGCAC CCCAGCTCTG |
| | 601 TTAGCCCCAT CTGAGTCTAA TGCTACCAGC ACTGCCAACT TTCCCAACAT TCCTGTGGCT |
| | 661 TCCACAAGTC AGCCTGCCAG TATACTGGGG GGCAGCCATA GTGAAGGACT GTTGCAGATA |
| | 721 GCATCAGGGC CTCAGCCAGG ACAGCAGCAG AATGGATTTA CTGGTCAGCC AGCTACTTAC |
| | 781 CATCATAACA GCACTACCAC CTGGACTGGA AGTAGGACTG CACCATACAC ACCTAATTTG |
| | 841 CCTCACCACC AAAACGGCCA TCTTCAGCAC CACCCGCCTA TGCCGCCCCA TCCCGGACAT |
| | 901 TACTGGCCTG TTCACAATGA GCTTGCATTC CAGCCTCCCA TTTCCAATCA TCCTGCTCCT |
| | 961 GAGTATTGGT GTTCCATTGC TTACTTTGAA ATGGATGTTC AGGTAGGAGA GACATTTAAG |
| | 1021 GTTCCTTCAA GCTGCCCTAT TGTTACTGTT GATGGATACG TGGACCCTTC TGGAGGAGAT |
| | 1081 CGCTTTTGTT TGGGTCAACT CTCCAATGTC CACAGGACAG AAGCCATTGA GAGAGCAAGG |
| | 1141 TTGCACATAG GCAAAGGTGT GCAGTTGGAA TGTAAAGGTG AAGGTGATGT TTGGGTCAGG |
| | 1201 TGCCTTAGTG ACCACGCGGT CTTTGTACAG AGTTACTACT TAGACAGAGA AGCTGGGCGT |
| | 1261 GCACCTGGAG ATGCTGTTCA TAAGATCTAC CCAAGTGCAT ATATAAAGGT CTTTGATTTG |
| | 1321 CGTCAGTGTC ATCGACAGAT GCAGCAGCAG GCGGCTACTG CACAAGCTGC AGCAGCTGCC |
| | 1381 CAGGCAGCAG CCGTGGCAGG AAACATCCCT GGCCCAGGAT CAGTAGGTGG AATAGCTCCA |
| | 1441 GCTATCAGTC TGTCAGCTGC TGCTGGAATT GGTGTTGATG ACCTTCGTCG CTTATGCATA |
| | 1501 CTCAGGATGA GTTTTGTGAA AGGCTGGGGA CCGGATTACC CAAGACAGAG CATCAAAGAA |
| | 1561 ACACCTTGCT GGATTGAAAT TCACTTACAC CGGGCCCTCC AGCTCCTAGA CGAAGTACTT |
| | 1621 CATACCATGC CGATTGCAGA CCCACAACCT TTAGACTGA |
| SMAD4 | Protein Sequence |
| (SEQ ID | 1 MDNMSITNTP TSNDACLSIV HSLMCHRQGG ESETFAKRAI ESLVKKLKEK KDELDSLITA |
| NO: 42) | 61 ITTNGAHPSK CVTIQRTLDG RLQVAGRKGF PHVIYARLWR WPDLHKNELK HVKYCQYAFD |
| | 121 LKCDSVCVNP YHYERVVSPG IDLSGLTLQS NAPSSMMVKD EYVHDFEGQP SLSTEGHSIQ |
| | 181 TIQHPPSNRA STETYSTPAL LAPSESNATS TANFPNIPVA STSQPASILG GSHSEGLLQI |
| | 241 ASGPQPGQQQ NGFTGQPATY HHNSTTTWTG SRTAPYTPNL PHHQNGHLQH HPPMPPHPGH |
| | 301 YWPVHNELAF QPPISNHPAP EYWCSIAYFE MDVQVGETFK VPSSCPIVTV DGYVDPSGGD |
| | 361 RFCLGQLSNV HRTEAIERAR LHIGKGVQLE CKGEGDVWVR CLSDHAVFVQ SYYLDREAGR |
| | 421 APGDAVHKIY PSAYIKVFDL RQCHRQMQQQ AATAQAAAAA QAAAVAGNIP GPGSVGGIAP |
| | 481 AISLSAAAGI GVDDLRRLCI LRMSFVKGWG PDYPRQSIKE TPCWIEIHLH RALQLLDEVL |
| | 541 HTMPIADPQP LD |
| ATM | DNA Sequence |
| (SEQ ID NO: | 1 ATGAGTCTAG TACTTAATGA TCTGCTTATC TGCTGCCGTC AACTAGAACA TGATAGAGCT |
| 43) | 61 ACAGAACGAA AGAAAGAAGT TGAGAAATTT AAGCGCCTGA TTCGAGATCC TGAAACAATT |
| | 121 AAACATCTAG ATCGGCATTC AGATTCCAAA CAAGGAAAAT ATTTGAATTG GGATGCTGTT |
| | 181 TTTAGATTTT TACAGAAATA TATTCAGAAA GAAACAGAAT GTCTGAGAAT AGCAAAACCA |
| | 241 AATGTATCAG CCTCAACACA AGCCTCCAGG CAGAAAAAGA TGCAGGAAAT CAGTAGTTTG |
| | 301 GTCAAATACT TCATCAAATG TGCAAACAGA AGAGCACCTA GGCTAAAATG TCAAGAACTC |
| | 361 TTAAATTATA TCATGGATAC AGTGAAAGAT TCATCTAATG GTGCTATTTA CGGAGCTGAT |
| | 421 TGTAGCAACA TACTACTCAA AGACATTCTT TCTGTGAGAA AATACTGGTG TGAAATATCT |
| | 481 CAGCAACAGT GGTTAGAATT GTTCTCTGTG TACTTCAGGC TCTATCTGAA ACCTTCACAA |
| | 541 GATGTTCATA GAGTTTTAGT GGCTAGAATA ATTCATGCTG TTACCAAAGG ATGCTGTTCT |
| | 601 CAGACTGACG GATTAAATTC CAAATTTTTG GACTTTTTTT CCAAGGCTAT TCAGTGTGCG |
| | 661 AGACAAGAAA AGAGCTCTTC AGGTCTAAAT CATATCTTAG CAGCTCTTAC TATCTTCCTC |
| | 721 AAGACTTTGG CTGTCAACTT TCGAATTCGA GTGTGTGAAT TAGGAGATGA AATTCTTCCC |
| | 781 ACTTTGCTTT ATATTTGGAC TCAACATAGG CTTAATGATT CTTTAAAAGA AGTCATTATT |
| | 841 GAATTATTTC AACTGCAAAT TTATATCCAT CATCCGAAAG GAGCCAAAAC CCAAGAAAAA |
| | 901 GGTGCTTATG AATCAACAAA ATGGAGAAGT ATTTTATACA ACTTATATGA TCTGCTAGTG |
| | 961 AATGAGATAA GTCATATAGG AAGTAGAGGA AAGTATTCTT CAGGATTTCG TAATATTGCC |
| | 1021 GTCAAAGAAA ATTTGATTGA ATTGATGGCA GATATCTGTC ACCAGGTTTT TAATGAAGAT |
| | 1081 ACCAGATCCT TGGAGATTTC TCAATCTTAC ACTACTACAC AAAGAGAATC TAGTGATTAC |
| | 1141 AGTGTCCCTT GCAAAAGGAA GAAAATAGAA CTAGGCTGGG AAGTAATAAA AGATCACCTT |
| | 1201 CAGAAGTCAC AGAATGATTT TGATCTTGTG CCTTGGCTAC AGATTGCAAC CCAATTAATA |
| | 1261 TCAAAGTATC CTGCAAGTTT ACCTAACTGT GAGCTGTCTC CATTACTGAT GATACTATCT |
| | 1321 CAGCTTCTAC CCCAACAGCG ACATGGGGAA CGTACACCAT ATGTGTTACG ATGCCTTACG |
| | 1381 GAAGTTGCAT TGTGTCAAGA CAAGAGGTCA AACCTAGAAA GCTCACAAAA GTCAGATTTA |
| | 1441 TTAAAACTCT GGAATAAAAT TTGGTGTATT ACCTTTCGTG GTATAAGTTC TGAGCAAATA |
| | 1501 CAAGCTGAAA ACTTTGGCTT ACTTGGAGCC ATAATTCAGG GTAGTTTAGT TGAGGTTGAC |
| | 1561 AGAGAATTCT GGAAGTTATT TACTGGGTCA GCCTGCAGAC CTTCATGTCC TGCAGTATGC |
| | 1621 TGTTTGACTT TGGCACTGAC CACCAGTATA GTTCCAGGAA CGGTAAAAAT GGGAATAGAG |
| | 1681 CAAAATATGT GTGAAGTAAA TAGAAGCTTT TCTTTAAAGG AATCAATAAT GAAATGGCTC |
| | 1741 TTATTCTATC AGTTAGAGGG TGACTTAGAA AATAGCACAG AAGTGCCTCC AATTCTTCAC |
| | 1801 AGTAATTTTC CTCATCTTGT ACTGGAGAAA ATTCTTGTGA GTCTCACTAT GAAAAACTGT |
| | 1861 AAAGCTGCAA TGAATTTTTT CCAAAGCGTG CCAGAATGTG AACACCACCA AAAAGATAAA |
| | 1921 GAAGAACTTT CATTCTCAGA AGTAGAAGAA CTATTTCTTC AGACAACTTT TGACAAGATG |
| | 1981 GACTTTTTAA CCATTGTGAG AGAATGTGGT ATAGAAAAGC ACCAGTCCAG TATTGGCTTC |
| | 2041 TCTGTCCACC AGAATCTCAA GGAATCACTG GATCGCTGTC TTCTGGGATT ATCAGAACAG |
| | 2101 CTTCTGAATA ATTACTCATC TGAGATTACA AATTCAGAAA CTCTTGTCCG GTGTTCACGT |
| | 2161 CTTTTGGTGG GTGTCCTTGG CTGCTACTGT TACATGGGTG TAATAGCTGA AGAGGAAGCA |
| | 2221 TATAAGTCAG AATTATTCCA GAAAGCCAAG TCTCTAATGC AATGTGCAGG AGAAAGTATC |
| | 2281 ACTCTGTTTA AAAATAAGAC AAATGAGGAA TTCAGAATTG GTTCCTTGAG AAATATGATG |
| | 2341 CAGCTATGTA CACGTTGCTT GAGCAACTGT ACCAAGAAGA GTCCAAATAA GATTGCATCT |
| | 2401 GGCTTTTTCC TGCGATTGTT AACATCAAAG CTAATGAATG ACATTGCAGA TATTTGTAAA |
| | 2461 AGTTTAGCAT CCTTCATCAA AAAGCCATTT GACCGTGGAG AAGTAGAATC AATGGAAGAT |
| | 2521 GATACTAATG GAAATCTAAT GGAGGTGGAG GATCAGTCAT CCATGAATCT ATTTAACGAT |
| | 2581 TACCCTGATA GTAGTGTTAG TGATGCAAAC GAACCTGGAG AGAGCCAAAG TACCATAGGT |
| | 2641 GCCATTAATC CTTTAGCTGA AGAATATCTG TCAAAGCAAG ATCTACTTTT CTTAGACATG |
| | 2701 CTCAAGTTCT TGTGTTTGTG TGTAACTACT GCTCAGACCA ATACTGTGTC CTTTAGGGCA |
| | 2761 GCTGATATTC GGAGGAAATT GTTAATGTTA ATTGATTCTA GCACGCTAGA ACCTACCAAA |
| | 2821 TCCCTCCACC TGCATATGTA TCTAATGCTT TTAAAGGAGC TTCCTGGAGA AGAGTACCCC |
| | 2881 TTGCCAATGG AAGATGTTCT TGAACTTCTG AAACCACTAT CCAATGTGTG TTCTTTGTAT |
| | 2941 CGTCGTGACC AAGATGTTTG TAAAACTATT TTAAACCATG TCCTTCATGT AGTGAAAAAC |
| | 3001 CTAGGTCAAA GCAATATGGA CTCTGAGAAC ACAAGGGATG CTCAAGGACA GTTTCTTACA |
| | 3061 GTAATTGGAG CATTTTGGCA TCTAACAAAG GAGAGGAAAT ATATATTCTC TGTAAGAATG |
| | 3121 GCCCTAGTAA ATTGCCTTAA AACTTTGCTT GAGGCTGATC CTTATTCAAA ATGGGCCATT |
| | 3181 CTTAATGTAA TGGGAAAAGA CTTTCCTGTA AATGAAGTAT TTACACAATT TCTTGCTGAC |
| | 3241 AATCATCACC AAGTTCGCAT GTTGGCTGCA GAGTCAATCA ATAGATTGTT CCAGGACACG |
| | 3301 AAGGGAGATT CTTCCAGGTT ACTGAAAGCA CTTCCTTTGA AGCTTCAGCA AACAGCTTTT |
| | 3361 GAAAATGCAT ACTTGAAAGC TCAGGAAGGA ATGAGAGAAA TGTCCCATAG TGCTGAGAAC |
| | 3421 CCTGAAACTT TGGATGAAAT TTATAATAGA AAATCTGTTT TACTGACGTT GATAGCTGTG |
| | 3481 GTTTTATCCT GTAGCCCTAT CTGCGAAAAA CAGGCTTTGT TTGCCCTGTG TAAATCTGTG |
| | 3541 AAAGAGAATG GATTAGAACC TCACCTTGTG AAAAAGGTTT TAGAGAAAGT TTCTGAAACT |
| | 3601 TTTGGATATA GACGTTTAGA AGACTTTATG GCATCTCATT TAGATTATCT GGTTTTGGAA |
| | 3661 TGGCTAAATC TTCAAGATAC TGAATACAAC TTATCTTCTT TTCCTTTTAT TTTATTAAAC |
| | 3721 TACACAAATA TTGAGGATTT CTATAGATCT TGTTATAAGG TTTTGATTCC ACATCTGGTG |
| | 3781 ATTAGAAGTC ATTTTGATGA GGTGAAGTCC ATTGCTAATC AGATTCAAGA GGACTGGAAA |
| | 3841 AGTCTTCTAA CAGACTGCTT TCCAAAGATT CTTGTAAATA TTCTTCCTTA TTTTGCCTAT |
| | 3901 GAGGGTACCA GAGACAGTGG GATGGCACAG CAAAGAGAGA CTGCTACCAA GGTCTATGAT |
| | 3961 ATGCTTAAAA GTGAAAACTT ATTGGGAAAA CAGATTGATC ACTTATTCAT TAGTAATTTA |
| | 4021 CCAGAGATTG TGGTGGAGTT ATTGATGACG TTACATGAGC CAGCAAATTC TAGTGCCAGT |
| | 4081 CAGAGCACTG ACCTCTGTGA CTTTTCAGGG GATTTGGATC CTGCTCCTAA TCCACCTCAT |
| | 4141 TTTCCATCGC ATGTGATTAA AGCAACATTT GCCTATATCA GCAATTGTCA TAAAACCAAG |
| | 4201 TTAAAAAGCA TTTTAGAAAT TCTTTCCAAA AGCCCTGATT CCTATCAGAA AATTCTTCTT |
| | 4261 GCCATATGTG AGCAAGCAGC TGAAACAAAT AATGTTTATA AGAAGCACAG AATTCTTAAA |
| | 4321 ATATATCACC TGTTTGTTAG TTTATTACTG AAAGATATAA AAAGTGGCTT AGGAGGAGCT |
| | 4381 TGGGCCTTTG TTCTTCGAGA CGTTATTTAT ACTTTGATTC ACTATATCAA CCAAAGGCCT |
| | 4441 TCTTGTATCA TGGATGTGTC ATTACGTAGC TTCTCCCTTT GTTGTGACTT ATTAAGTCAG |
| | 4501 GTTTGCCAGA CAGCCGTGAC TTACTGTAAG GATGCTCTAG AAAACCATCT TCATGTTATT |
| | 4561 GTTGGTACAC TTATACCCCT TGTGTATGAG CAGGTGGAGG TTCAGAAACA GGTATTGGAC |
| | 4621 TTGTTGAAAT ACTTAGTGAT AGATAACAAG GATAATGAAA ACCTCTATAT CACGATTAAG |
| | 4681 CTTTTAGATC CTTTTCCTGA CCATGTTGTT TTTAAGGATT TGCGTATTAC TCAGCAAAAA |
| | 4741 ATCAAATACA GTAGAGGACC CTTTTCACTC TTGGAGGAAA TTAACCATTT TCTCTCAGTA |
| | 4801 AGTGTTTATG ATGCACTTCC ATTGACAAGA CTTGAAGGAC TAAAGGATCT TCGAAGACAA |
| | 4861 CTGGAACTAC ATAAAGATCA GATGGTGGAC ATTATGAGAG CTTCTCAGGA TAATCCGCAA |
| | 4921 GATGGGATTA TGGTGAAACT AGTTGTCAAT TTGTTGCAGT TATCCAAGAT GGCAATAAAC |
| | 4981 CACACTGGTG AAAAAGAAGT TCTAGAGGCT GTTGGAAGCT GCTTGGGAGA AGTGGGTCCT |
| | 5041 ATAGATTTCT CTACCATAGC TATACAACAT AGTAAAGATG CATCTTATAC CAAGGCCCTT |
| | 5101 AAGTTATTTG AAGATAAAGA ACTTCAGTGG ACCTTCATAA TGCTGACCTA CCTGAATAAC |
| | 5161 ACACTGGTAG AAGATTGTGT CAAAGTTCGA TCAGCAGCTG TTACCTGTTT GAAAAACATT |
| | 5221 TTAGCCACAA AGACTGGACA TAGTTTCTGG GAGATTTATA AGATGACAAC AGATCCAATG |
| | 5281 CTGGCCTATC TACAGCCTTT TAGAACATCA AGAAAAAAGT TTTTAGAAGT ACCCAGATTT |
| | 5341 GACAAAGAAA ACCCTTTTGA AGGCCTGGAT GATATAAATC TGTGGATTCC TCTAAGTGAA |
| | 5401 AATCATGACA TTTGGATAAA GACACTGACT TGTGCTTTTT TGGACAGTGG AGGCACAAAA |
| | 5461 TGTGAAATTC TTCAATTATT AAAGCCAATG TGTGAAGTGA AAACTGACTT TTGTCAGACT |
| | 5521 GTACTTCCAT ACTTGATTCA TGATATTTTA CTCCAAGATA CAAATGAATC ATGGAGAAAT |
| | 5581 CTGCTTTCTA CACATGTTCA GGGATTTTTC ACCAGCTGTC TTCGACACTT CTCGCAAACG |
| | 5641 AGCCGATCCA CAACCCCTGC AAACTTGGAT TCAGAGTCAG AGCACTTTTT CCGATGCTGT |
| | 5701 TTGGATAAAA AATCACAAAG AACAATGCTT GCTGTTGTGG ACTACATGAG AAGACAAAAG |
| | 5761 AGACCTTCTT CAGGAACAAT TTTTAATGAT GCTTTCTGGC TGGATTTAAA TTATCTAGAA |
| | 5821 GTTGCCAAGG TAGCTCAGTC TTGTGCTGCT CACTTTACAG CTTTACTCTA TGCAGAAATC |
| | 5881 TATGCAGATA AGAAAAGTAT GGATGATCAA GAGAAAAGAA GTCTTGCATT TGAAGAAGGA |
| | 5941 AGCCAGAGTA CAACTATTTC TAGCTTGAGT GAAAAAAGTA AAGAAGAAAC TGGAATAAGT |
| | 6001 TTACAGGATC TTCTCTTAGA AATCTACAGA AGTATAGGGG AGCCAGATAG TTTGTATGGC |
| | 6061 TGTGGTGGAG GGAAGATGTT ACAACCCATT ACTAGACTAC GAACATATGA ACACGAAGCA |
| | 6121 ATGTGGGGCA AAGCCCTAGT AACATATGAC CTCGAAACAG CAATCCCCTC ATCAACACGC |
| | 6181 CAGGCAGGAA TCATTCAGGC CTTGCAGAAT TTGGGACTCT GCCATATTCT TTCCGTCTAT |
| | 6241 TTAAAAGGAT TGGATTATGA AAATAAAGAC TGGTGTCCTG AACTAGAAGA ACTTCATTAC |
| | 6301 CAAGCAGCAT GGAGGAATAT GCAGTGGGAC CATTGCACTT CCGTCAGCAA AGAAGTAGAA |
| | 6361 GGAACCAGTT ACCATGAATC ATTGTACAAT GCTCTACAAT CTCTAAGAGA CAGAGAATTC |
| | 6421 TCTACATTTT ATGAAAGTCT CAAATATGCC AGAGTAAAAG AAGTGGAAGA GATGTGTAAG |
| | 6481 CGCAGCCTTG AGTCTGTGTA TTCGCTCTAT CCCACACTTA GCAGGTTGCA GGCCATTGGA |
| | 6541 GAGCTGGAAA GCATTGGGGA GCTTTTCTCA AGATCAGTCA CACATAGACA ACTCTCTGAA |
| | 6601 GTATATATTA AGTGGCAGAA ACACTCCCAG CTTCTCAAGG ACAGTGATTT TAGTTTTCAG |
| | 6661 GAGCCTATCA TGGCTCTACG CACAGTCATT TTGGAGATCC TGATGGAAAA GGAAATGGAC |
| | 6721 AACTCACAAA GAGAATGTAT TAAGGACATT CTCACCAAAC ACCTTGTAGA ACTCTCTATA |
| | 6781 CTGGCCAGAA CTTTCAAGAA CACTCAGCTC CCTGAAAGGG CAATATTTCA AATTAAACAG |
| | 6841 TACAATTCAG TTAGCTGTGG AGTCTCTGAG TGGCAGCTGG AAGAAGCACA AGTATTCTGG |
| | 6901 GCAAAAAAGG AGCAGAGTCT TGCCCTGAGT ATTCTCAAGC AAATGATCAA GAAGTTGGAT |
| | 6961 GCCAGCTGTG CAGCGAACAA TCCCAGCCTA AAACTTACAT ACACAGAATG TCTGAGGGTT |
| | 7021 TGTGGCAACT GGTTAGCAGA AACGTGCTTA GAAAATCCTG CGGTCATCAT GCAGACCTAT |
| | 7081 CTAGAAAAGG CAGTAGAAGT TGCTGGAAAT TATGATGGAG AAAGTAGTGA TGAGCTAAGA |
| | 7141 AATGGAAAAA TGAAGGCATT TCTCTCATTA GCCCGGTTTT CAGATACTCA ATACCAAAGA |
| | 7201 ATTGAAAACT ACATGAAATC ATCGGAATTT GAAAACAAGC AAGCTCTCCT GAAAAGAGCC |
| | 7261 AAAGAGGAAG TAGGTCTCCT TAGGGAACAT AAAATTCAGA CAAACAGATA CACAGTAAAG |
| | 7321 GTTCAGCGAG AGCTGGAGTT GGATGAATTA GCCCTGCGTG CACTGAAAGA GGATCGTAAA |
| | 7381 CGCTTCTTAT GTAAAGCAGT TGAAAATTAT ATCAACTGCT TATTAAGTGG AGAAGAACAT |
| | 7441 GATATGTGGG TATTCCGACT TTGTTCCCTC TGGCTTGAAA ATTCTGGAGT TTCTGAAGTC |
| | 7501 AATGGCATGA TGAAGAGAGA CGGAATGAAG ATTCCAACAT ATAAATTTTT GCCTCTTATG |
| | 7561 TACCAATTGG CTGCTAGAAT GGGGACCAAG ATGATGGGAG GCCTAGGATT TCATGAAGTC |
| | 7621 CTCAATAATC TAATCTCTAG AATTTCAATG GATCACCCCC ATCACACTTT GTTTATTATA |
| | 7681 CTGGCCTTAG CAAATGCAAA CAGAGATGAA TTTCTGACTA AACCAGAGGT AGCCAGAAGA |
| | 7741 AGCAGAATAA CTAAAAATGT GCCTAAACAA AGCTCTCAGC TTGATGAGGA TCGAACAGAG |
| | 7801 GCTGCAAATA GAATAATATG TACTATCAGA AGTAGGAGAC CTCAGATGGT CAGAAGTGTT |
| | 7861 GAGGCACTTT GTGATGCTTA TATTATATTA GCAAACTTAG ATGCCACTCA GTGGAAGACT |
| | 7921 CAGAGAAAAG GCATAAATAT TCCAGCAGAC CAGCCAATTA CTAAACTTAA GAATTTAGAA |
| | 7981 GATGTTGTTG TCCCTACTAT GGAAATTAAG GTGGACCACA CAGGAGAATA TGGAAATCTG |
| | 8041 GTGACTATAC AGTCATTTAA AGCAGAATTT CGCTTAGCAG GAGGTGTAAA TTTACCAAAA |
| | 8101 ATAATAGATT GTGTAGGTTC CGATGGCAAG GAGAGGAGAC AGCTTGTTAA GGGCCGTGAT |
| | 8161 GACCTGAGAC AAGATGCTGT CATGCAACAG GTCTTCCAGA TGTGTAATAC ATTACTGCAG |
| | 8221 AGAAACACGG AAACTAGGAA GAGGAAATTA ACTATCTGTA CTTATAAGGT GGTTCCCCTC |
| | 8281 TCTCAGCGAA GTGGTGTTCT TGAATGGTGC ACAGGAACTG TCCCCATTGG TGAATTTCTT |
| | 8341 GTTAACAATG AAGATGGTGC TCATAAAAGA TACAGGCCAA ATGATTTCAG TGCCTTTCAG |
| | 8401 TGCCAAAAGA AAATGATGGA GGTGCAAAAA AAGTCTTTTG AAGAGAAATA TGAAGTCTTC |
| | 8461 ATGGATGTTT GCCAAAATTT TCAACCAGTT TTCCGTTACT TCTGCATGGA AAAATTCTTG |
| | 8521 GATCCAGCTA TTTGGTTTGA GAAGCGATTG GCTTATACGC GCAGTGTAGC TACTTCTTCT |
| | 8581 ATTGTTGGTT ACATACTTGG ACTTGGTGAT AGACATGTAC AGAATATCTT GATAAATGAG |
| | 8641 CAGTCAGCAG AACTTGTACA TATAGATCTA GGTGTTGCTT TTGAACAGGG CAAAATCCTT |
| | 8701 CCTACTCCTG AGACAGTTCC TTTTAGACTC ACCAGAGATA TTGTGGATGG CATGGGCATT |
| | 8761 ACGGGTGTTG AAGGTGTCTT CAGAAGATGC TGTGAGAAAA CCATGGAAGT GATGAGAAAC |
| | 8821 TCTCAGGAAA CTCTGTTAAC CATTGTAGAG GTCCTTCTAT ATGATCCACT CTTTGACTGG |
| | 8881 ACCATGAATC CTTTGAAAGC TTTGTATTTA CAGCAGAGGC CGGAAGATGA AACTGAGCTT |
| | 8941 CACCCTACTC TGAATGCAGA TGACCAAGAA TGCAAACGAA ATCTCAGTGA TATTGACCAG |
| | 9001 AGTTTCAACA AAGTAGCTGA ACGTGTCTTA ATGAGACTAC AAGAGAAACT GAAAGGAGTG |
| | 9061 GAAGAAGGCA CTGTGCTCAG TGTTGGTGGA CAAGTGAATT TGCTCATACA GCAGGCCATA |
| | 9121 GACCCCAAAA ATCTCAGCCG ACTTTTCCCA GGATGGAAAG CTTGGGTGTG A |
| ATM | Protein Sequence |
| (SEQ ID NO: | 1 MSLVLNDLLI CCRQLEHDRA TERKKEVEKF KRLIRDPETI KHLDRHSDSK QGKYLNWDAV |
| 44) | 61 FRFLQKYIQK ETECLRIAKP NVSASTQASR QKKMQEISSL VKYFIKCANR RAPRLKCQEL |
| | 121 LNYIMDTVKD SSNGAIYGAD CSNILLKDIL SVRKYWCEIS QQQWLELFSV YFRLYLKPSQ |
| | 181 DVHRVLVARI IHAVTKGCCS QTDGLNSKFL DFFSKAIQCA RQEKSSSGLN HILAALTIFL |
| | 241 KTLAVNFRIR VCELGDEILP TLLYIWTQHR LNDSLKEVII ELFQLQIYIH HPKGAKTQEK |
| | 301 GAYESTKWRS ILYNLYDLLV NEISHIGSRG KYSSGFRNIA VKENLIELMA DICHQVFNED |
| | 361 TRSLEISQSY TTTQRESSDY SVPCKRKKIE LGWEVIKDHL QKSQNDFDLV PWLQIATQLI |
| | 421 SKYPASLPNC ELSPLLMILS QLLPQQRHGE RTPYVLRCLT EVALCQDKRS NLESSQKSDL |
| | 481 LKLWNKIWCI TFRGISSEQI QAENFGLLGA IIQGSLVEVD REFWKLFTGS ACRPSCPAVC |
| | 541 CLTLALTTSI VPGTVKMGIE QNMCEVNRSF SLKESIMKWL LFYQLEGDLE NSTEVPPILH |
| | 601 SNFPHLVLEK ILVSLTMKNC KAAMNFFQSV PECEHHQKDK EELSFSEVEE LFLQTTFDKM |
| | 661 DFLTIVRECG IEKHQSSIGF SVHQNLKESL DRCLLGLSEQ LLNNYSSEIT NSETLVRCSR |
| | 721 LLVGVLGCYC YMGVIAEEEA YKSELFQKAK SLMQCAGESI TLFKNKTNEE FRIGSLRNMM |
| | 781 QLCTRCLSNC TKKSPNKIAS GFFLRLLTSK LMNDIADICK SLASFIKKPF DRGEVESMED |
| | 841 DTNGNLMEVE DQSSMNLFND YPDSSVSDAN EPGESQSTIG AINPLAEEYL SKQDLLFLDM |
| | 901 LKFLCLCVTT AQTNTVSFRA ADIRRKLLML IDSSTLEPTK SLHLHMYLML LKELPGEEYP |
| | 961 LPMEDVLELL KPLSNVCSLY RRDQDVCKTI LNHVLHVVKN LGQSNMDSEN TRDAQGQFLT |
| | 1021 VIGAFWHLTK ERKYIFSVRM ALVNCLKTLL EADPYSKWAI LNVMGKDFPV NEVFTQFLAD |
| | 1081 NHHQVRMLAA ESINRLFQDT KGDSSRLLKA LPLKLQQTAF ENAYLKAQEG MREMSHSAEN |
| | 1141 PETLDEIYNR KSVLLTLIAV VLSCSPICEK QALFALCKSV KENGLEPHLV KKVLEKVSET |
| | 1201 FGYRRLEDFM ASHLDYLVLE WLNLQDTEYN LSSFPFILLN YTNIEDFYRS CYKVLIPHLV |
| | 1261 IRSHFDEVKS IANQIQEDWK SLLTDCFPKI LVNILPYFAY EGTRDSGMAQ QRETATKVYD |
| | 1321 MLKSENLLGK QIDHLFISNL PEIVVELLMT LHEPANSSAS QSTDLCDFSG DLDPAPNPPH |
| | 1381 FPSHVIKATF AYISNCHKTK LKSILEILSK SPDSYQKILL AICEQAAETN NVYKKHRILK |
| | 1441 IYHLFVSLLL KDIKSGLGGA WAFVLRDVIY TLIHYINQRP SCIMDVSLRS FSLCCDLLSQ |
| | 1501 VCQTAVTYCK DALENHLHVI VGTLIPLVYE QVEVQKQVLD LLKYLVIDNK DNENLYITIK |
| | 1561 LLDPFPDHVV FKDLRITQQK IKYSRGPFSL LEEINHFLSV SVYDALPLTR LEGLKDLRRQ |
| | 1621 LELHKDQMVD IMRASQDNPQ DGIMVKLVVN LLQLSKMAIN HTGEKEVLEA VGSCLGEVGP |
| | 1681 IDFSTIAIQH SKDASYTKAL KLFEDKELQW TFIMLTYLNN TLVEDCVKVR SAAVTCLKNI |
| | 1741 LATKTGHSFW EIYKMTTDPM LAYLQPFRTS RKKFLEVPRF DKENPFEGLD DINLWIPLSE |
| | 1801 NHDIWIKTLT CAFLDSGGTK CEILQLLKPM CEVKTDFCQT VLPYLIHDIL LQDTNESWRN |
| | 1861 LLSTHVQGFF TSCLRHFSQT SRSTTPANLD SESEHFFRCC LDKKSQRTML AVVDYMRRQK |
| | 1921 RPSSGTIFND AFWLDLNYLE VAKVAQSCAA HFTALLYAEI YADKKSMDDQ EKRSLAFEEG |
| | 1981 SQSTTISSLS EKSKEETGIS LQDLLLEIYR SIGEPDSLYG CGGGKMLQPI TRLRTYEHEA |
| | 2041 MWGKALVTYD LETAIPSSTR QAGIIQALQN LGLCHILSVY LKGLDYENKD WCPELEELHY |
| | 2101 QAAWRNMQWD HCTSVSKEVE GTSYHESLYN ALQSLRDREF STFYESLKYA RVKEVEEMCK |
| | 2161 RSLESVYSLY PTLSRLQAIG ELESIGELFS RSVTHRQLSE VYIKWQKHSQ LLKDSDFSFQ |
| | 2221 EPIMALRTVI LEILMEKEMD NSQRECIKDI LTKHLVELSI LARTFKNTQL PERAIFQIKQ |
| | 2281 YNSVSCGVSE WQLEEAQVFW AKKEQSLALS ILKQMIKKLD ASCAANNPSL KLTYTECLRV |
| | 2341 CGNWLAETCL ENPAVIMQTY LEKAVEVAGN YDGESSDELR NGKMKAFLSL ARFSDTQYQR |
| | 2401 IENYMKSSEF ENKQALLKRA KEEVGLLREH KIQTNRYTVK VQRELELDEL ALRALKEDRK |
| | 2461 RFLCKAVENY INCLLSGEEH DMWVFRLCSL WLENSGVSEV NGMMKRDGMK IPTYKFLPLM |
| | 2521 YQLAARMGTK MMGGLGFHEV LNNLISRISM DHPHHTLFII LALANANRDE FLTKPEVARR |
| | 2581 SRITKNVPKQ SSQLDEDRTE AANRIICTIR SRRPQMVRSV EALCDAYIIL ANLDATQWKT |
| | 2641 QRKGINIPAD QPITKLKNLE DVVVPTMEIK VDHTGEYGNL VTIQSFKAEF RLAGGVNLPK |
| | 2701 IIDCVGSDGK ERRQLVKGRD DLRQDAVMQQ VFQMCNTLLQ RNTETRKRKL TICTYKVVPL |
| | 2761 SQRSGVLEWC TGTVPIGEFL VNNEDGAHKR YRPNDFSAFQ CQKKMMEVQK KSFEEKYEVF |
| | 2821 MDVCQNFQPV FRYFCMEKFL DPAIWFEKRL AYTRSVATSS IVGYILGLGD RHVQNILINE |
| | 2881 QSAELVHIDL GVAFEQGKIL PTPETVPFRL TRDIVDGMGI TGVEGVFRRC CEKTMEVMRN |
| | 2941 SQETLLTIVE VLLYDPLFDW TMNPLKALYL QQRPEDETEL HPTLNADDQE CKRNLSDIDQ |
| | 3001 SFNKVAERVL MRLQEKLKGV EEGTVLSVGG QVNLLIQQAI DPKNLSRLFP GWKAWV |
| CTNNB1 | DNA Sequence |
| (SEQ ID NO: | 1 ATGGCTACTC AAGCTGATTT GATGGAGTTG GACATGGCCA TGGAACCAGA CAGAAAAGCG |
| 45) | 61 GCTGTTAGTC ACTGGCAGCA ACAGTCTTAC CTGGACTCTG GAATCCATTC TGGTGCCACT |
| | 121 ACCACAGCTC CTTCTCTGAG TGGTAAAGGC AATCCTGAGG AAGAGGATGT GGATACCTCC |
| | 181 CAAGTCCTGT ATGAGTGGGA ACAGGGATTT TCTCAGTCCT TCACTCAAGA ACAAGTAGCT |
| | 241 GATATTGATG GACAGTATGC AATGACTCGA GCTCAGAGGG TACGAGCTGC TATGTTCCCT |
| | 301 GAGACATTAG ATGAGGGCAT GCAGATCCCA TCTACACAGT TTGATGCTGC TCATCCCACT |
| | 361 AATGTCCAGC GTTTGGCTGA ACCATCACAG ATGCTGAAAC ATGCAGTTGT AAACTTGATT |
| | 421 AACTATCAAG ATGATGCAGA ACTTGCCACA CGTGCAATCC CTGAACTGAC AAAACTGCTA |
| | 481 AATGACGAGG ACCAGGTGGT GGTTAATAAG GCTGCAGTTA TGGTCCATCA GCTTTCTAAA |
| | 541 AAGGAAGCTT CCAGACACGC TATCATGCGT TCTCCTCAGA TGGTGTCTGC TATTGTACGT |
| | 601 ACCATGCAGA ATACAAATGA TGTAGAAACA GCTCGTTGTA CCGCTGGGAC CTTGCATAAC |
| | 661 CTTTCCCATC ATCGTGAGGG CTTACTGGCC ATCTTTAAGT CTGGAGGCAT TCCTGCCCTG |
| | 721 GTGAAAATGC TTGGTTCACC AGTGGATTCT GTGTTGTTTT ATGCCATTAC AACTCTCCAC |
| | 781 AACCTTTTAT TACATCAAGA AGGAGCTAAA ATGGCAGTGC GTTTAGCTGG TGGGCTGCAG |
| | 841 AAAATGGTTG CCTTGCTCAA CAAAACAAAT GTTAAATTCT TGGCTATTAC GACAGACTGC |
| | 901 CTTCAAATTT TAGCTTATGG CAACCAAGAA AGCAAGCTCA TCATACTGGC TAGTGGTGGA |
| | 961 CCCCAAGCTT TAGTAAATAT AATGAGGACC TATACTTACG AAAAACTACT GTGGACCACA |
| | 1021 AGCAGAGTGC TGAAGGTGCT ATCTGTCTGC TCTAGTAATA AGCCGGCTAT TGTAGAAGCT |
| | 1081 GGTGGAATGC AAGCTTTAGG ACTTCACCTG ACAGATCCAA GTCAACGTCT TGTTCAGAAC |
| | 1141 TGTCTTTGGA CTCTCAGGAA TCTTTCAGAT GCTGCAACTA AACAGGAAGG GATGGAAGGT |
| | 1201 CTCCTTGGGA CTCTTGTTCA GCTTCTGGGT TCAGATGATA TAAATGTGGT CACCTGTGCA |
| | 1261 GCTGGAATTC TTTCTAACCT CACTTGCAAT AATTATAAGA ACAAGATGAT GGTCTGCCAA |
| | 1321 GTGGGTGGTA TAGAGGCTCT TGTGCGTACT GTCCTTCGGG CTGGTGACAG GGAAGACATC |
| | 1381 ACTGAGCCTG CCATCTGTGC TCTTCGTCAT CTGACCAGCC GACACCAAGA AGCAGAGATG |
| | 1441 GCCCAGAATG CAGTTCGCCT TCACTATGGA CTACCAGTTG TGGTTAAGCT CTTACACCCA |
| | 1501 CCATCCCACT GGCCTCTGAT AAAGGCTACT GTTGGATTGA TTCGAAATCT TGCCCTTTGT |
| | 1561 CCCGCAAATC ATGCACCTTT GCGTGAGCAG GGTGCCATTC CACGACTAGT TCAGTTGCTT |
| | 1621 GTTCGTGCAC ATCAGGATAC CCAGCGCCGT ACGTCCATGG GTGGGACACA GCAGCAATTT |
| | 1681 GTGGAGGGGG TCCGCATGGA AGAAATAGTT GAAGGTTGTA CCGGAGCCCT TCACATCCTA |
| | 1741 GCTCGGGATG TTCACAACCG AATTGTTATC AGAGGACTAA ATACCATTCC ATTGTTTGTG |
| | 1801 CAGCTGCTTT ATTCTCCCAT TGAAAACATC CAAAGAGTAG CTGCAGGGGT CCTCTGTGAA |
| | 1861 CTTGCTCAGG ACAAGGAAGC TGCAGAAGCT ATTGAAGCTG AGGGAGCCAC AGCTCCTCTG |
| | 1921 ACAGAGTTAC TTCACTCTAG GAATGAAGGT GTGGCGACAT ATGCAGCTGC TGTTTTGTTC |
| | 1981 CGAATGTCTG AGGACAAGCC ACAAGATTAC AAGAAACGGC TTTCAGTTGA GCTGACCAGC |
| | 2041 TCTCTCTTCA GAACAGAGCC AATGGCTTGG AATGAGACTG CTGATCTTGG ACTTGATATT |
| | 2101 GGTGCCCAGG GAGAACCCCT TGGATATCGC CAGGATGATC CTAGCTATCG TTCTTTTCAC |
| | 2161 TCTGGTGGAT ATGGCCAGGA TGCCTTGGGT ATGGACCCCA TGATGGAACA TGAGATGGGT |
| | 2221 GGCCACCACC CTGGTGCTGA CTATCCAGTT GATGGGCTGC CAGATCTGGG GCATGCCCAG |
| | 2281 GACCTCATGG ATGGGCTGCC TCCAGGTGAC AGCAATCAGC TGGCCTGGTT TGATACTGAC |
| | 2341 CTGTAA |
| CTNINB1 | Protein Sequence |
| (SEQ ID NO: | 1 MATQADLMEL DMAMEPDRKA AVSHWQQQSY LDSGIHSGAT TTAPSLSGKG NPEEEDVDTS |
| 46) | 61 QVLYEWEQGF SQSFTQEQVA DIDGQYAMTR AQRVRAAMFP ETLDEGMQIP STQFDAAHPT |
| | 121 NVQRLAEPSQ MLKHAVVNLI NYQDDAELAT RAIPELTKLL NDEDQVVVNK AAVMVHQLSK |
| | 181 KEASRHAIMR SPQMVSAIVR TMQNTNDVET ARCTAGTLHN LSHHREGLLA IFKSGGIPAL |
| | 241 VKMLGSPVDS VLFYAITTLH NLLLHQEGAK MAVRLAGGLQ KMVALLNKTN VKFLAITTDC |
| | 301 LQILAYGNQE SKLIILASGG PQALVNIMRT YTYEKLLWTT SRVLKVLSVC SSNKPAIVEA |
| | 361 GGMQALGLHL TDPSQRLVQN CLWTLRNLSD AATKQEGMEG LLGTLVQLLG SDDINVVTCA |
| | 421 AGILSNLTCN NYKNKMMVCQ VGGIEALVRT VLRAGDREDI TEPAICALRH LTSRHQEAEM |
| | 481 AQNAVRLHYG LPVVVKLLHP PSHWPLIKAT VGLIRNLALC PANHAPLREQ GAIPRLVQLL |
| | 541 VRAHQDTQRR TSMGGTQQQF VEGVRMEEIV EGCTGALHIL ARDVHNRIVI RGLNTIPLFV |
| | 601 QLLYSPIENI QRVAAGVLCE LAQDKEAAEA IEAEGATAPL TELLHSRNEG VATYAAAVLF |
| | 661 RMSEDKPQDY KKRLSVELTS SLFRTEPMAW NETADLGLDI GAQGEPLGYR QDDPSYRSFH |
| | 721 SGGYGQDALG MDPMMEHEMG GHHPGADYPV DGLPDLGHAQ DLMDGLPPGD SNQLAWFDTD |
| | 781 L |
| ERBB3 | DNA Sequence |
| (SEQ ID NO: | 1 ATGAGGGCGA ACGACGCTCT GCAGGTGCTG GGCTTGCTTT TCAGCCTGGC CCGGGGCTCC |
| 47) | 61 GAGGTGGGCA ACTCTCAGGC AGTGTGTCCT GGGACTCTGA ATGGCCTGAG TGTGACCGGC |
| | 121 GATGCTGAGA ACCAATACCA GACACTGTAC AAGCTCTACG AGAGGTGTGA GGTGGTGATG |
| | 181 GGGAACCTTG AGATTGTGCT CACGGGACAC AATGCCGACC TCTCCTTCCT GCAGTGGATT |
| | 241 CGAGAAGTGA CAGGCTATGT CCTCGTGGCC ATGAATGAAT TCTCTACTCT ACCATTGCCC |
| | 301 AACCTCCGCG TGGTGCGAGG GACCCAGGTC TACGATGGGA AGTTTGCCAT CTTCGTCATG |
| | 361 TTGAACTATA ACACCAACTC CAGCCACGCT CTGCGCCAGC TCCGCTTGAC TCAGCTCACC |
| | 421 GAGATTCTGT CAGGGGGTGT TTATATTGAG AAGAACGATA AGCTTTGTCA CATGGACACA |
| | 481 ATTGACTGGA GGGACATCGT GAGGGACCGA GATGCTGAGA TAGTGGTGAA GGACAATGGC |
| | 541 AGAAGCTGTC CCCCCTGTCA TGAGGTTTGC AAGGGGCGAT GCTGGGGTCC TGGATCAGAA |
| | 601 GACTGCCAGA CATTGACCAA GACCATCTGT GCTCCTCAGT GTAATGGTCA CTGCTTTGGG |
| | 661 CCCAACCCCA ACCAGTGCTG CCATGATGAG TGTGCCGGGG GCTGCTCAGG CCCTCAGGAC |
| | 721 ACAGACTGCT TTGCCTGCCG GCACTTCAAT GACAGTGGAG CCTGTGTACC TCGCTGTCCA |
| | 781 CAGCCTCTTG TCTACAACAA GCTAACTTTC CAGCTGGAAC CCAATCCCCA CACCAAGTAT |
| | 841 CAGTATGGAG GAGTTTGTGT AGCCAGCTGT CCCCATAACT TTGTGGTGGA TCAAACATCC |
| | 901 TGTGTCAGGG CCTGTCCTCC TGACAAGATG GAAGTAGATA AAAATGGGCT CAAGATGTGT |
| | 961 GAGCCTTGTG GGGGACTATG TCCCAAAGCC TGTGAGGGAA CAGGCTCTGG GAGCCGCTTC |
| | 1021 CAGACTGTGG ACTCGAGCAA CATTGATGGA TTTGTGAACT GCACCAAGAT CCTGGGCAAC |
| | 1081 CTGGACTTTC TGATCACCGG CCTCAATGGA GACCCCTGGC ACAAGATCCC TGCCCTGGAC |
| | 1141 CCAGAGAAGC TCAATGTCTT CCGGACAGTA CGGGAGATCA CAGGTTACCT GAACATCCAG |
| | 1201 TCCTGGCCGC CCCACATGCA CAACTTCAGT GTTTTTTCCA ATTTGACAAC CATTGGAGGC |
| | 1261 AGAAGCCTCT ACAACCGGGG CTTCTCATTG TTGATCATGA AGAACTTGAA TGTCACATCT |
| | 1321 CTGGGCTTCC GATCCCTGAA GGAAATTAGT GCTGGGCGTA TCTATATAAG TGCCAATAGG |
| | 1381 CAGCTCTGCT ACCACCACTC TTTGAACTGG ACCAAGGTGC TTCGGGGGCC TACGGAAGAG |
| | 1441 CGACTAGACA TCAAGCATAA TCGGCCGCGC AGAGACTGCG TGGCAGAGGG CAAAGTGTGT |
| | 1501 GACCCACTGT GCTCCTCTGG GGGATGCTGG GGCCCAGGCC CTGGTCAGTG CTTGTCCTGT |
| | 1561 CGAAATTATA GCCGAGGAGG TGTCTGTGTG ACCCACTGCA ACTTTCTGAA TGGGGAGCCT |
| | 1621 CGAGAATTTG CCCATGAGGC CGAATGCTTC TCCTGCCACC CGGAATGCCA ACCCATGGAG |
| | 1681 GGCACTGCCA CATGCAATGG CTCGGGCTCT GATACTTGTG CTCAATGTGC CCATTTTCGA |
| | 1741 GATGGGCCCC ACTGTGTGAG CAGCTGCCCC CATGGAGTCC TAGGTGCCAA GGGCCCAATC |
| | 1801 TACAAGTACC CAGATGTTCA GAATGAATGT CGGCCCTGCC ATGAGAACTG CACCCAGGGG |
| | 1861 TGTAAAGGAC CAGAGCTTCA AGACTGTTTA GGACAAACAC TGGTGCTGAT CGGCAAAACC |
| | 1921 CATCTGACAA TGGCTTTGAC AGTGATAGCA GGATTGGTAG TGATTTTCAT GATGCTGGGC |
| | 1981 GGCACTTTTC TCTACTGGCG TGGGCGCCGG ATTCAGAATA AAAGGGCTAT GAGGCGATAC |
| | 2041 TTGGAACGGG GTGAGAGCAT AGAGCCTCTG GACCCCAGTG AGAAGGCTAA CAAAGTCTTG |
| | 2101 GCCAGAATCT TCAAAGAGAC AGAGCTAAGG AAGCTTAAAG TGCTTGGCTC GGGTGTCTTT |
| | 2161 GGAACTGTGC ACAAAGGAGT GTGGATCCCT GAGGGTGAAT CAATCAAGAT TCCAGTCTGC |
| | 2221 ATTAAAGTCA TTGAGGACAA GAGTGGACGG CAGAGTTTTC AAGCTGTGAC AGATCATATG |
| | 2281 CTGGCCATTG GCAGCCTGGA CCATGCCCAC ATTGTAAGGC TGCTGGGACT ATGCCCAGGG |
| | 2341 TCATCTCTGC AGCTTGTCAC TCAATATTTG CCTCTGGGTT CTCTGCTGGA TCATGTGAGA |
| | 2401 CAACACCGGG GGGCACTGGG GCCACAGCTG CTGCTCAACT GGGGAGTACA AATTGCCAAG |
| | 2461 GGAATGTACT ACCTTGAGGA ACATGGTATG GTGCATAGAA ACCTGGCTGC CCGAAACGTG |
| | 2521 CTACTCAAGT CACCCAGTCA GGTTCAGGTG GCAGATTTTG GTGTGGCTGA CCTGCTGCCT |
| | 2581 CCTGATGATA AGCAGCTGCT ATACAGTGAG GCCAAGACTC CAATTAAGTG GATGGCCCTT |
| | 2641 GAGAGTATCC ACTTTGGGAA ATACACACAC CAGAGTGATG TCTGGAGCTA TGGTGTGACA |
| | 2701 GTTTGGGAGT TGATGACCTT CGGGGCAGAG CCCTATGCAG GGCTACGATT GGCTGAAGTA |
| | 2761 CCAGACCTGC TAGAGAAGGG GGAGCGGTTG GCACAGCCCC AGATCTGCAC AATTGATGTC |
| | 2821 TACATGGTGA TGGTCAAGTG TTGGATGATT GATGAGAACA TTCGCCCAAC CTTTAAAGAA |
| | 2881 CTAGCCAATG AGTTCACCAG GATGGCCCGA GACCCACCAC GGTATCTGGT CATAAAGAGA |
| | 2941 GAGAGTGGGC CTGGAATAGC CCCTGGGCCA GAGCCCCATG GTCTGACAAA CAAGAAGCTA |
| | 3001 GAGGAAGTAG AGCTGGAGCC AGAACTAGAC CTAGACCTAG ACTTGGAAGC AGAGGAGGAC |
| | 3061 AACCTGGCAA CCACCACACT GGGCTCCGCC CTCAGCCTAC CAGTTGGAAC ACTTAATCGG |
| | 3121 CCACGTGGGA GCCAGAGCCT TTTAAGTCCA TCATCTGGAT ACATGCCCAT GAACCAGGGT |
| | 3181 AATCTTGGGG AGTCTTGCCA GGAGTCTGCA GTTTCTGGGA GCAGTGAACG GTGCCCCCGT |
| | 3241 CCAGTCTCTC TACACCCAAT GCCACGGGGA TGCCTGGCAT CAGAGTCATC AGAGGGGCAT |
| | 3301 GTAACAGGCT CTGAGGCTGA GCTCCAGGAG AAAGTGTCAA TGTGTAGGAG CCGGAGCAGG |
| | 3361 AGCCGGAGCC CACGGCCACG CGGAGATAGC GCCTACCATT CCCAGCGCCA CAGTCTGCTG |
| | 3421 ACTCCTGTTA CCCCACTCTC CCCACCCGGG TTAGAGGAAG AGGATGTCAA CGGTTATGTC |
| | 3481 ATGCCAGATA CACACCTCAA AGGTACTCCC TCCTCCCGGG AAGGCACCCT TTCTTCAGTG |
| | 3541 GGTCTCAGTT CTGTCCTGGG TACTGAAGAA GAAGATGAAG ATGAGGAGTA TGAATACATG |
| | 3601 AACCGGAGGA GAAGGCACAG TCCACCTCAT CCCCCTAGGC CAAGTTCCCT TGAGGAGCTG |
| | 3661 GGTTATGAGT ACATGGATGT GGGGTCAGAC CTCAGTGCCT CTCTGGGCAG CACACAGAGT |
| | 3721 TGCCCACTCC ACCCTGTACC CATCATGCCC ACTGCAGGCA CAACTCCAGA TGAAGACTAT |
| | 3781 GAATATATGA ATCGGCAACG AGATGGAGGT GGTCCTGGGG GTGATTATGC AGCCATGGGG |
| | 3841 GCCTGCCCAG CATCTGAGCA AGGGTATGAA GAGATGAGAG CTTTTCAGGG GCCTGGACAT |
| | 3901 CAGGCCCCCC ATGTCCATTA TGCCCGCCTA AAAACTCTAC GTAGCTTAGA GGCTACAGAC |
| | 3961 TCTGCCTTTG ATAACCCTGA TTACTGGCAT AGCAGGCTTT TCCCCAAGGC TAATGCCCAG |
| | 4021 AGAACGTAA |
| ERBB3 | Protein Sequence |
| (SEQ ID NO: | 1 MRANDALQVL GLLFSLARGS EVGNSQAVCP GTLNGLSVTG DAENQYQTLY KLYERCEVVM |
| 48) | 61 GNLEIVLTGH NADLSFLQWI REVTGYVLVA MNEFSTLPLP NLRVVRGTQV YDGKFAIFVM |
| | 121 LNYNTNSSHA LRQLRLTQLT EILSGGVYIE KNDKLCHMDT IDWRDIVRDR DAEIVVKDNG |
| | 181 RSCPPCHEVC KGRCWGPGSE DCQTLTKTIC APQCNGHCFG PNPNQCCHDE CAGGCSGPQD |
| | 241 TDCFACRHFN DSGACVPRCP QPLVYNKLTF QLEPNPHTKY QYGGVCVASC PHNFVVDQTS |
| | 301 CVRACPPDKM EVDKNGLKMC EPCGGLCPKA CEGTGSGSRF QTVDSSNIDG FVNCTKILGN |
| | 361 LDFLITGLNG DPWHKIPALD PEKLNVFRTV REITGYLNIQ SWPPHMHNFS VFSNLTTIGG |
| | 421 RSLYNRGFSL LIMKNLNVTS LGFRSLKEIS AGRIYISANR QLCYHHSLNW TKVLRGPTEE |
| | 481 RLDIKHNRPR RDCVAEGKVC DPLCSSGGCW GPGPGQCLSC RNYSRGGVCV THCNFLNGEP |
| | 541 REFAHEAECF SCHPECQPME GTATCNGSGS DTCAQCAHFR DGPHCVSSCP HGVLGAKGPI |
| | 601 YKYPDVQNEC RPCHENCTQG CKGPELQDCL GQTLVLIGKT HLTMALTVIA GLVVIFMMLG |
| | 661 GTFLYWRGRR IQNKRAMRRY LERGESIEPL DPSEKANKVL ARIFKETELR KLKVLGSGVF |
| | 721 GTVHKGVWIP EGESIKIPVC IKVIEDKSGR QSFQAVTDHM LAIGSLDHAH IVRLLGLCPG |
| | 781 SSLQLVTQYL PLGSLLDHVR QHRGALGPQL LLNWGVQIAK GMYYLEEHGM VHRNLAARNV |
| | 841 LLKSPSQVQV ADFGVADLLP PDDKQLLYSE AKTPIKWMAL ESIHFGKYTH QSDVWSYGVT |
| | 901 VWELMTFGAE PYAGLRLAEV PDLLEKGERL AQPQICTIDV YMVMVKCWMI DENIRPTFKE |
| | 961 LANEFTRMAR DPPRYLVIKR ESGPGIAPGP EPHGLTNKKL EEVELEPELD LDLDLEAEED |
| | 1021 NLATTTLGSA LSLPVGTLNR PRGSQSLLSP SSGYMPMNQG NLGESCQESA VSGSSERCPR |
| | 1081 PVSLHPMPRG CLASESSEGH VTGSEAELQE KVSMCRSRSR SRSPRPRGDS AYHSQRHSLL |
| | 1141 TPVTPLSPPG LEEEDVNGYV MPDTHLKGTP SSREGTLSSV GLSSVLGTEE EDEDEEYEYM |
| | 1201 NRRRRHSPPH PPRPSSLEEL GYEYMDVGSD LSASLGSTQS CPLHPVPIMP TAGTTPDEDY |
| | 1261 EYMNRQRDGG GPGGDYAAMG ACPASEQGYE EMRAFQGPGH QAPHVHYARL KTLRSLEATD |
| | 1321 SAFDNPDYWH SRLFPKANAQ RT |
| GNAS | DNA Sequence |
| (SEQ ID NO: | 1 ATGGGCTGCC TCGGGAACAG TAAGACCGAG GACCAGCGCA ACGAGGAGAA GGCGCAGCGT |
| 49) | 61 GAGGCCAACA AAAAGATCGA GAAGCAGCTG CAGAAGGACA AGCAGGTCTA CCGGGCCACG |
| | 121 CACCGCCTGC TGCTGCTGGG TGCTGGAGAA TCTGGTAAAA GCACCATTGT GAAGCAGATG |
| | 181 AGGATCCTGC ATGTTAATGG GTTTAATGGA GAGGGCGGCG AAGAGGACCC GCAGGCTGCA |
| | 241 AGGAGCAACA GCGATGGTGA GAAGGCAACC AAAGTGCAGG ACATCAAAAA CAACCTGAAA |
| | 301 GAGGCGATTG AAACCATTGT GGCCGCCATG AGCAACCTGG TGCCCCCCGT GGAGCTGGCC |
| | 361 AACCCCGAGA ACCAGTTCAG AGTGGACTAC ATCCTGAGTG TGATGAACGT GCCTGACTTT |
| | 421 GACTTCCCTC CCGAATTCTA TGAGCATGCC AAGGCTCTGT GGGAGGATGA AGGAGTGCGT |
| | 481 GCCTGCTACG AACGCTCCAA CGAGTACCAG CTGATTGACT GTGCCCAGTA CTTCCTGGAC |
| | 541 AAGATCGACG TGATCAAGCA GGCTGACTAT GTGCCGAGCG ATCAGGACCT GCTTCGCTGC |
| | 601 CGTGTCCTGA CTTCTGGAAT CTTTGAGACC AAGTTCCAGG TGGACAAAGT CAACTTCCAC |
| | 661 ATGTTTGACG TGGGTGGCCA GCGCGATGAA CGCCGCAAGT GGATCCAGTG CTTCAACGAT |
| | 721 GTGACTGCCA TCATCTTCGT GGTGGCCAGC AGCAGCTACA ACATGGTCAT CCGGGAGGAC |
| | 781 AACCAGACCA ACCGCCTGCA GGAGGCTCTG AACCTCTTCA AGAGCATCTG GAACAACAGA |
| | 841 TGGCTGCGCA CCATCTCTGT GATCCTGTTC CTCAACAAGC AAGATCTGCT CGCTGAGAAA |
| | 901 GTCCTTGCTG GGAAATCGAA GATTGAGGAC TACTTTCCAG AATTTGCTCG CTACACTACT |
| | 961 CCTGAGGATG CTACTCCCGA GCCCGGAGAG GACCCACGCG TGACCCGGGC CAAGTACTTC |
| | 1021 ATTCGAGATG AGTTTCTGAG GATCAGCACT GCCAGTGGAG ATGGGCGTCA CTACTGCTAC |
| | 1081 CCTCATTTCA CCTGCGCTGT GGACACTGAG AACATCCGCC GTGTGTTCAA CGACTGCCGT |
| | 1141 GACATCATTC AGCGCATGCA CCTTCGTCAG TACGAGCTGC TCTAA |
| GNAS | Protein Sequence |
| (SEQ ID NO: | 1 MGCLGNSKTE DQRNEEKAQR EANKKIEKQL QKDKQVYRAT HRLLLLGAGE SGKSTIVKQM |
| 50) | 61 RILHVNGFNG EGGEEDPQAA RSNSDGEKAT KVQDIKNNLK EAIETIVAAM SNLVPPVELA |
| | 121 NPENQFRVDY ILSVMNVPDF DFPPEFYEHA KALWEDEGVR ACYERSNEYQ LIDCAQYFLD |
| | 181 KIDVIKQADY VPSDQDLLRC RVLTSGIFET KFQVDKVNFH MFDVGGQRDE RRKWIQCFND |
| | 241 VTAIIFVVAS SSYNMVIRED NQTNRLQEAL NLFKSIWNNR WLRTISVILF LNKQDLLAEK |
| | 301 VLAGKSKIED YFPEFARYTT PEDATPEPGE DPRVTRAKYF IRDEFLRIST ASGDGRHYCY |
| | 361 PHFTCAVDTE NIRRVFNDCR DIIQRMHLRQ YELL |
| CRC DM | DNA Sequence |
| construct 1 | 1 ATGACCACCA TCCACTACAA CTACATGTGC AACAGCAGCT GCATGGGCAG CATGAACTGG |
| insert | 61 CGGCCTATCC TGACCATCAT CACCCTGGAA GATAGCCGGG GCAGAAAGCG GAGAAGCGTG |
| (SEQ ID NO: | 121 GCCATGAACG AGTTCAGCAC ACTGCCCCTG CCTAACCTGA GAATGGTTCG AGGCACCCAG |
| 51) | 181 GTGTACGACG GCAAGTTCGC CATCTTTGTG CGCGGCAGAA AGAGGCGGAG CTACCTGGAT |
| | 241 TCTGGCATCC ACTCTGGCGC TACAACAACA GCCCCATTCC TGAGCGGCAA GGGCAACCCC |
| | 301 GAAGAGGAAG ATGTGGATAC CAGCAGAGGC CGGAAGAGAA GATCCGACGT GGAAACCGGC |
| | 361 AACTGCATCC ACACACTGAC AGGCCACCAG CTGCTGACCT CTGGCATGGA ACTGAAGGAC |
| | 421 AACATCCTGG TGTCCGGCAG AGGAAGAAAG CGCAGATCTA CCGGCGAGTG CATTCACACC |
| | 481 CTGTATGGCC ACACCAGCAC CGTGCACTGC ATGCATCTGC ACGAGAAGAG AGTGGTGTCT |
| | 541 GGCAGCAGAG ACAGAGGACG CAAGCGGAGA TCCGAGCAAG AGGCCCTGGA ATACTTTATG |
| | 601 AAGCAGATCA ACGACGCCTA CCACGGCGGC TGGACTACCA AGATGGACTG GATCTTCCAC |
| | 661 ACCATCCGCG GACGCAAGAG AAGAAGCGTG ACACAAGAGG CCGAGCGGGA AGAGTTCTTC |
| | 721 GACGAGACAA GACAGCTGTG CGACCTGCGG CTGTTCCAGC CTTTCCTGAA AGTGATCGAG |
| | 781 CGCGGACGGA AAAGACGGTC CACCGAGTAT AAGCTGGTGG TCGTGGGAGC TTGTGGCGTG |
| | 841 GGAAAAAGCG CCCTGACAAT CCAGCTGATC CAGAACCACT TCGTGCGGGG AAGAAAACGG |
| | 901 CGGAGCATGG CCATCTACAA GCAGAGCCAG CACATGACCG AGGTCGTGCG GCACTGTCCT |
| | 961 CACCACGAGA GATGTAGCGA TAGCGACGGA CTGGCCCCTT GATGA |
| CRC DM | Protein Sequence* |
| construct1 | 1 MTTIHYNYMC NSSCMGSMNW RPILTIITLE DSRGRKRRSV AMNEFSTLPL PNLRMVRGTQ |
| insert | 61 VYDGKFAIFV RGRKRRSYLD SGIHSGATTT APFLSGKGNP EEEDVDTSRG RKRRSDVETG |
| (SEQ ID NO: | 121 NCIHTLTGHQ LLTSGMELKD NILVSGRGRK RRSTGECIHT LYGHTSTVHC MHLHEKRVVS |
| 52) | 181 GSRDRGRKRR SEQEALEYFM KQINDAYHGG WTTKMDWIFH TIRGRKRRSV TQEAEREEFF |
| | 241 DETRQLCDLR LFQPFLKVIE RGRKRRSTEY KLVVVGACGV GKSALTIQLI QNHFVRGRKR |
| | 301 RSMAIYKQSQ HMTEVVRHCP HHERCSDSDG LAP |
| CRC DM | DNA Sequence |
| construct 2 | 1 ATGGAAGATA GCAGCGGCAA TCTGCTGGGC AGAAACAGCT TCGAAGTGTG CGTGTGTGCC |
| insert | 61 TGTCCTGGCA GAGACAGAAG AACCGAGGAA GAGAACCGGG GCAGAAAGCG GAGAAGCGAC |
| (SEQ ID NO: | 121 AAAGAGCAGC TGAAGGCCAT CAGCACCAGA GATCCTCTGA GCAAGATCAC AGAGCAAGAG |
| 53) | 181 AAGGACTTCC TGTGGTCCCA CCGGCACTAC AGAGGCCGGA AGAGAAGATC TACCGGCCAG |
| | 241 TGTCTGCACG TCCTGATGGG ACATGTGGCC GCCGTGTGTT GCGTGCAGTA CGATGGCAGA |
| | 301 AGAGTGGTTT CCGGCGCCTA CGACAGAGGA AGAAAAAGGC GGTCCCCTAT CGTGACCGTG |
| | 361 GACGGCTATG TTGATCCCTC TGGCGGCGAT CACTTCTGCC TGGGCCAGCT GTCTAACGTG |
| | 421 CACAGAACCG AAGCCATCAG AGGACGGAAG CGGAGATCCG AGATCAGCCA CATCGGCAGC |
| | 481 AGAGGCAAGT ACAGCAGCGG CTTCTGCAAT ATCGCCGTGA AAGAGAACCT GATCGAACTG |
| | 541 ATGGCCGACA TCAGAGGTAG AAAGCGGCGG AGCAAGCAGG CCGATTACGT GCCATCTGAC |
| | 601 CAGGACCTGC TGAGATGCCA CGTGCTGACC AGCGGCATCT TCGAGACAAA GTTCCAGGTG |
| | 661 GACAAGTGAT GA |
| CRC DM | Protein Sequence* |
| construct 2 | 1 MEDSSGNLLG RNSFEVCVCA CPGRDRRTEE ENRGRKRRSD KEQLKAISTR DPLSKITEQE |
| insert | 61 KDFLWSHRHY RGRKRRSTGQ CLHVLMGHVA AVCCVQYDGR RVVSGAYDRG RKRRSPIVTV |
| (SEQ ID NO: | 121 DGYVDPSGGD HFCLGQLSNV HRTEAIRGRK RRSEISHIGS RGKYSSGFCN IAVKENLIEL |
| 54) | 181 MADIRGRKRR SKQADYVPSD QDLLRCHVLT SGIFETKFQV DK |

*Driver mutation is highlighted in bold. The furin cleavage sequence is underlined.

### Immune Responses to Driver Mutations Induced by the CRC Vaccine-B RKO Cell Line (CRC Construct 1 SEQ ID NO: 52))

CRC vaccine-B cell line RKO modified to reduce expression of CD276 and TGFβ1, and express GM-CSF, membrane bound CD40L, IL-12 was transduced with lentiviral particles expressing to three TP53 driver mutations, one KRAS driver mutation, three PIK3CA driver mutations, two FBXW7 driver mutations, one CTNNB1 driver mutation and one ERBB3 driver mutations encoded by nine peptide sequences separated by the furin cleavage sequence RGRKRRS (SEQ ID NO: 32) as described above.

Immune responses to the inserted TP53, KRAS, PIK3CA, FBXW7, CTNNB1 and ERBB3 driver mutations were evaluated by IFNγ ELISpot as described above and herein. Specifically, 1.5×10⁶ of unmodified RKO or RKO modified to express driver mutations peptides were co-cultured with 1.5×10⁶ iDCs from six HLA diverse donors (n=4/donor). HLA-A, HLA-B, and HLA-C alleles for each of the six donors are in Table 2-20. Peptides, 15-mers overlapping by 9 amino acids, were designed to cover the full amino acid sequences of the twelve individual driver mutations peptides. Only the 15-mer peptides containing the mutations were used to stimulate PBMCs in the IFNγ ELISpot assay.

TABLE 2-20
Donor MHC-I HLA Alleles

| Donor # | HLA-A | HLA-B | HLA-C |
|---|---|---|---|
| 1 | *02:01 *33:01 | *07:02 14:02 | *07:02 *08:02 |
| 2 | *03:01 *25:01 | *15:01 44:02 | *03:03 *05:01 |
| 3 | *02:01 *25:01 | *18:01 *44:03 | *12:03 *06:01 |
| 4 | *03:01 *11:01 | *18:01 *51:01 | *06:02 *07:01 |
| 5 | *01:01 *03:01 | *07:02 *44:02 | *05:01 *07:02 |
| 6 | *03:01 *31:01 | *35:01 *40:01 | *04:01 *07:02 |

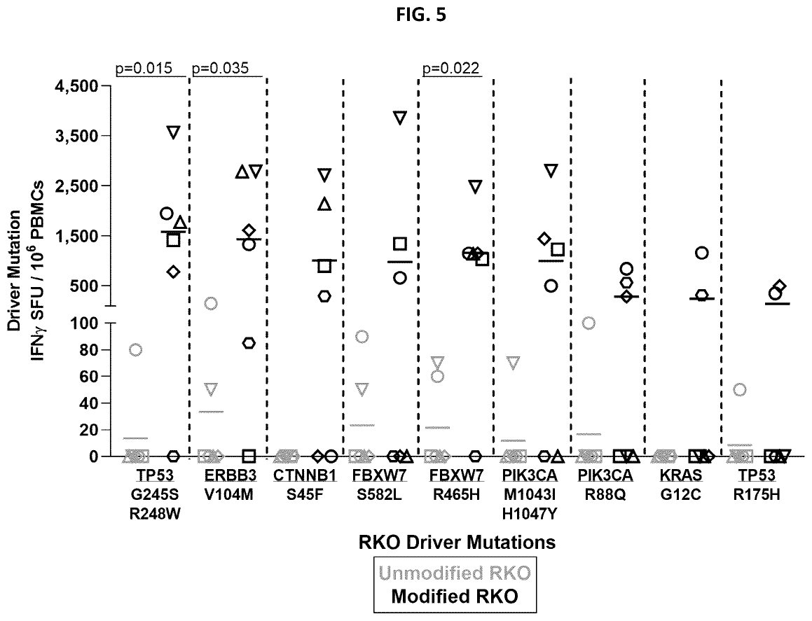

FIG. 5 demonstrates immune responses against nine driver mutation encoding peptides expressed by the CRC vaccine-B RKO cell line for six HLA-diverse donors by IFNγ ELISpot. CRC vaccine-B RKO induced IFNγ responses against all inserted driver mutation encoding peptides greater in magnitude relative to the unmodified RKO cell line (Table 2-21). The magnitude of IFNγ responses induced by CRC vaccine-B RKO cell line significantly increased against the inserted driver mutation peptides encoding TP53 G245S R248W (p=0.015), ERBB3 V104M (p=0.035), and FBXW7 R465H (p=0.022) compared to the unmodified RKO cell line. Statistical significance was determined using the Mann-Whitney U test.

TABLE 2-21
Immune responses to TP53, KRAS, PIK3CA, FBXW7, CTNNB1 and ERBB3 driver mutations expressed by
the CRC vaccine-B RKO cell line

| | Unmodified RKO (SFU ±SEM) | | | | | | | | |
|---|---|---|---|---|---|---|---|---|---|
| CRC | | | | | | | PIK3CA | | |
| Driver | | TP53 G245S | ERBB3 | CTNNB1 | FBXW7 | FBXW7 | M1043I | PIK3CA | KRAS |
| Mutation | TP53 R175H | R248W | V216M | S45F | S582L | R465H | H1047Y | R88Q | G12C |
| Donor 1 | 50 ± 30 | 80 ± 57 | 150 ± 53 | 0 ± 0 | 90 ± 77 | 60 ± 35 | 0 ± 0 | 100 ± 26 | 0 ± 0 |
| Donor 2 | 0 ± 0 | 0 ± 0 | 0 ± 0 | 0 ± 0 | 0 ± 0 | 0 ± 0 | 0 ± 0 | 0 ± 0 | 0 ± 0 |
| Donor 3 | 0 ± 0 | 0 ± 0 | 0 ± 0 | 0 ± 0 | 0 ± 0 | 0 ± 0 | 0 ± 0 | 0 ± 0 | 0 ± 0 |
| Donor 4 | 0 ± 0 | 0 ± 0 | 0 ± 0 | 0 ± 0 | 0 ± 0 | 0 ± 0 | 0 ± 0 | 0 ± 0 | 0 ± 0 |
| Donor 5 | 0 ± 0 | 0 ± 0 | 0 ± 0 | 0 ± 0 | 0 ± 0 | 0 ± 0 | 0 ± 0 | 0 ± 0 | 0 ± 0 |
| Donor 6 | 0 ± 0 | 0 ± 0 | 50 ± 19 | 0 ± 0 | 50 ± 38 | 70 ± 34 | 70 ± 37 | 0 ± 0 | 0 ± 0 |
| Average | 8 ± 8 | 13 ± 13 | 33 ± 25 | 0 ± 0 | 23 ± 16 | 22 ± 14 | 12 ± 12 | 17 ± 17 | 0 ± 0 |
| | Modified RKO (SFU ± SEM) | | | | | | | | |
| CRC | | | | | | | PIK3CA | | |
| Driver | | TP53 G245S | ERBB3 | CTNNB1 | FBXW7 | FBXW7 | M10431 | PIK3CA | KRAS |
| mutation | TP53 R175H | R248W | V216M | S45F | S582L | R465H | H1047Y | R88Q | G12C |
| Donor 1 | 350 ± 311 | 1,950 ± 595 | 1,330 ± 804 | 0 ± 0 | 660 ± 491 | 1,150 ± 461 | 500 ± 22 | 840 ± 788 | 1,160 ± 1,056 |
| Donor 2 | 0 ± 0 | 1,413 ± 1,033 | 0 ± 0 | 900 ± 520 | 1,343 ± 696 | 1,035 ± 922 | 1,228 ± 583 | 0 ± 0 | 0 ± 0 |
| Donor 3 | 0 ± 0 | 1,178 ± 609 | 2,785 ± 932 | 2,143 ± 1,150 | 0 ± 0 | 1140 ± 661 | 0 ± 0 | 0 ± 0 | 0 ± 0 |
| Donor 4 | 495 ± 314 | 785 ± 469 | 1,610 ± 1,131 | 0 ± 0 | 0 ± 0 | 1,148 ± 446 | 1,440 ± 833 | 288 ± 167 | 0 ± 0 |
| Donor 5 | 0 ± 0 | 0 ± 0 | 85 ± 59 | 295 ± 210 | 0 ± 0 | 0 ± 0 | 0 ± 0 | 565 ± 365 | 315 ± 224 |
| Donor 6 | 0 ± 0 | 3,565 ± 1,535 | 2,790 ± 1,322 | 2,710 ± 1,204 | 3,860 ± 1,467 | 2,480 ± 1,248 | 2,800 ± 1,232 | 0 ± 0 | 0 ± 0 |
| Average | 141 ± 91 | 1,582 ± 492 | 1,433 ± 503 | 1,008 ± 474 | 977 ± 617 | 1,159 ± 322 | 995 ± 437 | 282 ± 145 | 246 ± 190 |

### Immune Responses to Driver Mutations Induced by the CRC Vaccine-A HuTu8 Cell Line (CRC Construct 2 SEQ ID NO: 54))

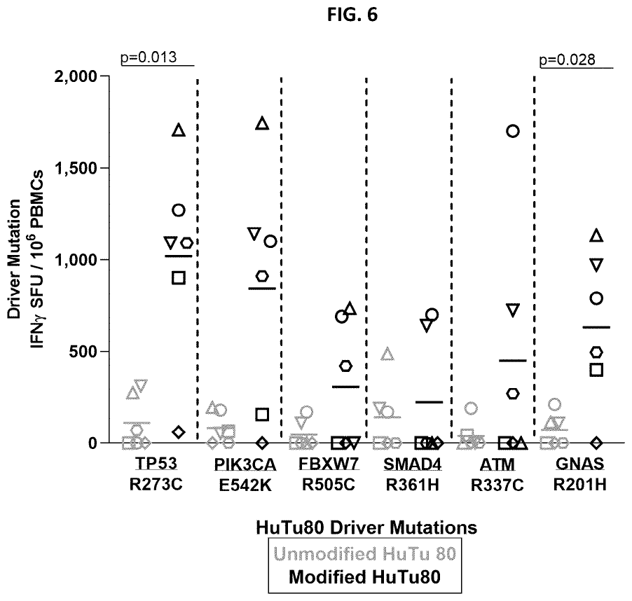

Immune responses to six driver mutation encoding peptides expressed by CRC vaccine-A cell line HuTu8 were determined for six HLA-diverse donors (Table 2-20) by IFNγ ELISpot. CRC vaccine-A HuTu80 induced IFNγ responses against all inserted driver mutation encoding peptides greater in magnitude relative to unmodified HuTu80. FIG. 6 describes immune responses against the six driver mutation encoding peptides inserted into CRC vaccine-A cell line HuTu80 induced IFNγ responses against all inserted driver mutation encoding peptides greater in magnitude relative to the unmodified HuTu80 cell line The magnitude of IFNγ responses induced by CRC vaccine-A HuTu80 cell line significantly increased against the inserted driver mutation peptides encoding TP53 R273C (p=0.03) and GNAS R231H (p=0.028) compared to the unmodified HuTu80 cell line (Table 2-22). Statistical significance was determined using the Mann-Whitney U test.

TABLE 2-22
Immune responses to TP53, PIK3CA, FBXW7, SMAD4, ATM and GNAS driver mutations expressed by the
CRC vaccine-A Hutu80 cell line

| CRC | Unmodified HuTu80 (SFU ± SEM) | | | | | |
|---|---|---|---|---|---|---|
| Driver | | PIK3CA | FBXW7 | SMAD4 | ATM | GNAS |
| Mutation | TP53 R273C | E542K | R505C | R361H | R337C | R201H |
| Donor 1 | 0 ± 0 | 180 ± 74 | 170 ± 87 | 170 ± 62 | 190 ± 164 | 210 ± 91 |
| Donor 2 | 0 ± 0 | 65 ± 38 | 0 ± 0 | 0 ± 0 | 43 ± 17 | 0 ± 0 |
| Donor 3 | 275 ± 161 | 195 ± 123 | 0 ± 0 | 488 ± 405 | 0 ± 0 | 115 ± 68 |
| Donor 4 | 0 ± 0 | 0 ± 0 | 0 ± 0 | 0 ± 0 | 0 ± 0 | 0 ± 0 |
| Donor 5 | 70 ± 41 | 0 ± 0 | 0 ± 0 | 0 ± 0 | 0 ± 0 | 0 ± 0 |
| Donor 6 | 310 ± 254 | 53 ± 24 | 110 ± 72 | 190 ± 117 | 0 ± 0 | 110 ± 064 |
| Average | 109 ± 59 | 82 ± 35 | 47 ± 31 | 141 ± 78 | 39 ± 31 | 73 ± 36 |
| CRC | Modified HuTu80 (SFU ± SEM) | | | | | |
| Driver | | PIK3CA | FBXW7 | SMAD4 | ATM | GNAS |
| mutation | TP53 R273C | E542K | R505C | R361H | R337C | R201H |
| Donor 1 | 1,270 ± 579 | 1,100 ± 385 | 690 ± 323 | 700 ± 356 | 1,700 ± 228 | 790 ± 335 |
| Donor 2 | 900 ± 340 | 155 ± 95 | 0 ± 0 | 0 ± 0 | 0 ± 0 | 400 ± 288 |
| Donor 3 | 1,708 ± 623 | 1,745 ± 1,323 | 735 ± 437 | 0 ± 0 | 0 ± 0 | 1,133 ± 568 |
| Donor 4 | 60 ± 42 | 0 ± 0 | 0 ± 0 | 0 ± 0 | 0 ± 0 | 0 ± 0 |
| Donor 5 | 1,090 ± 402 | 910 ± 308 | 420 ± 247 | 0 ± 0 | 270 ± 257 | 495 ± 422 |
| Donor 6 | 1,090 ± 180 | 1,140 ± 239 | 0 ± 0 | 640 ± 262 | 725 ± 446 | 970 ± 345 |
| Average | 1,020 ± 222 | 842 ± 268 | 308 ± 144 | 223 ± 141 | 449 ± 276 | 631 ± 169 |

Genetic modifications completed for CRC vaccine-A and CRC vaccine-B cell lines are described in Table 2-24 below and herein. The CD276 gene was knocked out (KO) by electroporation of zinc-finger nucleases (ZFN) (SEQ ID NO: 25) as described above. All other genetic modifications were completed by lentiviral transduction.

### CRC Vaccine-A

HCT-15 (ATCC, CCL-225) is modified to reduce expression of CD276, reduce secretion of transforming growth factor-beta 1 (TGFβ1), and express granulocyte macrophage—colony stimulating factor (GM-CSF) (SEQ ID NO: 7, SEQ ID NO: 8), membrane-bound CD40L (mCD40L) (SEQ ID NO: 2, SEQ ID NO: 3), interleukin 12 p70 and (IL-12) (SEQ ID NO: 9, SEQ ID NO: 10);

HuTu80 (ATCC, HTB-40) is modified to reduce expression of CD276, reduce secretion of TGFβ1 and transforming growth factor-beta 1 (TGFβ2), and express GM-CSF (SEQ ID NO: 7, SEQ ID NO: 8), membrane bound CD40L (SEQ ID NO: 2, SEQ ID NO: 3), IL-12 (SEQ ID NO: 9, SEQ ID NO: 10), modPSMA (SEQ ID NO: 19, SEQ ID NO: 20); and express peptides containing TP53 (SEQ ID NO: 35, SEQ ID NO: 36) driver mutations R273C, PIK3CA (SEQ ID NO: 37, SEQ ID NO: 38) driver mutations E542K, SMAD4 (SEQ ID NO: 41, SEQ ID NO: 42) driver mutation R361H, GNAS (SEQ ID NO: 49, SEQ ID NO: 50) driver mutation R201H, FBXW7 (SEQ ID NO: 39, SEQ ID NO: 40) driver mutation R505C, and ATM (SEQ ID NO: 43, SEQ ID NO: 44) driver mutation R337C as provided in SEQ ID NO: 54;

LS411N (ATCC, CRL-2159) is modified to reduce expression of CD276, reduced secretion of TGFβ1 and express GM-CSF (SEQ ID NO: 7, SEQ ID NO: 8), membrane bound CD40L (SEQ ID NO: 2, SEQ ID NO: 3), IL-12 (SEQ ID NO: 9, SEQ ID NO: 10).

### CRC Vaccine-B

HCT-116 (ATCC, CCL-247) modified to reduced expression of CD276, reduce secretion of TGFβ1, and express GM-CSF (SEQ ID NO: 7, SEQ ID NO: 8), membrane bound CD40L (SEQ ID NO: 2, SEQ ID NO: 3), IL-12 (SEQ ID NO: 9, SEQ ID NO: 10), modTBXT (SEQ ID NO: 17, SEQ ID NO: 18), modWT1 (SEQ ID NO: 17, SEQ ID NO: 18), and peptides comprising one or more KRAS (SEQ ID NO: 17, SEQ ID NO: 18) driver mutations selected from the group consisting of G12D (SEQ ID NO: 22) and G12V (SEQ ID NO: 24) as set out in SEQ ID NO: 18;

RKO (ATCC, CRL-2577) modified to reduce expression of CD276, reduce secretion of TGFβ1, and express GM-CSF (SEQ ID NO: 7, SEQ ID NO: 8), membrane bound CD40L (SEQ ID NO: 2, SEQ ID NO: 3), IL-12 (SEQ ID NO: 9, SEQ ID NO: 10), and peptides comprising one or more TP53 (SEQ ID NO: 35, SEQ ID NO: 36) driver mutations selected from the group consisting R175H, G245S, and R248W, express a peptide containing the KRAS (SEQ ID NO: 33, SEQ ID NO: 34) driver mutation G12C, express peptides comprising one or more PIK3CA (SEQ ID NO: 37, SEQ ID NO: 38) driver mutations selected from the group consisting of R88Q, M10431, and H1047Y, express peptides comprising one or more FBXW7 (SEQ ID NO: 39, SEQ ID NO: 40) driver mutations selected from the group consisting of S582L and R465H, express a peptide containing CTNNB1 (SEQ ID NO: 45, SEQ ID NO: 46) driver mutation S45F, and express a peptide containing ERBB3 (SEQ ID NO: 47, SEQ ID NO: 48) driver mutation V104M as set out in SEQ ID NO: 52;

DMS 53 (ATCC, CRL-2062) modified to reduce expression of CD276, reduce secretion of TGFβ1 and TGFβ2, and to express GM-CSF (SEQ ID NO: 7, SEQ ID NO: 8), membrane bound CD40L (SEQ ID NO: 2, SEQ ID NO: 3) and IL-12 (SEQ ID NO: 9, SEQ ID NO: 10).

TABLE 2-23
Colorectal cancer vaccine cell line nomenclature and genetic modifications

| | | CD276 | TGFβ1 | TGFβ2 | | | | Tumor-Associated | |
|---|---|---|---|---|---|---|---|---|---|
| Cocktail | Cell Line | KO | KD | KD | GM-CSF | mCD40L | IL-12 | Antigens (TAAs) | Driver Mutations |
| A | HCT-15 | SEQ ID | SEQ ID | — | SEQ ID | SEQ ID | SEQ ID | — | — |
| | | NO: 25 | NO: 26 | | NO: 8 | NO: 3 | NO: 10 | | |
| A | HuTu80 | SEQ ID | SEQ ID | SEQ ID | SEQ ID | SEQ ID | SEQ ID | modPSMA | TP53, PIK3CA, |
| | | NO: 25 | NO: 26 | NO: 27 | NO: 8 | NO: 3 | NO: 10 | (SEQ ID NO: 20) | FBXW7, SMAD4, |
| | | | | | | | | | GNAS, ATM |
| | | | | | | | | | (SEQ ID NO: 54) |
| A | LS411N | SEQ ID | SEQ ID | — | SEQ ID | SEQ ID | SEQ ID | — | — |
| | | NO: 25 | NO: 26 | | NO: 8 | NO: 3 | NO: 10 | | |
| B | HCT-116 | SEQ ID | SEQ ID | — | SEQ ID | SEQ ID | SEQ ID | modTBXT | KRAS |
| | | NO: 25 | NO: 26 | | NO: 8 | NO: 3 | NO: 10 | modWT1 | (SEQ ID NO: 22 and |
| | | | | | | | | (SEQ ID NO: 18) | (SEQ ID NO: 24; |
| | | | | | | | | | SEQ ID NO: 18) |
| B | RKO | SEQ ID | SEQ ID | — | SEQ ID | SEQ ID | SEQ ID | — | TP53, KRAS, |
| | | NO: 25 | NO: 26 | | NO: 8 | NO: 3 | NO: 10 | | PIK3CA, FBXW7 |
| | | | | | | | | | CTNNB1, ERBB3 |
| | | | | | | | | | (SEQ ID NO: 52) |
| B | DMS 53* | SEQ ID | SEQ ID | SEQ ID | SEQ ID | SEQ ID | SEQ ID | — | — |
| | | NO: 25 | NO: 26 | NO: 27 | NO: 8 | NO: 3 | NO: 10 | | |

—, not completed /not required.
*Cell line identified as CSC-like.
mCD40L, membrane bound CD40L.

1. A composition comprising a therapeutically effective amount of at least 1 modified colorectal cancer cell line, wherein the cell line or a combination of the cell lines comprises cells that express at least 5 tumor associated antigens (TAAs) associated with colorectal cancer, and wherein said composition is capable of eliciting an immune response specific to the at least 5 TAAs, and wherein the cell line or combination of the cell lines have been modified to express at least 1 peptide comprising at least 1 oncogene driver mutation.

2.-5. (canceled)

6. The composition of claim 1, wherein the cell line or a combination of the cell lines are modified to (i) express or increase expression of at least 1 immunostimulatory factor, and (ii) inhibit or decrease expression of at least 1 immunosuppressive factor.

7. The composition of claim 1, wherein the cell line or a combination of the cell lines are modified to express or increase expression of at least 1 TAA that is either not expressed or minimally expressed by one or all of the cell lines.

8. The composition of claim 1, wherein the composition is capable of stimulating an immune response in a subject receiving the composition.

9. The composition of claim 7, wherein the cell line or a combination of the cell lines are modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, (ii) express or increase expression of 1, 2, 3, 4, 5, 6, 7, 8, 9 or 10 immunostimulatory factors, (iii) inhibit or decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines, and wherein at least one of the cell lines is a cancer stem cell line.

10. (canceled)

11. The composition of claim 1, wherein the colorectal cancer cell line or cell lines are selected from the group consisting of LS123, HCT15, SW1463, RKO, HUTU80, HCT116, LOVO, T84, LS411N, SW48, C2BBel, Caco-2, SNU-1033, COLO 201, GP2d, CL-14, SW403, SW1116, SW837, SK-CO-1, CL-34, NCI-H508, CCK-81, SNU-C2A, GP2d, HT-55, MDST8, RCM-1, CL-40, COLO 678, and LS180.

12. The composition of claim 11, wherein the cell lines are selected from the group consisting of HCT15, RKO, HUTU80, HCT116, and LS411N.

13. The composition of claim 1, wherein the oncogene driver mutation is in one or more oncogenes selected from the group consisting of APC, TP53, KRAS, PIK3CA, FAT4, LRP1B, FBXW7, BRAF, SMAD4, PCLO, KMT2C, KMT2D, ATM, RNF213, ZFHX3, AMER1, TRRAP, ARID1A, FAT1, EP400, SOX9, RNF43, MKI67, RELN, PTPRS, PDE4DIP, CHD4, PTPRT, ANKRD11, ROBO1, MTOR, CREBBP, LRRK2, TCF7L2, KMT2B, PRKDC, UBR5, ACVR2A, ERBB4, PREX2, CARD11, NOTCH1, PTEN, NCOR2, GRIN2A, KMT2A, ATRX, CACNAlD, ALK, MYH9, NOTCH3, POLE, BCORL1, SPEN, BCL9L, BRCA2, CUX1, ARIDIB, CTNNB1, MYH11, SMARCA4, NF1, PIK3CG, PLCG2, AXIN2, MGA, SLX4, FLT4, ERBB3, POLQ, ASXL1, CAD, PTPRK, ARID2, CIC, EP300, EPHA5, NUMA1, CAMTA1, GNAS, LRP5, BCL9, PTPRD, RANBP2, IRS1, MYO5A, ROS1, IRS4, SETD1A, PIK3R1, PTPRC, COL1A1, TP53BP1, DICERI, SETBP1, ZBTB20, KDM2B, B2M, AFDN, ZNF521, and LARP4B.

14.-15. (canceled)

16. The composition of claim 1, wherein (a) the at least one immunostimulatory factor is selected from the group consisting of GM-CSF, membrane-bound CD40L, GITR, IL-15, IL-23, and IL-12, and (b) wherein the at least one immunosuppressive factor is selected from the group consisting of CD276, CD47, CTLA4, HLA-E, HLA-G, IDO1, IL-10, TGFβ1, TGFβ2, and TGFβ3.

17.-26. (canceled)

27. A composition comprising 3 colorectal cancer cell lines, wherein 1, 2 or all 3 of the cell lines is modified in vitro to (i) express at least one immunostimulatory factor; and (ii) decrease expression of at least one immunosuppressive factor; wherein at least 1 of the cell lines is modified to express at least one TAA that is either not expressed or minimally expressed by the cell line; and wherein at least 1 of the cell lines modified in vitro to express at least 1 peptide comprising at least 1 oncogene driver mutation.

28. (canceled)

29. A composition comprising 2 colorectal cancer cell lines and one cancer stem cell line, wherein 1, 2 or all 3 of the cell lines is modified in vitro to (i) express at least one immunostimulatory factor; and (ii) decrease expression of at least one immunosuppressive factor; wherein at least 1 of the colorectal cancer cell lines is modified to express at least one TAA that is either not expressed or minimally expressed by the colorectal cancer cell line; and wherein at least 1 of the colorectal cell lines modified in vitro to express at least 1 peptide comprising at least 1 oncogene driver mutation.

30.-38. (canceled)

39. A unit dose of a medicament for treating colorectal cancer comprising at least 5 compositions of different cancer cell lines, wherein at least 2 compositions comprise a cell line that is modified to (i) express or increase expression of at least 2 immunostimulatory factors, (ii) inhibit or decrease expression of at least 2 immunosuppressive factors, and (iii) express at least 1 peptide comprising at least 1 oncogene driver mutation.

40.-44. (canceled)

45. A method of preparing a composition comprising a modified colorectal cancer cell line, said method comprising the steps of:
(a) identifying one or more mutated oncogenes with >5% mutation frequency in colorectal cancer;
(b) identifying one or more driver mutations occurring in ≥0.5% of profiled colorectal patient samples in the mutated oncogenes identified in (a);
(c) determining whether a peptide sequence comprising non-mutated oncogene amino acids and the driver mutation identified in (b) comprises a CD4 epitope, a CD8 epitope, or both CD4 and CD8 epitopes;
(d) inserting a nucleic acid sequence encoding the peptide sequence comprising the driver mutation of (c) into a lentiviral vector; and
(e) introducing the lentiviral vector into a cancer cell line, thereby producing a composition comprising a modified cancer cell line.

46.-77. (canceled)

78. A method of stimulating an immune response in a patient comprising administering to said patient a therapeutically effective amount of a unit dose of a colorectal cancer vaccine, wherein said unit dose comprises a composition comprising a cancer stem cell line and at least 3 compositions each comprising a different colorectal cancer cell line; wherein the cell lines are optionally modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, and/or (ii) express or increase expression of 1, 2, 3, 4, 5, 6, 7, 8, 9 or 10 immunostimulatory factors, and/or (iii) inhibit or decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines.

79. A method of treating colorectal cancer in a patient comprising administering to said patient a therapeutically effective amount of a unit dose of a colorectal cancer vaccine, wherein said unit dose comprises a composition comprising a cancer stem cell line and at least 3 compositions each comprising a different colorectal cancer cell line; wherein the cell lines are optionally modified to (i) express at least 1, 2, 3, 4, 5, 6, 7, 8, 9, 10, 11, 12, 13, 14, 15, 16, 17, 18, 19 or 20 or more peptides, wherein each peptide comprises at least 1 oncogene driver mutation, and/or (ii) express or increase expression of 1, 2, 3, 4, 5, 6, 7, 8, 9 or 10 immunostimulatory factors, and/or (iii) inhibit or decrease expression of 1, 2, 3, 4, 5, 6, 7, 8, 9 or 10 immunosuppressive factors, and/or (iv) express or increase expression of 1, 2, 3, 4, 5, 6, 7, 8, 9 or 10 TAAs that are either not expressed or minimally expressed by one or all of the cell lines.

80.-85. (canceled)
